(19) United States
(12) Patent Application Publication
(10) Pub. No.: US 2025/0019713 A1
(43) Pub. Date: Jan. 16, 2025

(54) SHORT STATURE CORN PLANTS WITH IMPROVED SILAGE TRAITS

(71) Applicant: MONSANTO TECHNOLOGY LLC, Saint Louis, MO (US)

(72) Inventors: Ty BARTEN, Ankeny, IA (US); William BOURDONCLE, Dulsburg (DE); Edward CARGILL, Chesterfield, MO (US); Bryce LEMKE, Nevada, IA (US); Joseph F. MALONEY, Galena, MD (US); Peter MALONEY, Easton, MD (US); Jean-Luc PELLET, Cremona (IT)

(21) Appl. No.: 18/713,008

(22) PCT Filed: Nov. 22, 2022
(86) PCT No.: PCT/US2022/080369

§ 371 Date: May 23, 2024

Related U.S. Application Data
(60) Provisional application No. 63/323,476, filed on Mar. 24, 2022.
(60) Provisional application No. 63/283,080, filed on Nov. 24, 2021.

(51) Int. Cl. C12N 15/82; C07K 14/415; C12N 9/02

(52) U.S. Cl. CPC C12N 15/8218; C07K 14/415; C12N 9/0071; C12N 15/8242; C12Y 114/11015

(57) ABSTRACT

A maize plant or maize plant part, and methods of making and using the same, are provided herein, wherein the maize plant or maize plant part comprises a mutant allele of an endogenous brachytic 2 (Z?r2) gene, a mutant allele of an endogenous GA20 oxidase or GA3 oxidase gene, or a transgene encoding a non-coding RNA molecule that targets an endogenous GA oxidase gene for suppression, and/or a mutant allele of an endogenous brown midrib 3 (bin3) gene. The maize plants or maize plant parts may be homozygous or heterozygous for the transgene or the mutant alleles. Additionally, the maize plant or maize plant part may be part of a silage product, where the harvest product is formed by harvesting an above-ground biomass of a population of the maize plants or a population of intercropped silage plants including such maize plants and making a silage product from the above-ground biomass.

### CROSS-REFERENCE TO RELATED APPLICATIONS

This application claims the benefit of, and priority to, U.S. Provisional Application No. 63/283,080 filed on Nov. 24, 2021, and U.S. Provisional Application No. 63/323,476 filed on Mar. 24, 2022. The entire disclosure of each of the above-referenced applications is incorporated herein by reference.

### FIELD

The present disclosure generally relates to plants (and/or plant parts) (e.g., corn or maize plants and/or parts thereof, etc.) having brown mid-rib and/or short stature corn/maize traits, for example, a mutant allele(s) of the endogenous brachytic 2 (br2) gene, a mutant allele(s) of endogenous GA oxidase gene(s), or a suppression construct for endogenous GA oxidase gene(s), and/or a mutant allele(s) of the endogenous brown midrib 3 (bm3) gene, and to uses (e.g., as part of silage products, etc.) and methods related thereto.

### SEQUENCE LISTING

This application contains references to amino acid sequences and/or nucleic acid sequences which have been submitted concurrently herewith as the sequence listing text file entitled “125-WO-POA_SequenceListing.XML”, file size 214 KiloBytes (KB), created on Nov. 7, 2022. The aforementioned sequence listing is hereby incorporated by reference in its entirety pursuant to 37 C.F.R. § 1.52 (e) (5).

### BACKGROUND

Above-ground biomass from corn plants may be used to produce silage, which may then be used to feed livestock or other animals. Additionally, biomass from corn plants may be used to harvest seed and/or produce ethanol through fermentation or biogas for electricity, etc., which may be additional benefits of silage or dual-purpose (DP) corn or maize plants. What is needed in the art are corn or maize plants having improved harvest and silage qualities, such as improved standability and nutritional characteristics.

### SUMMARY

This section provides a general summary of the disclosure, and is not a comprehensive disclosure of its full scope or all of its features.

New and useful plants and/or plant parts, including germplasms thereof, are provided herein where the plants and/or plant parts comprise a mutant allele of the endogenous brachytic 2 (br2) gene, a mutant allele of an endogenous GA oxidase gene, or a suppression construct for an endogenous GA oxidase gene, and/or a mutant allele of the endogenous brown midrib 3 (bm3) gene. In some example embodiments, the plants and/or plant parts comprise a mutant allele of the endogenous br2 gene. In some example embodiments, the plants and/or plant parts comprise a mutant allele of an endogenous GA oxidase gene. In some example embodiments, the plants and/or plant parts comprise a suppression construct targeting an endogenous GA oxidase gene. In some example embodiments, the plants and/or plant parts comprise a mutant allele of the endogenous bm3 gene. In some example embodiments, the plants and/or plant parts comprise both a mutant allele of the endogenous br2 gene and a mutant allele of the endogenous bm3 gene. In some example embodiments, the plants and/or plant parts comprise both a mutant allele of the endogenous GA oxidase gene and a mutant allele of the endogenous bm3 gene. And, in some example embodiments, the plants and/or plant parts comprise a mutant allele of the endogenous br2 gene, a mutant allele of the endogenous GA oxidase gene, and a mutant allele of the endogenous bm3 gene. In addition, in some example embodiments, the plants or plant parts may include maize plants, or parts thereof, including, for example, maize hybrid plants or parts thereof, etc. In some example embodiments, the plant parts may include plant seeds (e.g., maize plant seeds, seeds of other plant types, etc.).

In one aspect of the present disclosure, a plant or plant part herein comprises a mutant allele of the endogenous br2 gene. A mutant allele of the endogenous br2 gene may comprise an insertion, deletion or substitution of one or more nucleotides, or any combination thereof, in the endogenous br2 gene. In addition, or alternatively, a mutant allele of the endogenous br2 gene may be the br2-23 allele, br2-708/allele, br2-7861 allele, br2-qph1 allele, br2-qpa1 allele, or br2-NC238 allele, or a mutant allele of the endogenous br2 gene may be an edited allele of the endogenous br2 gene, such as the br2-1005 allele. The mutant allele of the endogenous br2 gene may comprise one mor more mutations relative to SEQ ID NO: 90, SEQ ID NO: 91, and/or SEQ ID NO: 92. The expression level and/or activity of the mRNA and/or proteins encoded by the mutant allele of the endogenous br2 gene may altered, such as reduced, in the modified maize plant relative to the expression level and/or activity of the mRNA and/or protein encoded by a wild-type br2 gene allele. Moreover, the plant or plant part may be homozygous for a mutant allele of the endogenous br2 gene, or the plant or plant part may be heterozygous for a mutant allele of the endogenous br2 gene. Alternatively, the plant or plant part may be heteroallelic for the endogenous br2 gene and may comprise a first mutant allele of the endogenous br2 gene on one chromosome and a second mutant allele at the same locus on a second homologous chromosome of the endogenous br2 gene (e.g., wherein the first mutant br2 allele and second mutant br2 allele are different, etc.). Additionally, or alternatively, the plant or plant part may comprise a mutant allele of the endogenous br2 gene with multiple mutations in the endogenous br2 gene, and the plant or plant part may be homozygous, heterozygous, and/or heteroallelic for such mutant allele. As used herein, maize plants comprising a mutant allele of the br2 gene may also be referred to as short stature maize, semi dwarf maize, brachytic maize, short stature corn, semi dwarf corn, or brachytic corn.

In another aspect of the present disclosure, a plant or plant part herein comprises a mutant allele of the endogenous br2 gene and/or a mutant allele of the endogenous bm3 gene. In connection therewith, a mutant allele of the endogenous br2 gene may be as described above. A mutant allele of the endogenous bm3 gene may comprise an insertion, deletion or substitution of one or more nucleotides, or any combination thereof, in the endogenous bm3 gene. In addition, or alternatively, a mutant allele of the endogenous bm3 gene may be the bm3-1 allele, bm3-2 allele, or bm3-3 allele, or a mutant allele of the endogenous bm3 gene may be an edited allele of the endogenous bm3 gene. The mutant allele of the endogenous bm3 gene may comprise one more more mutations relative to SEQ ID NO: 94, SEQ ID NO: 95, and/or SEQ ID NO: 96. The expression level and/or activity of the mRNA and/or proteins encoded by the mutant allele of the endogenous bm3 gene may altered, such as reduced, in the modified maize plant relative to the expression level and/or activity of the mRNA and/or protein encoded by a wild-type bm3 gene allele. Additionally, the plant or plant part may be homozygous for a mutant allele of the endogenous bm3 gene, or the plant or plant part may be heterozygous for a mutant allele of the endogenous bm3 gene. Alternatively, the plant or plant part may be heteroallelic for the endogenous bm3 gene and may comprise a first mutant allele of the endogenous bm3 gene on one chromosome and a second mutant allele at the same locus on a second homologous chromosome of the endogenous bm3 gene (e.g., wherein a first bm3 mutant allele and a second bm3 mutant allele different, etc.). Additionally, or alternatively, the plant or plant part may comprise a mutant allele of the endogenous bm3 gene with multiple mutations in the endogenous bm3 gene, and the plant or plant part may be homozygous, heterozygous, and/or heteroallelic for such mutant allele.

In another aspect of the present disclosure, a plant or plant part may comprise a non-coding RNA molecule encodes an endogenous GA oxidase protein and/or an endogenous GA20 oxidase protein. The RNA molecule may be a molecule that may be at least 80% complementary to at least 15 nucleotides of an mRNA molecule encoding an endogenous GA oxidase protein and/or an endogenous GA20 oxidase protein. In maize plants or maize plant parts comprising an endogenous br2 gene and a mutant allele of an endogenous GA20 oxidase gene or a mutant allele of an endogenous GA3 oxidase gene, the endogenous br2, GA20 oxidase, and GA3 oxidase genes may comprise an insertion, deletion, and/or substation of one or more nucleotides, or any combination thereof in each of the br2, GA20 oxidase, and GA3 oxidase genes.

In still another aspect of the present disclosure, a plant seed (e.g., a maize plant seed, a maize hybrid plant seed, etc.) comprises a mutant allele of the endogenous br2 gene. In addition, in some embodiments, the plant seed may further comprise a mutant allele of the endogenous bm3 gene. In addition, the plant seed may be included in (or may be part of) a plurality of maize plant seeds where a majority of, or all of, the maize plant seeds in the plurality of the maize plant seeds comprises a mutant allele of the endogenous br2 gene and/or a mutant allele of the endogenous bm3 gene. Each of the maize plant seeds in the plurality of maize plant seeds may comprise a mutant allele of the endogenous br2 gene and/or a mutant allele of the endogenous bm3 gene. Additionally or alternatively, the plant seed may further comprise a mutant allele of an endogenous GA20 oxidase gene, and/or a mutant allele of an endogenous GA3 oxidase gene, and/or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter. One or more plants, then, may be grown from seeds in the population.

The mutant alleles of the br2, GA20 oxidase, GA3 oxidase, and/or bm3 genes may be present in a majority of a plurality of maize plant seeds. The plurality of maize plant seeds may comprise a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or a mutant allele of an endogenous brown midrib 3 (bm3) gene.

In another aspect of the present disclosure, a population of the plants herein (e.g., a population of maize plants herein, a population of maize hybrid plants herein, etc.) is provided, where a majority of, or each or all of, the plants in the population comprises a mutant allele of the endogenous br2 gene. The population of maize plants comprising a majority of maize plants comprising a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or optionally a mutant allele of an endogenous brown midrib 3 (bm3) gene may have one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In addition, in some embodiments, a majority of, or each or all of, the plants in the population may further comprise a mutant allele of the endogenous bm3 gene. In connection therewith, the population of plants may be grown in a greenhouse (or multiple greenhouses) or other controlled environment(s), or in another growing space (e.g., a field or multiple fields, a plot or multiple plots, a test plot or multiple test plots, etc.) (all, broadly, a growing space).

The plant(s) and/or population of plants provided herein, which may comprise a mutant allele of the endogenous br2 gene, or a mutant allele of the endogenous br2 gene and a mutant allele of the endogenous bm3 gene, and which may be grown from one or more seed(s) as provided herein, may have a relatively shorter plant height (e.g., a shorter average plant height, etc.) as compared to a wild-type or control plant and/or population of wild-type or control plants (e.g., about 10% or more shorter, about 15% or more shorter, about 20% or more shorter, about 30% or more shorter, about 40% or more shorter, etc.). The plant(s) and/or population of plants may also have increased resistance to lodging (e.g., root and/or stalk lodging) and/or green snap, as compared to the wild-type or control plant that does not have a mutant allele of the endogenous br2 gene and/or a mutant allele of the endogenous bm3 gene.

The population of plants and/or population of intercropped plants herein may be planted (e.g., in a field or growing space) at a planting density of about 13,000 seeds/Ha or more, about 27,000 seeds/Ha or more, about 40,000 seeds/Ha or more, about 53,000 plants/Ha or more, about 67,000 seeds/Ha or more, about 100,000 seeds/Ha or more, about 133,000 seeds/Ha or more, about 167,000 seeds/Ha or more, about 200,000 seeds/Ha or more, about 233,000 seeds/Ha or more, about 267,000 seeds/Ha or more, about 333,000 seeds/Ha or more, about 400,000 seeds/Ha or more, about 466,000 seeds/Ha or more, about 533,000 seeds/Ha or more, about 600,000 seeds/Ha or more, about 667,000 seeds/Ha or more, etc. In addition, or alternatively, the population of plants herein may be grown (e.g., in a field or growing space) at a growing density of about 10,000 plants/Ha or more, about 20,000 plants/Ha or more, about 30,000 plants/Ha or more, about 40,000 plants/Ha or more, about 50,000 plants/Ha or more, about 75,000 plants/Ha or more, about 100,000 plants/Ha or more, about 125,000 plants/Ha or more, about 150,000 plants/Ha or more, about 175,000 plants/Ha or more, about 200,000 plants/Ha or more, about 250,000 plants/Ha or more, about 300,000 plants/Ha or more, about 350,000 plants/Ha or more, about 400,000 plants/Ha or more, about 450,000 plants/Ha or more, about 500,000 plants/Ha or more, etc. Further, the population of plants may be located (or spaced) and/or may be planted and grown in a plurality of parallel rows (e.g., in a field or growing space), where an average spacing between adjacent rows is about 80 cm or less, about 70 cm or less, about 60 cm or less, about 50 cm or less, about 40 cm or less, about 35 cm or less, or about 30 cm or less, or about 76 cm, about 51 cm, about 38 cm, or about 30 cm. Still further, in some embodiments, the population of plants may be located in arrangements other than rows.

The plant(s) or plant part(s) and/or population of plants and/or population of intercropped plants herein may also be used as part of a silage product (e.g., included in a silage product, be used to produce or make a silage product, etc.), where the plant(s) or plant part(s) and/or population of plants and/or population of intercropped plants and/or the silage product may have one or more of the following traits: improved (e.g., increased, etc.) protein content (and/or N-stover) (e.g., about 2% or more, about 4% or more, about 5% or more, about 6% or more, about 8% or more, about 10% or more, about 12% or more, about 15% or more, about 18% or more, about 20% or more, between about 6% and about 20%, between about 10% and about 20%, etc.), reduced lignin content (e.g., about 6% or less, about 5.5% or less, about 5% or less, about 4.5% or less, about 4% or less, about 3.5% or less, or about 3% or less, or between about 2% and about 6%, between about 2% and about 5%, between about 2% and about 4%, or between about 2% and about 3%, etc.), improved fiber digestibility, reduced acid detergent fiber (ADF) (e.g., about 50% or less, about 45% or less, about 40% or less, about 35% or less, about 30% or less, about 25% or less, between about 25% and about 50%, between about 25% and about 45%, or between about 25% and about 40%, etc.), increased starch content (e.g., about 20% or more, about 30% or more, about 40% or more, about 50% or more, between about 25% and about 50%, between about 30% and about 50%, between about 30% and about 40%, etc.), increased neutral detergent fiber digestibility (NDFD) (also termed, cell wall digestibility (DCW)) (e.g., about 50% or more, about 55% or more, about 60% or more, about 65% or more, about 70% or more, or between about 50% and about 70%, between about 55% and about 65%, between about 55% and about 60%, etc.), reduced root lodging, increased biomass (e.g., a dry matter biomass (DMB) of about 0.5 kg/m² or more, of about 2 kg/m² or less, or of between about 0.5 kg/m² and about 2 kg/m², etc.), increased milk output (e.g., a milk per acre output of about 10,000 lbs/acre or more, about 15,000 lbs/acre or more, about 20,000 lbs/acre or more, about 25,000 lbs/acre or more, about 30,000 lbs/acre or more, about 35,000 lbs/acre or more, about 40,000 lbs/acre or more, or about 45,000 lbs/acre or more (and/or a milk per ton output of about 2,000 lbs/ton or more, about 2,500 lbs/ton or more, about 3,000 lbs/ton or more, about 3,200 lbs/ton or more, about 3,400 lbs/ton or more, about 3,600 lbs/ton or more, about 3,800 lbs/ton or more, or about 4,000 lbs/ton or more, etc.), etc.), increased silage yield (e.g., about 5 tons/acre or more, about 7 tons/acre or more, about 10 tons/acre or more, about 12 tons/acre or more, or about 15 tons/acre or more, or between about 7 tons/acre and about 12 tons/acre, etc.), increased grain yield, increased stem cross-section area, and improved standability, as compared, for example, to a wild-type or control maize plant or plant part (e.g., a control plant or plant part not having a mutant allele of the endogenous br2 gene or a mutant allele of the endogenous bm3 gene) and/or a silage product of a wild-type or control maize plant or plant part.

In another aspect of the present disclosure, a population of intercropped plants is provided, for example, in connection with using the plants herein as part of a silage product, for energy production, etc. The population of intercropped plants may include the population of plants herein (e.g., plants comprising a mutant allele of the endogenous br2 gene and/or a mutant allele of the endogenous bm3 gene, etc.) in combination with a population of one or more companion crop plants (e.g., wheat, barley, oat, alfalfa, rye, clover, grass, triticale, cercal, legume, bean, pea, and soybean, etc.), for example, grown together in a growing space (e.g., a field, etc.).

The population of intercropped plants herein may include a DMB of about 0.5 kg/m² or more, of about 2 kg/m² or less, or of between about 0.5 kg/m² and about 2 kg/m². In addition, the population of intercropped plants may have a yield of about 5 tons/acre or more, about 7 tons/acre or more, about 10 tons/acre or more, about 12 tons/acre or more, or about 15 tons/acre or more, or between about 7 tons/acre and about 12 tons/acre. In addition (or alternatively), the population of plants herein may include a milk per acre output of about 20,000 lbs/acre or more, about 25,000 lbs/acre or more, about 30,000 lbs/acre or more, about 35,000 lbs/acre or more, about 40,000 lbs/acre or more, or about 45,000 lbs/acre or more (and/or a milk per ton output of about 3,000 lbs/ton or more, about 3,200 lbs/ton or more, about 3,400 lbs/ton or more, about 3,600 lbs/ton or more, about 3,800 lbs/ton or more, or about 4,000 lbs/ton or more, etc.).

In addition, in the population of intercropped plants herein, the population of plants (e.g., the plants comprising a mutant allele of the endogenous br2 gene and/or a mutant allele of the endogenous bm3 gene, etc.) may be planted and grown in a plurality of rows (e.g., parallel rows, etc.), and the population of companion crop plants may be planted and grown between adjacent rows of the population of plants (e.g., in a plurality of rows (e.g., parallel rows, etc.), etc.). Further, an average spacing between adjacent rows of the intercropped plants (e.g., the plants comprising a mutant allele of the endogenous br2 gene and/or a mutant allele of the endogenous bm3 gene as well as the companion crop plants, etc.) may be about 80 cm or less, about 70 cm or less, about 60 cm or less, about 50 cm or less, about 40 cm or less, about 35 cm or less, or about 30 cm or less, or about 76 cm, about 51 cm, about 38 cm, or about 30 cm.

In connection therewith, the population of companion crop plants may be planted in the rows at a planting density of about 13,000 seeds/Ha or more, about 27,000 seeds/Ha or more, about 40,000 seeds/Ha or more, about 53,000 plants/Ha or more, about 67,000 seeds/Ha or more, about 100,000 seeds/Ha or more, about 133,000 seeds/Ha or more, about 167,000 seeds/Ha or more, about 200,000 seeds/Ha or more, about 233,000 seeds/Ha or more, about 267,000 seeds/Ha or more, etc. In addition, or alternatively, the population of companion crop plants may be grown at a growing density of about 10,000 plants/Ha or more, about 20,000 plants/Ha or more, about 30,000 plants/Ha or more, about 40,000 plants/Ha or more, about 50,000 plants/Ha or more, about 75,000 plants/Ha or more, about 100,000 plants/Ha or more, about 125,000 plants/Ha or more, about 150,000 plants/Ha or more, about 175,000 plants/Ha or more, or about 200,000 plants/Ha or more. Further, the population of intercropped plants may be planted at a planting density of about 67,000 seeds/Ha or more, about 100,000 seeds/Ha or more, about 133,000 seeds/Ha or more, about 167,000 seeds/Ha or more, about 200,000 seeds/Ha or more, about 233,000 seeds/Ha or more, about 267,000 seeds/Ha or more, about 333,000 seeds/Ha or more, about 400,000 seeds/Ha or more, about 466,000 seeds/Ha or more, about 533,000 seeds/Ha or more, about 600,000 seeds/Ha or more, about 667,000 seeds/Ha or more, etc. In addition, or alternatively, the population of intercropped plants may be grown at a growing density of about 50,000 plants/Ha or more, about 75,000 plants/Ha or more, about 100,000 plants/Ha or more, about 125,000 plants/Ha or more, about 150,000 plants/Ha or more, about 175,000 plants/Ha or more, about 200,000 plants/Ha or more, about 250,000 plants/Ha or more, about 300,000 plants/Ha or more, about 350,000 plants/Ha or more, about 400,000 plants/Ha or more, about 450,000 plants/Ha or more, or about 500,000 plants/Ha or more.

Another aspect of the present disclosure is directed to a silage product that includes, or is made from, a population of intercropped plants herein, for example, where the population of intercropped silage plants are harvested and/or processed to form the silage product, etc. In connection therewith, the silage product may include one or more of the traits, characteristics, etc., described herein, for example: improved (e.g., increased, etc.) protein content (and/or N-stover), reduced lignin content, improved fiber digestibility, reduced ADF, increased starch content, increased NDFD or DCW, increased biomass, increased milk output, increased silage yield, increased grain yield, or increased stem cross-section area, as compared, for example, to a silage product produced from a wild-type control maize plant or maize plant part or population of wild-type or control plants (e.g., control plants not having a mutant allele of the endogenous br2 gene or a mutant allele of the endogenous bm3 gene, etc.) or plant parts therefrom.

In still another aspect of the present disclosure, a method is provided for producing silage (e.g., from the plants or plant parts herein comprising a mutant allele of the endogenous br2 gene, or comprising both a mutant allele of the endogenous br2 gene and a mutant allele of the endogenous bm3 gene; from the intercropped plants or plant parts herein; etc.). In one example embodiment, the method generally includes: (i) harvesting an above-ground biomass of the population of plants herein comprising a mutant allele of the endogenous br2 gene and/or a mutant allele of the endogenous bm3 gene, or of the population of intercropped silage plants herein. This may be done, for example, when a moisture content of the population of plants or the population of intercropped silage plants is between about 50% and about 80%, between about 55% and about 75%, between about 60% and about 70%, or between about 65% and about 70%, etc. The method then includes (ii) forming (e.g., chopping, grinding, otherwise processing, etc.) the above-ground biomass into pieces to form a silage product.

The method of producing the silage may also include (iii) planting, in a field, a plurality of plant seeds (e.g., plant seeds comprising a mutant allele of the endogenous br2 gene and/or a mutant allele of the endogenous bm3 gene, etc.) or a plurality of intercropped silage plant seeds (e.g., plant seeds comprising a mutant allele of the endogenous br2 gene and/or a mutant allele of the endogenous bm3 gene in combination with companion crop plant seeds, etc.), and (iv) growing the population of plants or the population of intercropped silage plants from the plant seeds or the intercropped silage plant seeds. The population of plants or the population of intercropped silage plants herein, as grown in accordance with the method of this embodiment (e.g., comprising a mutant allele of the endogenous br2 gene and/or a mutant allele of the endogenous bm3 gene, etc.), may have reduced lodging and/or green snap in comparison to a population of wild-type or control plants (as well as one or more of the other traits described herein).

In another example embodiment, the method for producing silage generally includes (i) planting, in a field, a plurality of maize plant seeds (e.g., maize plant seeds comprising a mutant allele of the endogenous br2 gene and/or a mutant allele of the endogenous bm3 gene, etc.); (ii) growing a plurality of maize plants from the plurality of maize plant seeds; (iii) harvesting an above-ground biomass of the population of maize plants (e.g., when a moisture content of the population of maize plants is between about 50% and about 80%, between about 55% and about 75%, between about 60% and about 70%, or between about 65% and about 70%, etc.); and (iv) forming (e.g., chopping, grinding, otherwise processing, etc.) the above-ground biomass into pieces to form a silage product. The maize plants in the silage product may comprise a mutant allele of an endogenous br2 gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or a mutant allele of an endogenous bm3 gene. The method of this embodiment may also include planting a plurality of companion crop plant seeds with the plurality of maize plant seeds and then growing companion crop plants together with the maize plants. The population of maize plants, as grown in accordance with the method of this embodiment, may have reduced lodging and/or green snap in comparison to a population of wild-type or control maize plants (as well as one or more of the other traits described herein).

In the above embodiments, the method for producing silage herein may further include one or more of: storing the silage product for a time period (e.g., from about 1 day to about 2 years, or from about 1 week to about 2 years, or from about 1 week to about 1 year, or from about 2 weeks to about 6 months, or from about 2 weeks to about 3 months, etc.); fermenting the silage product for a time period (e.g., from about 1 day to about 2 years, from about 1 week to about 2 years, or from about 1 week to about 1 year, or from about 1 week to about 6 months, or from about 2 weeks to about 6 months, or from about 2 weeks to about 3 months, etc.); and/or feeding the silage product to one or more livestock animals.

In another aspect of the present disclosure, a method is provided for producing maize plant seeds. In one example embodiment, the method generally includes: (i) crossing a first maize plant to a second maize plant, wherein either: (a) the first maize plant comprises a mutant allele of the endogenous br2 gene and a mutant allele of the endogenous bm3 gene; (b) the first maize plant comprises a mutant allele of the endogenous br2 gene, and the second maize plant comprises a mutant allele of the endogenous bm3 gene; (c) the first maize plant comprises a mutant allele of the endogenous bm3 gene, and the second maize plant comprises a mutant allele of the endogenous br2 gene; or (d) the second maize plant comprises a mutant allele of the endogenous br2 gene and a mutant allele of the endogenous bm3 gene; and wherein either: (a) the first plant is the female plant, and second plant is the male plant; or (b) the second plant is the female plant, and first plant is the male plant; and (ii) harvesting one or more maize plant progeny seeds (e.g., one or more maize hybrid plant seeds, etc.) from the female maize plant, wherein the maize plant progeny seeds comprise a mutant allele of the endogenous br2 gene and a mutant allele of the endogenous bm3 gene. The method may further include (iii) planting the one or more maize plant progeny seeds in a field, greenhouse, or controlled environment (broadly, in a growing space); and (iv) growing one or more maize progeny plants (e.g., maize hybrid plants, etc.) from the maize plant progeny seeds.

In the method for producing the maize plant seeds, the maize plant parent and/or progeny seeds may be homozygous for a mutant allele of the endogenous bm3 gene, or the maize plant parent and/or progeny seeds may be heterozygous for a mutant allele of the endogenous bm3 gene. Alternatively, the maize plant parent and/or progeny seeds may be heteroallelic for the endogenous bm3 gene and may comprise a first mutant allele (e.g., on one chromosome, etc.) of the endogenous bm3 gene and a second mutant allele (e.g., at the same locus on a second homologous chromosome, etc.) of the endogenous bm3 gene. In addition, or alternatively, the maize plant parent and/or progeny seeds may be homozygous for a mutant allele of the endogenous br2 gene, or the maize plant parent and/or progeny seeds may be heterozygous for a mutant allele of the endogenous br2 gene. Further, the maize plant parent and/or progeny seeds may be heteroallelic for the endogenous br2 gene and may comprise a first mutant allele (e.g., on one chromosome, etc.) of the endogenous br2 gene and a second mutant allele (e.g., at the same locus on a second homologous chromosome, etc.) of the endogenous br2 gene. In connection therewith, the method may then further include selecting one or more parent and/or progeny maize plant seeds that are homozygous, heterozygous, and/or heteroallelic for a mutant allele of the endogenous bm3 gene and/or homozygous, heterozygous and/or heteroallelic for a mutant allele of the endogenous br2 gene.

In another aspect of the present disclosure, a population of seeds is provided in which the population includes at least one maize plant seed, and where the at least one maize plant seed comprises a mutant allele of an endogenous brachytic 2 (br2) gene, a mutant allele of an endogenous brown midrib 3 (bm3) gene, and/or a mutant allele of an endogenous GA20 oxidase gene or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter. In some embodiments, the at least one maize plant seed may comprise both a mutant allele of the endogenous br2 gene or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter and a mutant allele of the endogenous bm3 gene. Further, in some embodiments, the population of seeds may include a plurality of maize plant seeds, where a majority of the maize plant seeds comprises a mutant allele of the endogenous br2 gene, a mutant allele of the endogenous GA20 oxidase gene, GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter and/or a mutant allele of the endogenous bm3 gene. In some embodiments all of the maize plant seeds in the population may comprise a mutant allele of the endogenous br2 gene, a mutant allele of the endogenous GA20 oxidase gene, GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter and/or a mutant allele of the endogenous bm3 gene.

In some embodiments of the population of seeds, the maize plant seed(s) may be homozygous for the mutant allele of the endogenous br2 gene, the endogenous GA20 oxidase gene, and/or the endogenous GA3 oxidase gene. Moreover, the maize plant seed(s) may be heteroallelic for the endogenous br2 gene, the endogenous GA20 oxidase gene, and/or the endogenous GA3 oxidase gene and comprise(s) a first mutant allele of the endogenous br2 gene, the endogenous GA20 oxidase gene, and/or the endogenous GA3 oxidase gene and a second mutant allele of the endogenous br2 gene, the endogenous GA20 oxidase gene, and/or the endogenous GA3 oxidase gene. Additionally in the population, or alternatively, the maize plant seed(s) may be homozygous for the mutant allele of the endogenous bm3 gene. Moreover, the maize plant seed(s) is/are heteroallelic for the endogenous bm3 gene and comprise(s) a first mutant allele of the endogenous bm3 gene and a second mutant allele of the endogenous bm3 gene.

Further, in some example embodiments, the population may additionally include at least one companion crop plant seed (as generally described herein).

In another aspect of the present disclosure, a method is provided for producing silage. In one example embodiment, the method generally includes: (i) planting in a field a population of seeds as described herein (e.g., a population of seeds including at least one maize plant seed, and where the at least one maize plant seed comprises a mutant allele of an endogenous brachytic 2 (br2) gene and/or a mutant allele of an endogenous brown midrib 3 (bm3) gene, etc.); (ii) growing plants from the seeds, wherein a plurality of the growing plants include maize plants comprising a mutant allele of an endogenous br2 gene and/or a mutant allele of an endogenous bm3 gene; (iii) harvesting an above-ground biomass of the plants; and (iv) chopping the above-ground biomass into pieces to form a silage product. In addition, in some example embodiments, the growing plants may further include companion crop plants (as generally described herein).

In some embodiments, the method of producing silage may further include one or more of: (v) storing the silage product for a time period selected from the group consisting of about 1 day to about 2 years, from about 1 week to about 2 years, from about 1 week to about 1 year, from about 2 weeks to about 6 months, and from about 2 weeks to about 3 months; (vi) fermenting the silage product for a time period selected from the group consisting of about 1 day to about 2 years, from about 1 week to about 2 years, from about 1 week to about 1 year, from about 2 weeks to about 6 months, and from about 2 weeks to about 3 months; and/or (vii) feeding the silage product to one or more livestock animals.

In another aspect of the present disclosure, a method is provided for using maize plants and/or maize plant parts as described herein, maize plant seeds as described herein, a population of maize plants as described herein, a population of intercropped silage plants as described herein, and/or a population of seeds as described herein to produce a silage product.

In some example embodiments, the silage product produced from such use herein may have one or more characteristics selected from reduced lignin content, improved fiber digestibility, reduced ADF, increased starch content, increased NDFD or DCW, increased biomass, increased protein content (N-stover), increased milk output, increased silage yield, increased grain yield, or increased stem cross-section area, as compared to a silage product comprising a wild-type or control maize plant or maize plant part. In some embodiments, the silage product may have a protein content of about 4% or more, about 6% or more, about 8% or more, about 10% or more, about 12% or more, about 14% or more, about 16% or more, about 18% or more, about 20% or more, between about 6% and about 20% or between about 10% and about 20%. In some embodiments, the silage product may have an increased protein content over a silage product including the wild-type or control maize plant or maize plant part of about 2% or more, about 4% or more, about 6% or more, about 8% or more, about 10% or more, or about 12% or more.

In some example embodiments, the silage product produced from such use herein may have a lignin content of about 6% or less, about 5.5% or less, about 5% or less, about 4.5% or less, about 4% or less, about 3.5% or less, about 3% or less, between about 2% and about 6%, between about 2% and about 5%, between about 2% and about 4%, or between about 2% and about 3%. In some embodiments, the silage product may have a reduced lignin content over a silage product including the wild-type or control maize plant or plant part of about 1% or more, about 5% or more, about 10% or more, about 15% or more, about 20% or more, about 25% or more, or about 30% or more.

In some example embodiments, the silage product produced from such use herein may have a starch content of about 20% or more, about 30% or more, about 40% or more, about 50% or more, between about 25% and about 50%, between about 30% and about 50%, or between about 30% and about 40%. In some embodiments, the silage product may have an increased starch content over a silage product including the wild-type or control maize plant or maize plant part of about 5% or more, about 10% or more, about 15% or more, about 20% or more, about 25% or more, or about 30% or more.

In some example embodiments, the silage product produced from such use herein may have a NDFD of about 50% or more, about 55% or more, about 60% or more, about 65% or more, about 70% or more, between about 50% and about 70%, between about 55% and about 65%, or between about 55% and about 60%. In some embodiments, the silage product may have an increased NDFD over a silage product including the wild-type or control maize plant or plant part of about 1% or more, about 2% or more, about 3% or more, about 5% or more, about 10% or more, or about 15% or more.

In some example embodiments, the silage product produced from such use herein may have an ADF of about 50% or less, about 45% or less, about 40% or less, about 35% or less, about 30% or less, about 25% or less, between about 25% and about 50%, between about 25% and about 45%, or between about 25% and about 40%.

In another aspect of the present disclosure, a population of maize plants is provided, where a majority of the maize plants of the population comprises a mutant allele of an endogenous brachytic 2 (br2) gene and a mutant allele of an endogenous brown midrib gene. In some embodiments, each of the maize plants of the population comprises a mutant allele of an endogenous brachytic 2 (br2) gene and a mutant allele of an endogenous brown midrib gene. In some embodiments, the mutant allele of the endogenous brown midrib gene incudes a mutant allele of an endogenous brown midrib 3 (bm3) gene.

In some example embodiments, the maize plants of the population may be homozygous for the mutant allele of the endogenous bm3 gene. In some example embodiments, the maize plants of the population may be heteroallelic for the endogenous bm3 gene and may comprise a first mutant allele of the endogenous bm3 gene and a second mutant allele of the endogenous bm3 gene. In some example embodiments, the maize plants of the population may be homozygous for the mutant allele of the endogenous br2 gene. In some example embodiments, the maize plants of the population may be heteroallelic for the endogenous br2 gene and may comprise a first mutant allele of the endogenous br2 gene and a second mutant allele of the endogenous br2 gene.

In some example embodiments, the population of maize plants may have one or more traits selected from reduced lignin content, improved fiber digestibility, reduced ADF, increased starch content, increased NDFD, increased biomass, increased protein content (N-stover), increased milk per acre output, and increased silage yield, as compared to a population of wild-type or control maize plants.

In another aspect of the present disclosure, a population of seeds is provided in which the population includes maize plant seeds, and where a majority of the maize plant seeds of the population comprises a mutant allele of an endogenous brachytic 2 (br2) gene and a mutant allele of an endogenous brown midrib gene. In some example embodiments, each of the maize plant seeds of the population may comprises a mutant allele of an endogenous brachytic 2 (br2) gene and a mutant allele of an endogenous brown midrib gene.

In some example embodiments, in such a population, the mutant allele of the endogenous brown midrib gene may include a mutant allele of an endogenous brown midrib 3 (bm3) gene. Additionally, in some example embodiments, the maize plant seeds comprising the mutant allele of the endogenous bm3 gene may be homozygous for the mutant allele of the endogenous bm3 gene. Further, in some example embodiments, the maize plant seeds comprising the mutant allele of the endogenous bm3 gene of the population may be heteroallelic for the endogenous bm3 gene and may comprise a first mutant allele of the endogenous bm3 gene and a second mutant allele of the endogenous bm3 gene. In addition, or alternatively, in some example embodiments, the maize plant seeds comprising the mutant allele of the endogenous br2 gene may be homozygous for the mutant allele of the endogenous br2 gene. Further, in some example embodiments, the maize plant seeds comprising the mutant allele of the endogenous br2 gene may be heteroallelic for the endogenous br2 gene and may comprise a first mutant allele of the endogenous br2 gene and a second mutant allele of the endogenous br2 gene.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 shows an example of a maize plant of the present disclosure (right) homozygous for a mutant allele of the endogenous brachytic 2 (br2) gene next to a wild-type or control maize plant (left);

FIG. 2 is a graph showing the 30-hour neutral detergent fiber (NDF) digestibility for multiple samples from hybrid maize plants homozygous for a mutant allele of the endogenous brachytic 2 (br2) gene;

FIG. 3 is a graph showing the starch content for multiple samples from hybrid maize plants homozygous for a mutant allele of the endogenous brachytic 2 (br2) gene;

FIG. 4 is a graph showing the lignin content for multiple samples from hybrid maize plants homozygous for a mutant allele of the endogenous brachytic 2 (br2) gene;

FIG. 5 is a graph showing the milk output, in pounds of milk per ton of biomass (lbs/ton), for multiple samples from hybrid maize plants homozygous for a mutant allele of the endogenous brachytic 2 (br2) gene;

FIG. 6 is a graph showing the silage yield for multiple samples from hybrid maize plants homozygous for a mutant allele of the endogenous brachytic 2 (br2) gene;

FIG. 7 is a graph showing the milk output, in pounds of milk per acre (lbs/acre), for multiple samples from hybrid maize plants homozygous for a mutant allele of the endogenous brachytic 2 (br2) gene;

FIG. 8 is a graph showing the cell wall digestibility of 20 sets of isogenic inbreds from maize plants either wild-type or homozygous for a mutant allele of the endogenous brachytic 2 (br2);

FIG. 9 is a graph showing the cell wall digestibility of 20 sets of isogenic hybrids from maize plants either wild-type or homozygous for a mutant allele of the endogenous brachytic 2 (br2);

FIG. 10 are graphs showing the quantification of mean red intensity in a histological stem section from a maize plant of a conventional hybrid versus a maize plant homozygous for a mutant allele of the endogenous brachytic 2 (br2);

FIG. 11 is a graph of pooled isogenic hybrid comparisons of lignin content for a first growing season in connection with an example embodiment herein;

FIG. 12 is a graph of pooled isogenic hybrid comparisons of lignin content for a second growing season in connection with an example embodiment herein;

FIG. 13 is a graph of pooled isogenic hybrid comparisons of 30-hour NDF digestibility for a first growing season in connection with an example embodiment herein;

FIG. 14 is a graph of pooled isogenic hybrid comparisons of 30-hour NDF digestibility for a second growing season in connection with an example embodiment herein;

FIG. 15 illustrates a field of corn plants and corn plant lodging in the field; and

FIG. 16 is a set of graphs for silage characteristics with a transgenic short stature trait showing relative amounts of lignin content (LIGP), Neutral Detergent Fiber after 30-hour (NDF30), Acid Detergent Fiber (ADF), and milk content in pound per ton of biomass (MPT) between WT, e-bmr3, tSSC, and tSSC/e-bmr3 plants.

### DETAILED DESCRIPTION

Example embodiments will now be described more fully with reference to the accompanying drawings. The description and specific examples included herein are intended for purposes of illustration only and are not intended to limit the scope of the present disclosure.

At the outset, in the context of the present disclosure, the following is provided for reference with regard to various terms and/or abbreviations and/or descriptions herein.

As commonly understood, a “corn plant” or “maize plant” refers to any plant of species Zea mays and includes all plant varieties that can be bred with corn or maize, including wild maize species. Examples of maize (or corn) plants may include plants from the subspecies Zea mays L. ssp. Mays. In connection therewith, maize plants may be selected from the group Zea mays L. subsp. Mays Indentata, the group Zea mays L. subsp. Mays Indurata, the group Zea mays L. subsp. Mays Saccharata, the group Zea mays L. subsp. Mays Amylacea, the group Zea mays L. subsp. Mays Everta. The above listing is provided as an example only (and without limitation), as still other examples of maize plants may be provided and/or may be within the scope of the present disclosure (e.g., hybrids (e.g., progeny of mating (e.g., single crosses, modified single crosses, double modified single crosses, three-way crosses, modified three-way crosses, double crosses, etc.) between at least two genetically dissimilar parents (e.g., wherein at least one parent in a modified cross is the progeny of a cross between sister lines, etc.), etc.), inbreds (e.g., lines bred for genetic homogeneity, etc.), partial inbreds, or members of defined or undefined populations, etc.).

A wild-type gene or wild-type allele may refer to a gene or allele having a sequence or genotype that is most common in a particular plant species, or another sequence or genotype with natural variations, polymorphisms, or other silent mutations relative to the most common sequence or genotype that do not significantly impact the expression and activity of the gene or allele. In connection therewith, a wild-type gene or allele may contain no variation, polymorphism, or any other type of mutation that substantially affects the normal function, activity, expression, or phenotypic consequence of the gene or allele.

As used herein, the term “control plant” (or likewise a “control” plant seed, plant part, plant cell and/or plant genome) may refer to a plant (plant seed, plant part, plant cell and/or plant genome) that is used for comparison to a maize plant (or maize plant seed, plant part, plant cell and/or plant genome) and has the same or similar genetic background (e.g., same parental lines, hybrid cross, inbred line, testers, etc.) as the maize plant (or maize plant seed, plant part, plant cell and/or plant genome), except for a transgene(s), mutation(s) or genome edit(s) of the maize plant (or maize plant seed, plant part, plant cell and/or plant genome), such as a mutant allele(s) of one or more GA oxidase genes, the br2 gene or a transgene comprising a suppression construct targeting one or more GA oxidase gene(s), and/or a mutant allele(s) of the bm3 gene. For example, a control plant may be an inbred line that is the same as the inbred line used to make the maize plant, or a control plant may be the product of the same hybrid cross of inbred parental lines as the modified plant, except for the absence in the control plant of the transgene, edit or mutation of the maize plant of the present disclosure. Similarly, an unmodified control plant may refer to a plant that shares a substantially similar or essentially identical genetic background as a maize plant of the present disclosure, but without the one or more engineered changes or modifications to the genome (e.g., transgene, mutation or edit) of the maize plant. For purposes of comparison to a maize plant, plant seed, plant part, plant cell and/or plant genome of the present disclosure, a “wild-type plant” (or likewise a “wild-type” plant seed, plant part, plant cell and/or plant genome) may refer to a non-transgenic and non-genome edited control plant, plant seed, plant part, plant cell and/or plant genome. As used herein, a “control” plant, plant seed, plant part, plant cell and/or plant genome may also be a plant, plant seed, plant part, plant cell and/or plant genome having a similar (but not the same or identical) genetic background to a maize plant, plant seed, plant part, plant cell and/or plant genome of the present disclosure, if deemed sufficiently similar for comparison of the characteristics or traits to be analyzed. In some examples herein, a wild-type plant, plant seed, plant part, plant cell and/or plant genome may be a control plant, plant seed, plant part, plant cell and/or plant genome as used herein.

For purposes of the present disclosure, a “plant” includes an explant, plant part, seedling, plantlet or whole plant at any stage of regeneration or development. As used herein, a “transgenic plant” refers to a plant whose genome has been altered by the integration or insertion of a recombinant DNA molecule, construct, or sequence. A transgenic plant includes an R₀ plant developed or regenerated from an originally transformed plant cell(s) as well as progeny transgenic plants in later generations or crosses from the R₀ transgenic plant.

As used herein, a “plant part” refers to any organ or intact tissue of a plant, such as a meristem, shoot organ/structure (e.g., leaf, stem or node), root, flower or floral organ/structure (e.g., bract, sepal, petal, stamen, carpel, anther and ovule), seed (e.g., embryo, endosperm, and seed coat), fruit (e.g., the mature ovary), propagule, or other plant tissues (e.g., vascular tissue, dermal tissue, ground tissue, and the like), or any portion thereof. Plant parts of the present disclosure may be viable, nonviable, regenerable, and/or non-regenerable. A “propagule” may include any plant part that can grow into an entire plant.

A control plant or plant part may include a plant or plant part having a similar (but not the same or identical) genetic background to a modified plant or plant part, if deemed sufficiently similar for comparison of the characteristics or traits to be analyzed. For purposes of comparison to a modified or mutant plant or plant part, a wild-type or control (or likewise a wild-type plant or plant part) may refer to a non-transgenic, non-mutagenized and non-genome edited control plant, plant seed, plant part, plant cell and/or plant genome. Further, a population of wild-type or control maize plants may include a population of wild-type dual purpose (DP) control maize plants.

A locus is a chromosome region where a polymorphic nucleic acid, trait determinant, gene or marker is located. A locus may comprise one or more polymorphisms in a population, for example, where alternative alleles are present in some plants. A gene locus is a specific chromosome location in the genome of a species where a specific gene can be found. Such a locus may comprise a wild-type and/or mutant allele(s) of an endogenous gene. As used herein, a “mutant allele” is an allele of a gene or genic locus comprising one or more non-silent mutations and/or edits. The one or more mutations and/or edits may be created or introduced in in the gene or genic locus by a mutagenesis or genome editing technique. As used herein, a “mutation” can include any mutation or edit (e.g., an insertion, deletion, substitution, or translocation), which may be introduced or created using a mutagenesis or genome editing technique. As used herein, a “non-silent” mutation in a gene or genic locus is a mutation that alters, affects, or modifies the expression level and/or activity of the mRNA and/or protein product of the gene or locus. A “loss-of-function” mutation in a gene or genic locus is a mutation that lowers, reduces, or eliminates the expression level and/or activity of the mRNA and/or protein product of the gene or locus. Such a mutant allele of a gene will generally have an altered or modified expression pattern and/or level and/or an altered or modified activity of the RNA and/or protein product encoded and produced by the mutant allele of the gene or genic locus relative to a wild-type allele. Such a mutant allele will typically produce or result in at least one modified or altered trait, characteristic, property or phenotype of a plant, plant part, plant seed, plant tissue or plant cell in which the mutant allele is present in a homozygous or heterozygous state, which may depend on whether the mutant allele is dominant, semi-dominant, or recessive, relative to a wild-type allele. The presence of a marker(s) may be used to identify corn plants with desired locus, traits, targeted edits, transgenes, mutations, and/or alleles. For example, corn plants that have been subjected to a mutagenesis or genome editing treatment may be screened and selected based on an observable phenotype (e.g., any phenotype described herein, such as shorter plant height, etc.; etc.), or using a selection agent with a selectable marker (e.g., herbicide, etc.), a screenable marker, or a molecular technique. Such screening and/or selecting techniques may be used to identify and select plants comprising mutant alleles as described herein. Nucleic acids can be isolated and detected using techniques known in the art, such as any known molecular biology or recombinant nucleic acid technology.

An allele generally refers to an alternative nucleic acid sequence at a particular gene locus. For example, a first allele may occur on one chromosome, while a second allele may occur on a second homologous chromosome, for instance, as occurs for different chromosomes of a heterozygous plant (or plant part), or between different homozygous or heterozygous plants (or plant parts) in a population. In connection therewith, a brachytic allele (e.g., a mutant allele of the endogenous brachytic 2 (br2) gene, etc.) may include an allele at a particular locus that confers, or contributes to, a brachytic or semi-dwarf phenotype. For instance, a brachytic allele of a marker can be an allele that segregates with a brachytic or semi-dwarf phenotype. A brachytic phenotype includes a phenotype wherein the plant height or stature is shorter than that of a control plant lacking the brachytic phenotype.

A mutation may include an alteration of a nucleotide sequence of a genome of an organism (e.g., a plant, etc.) relative to a wild-type nucleotide sequence, and may include an insertion, deletion, or substitution of one or more nucleotides, or any combination thereof. In connection therewith, an insertion mutation may include the addition of one or more extra nucleotides into DNA of the organism. Insertions in the coding region of a gene may alter splicing of the mRNA (splice site mutation) or cause a shift in the reading frame (frameshift), both of which may alter the gene product. A deletion mutation may include the removal of one or more nucleotides from the DNA. Like insertion mutations, these mutations may alter the reading frame of the gene. A substitution mutation may include an exchange of a single nucleotide for another. A mutation may be made by any mutagenesis or genome editing technique known in the art.

An elite line or variety may include any line (e.g., of plants, etc.) that has resulted from breeding and selection for superior agronomic performance. Similarly, an elite germplasm or elite strain of germplasm may include an agronomically superior germplasm. Numerous elite lines or varieties are generally available and are generally known to those of skill in the art of plant breeding and may include many commercial lines or varieties.

As used herein, a modified plant, plant seed, plant part, plant cell, and/or plant genome, may include a corn or maize plant, plant seed, plant part, plant cell, and/or plant genome comprising one or more engineered changes or modifications to the genome (e.g., transgene, mutation or edit). For example, a modified plant may comprise one or more mutant allele(s) of one or more GA oxidase gene(s) or the br2 gene, or a transgene comprising a suppression construct targeting one or more GA oxidase gene(s), such as one or more GA20 oxidase and/or GA3 oxidase gene(s), and/or may comprise one or more mutant allele(s) of the bm3 gene.

In some embodiments, a maize plant, plant seed, plant part, plant cell, and/or plant genome may comprise an engineered change in the expression level and/or coding sequence of one or more GA oxidase gene(s) relative to a wild-type or control plant, plant seed, plant part, plant cell, and/or plant genome. For example, a modified plant may comprise a transgene or genome edit(s), such as (i) a transgenic event comprising a suppression construct or transcribable DNA sequence encoding a non-coding RNA that targets one or more GA3 and/or GA20 oxidase gene(s) for suppression, or (ii) a genome edit or mutation affecting (e.g., reducing or eliminating, etc.) the expression level or activity of one or more endogenous br2 or GA3 and/or GA20 oxidase genes. In connection therewith, such maize plant, plant seed, plant part, plant cell, and/or plant genome may have one or more mutations or edits affecting expression of one or more endogenous brachytic or GA oxidase genes, such as one or more endogenous br2 or GA3 and/or GA20 oxidase genes, introduced through chemical mutagenesis, transposon insertion or excision, or any other known mutagenesis technique, or introduced through genome editing. Further, such maize plants, plant parts, seeds, etc., may contain various molecular changes that affect expression of a brachytic or GA oxidase gene(s), such as br2, GA3, and/or GA20 oxidase gene(s), including genetic and/or epigenetic modifications. Such maize plants, plant parts, seeds, etc., may have been subjected to mutagenesis, genome editing or site directed integration (e.g., without being limiting, via methods using site-specific nucleases, etc.), genetic transformation (e.g., without being limiting, via methods of Agrobacterium transformation or microprojectile bombardment, etc.), or a combination thereof. Such maize plants, plant seeds, plant parts, and plant cells may include plants, plant seeds, plant parts, and plant cells that are offspring or derived from maize plants, plant seeds, plant parts, and plant cells that retain the molecular change or modification (e.g., change in expression level and/or activity, etc.) to the one or more br2 or GA oxidase genes.

In some embodiments, a maize plant may comprise an engineered change in the br2 or GA oxidase gene(s), or an engineered suppression of one or more GA oxidase gene(s), and/or an engineered change in the bm3 gene relative to a wild-type or control plant, plant seed, plant part, plant cell, and/or plant genome. For example, a maize plant may comprise one or more mutations or edits affecting expression of endogenous br2 or GA oxidase gene(s), or an engineered suppression of one or more endogenous GA oxidase gene(s), and/or one or more mutations or edits affecting expression of an endogenous bm3 gene, introduced through chemical mutagenesis, transposon insertion or excision, or any other known mutagenesis technique, or introduced through genome editing. In some embodiments, a maize plant may comprise an engineered change in one or more GA oxidase genes and/or the bm3 gene relative to a wild-type or control plant, plant seed, plant part, plant cell, and/or plant genome. For example, a maize plant may comprise one or more mutations or edits affecting expression of endogenous GA20 or GA3 oxidase, or an engineered suppression of one or more GA oxidase gene(s), and/or one or more mutations or edits affecting expression of an endogenous bm3 gene, introduced through chemical mutagenesis, transposon insertion or excision, or any other known mutagenesis technique, or introduced through genome editing.

Additionally or alternatively, the maize plant (or maize plant part) may comprise a non-coding RNA molecule comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein in a maize plant or maize plant part. The endogenous GA oxidase protein may be at least 80%, at least 85%, at least 90%, at least 95%, at least 96%, at least 97%, at least 98%, at least 99%, at least 99.5%, or 100% identical to SEQ ID NO: 9, 12, 15, 30, 33 or 89. Alternatively, the maize plant (or maize plant part) may comprise a non-coding RNA molecule comprising a sequence that is (i) at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a first mRNA molecule encoding a first endogenous GA20 oxidase protein in the maize plant or maize plant part, where the first endogenous GA20 oxidase protein may be at least 80%, at least 85%, at least 90%, at least 95%, at least 96%, at least 97%, at least 98%, at least 99%, at least 99.5%, or 100% identical to SEQ ID NO: 9; and/or (ii) at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a second mRNA molecule encoding a second endogenous GA20 oxidase protein in the maize plant or maize plant part, where the second endogenous GA20 oxidase protein may be at least 80%, at least 85%, at least 90%, at least 95%, at least 96%, at least 97%, at least 98%, at least 99%, at least 99.5%, or 100% identical to SEQ ID NO: 15.

Such maize plants, plant seeds, plant parts, and plant cells may include plants, plant seeds, plant parts, and plant cells that are offspring or derived from maize plants, plant seeds, plant parts, and plant cells that retain the genetic or molecular change. A maize seed provided herein may give rise to a maize plant provided herein. A maize plant, plant seed, plant part, plant cell, or plant genome provided herein may comprise a recombinant DNA construct or vector or mutation or genome edit as provided herein. A maize plant product may be any product made from a maize plant, plant part, plant seed, plant cell, or plant chromosome provided herein, or any portion or component thereof. The maize plant or plant part may comprise a plant-expressible promoter, such as a vascular promoter, a RTBV promoter, a leaf promoter, or a leaf promoter. The non-coding RNA molecule may be encoded by a transcribable DNA sequence that is a precursor miRNA or siRNA which is process and/or cleaved in a plant cell to form a mature miRNA and/or siRNA.

Introgression may include transmission of a desired trait, such as a transgene or a mutant allele of a genetic locus, from one genetic background to another.

As commonly understood in the art, crossing may include producing progeny via fertilization (e.g., cells, seeds or plants, etc.) and may include crosses between plants (sexual) as well as self-fertilization (selfing). Backcrossing may include a process whereby a progeny plant is crossed one or more times back to one of its parents. In a backcrossing scheme, a donor parent may include a parental plant with the desired gene or locus to be introgressed. And, a recipient parent (used one or more times) or recurrent parent (used two or more times) may include a parental plant into which the gene or locus is being introgressed. In some instances, a backcross may be performed repeatedly, with a progeny individual of each successive backcross generation being itself backcrossed to the same parental genotype.

A phenotype, or phenotypic trait, or a trait, or a property, or a characteristic may refer to one or more detectable characteristics of a plant cell, plant tissue, plant part, plant or population or plurality of plants which can be influenced by genotype. The phenotype can be observable to the naked eye, or by any other means of evaluation known in the art such as, for example, microscopy, biochemical analysis, genomic analysis, an assay for a particular disease or other tolerance, etc. In some examples, a phenotype may be controlled (or controllable) by a single gene or genetic locus, for example, a single gene trait (or mutated gene trait, etc.). In other examples, a phenotype may be the result of several genes (or several mutated genes, etc.).

A population of plants, or plant population, or population of seeds, or seed population may include a set comprising any number of plants, seeds, plant parts, etc. (e.g., which may be subsequently used as desired, for example, for sampling, advancement in a pipeline, advancement for commercial use, advancement for non-commercial use, etc.). In some examples, a population may relate to a breeding population of plants from which members may be selected and crossed to produce progeny in a breeding program. In some examples, a population may relate to a population of plants grown (e.g., in a field, etc.) for use in producing grain, for use in producing biomass for a silage product, for use in producing biomass for energy production, etc. In addition, a population of plants may include the progeny of a single breeding cross or a plurality of breeding crosses, and may be either actual plants or plant derived material, or in silico representations of the plants. However, population members need not be identical to population members selected for use in subsequent cycles of analyses or those ultimately selected to obtain final progeny plants. In some examples, a plant population may be derived from a single cross, or from two or more crosses between the same or different parents. In some examples, a population may relate to a population of seeds selected for subsequent growing, etc. Although a population of plants or seeds herein may include any number of plants or seeds, those of ordinary skill in the art will recognize that plant breeders commonly use population sizes ranging from one or two hundred plants or seeds to several thousand (or more or less), and that a highest performing 5-20% of a population, for example, or more or less, may be what is commonly selected to be used in subsequent crosses in order to improve the performance of subsequent generations of the population.

Germplasm may refer to a non-transgenic or non-traited genetic background of a plant or, more generally, to living sources of genetic material. The germplasm may be part of the plant (broadly, organism), or may be separate from the plant. In general, germplasm provides genetic material with a specific molecular makeup that may provide a physical foundation for some or all of the hereditary qualities of a plant. To that end, germplasm may include cells, seed or tissues from which new plants may be grown, or plant parts, such as leaves, stems, pollen, or cells that can be cultured into a whole plant.

Silage (or a silage product) may include a crop harvested green and preserved in a succulent condition possibly by partial fermentation in a generally airtight container or storage (e.g., via an ensiling process, etc.). Corn (or maize) silage, for example, may include above-ground biomass of corn (or maize) plants (or of a corn (or maize) crop) such as, for example, car, stalks, and leaves from the plants. In connection therewith, farmers may use silage from corn and other grains to feed livestock or other animals (e.g., cattle, etc.).

The plants herein may be grown (e.g., from planted seeds, etc.) in and harvested from a growing space. The growing space may include a field or plot or multiple fields or plots, one or more controlled growing environments (e.g., a green house, growth chamber, or any other generally closed ecosystem or environment, etc.), or any other space(s) suitable for growing plant plants. In connection therewith, a field, for example, may include an area of land that may be at least partially enclosed, covered or screened or not enclosed, covered or screened, and that is used generally for agricultural purposes (e.g., cultivating crops, etc.). The field may additionally include one or more boundaries or borders defining the field, for example, fences, roads, water boundaries, other land boundaries, other agricultural boundaries (e.g., trees, shrubs, other vegetation, etc.), etc.

Example embodiments of the present disclosure are generally directed to a maize plant and/or a population of maize plants (and/or to a part(s) of the maize plant(s)) that include(s) one or more mutant allele(s) of the endogenous brachytic 2 (br2) gene, one or more mutant allele(s) of an endogenous GA oxidase gene(s), or a transgene comprising transcribable DNA sequence encoding a small RNA molecule that targets one or more GA oxidase gene(s) for suppression, and/or one or more mutant allele(s) of the endogenous brown midrib 3 (bm3) gene. For instance, in some example embodiments, the maize plant and/or population of maize plants (and/or part(s) thereof) may include one or more mutant allele(s) of the endogenous br2 gene. In some example embodiments, the maize plant and/or population of maize plants (and/or part(s) thereof) may include one or more mutant allele(s) of one or more endogenous GA20 or GA3 oxidase gene(s). In some example embodiments, the maize plant and/or population of maize plants (and/or part(s) thereof) may include a transgene comprising transcribable DNA sequence encoding a small RNA molecule that targets one or more GA20 or GA3 oxidase gene(s) for suppression. In some example embodiments, the maize plant and/or population of maize plants (and/or part(s) thereof) may include one or more mutant allele(s) of the endogenous bm3 gene. In some example embodiments, the maize plant and/or population of maize plants (and/or part(s) thereof) may include one or more mutant allele(s) of the endogenous br2 gene and one or more mutant allele(s) of the endogenous bm3 gene. In some example embodiments, the maize plant and/or population of maize plants (and/or part(s) thereof) may include one or more mutant allele(s) of an endogenous GA3 or GA20 oxidase gene and one or more mutant allele(s) of the endogenous bm3 gene. In some example embodiments, the maize plant and/or population of maize plants (and/or part(s) thereof) may include a transgene comprising transcribable DNA sequence encoding a small RNA molecule that targets one or more GA20 or GA3 oxidase gene(s) for suppression and one or more mutant allele(s) of the endogenous bm3 gene. In some embodiments, the maize plant and/or population of maize plants (and/or part(s) thereof) may be homozygous or heterozygous for one or more mutant allele(s) of the endogenous br2 gene, homozygous or heterozygous for one or more mutant allele(s) of an endogenous GA20 or GA3 oxidase gene, and/or homozygous or heterozygous for one or more mutant allele(s) of the endogenous bm3 gene. As a result, the maize plant(s) (and/or plant part(s) thereof) may have one or more trait(s), characteristic(s), etc. that make it (them) suitable for grain and/or forage (e.g., silage, etc.) production, energy production, etc. While the description herein is provided in terms of a corn or maize plant (or population of maize plants, or part(s) thereof, etc.), it should be appreciated that the description may similarly apply to one or more parts of the corn or maize plant(s) (e.g., maize plant part(s), etc.) and not only the maize plant itself or as a whole.

In some example embodiments, a maize plant herein (or a plant part, etc., thereof) comprises a mutant allele of the endogenous br2 gene. In connection therewith, the endogenous br2 gene encodes an ATP binding cassette type B (ABCB) auxin transporter, and its expression may influence height of a maize plant. In connection therewith, mutations of the br2 gene may affect height of the maize plant (e.g., may limit a height of the maize plant, etc.), for example, through shorter lower internode(s) with normal or near normal upper internode(s), etc. Different alleles (or mutant alleles) of the endogenous br2 gene may have different degrees or effects on the height of the maize plant. Such mutant alleles may result in a loss of gene function, gain of gene function, no change in gene function, or other changes in gene expression in the maize plant. In general, loss-of-function mutations or mutant alleles of the endogenous br2 gene, an endogenous GA20 oxidase gene, or an endogenous GA3 oxidase gene result in a shorter stature or semi-dwarf phenotype when present in the right genetic zygosity and combination, relative to a wild-type control plant. Maize plants homozygous for one or more loss-of-function mutation(s) or mutant allele(s) of the endogenous br2 gene or a GA3 oxidase gene may have a shorter stature or semi-dwarf phenotype. Maize plants homozygous for one or more loss-of-function mutation(s) or mutant allele(s) of the endogenous GA20 oxidase_3 gene and homozygous for one or more loss-of-function mutation(s) or mutant allele(s) of the endogenous GA20 oxidase_5 gene may have a shorter stature or semi-dwarf phenotype. Maize plants homozygous for one or more loss-of-function mutation(s) or mutant allele(s) of the endogenous GA20 oxidase_3 gene and heterozygous for loss-of-function mutation(s) or mutant allele of the endogenous GA20 oxidase_5 gene may have a shorter stature or semi-dwarf phenotype. Maize plants heterozygous for one or more loss-of-function mutation(s) or a mutant allele of the endogenous GA20 oxidase_3 gene and homozygous for one or more loss-of-function mutation(s) or mutant allele(s) of the endogenous GA20 oxidase_5 gene may also have a shorter stature or semi-dwarf phenotype. Sec, e.g., WO 2019/161147 and WO 2019/161149, the contents and disclosures of which are incorporated herein by reference.

A wild-type genomic DNA sequence of the br2 locus from a reference genome of corn or maize is provided in SEQ ID NO: 90. A wild-type cDNA sequence of the br2 locus from the reference genome is provided in SEQ ID NO: 91. A wild-type coding sequence (CDS) sequence of the br2 locus from the reference genome is provided in SEQ ID NO: 92. A wild-type amino acid or protein sequence encoded by SEQ ID NO: 91 and 92 is provided in SEQ ID NO: 93.

For the br2 gene in corn or maize, SEQ ID NO: 90 provides 954 nucleotides upstream of the br2 5′-UTR; nucleotides 955-1000 correspond to the 5′-UTR; nucleotides 1001-1604 correspond to the first exon; nucleotides 1605-1747 correspond to the first intron; nucleotides 1748-2384 correspond to the second exon; nucleotides 2385-2473 correspond to the second intron; nucleotides 2474-2784 correspond to the third exon; nucleotides 2785-3410 correspond to the third intron; nucleotides 3411-3640 correspond to the fourth exon; nucleotides 3641-5309 correspond to the fourth intron; nucleotides 5310-7667 correspond to the fifth exon; and nucleotides 7668-8029 correspond to the 3′-UTR. SEQ ID NO: 90 also provides 638 nucleotides downstream of the end of the 3′-UTR (nucleotides 8030-8667).

A mutant allele of the endogenous br2 gene of the maize plant herein (or plant part thereof) may comprise one or more mutation(s), such as an insertion, deletion or substitution of one or more nucleotides, or any combination thereof, in the endogenous br2 gene. In an aspect, a mutation or a mutant allele of an endogenous br2 gene may comprise one or more mutation(s) of an endogenous br2 locus, such as an insertion, deletion and/or substitution of one or more nucleotides, or a combination thereof, of at least one exon, at least one intron, at least one promoter, regulatory or upstream region, and/or at least one 5′ or 3′ untranslated region (UTR) of an endogenous br2 locus, as compared to a wild-type br2 sequence and/or as compared to SEQ ID NO: 90, 91 and/or 92. For example, a mutant allele of the endogenous br2 gene may be the br2-23 allele, br2-708/allele, br2-7861 allele, br2-qph1 allele, br2-qpa1 allele, br2-NC238 allele, or br2-1005 allele (see, e.g., Bage et al., Plant Gene 21 (2020), etc.). See also, e.g., PCT/US2016/029492, the entire content and disclosure of which are incorporated herein by reference. In connection therewith, mutant allele br2-23 is characterized by an 8 bp deletion; mutant allele br2-7081 and br2-7861 are each characterized by spontaneous transposon mutation; mutant allele br2-qph1 is characterized by a missense mutation; mutant allele br2-qpa1 is characterized by a 241 bp deletion; mutant allele br2-NC238 is characterized by a MITE transposon insertion; and mutant allele br2-1005 is characterized by CRISPR-Cas9 genome editing technology. In the latter, to synthesize the br2-1005 mutant allele, the CRISPR-Cas9 system induced a double strand break (DSB) in exon 5 of the br2 gene. Non-homologous end joining repair then introduced a one nucleotide frameshift causing a premature stop codon. As described herein, desired mutant alleles of the br2 gene may be selected based on an observable phenotype or using a selection agent with a selectable marker, a screenable marker, or a molecular technique. According to some embodiments, an endogenous br2 gene can be edited or mutated to express a truncated protein relative to a wild-type protein by the introduction of a premature stop codon into the coding sequence and the encoded mRNA transcript of the endogenous br2 gene.

A mutant allele of the endogenous br2 gene may occur in the maize plant (or plant part thereof) through introgression, such as from an existing line, or a mutant allele of the endogenous br2 gene may be a mutagenized or an edited allele of the endogenous br2 gene (e.g., via targeted genome editing, etc.). Sec, e.g., U.S. Pat. No. 10,472,684 and PCT Application No. PCT/US2017/067888 (describing various methods for genetically modifying maize plants with respect to the br2 gene, and which is incorporated herein by reference in its entirety). In the latter, where the mutant allele of the endogenous br2 gene is an edited allele of the endogenous br2 gene, the edited allele of the br2 gene may then be introduced into the maize plant (e.g., into a non-brachytic background, etc.). In connection therewith, one or more edited allele(s) of the endogenous br2 gene may be synthesized via genome editing techniques that utilize genome modification enzymes such as, for example, Zinc finger nucleases (ZFNs), engineered or native meganucleases, Transcription activator-like effector nucleases (TALENs), RNA-guided endonucleases (e.g., making use of clustered regularly interspaced short palindromic repeats (CRISPR) technology, etc.), etc., and then introduced into the maize plant. Sec, e.g., Gaj et al., Trends in Biotechnology, 31 (7): 397-405 (2013). Examples of edited mutant br2 alleles are discussed, for example, in U.S. Pat. No. 10,472,684 and PCT Application No. PCT/US2017/067888 (which are incorporated herein by reference).

In some embodiments, the maize plant (or plant part thereof) may be homozygous for a mutant allele of the endogenous br2 gene. If both alleles of a gene or at a locus on homologous chromosomes are mutant alleles, whether or not the two mutant alleles are the same or different, then the plant is described as being homozygous for the mutant alleles. In some embodiments, the maize plant may be heterozygous for a mutant allele of the endogenous br2 gene. If one allele at a locus on one chromosome of a plant is a mutant allele and the other corresponding allele on the homologous chromosome of the plant is wild-type, then the plant is described as being heterozygous for a mutant allele. In some embodiments, for diploid organisms such as maize/corn, the maize plant may be heteroallelic for the endogenous br2 gene and may comprise a first mutant allele on one chromosome of the endogenous br2 gene and a second mutant allele at the same locus on a second homologous chromosome of the endogenous br2 gene. In connection therewith, where the maize plant is heteroallelic for the endogenous br2 gene, for instance, the maize plant may include two different mutant br2 gene alleles at the same locus. A diploid plant homozygous for mutant alleles at a locus may comprise the same mutant allele or different mutant alleles if heteroallelic.

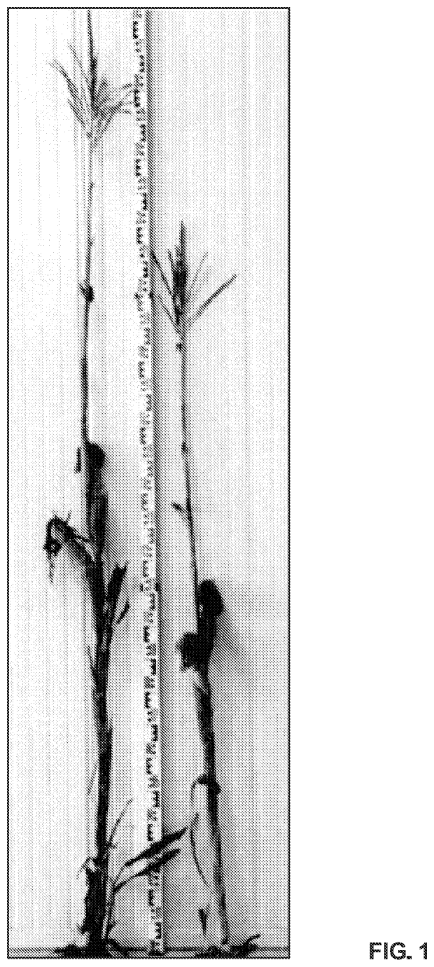

Further, in some embodiments the maize plant (or plant part thereof) may be an inbred plant. In some embodiments, the maize plant (or plant part thereof) may be a hybrid plant. In some embodiments, the maize plant (or plant part thereof) may be a short stature, semi-dwarf, or brachytic maize (or corn) plant. In some embodiments, the maize plant (or plant part thereof) may have a mutant allele of the endogenous br2 gene. In some embodiments, the maize plant (or plant part thereof) may have a reduced level of Br2 mRNA and/or protein compared to a wild-type or control maize plant not having a mutated br2 gene or mutant allele of the br2 gene. In some embodiments, the maize plant (or plant part thereof) may comprise reduced Br2 protein expression and/or activity as compared to a wild-type or control maize plant not having a mutated br2 gene allele. In some embodiments, the maize plant (or plant part thereof) may have a mutant allele of an endogenous GA oxidase gene or a transgene comprising a transcribable DNA sequence encoding a RNA molecule that targets one or more GA oxidase gene(s) for suppression. In some embodiments, the maize plant (or plant part thereof) may have a reduced level of GA oxidase mRNA and/or protein compared to a wild-type or control maize plant not having a mutated GA oxidase gene or a transgene targeting a GA oxidase gene for suppression. In some embodiments, the maize plant (or plant part thereof) may comprise reduced GA oxidase protein expression and/or activity as compared to a wild-type or control maize plant not having a mutated GA oxidase gene allele or a transgene targeting a GA oxidase gene for suppression. In some embodiments, the maize plant (or plant part thereof) may have a reduced plant height (or shorter plant height) as compared to a wild-type or control maize plant (e.g., a wild-type or control maize plant not having a mutated br2 or GA oxidase gene allele or a transgene targeting a GA oxidase gene for suppression, and/or not having a mutated bm3 gene allele, etc.), for example, about 10% or more, about 15% or more, about 20% or more, about 30% or more, about 40% or more, about 50% or more, about 60% or more, about 70% or more, between about 10% and about 70%, etc., shorter than the wild-type or control maize plant not having a mutated br2 gene allele (see, e.g., FIG. 1, etc.). In addition, in some embodiments the maize plant (or plant part thereof) may have increased resistance to root and/or stalk lodging and/or green snap, as compared to a wild-type or control maize plant not having a mutated br2 gene allele and/or not having a mutated bm3 gene allele.

According to some embodiments, a corn or maize plant, plant part, etc., may comprise a mutation or transgene other than a mutation in an endogenous br2 or GA oxidase gene to give rise to a short stature phenotype and/or improved silage characteristics. Such a corn or maize plant, plant part, etc., may have a reduced level of one or more auxin or active gibberellic acid (GA) molecules. Active or bioactive gibberellic acid molecules (i.e., “active gibberellins”, “active gibberellic acids” or “active GAs”) are known in the art for a given plant species, as distinguished from inactive GAs. For example, active GAs in corn or maize plants include the following: GA1, GA3, GA4, and GA7. Certain biosynthetic enzymes (e.g., GA20 oxidase and GA3 oxidase) and catabolic enzymes (e.g., GA2 oxidase) in the GA pathway participate in GA synthesis and degradation, respectively, to affect active GA levels in plant tissues. Thus, suppression of certain GA20 oxidase and/or GA3 oxidase genes, or overexpression or transgenic expression of one or more GA2 oxidase genes, in a constitutive or tissue-specific or tissue-preferred manner can produce corn or maize plants having a short stature phenotype and increased lodging resistance, with potentially increased yield and/or improved silage characteristics. Likewise, mutation of certain GA20 oxidase and/or GA3 oxidase genes can produce corn or maize plants having a short stature phenotype and increased lodging resistance, with potentially increased yield and/or improved silage characteristics. Any mutation of a br2 gene, a GA20 oxidase gene, or a GA3 oxidase gene may be achieved by any known mutagenesis or genome editing technique.

A corn or maize plant or plant part can comprise an expression cassette or transgene comprising a transcribable polynucleotide or DNA sequence encoding a non-coding RNA molecule that targets one or more GA20 or GA3 oxidase gene(s) for suppression, wherein the transcribable sequence is operably linked to a plant-expressible promoter. In an aspect, the non-coding RNA molecule can target one or more GA20 oxidase gene(s) for suppression, such as a GA20 oxidase_3 gene, a GA20 oxidase_4 gene, a GA20 oxidase_5 gene, or any combination thereof, such as targeting the GA20 oxidase_3 gene and the GA20 oxidase_5 gene. In an aspect, the non-coding RNA molecule can target one or more GA3 oxidase gene(s) for suppression, such as the GA3 oxidase_1 gene, GA3 oxidase_2 and/or GA3 oxidase_3 gene. Alternatively, a corn or maize plant, plant part, etc., may comprise a mutation(s) or mutant allele(s) of one or more GA20 or GA3 oxidase gene(s), and such corn or maize plant, plant part, etc., may be homozygous, heterozygous, and/or heteroallelic for such mutation(s) or mutant allele(s). Such mutation(s) or mutant allele(s) of the GA20 or GA3 oxidase gene(s) may be dominant, semi-dominant or recessive. In an aspect, a mutation or mutant allele of a GA20 oxidase or GA3 oxidase gene may comprise an inversion or antisense sequence or a deletion bringing the promoter and/or regulatory elements of a neighboring gene into proximity to the GA20 oxidase or GA3 oxidase gene or the br2 gene to produce an antisense transcript or sequence to cause suppression or a dominant negative effect on the GA20 oxidase or the GA3 oxidase gene or the br2 gene, respectively. A corn or maize plant or plant part can comprise an expression cassette or transgene comprising a transcribable sequence encoding a GA2 oxidase gene(s), wherein the transcribable sequence is operably linked to a plant-expressible promoter. See, e.g., PCT Application Nos. PCT/US2017/047405, PCT/US2019/018131, PCT/US2019/018133, PCT/US2020/034933, and PCT/US2020/034996, the entire contents and disclosures of which are incorporated herein by reference.

Suppression of GA20 and/or GA3 oxidase gene(s) can be effective in achieving a short stature, semi-dwarf phenotype with increased resistance to lodging, but without reproductive off-types in the car. Suppression of GA20 and/or GA3 oxidase gene(s) through constitutive expression, or in active GA-producing tissues, such as the vascular and/or leaf tissues of the plant, can produce a short-stature plant with increased lodging resistance, but without significant off-types in reproductive tissues. As used herein, an “active GA-producing tissue” is a plant tissue that produces one or more active GAs. Expression of a GA20 or GA3 oxidase suppression element using a constitutive, vascular and/or leaf promoter can be sufficient and effective at producing plants with the short stature phenotype, while avoiding potential off-types in reproductive tissues. For example, GA20 and/or GA3 oxidase gene(s) can be targeted for suppression using a vascular promoter, such as a rice tungro bacilliform virus (RTBV) promoter, that drives expression in vascular tissues of plants. The expression pattern of the RTBV promoter is enriched in vascular tissues of corn plants relative to non-vascular tissues, which is sufficient to produce a semi-dwarf phenotype in corn plants when operably linked to a suppression element targeting GA20 and GA3 oxidase gene(s).

Lowering of active GA levels in tissue(s) of a corn or maize plant, such as in the stalk, stem, or internode(s) of the corn or maize plant, can reduce plant height and increase lodging resistance, while avoiding off-types in the reproductive tissues of the plant, such as in the female (car) or male (tassel) tissues of the plant. Since active GAs can move through the corn or maize plant, reduction of active GAs in an active GA-producing tissue can result in a reduction of active GAs in other tissue(s) of the plant, such as the stalk, stem, or internode(s) of corn plant, to produce a short stature phenotype. Thus, for example, active GAs may be reduced in leaf tissue of a corn or maize plant and cause reduced plant height, which is due to a shortening of the stalk, stem, and internode(s) of the plant, through expression of a GA oxidase gene suppression element under the control of a leaf promoter presumably due to the ability of the active GAs to move through the plant.

The term “suppression” as used herein, refers to a lowering, reduction or elimination of the expression level of a mRNA and/or protein encoded by a target gene in a plant, plant cell or plant tissue at one or more stage(s) of plant development, as compared to the expression level of such target mRNA and/or protein in a wild-type or control plant, cell or tissue at the same stage(s) of plant development. According to some embodiments, a corn or maize plant is provided having a GA20 oxidase gene expression level (e.g., GA20 oxidase_3, GA20 oxidase_4 and/or GA20 oxidase_5 mRNA and/or protein expression level(s)) that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a wild-type or control plant. According to some embodiments, a corn or maize plant is provided having a GA3 oxidase gene expression level (e.g., GA3 oxidase_1, GA3 oxidase_2 and/or GA3 oxidase_3 mRNA and/or protein expression level(s)) that is reduced in at least one plant tissue by at least 5%, at least 10%, at least 20%, at least 25%, at least 30%, at least 40%, at least 50%, at least 60%, at least 70%, at least 75%, at least 80%, at least 90%, or 100%, as compared to a wild-type or control plant. According to some embodiments, a corn or maize plant is provided having a GA20 oxidase gene expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, a corn or maize plant is provided having a GA3 oxidase gene expression level that is reduced in at least one plant tissue by 5%-20%, 5%-25%, 5%-30%, 5%-40%, 5%-50%, 5%-60%, 5%-70%, 5%-75%, 5%-80%, 5%-90%, 5%-100%, 75%-100%, 50%-100%, 50%-90%, 50%-75%, 25%-75%, 30%-80%, or 10%-75%, as compared to a control plant. According to some embodiments, the at least one tissue of a corn or maize plant having a reduced expression level of a GA20 oxidase and/or GA3 oxidase gene(s) includes one or more active GA producing tissue(s) of the plant, such as the vascular and/or leaf tissue(s) of the plant, during one or more vegetative stage(s) of development.

Any method known in the art for suppression of a target gene may be used to suppress endogenous GA oxidase gene(s) according to embodiments of the present disclosure including expression of antisense RNAs, double stranded RNAs (dsRNAs) or inverted repeat RNA sequences, or via co-suppression or RNA interference (RNAi) through expression of small interfering RNAs (siRNAs), short hairpin RNAs (shRNAs), trans-acting siRNAs (ta-siRNAs), or micro RNAs (miRNAs). Furthermore, sense and/or antisense RNA molecules may be used that target the coding and/or non-coding genomic sequences or regions within or near a GA oxidase or br2 gene(s) to cause silencing of the gene. Accordingly, any of these methods may be used for the targeted suppression of an endogenous GA20 oxidase and/or GA3 oxidase gene(s) in a tissue-specific or tissue-preferred manner. See, e.g., U.S. Patent Application Publication Nos. 2009/0070898, 2011/0296555, and 2011/0035839, the contents and disclosures of which are incorporated herein by reference.

According to embodiments of the present disclosure, a corn or maize plant, plant part, etc., is provided comprising a recombinant DNA construct comprising an expression cassette, transcribable DNA sequence or suppression element targeting one or more GA20 oxidase or GA3 oxidase target gene(s) for suppression. In some embodiments, the corn or maize plant, plant part, etc., may further comprise a mutant allele of the endogenous bm3 gene, and/or which may be homozygous or heterozygous for one or more mutant allele(s) of the endogenous bm3 gene. According to embodiments of the present disclosure, a recombinant DNA molecule, construct, or vector is provided comprising a suppression element for GA20 oxidase or GA3 oxidase target gene(s) that is operably linked to a plant-expressible constitutive, tissue-specific, or tissue preferred promoter. The suppression element may comprise a transcribable DNA sequence of at least 19 nucleotides in length, such as from about 19 nucleotides in length to about 27 nucleotides in length, or 19, 20, 21, 22, 23, 24, 25, 26, or 27 nucleotides in length, wherein the transcribable DNA sequence corresponds to at least a portion of the GA oxidase target gene to be suppressed, and/or to a DNA sequence complementary to the target gene. The suppression element may be 19-30, 19-50, 19-100, 19-200, 19-300, 19-500, 19-1000, 19-1500, 19-2000, 19-3000, 19-4000, or 19-5000 nucleotides in length. The suppression element may be at least 19, at least 20, at least 21, at least 22, or at least 23 nucleotides or more in length (e.g., at least 25, at least 30, at least 50, at least 100, at least 200, at least 300, at least 500, at least 1000, at least 1500, at least 2000, at least 3000, at least 4000, or at least 5000 nucleotides in length). Depending on the length and sequence of a suppression element, one or more sequence mismatches or non-complementary bases, such as 1, 2, 3, 4, 5, 6, 7, 8 or more mismatches, may be tolerated without a loss of suppression if the non-coding RNA molecule encoded by the suppression element is still able to sufficiently hybridize and bind to the target mRNA molecule of the GA20 oxidase or GA3 oxidase gene(s). Even shorter RNAi suppression elements ranging from about 19 nucleotides to about 27 nucleotides in length may have one or more mismatches or non-complementary bases, yet still be effective at suppressing a target GA oxidase gene. Accordingly, a sense or anti-sense suppression element sequence may be at least 80%, at least 85%, at least 90%, at least 95%, at least 96%, at least 97%, at least 98%, at least 99%, at least 99.5% or 100% identical to a corresponding sequence of at least a segment or portion of the GA oxidase target gene, or its complementary sequence, respectively.

A suppression element or transcribable DNA sequence of the present disclosure for targeted suppression of GA oxidase gene(s) may include one or more of the following: (a) a DNA sequence that includes at least one anti-sense DNA sequence that is anti-sense or complementary to at least one segment or portion of the targeted GA oxidase gene; (b) a DNA sequence that includes multiple copies of at least one anti-sense DNA sequence that is anti-sense or complementary to at least one segment or portion of the targeted GA oxidase gene; (c) a DNA sequence that includes at least one sense DNA sequence that comprises at least one segment or portion of the targeted GA oxidase gene; (d) a DNA sequence that includes multiple copies of at least one sense DNA sequence that each comprise at least one segment or portion of the targeted GA oxidase gene; (c) a DNA sequence that includes an inverted repeat of a segment or portion of a targeted GA oxidase gene and/or transcribes into RNA for suppressing the targeted GA oxidase gene by forming double-stranded RNA, wherein the transcribed RNA includes at least one anti-sense DNA sequence that is anti-sense or complementary to at least one segment or portion of the targeted GA oxidase gene and at least one sense DNA sequence that comprises at least one segment or portion of the targeted GA oxidase gene; (f) a DNA sequence that is transcribed into RNA for suppressing the targeted GA oxidase gene by forming a single double-stranded RNA and includes multiple serial anti-sense DNA sequences that are each anti-sense or complementary to at least one segment or portion of the targeted GA oxidase gene and multiple serial sense DNA sequences that each comprise at least one segment or portion of the targeted GA oxidase gene; (g) a DNA sequence that is transcribed into RNA for suppressing the targeted GA oxidase gene by forming multiple double strands of RNA and includes multiple anti-sense DNA sequences that are each anti-sense or complementary to at least one segment or portion of the targeted GA oxidase gene and multiple sense DNA sequences that each comprise at least one segment or portion of the targeted GA oxidase gene, wherein the multiple anti-sense DNA segments and multiple sense DNA segments are arranged in a series of inverted repeats; (h) a DNA sequence that includes nucleotides derived from a miRNA, preferably a plant miRNA; (i) a DNA sequence that includes a miRNA precursor that encodes an artificial miRNA complementary to at least one segment or portion of the targeted GA oxidase gene; (j) a DNA sequence that includes nucleotides of a siRNA; (k) a DNA sequence that is transcribed into an RNA aptamer capable of binding to a ligand; and (1) a DNA sequence that is transcribed into an RNA aptamer capable of binding to a ligand and DNA that transcribes into a regulatory RNA capable of regulating expression of the targeted GA oxidase gene, wherein the regulation of the targeted GA oxidase gene is dependent on the conformation of the regulatory RNA, and the conformation of the regulatory RNA is allosterically affected by the binding state of the RNA aptamer by the ligand. Any of these gene suppression elements, whether transcribed into a single stranded or double-stranded RNA, may be designed to suppress more than one GA oxidase target gene, depending on the number and sequence of the suppression element(s).

Multiple sense and/or anti-sense suppression elements for more than one GA oxidase target may be arranged serially in tandem or arranged in tandem segments or repeats, such as tandem inverted repeats, which may be interrupted by one or more spacer sequence(s), and the sequence of each suppression element may target one or more GA oxidase gene(s). Furthermore, the sense or anti-sense sequence of the suppression element may not be perfectly matched or complementary to the targeted GA oxidase gene sequence, depending on the sequence and length of the suppression element. Even shorter RNAi suppression elements from about 19 nucleotides to about 27 nucleotides in length may have one or more mismatches or non-complementary bases, yet still be effective at suppressing the target GA oxidase gene. Accordingly, a sense or anti-sense suppression element sequence may be at least 80%, at least 85%, at least 90%, at least 95%, at least 96%, at least 97%, at least 98%, at least 99%, at least 99.5% or 100% identical to a corresponding sequence of at least a segment or portion of the GA oxidase target gene, or its complementary sequence, respectively.

For anti-sense suppression, the transcribable DNA sequence or suppression element comprises a sequence that is anti-sense or complementary to at least a portion or segment of the GA oxidase target gene. The suppression element may comprise multiple anti-sense sequences that are complementary to one or more portions or segments of the GA oxidase targeted gene(s), or multiple copies of an anti-sense sequence that is complementary to a GA oxidase target gene. The anti-sense suppression element sequence may be at least 80%, at least 85%, at least 90%, at least 95%, at least 96%, at least 97%, at least 98%, at least 99%, at least 99.5% or 100% identical to a DNA sequence that is complementary to at least a segment or portion of the targeted GA oxidase gene. In other words, the anti-sense suppression element sequence may be at least 80%, at least 85%, at least 90%, at least 95%, at least 96%, at least 97%, at least 98%, at least 99%, at least 99.5% or 100% complementary to the GA oxidase target gene.

For suppression of GA oxidase gene(s) using an inverted repeat or a transcribed dsRNA, a transcribable DNA sequence or suppression element may comprise a sense sequence that comprises a segment or portion of a targeted GA oxidase gene and an anti-sense sequence that is complementary to a segment or portion of the targeted GA oxidase gene, wherein the sense and anti-sense DNA sequences are arranged in tandem. The sense and/or anti-sense sequences, respectively, may each be less than 100% identical or complementary to a segment or portion of the targeted GA oxidase gene as described above. The sense and anti-sense sequences may be separated by a spacer sequence, such that the RNA molecule transcribed from the suppression element forms a stem, loop or stem-loop structure between the sense and anti-sense sequences. The suppression element may instead comprise multiple sense and anti-sense sequences that are arranged in tandem, which may also be separated by one or more spacer sequences. Such suppression elements comprising multiple sense and anti-sense sequences may be arranged as a series of sense sequences followed by a series of anti-sense sequences, or as a series of tandemly arranged sense and anti-sense sequences. Alternatively, one or more sense DNA sequences may be expressed separately from the one or more anti-sense sequences (i.e., one or more sense DNA sequences may be expressed from a first transcribable DNA sequence, and one or more anti-sense DNA sequences may be expressed from a second transcribable DNA sequence, wherein the first and second transcribable DNA sequences are expressed as separate transcripts).

For suppression of GA oxidase gene(s) using a microRNA (miRNA), the transcribable DNA sequence or suppression element may comprise a DNA sequence derived from a miRNA sequence native to a virus or eukaryote, such as an animal or plant, or modified or derived from such a native miRNA sequence. Such native or native-derived miRNA sequences may form a fold back structure and serve as a scaffold for the precursor miRNA (pre-miRNA), and may correspond to the stem region of a native miRNA precursor sequence, such as from a native (or native-derived) primary-miRNA (pri-miRNA) or pre-miRNA sequence. However, in addition to these native or native-derived miRNA scaffold or preprocessed sequences, engineered or synthetic miRNAs of the present embodiments further comprise a sequence corresponding to a segment or portion of the targeted GA oxidase gene(s). Thus, in addition to the pre-processed or scaffold miRNA sequences, the suppression element may further comprise a sense and/or anti-sense sequence that corresponds to a segment or portion of a targeted GA oxidase gene, and/or a sequence that is complementary thereto, although one or more sequence mismatches may be tolerated.

Engineered miRNAs are useful for targeted gene suppression with increased specificity. Sec, e.g., Parizotto et al., Genes Dev. 18:2237-2242 (2004), and U.S. Patent Application Publication Nos. 2004/0053411, 2004/0268441, 2005/0144669, and 2005/0037988, the contents and disclosures of which are incorporated herein by reference. miRNAs are non-protein coding RNAs. When a miRNA precursor molecule is cleaved, a mature miRNA is formed that is typically from about 19 to about 25 nucleotides in length (commonly from about 20 to about 24 nucleotides in length in plants), such as 19, 20, 21, 22, 23, 24, or 25 nucleotides in length, and has a sequence corresponding to the gene targeted for suppression and/or its complement. The mature miRNA hybridizes to target mRNA transcripts and guides the binding of a complex of proteins to the target transcripts, which may function to inhibit translation and/or result in degradation of the transcript, thus negatively regulating or suppressing expression of the targeted gene. miRNA precursors are also useful in plants for directing in-phase production of siRNAs, trans-acting siRNAs (ta-siRNAs), in a process that requires a RNA-dependent RNA polymerase to cause suppression of a target gene. See, e.g., Allen et al., Cell 121:207-221 (2005), Vaucheret Science STKE, 2005:pe43 (2005), and Yoshikawa et al. Genes Dev., 19:2164-2175 (2005), the contents and disclosures of which are incorporated herein by reference.

Transgenic expression of miRNAs (whether a naturally occurring sequence or an artificial sequence) can be employed to regulate expression of the miRNA's target gene or genes. Recognition sites of miRNAs have been validated in all regions of a mRNA, including the 5′ untranslated region, coding region, intron region, and 3′ untranslated region, indicating that the position of the miRNA target or recognition site relative to the coding sequence may not necessarily affect suppression (see, e.g., Jones-Rhoades and Bartel (2004). Mol. Cell, 14:787-799, Rhoades et al. (2002) Cell, 110:513-520, Allen et al. (2004) Nat. Genet., 36:1282-1290, Sunkar and Zhu (2004) Plant Cell, 16:2001-2019). miRNAs are important regulatory elements in eukaryotes, and transgenic suppression with miRNAs is a useful tool for manipulating biological pathways and responses. A description of native miRNAs, their precursors, recognition sites, and promoters is provided in U.S. Patent Application Publication No. 2006/0200878, the contents and disclosures of which are incorporated herein by reference.

Designing an artificial miRNA sequence can be achieved by substituting nucleotides in the stem region of a miRNA precursor with a sequence that is complementary to the intended target, as demonstrated, for example, by Zeng et al. (2002) Mol. Cell, 9:1327-1333. According to many embodiments, the target may be a sequence of a GA20 oxidase gene or a GA3 oxidase gene. One non-limiting example of a general method for determining nucleotide changes in a native miRNA sequence to produce an engineered miRNA precursor for a target of interest includes the following steps: (a) Selecting a unique target sequence of at least 18 nucleotides specific to the target gene, e.g., by using sequence alignment tools such as BLAST (see, for example, Altschul et al. (1990) J. Mol. Biol., 215:403-410; Altschul et al. (1997) Nucleic Acids Res., 25:3389-3402); cDNA and/or genomic DNA sequences may be used to identify target transcript orthologues and any potential matches to unrelated genes, thereby avoiding unintentional silencing or suppression of non-target sequences; (b) Analyzing the target gene for undesirable sequences (e.g., matches to sequences from non-target species), and score each potential target sequence for GC content, Reynolds score (see Reynolds et al. (2004) Nature Biotechnol., 22:326-330), and functional asymmetry characterized by a negative difference in free energy (“ΔΔG”) (see Khvorova et al. (2003) Cell, 115:209-216). Preferably, target sequences (e.g., 19-mers) may be selected that have all or most of the following characteristics: (1) a Reynolds score >4, (2) a GC content between about 40% to about 60%, (3) a negative ΔΔG. (4) a terminal adenosine, (5) lack of a consecutive run of 4 or more of the same nucleotide; (6) a location near the 3′ terminus of the target gene; (7) minimal differences from the miRNA precursor transcript. In one aspect, a non-coding RNA molecule used here to suppress a target gene (e.g., a GA20 or GA3 oxidase gene) is designed to have a target sequence exhibiting one or more, two or more, three or more, four or more, or five or more of the foregoing characteristics. Positions at every third nucleotide of a suppression element may be important in influencing RNAi efficacy; for example, an algorithm, “siExplorer” is publicly available at ma.chem.t.u-tokyo.ac.jp/siexplorer.htm (see Katoh and Suzuki (2007) Nucleic Acids Res., 10.1093/nar/gkl1120); (c) Determining a reverse complement of the selected target sequence (e.g., 19-mer) to use in making a modified mature miRNA. Relative to a 19-mer sequence, an additional nucleotide at position 20 may be matched to the selected target or recognition sequence, and the nucleotide at position 21 may be chosen to either be unpaired to prevent spreading of silencing on the target transcript or paired to the target sequence to promote spreading of silencing on the target transcript; and (d) Transforming the artificial miRNA into a plant.

According to embodiments of the present disclosure, a recombinant DNA molecule, construct or vector is provided comprising a transcribable DNA sequence or suppression element encoding a miRNA or precursor miRNA molecule for targeted suppression of a GA oxidase gene(s). Such a transcribable DNA sequence and suppression element may comprise a sequence of at least 19 nucleotides in length that corresponds to one or more GA oxidase gene(s) and/or a sequence complementary to one or more GA oxidase gene(s), although one or more sequence mismatches or non-base-paired nucleotides may be tolerated.

GA oxidase gene(s) may also be suppressed using one or more small interfering RNAs (siRNAs). The siRNA pathway involves the non-phased cleavage of a longer double-stranded RNA intermediate (“RNA duplex”) into small interfering RNAs (siRNAs). The size or length of siRNAs ranges from about 19 to about 25 nucleotides or base pairs, but common classes of siRNAs include those containing 21 or 24 base pairs. Thus, a transcribable DNA sequence or suppression element may encode an RNA molecule that is at least about 19 to about 25 nucleotides (or more) in length, such as at least 19, 20, 21, 22, 23, 24, or 25 nucleotides in length. For siRNA suppression, a recombinant DNA molecule, construct or vector is thus provided comprising a transcribable DNA sequence and suppression element encoding a siRNA molecule for targeted suppression of a GA oxidase gene(s). Such a transcribable DNA sequence and suppression element may be at least 19 nucleotides in length and have a sequence corresponding to one or more GA oxidase gene(s), and/or a sequence complementary to one or more GA oxidase gene(s).

GA oxidase gene(s) may also be suppressed using one or more trans-acting small interfering RNAs (ta-siRNAs). In the ta-siRNA pathway, miRNAs serve to guide in-phase processing of siRNA primary transcripts in a process that requires an RNA-dependent RNA polymerase for production of a double-stranded RNA precursor. ta-siRNAs are defined by lack of secondary structure, a miRNA target site that initiates production of double-stranded RNA, requirements of DCL4 and an RNA-dependent RNA polymerase (RDR6), and production of multiple perfectly phased ˜21-nt small RNAs with perfectly matched duplexes with 2-nucleotide 3′ overhangs (see Allen et al. (2005) Cell, 121:207-221). The size or length of ta-siRNAs ranges from about 20 to about 22 nucleotides or base pairs but are mostly commonly 21 base pairs. Thus, a transcribable DNA sequence or suppression element of the present disclosure may encode a RNA molecule that is at least about 20 to about 22 nucleotides in length, such as 20, 21, or 22 nucleotides in length. For ta-siRNA suppression, a recombinant DNA molecule, construct or vector is thus provided comprising a transcribable DNA sequence or suppression element encoding a ta-siRNA molecule for targeted suppression of a GA oxidase gene(s). Such a transcribable DNA sequence and suppression element may be at least 20 nucleotides in length and have a sequence corresponding to one or more GA oxidase gene(s) and/or a sequence complementary to one or more GA oxidase gene(s). For methods of constructing suitable ta-siRNA scaffolds, see, e.g., U.S. Pat. No. 9,309,512, which is incorporated herein by reference in its entirety.

According to embodiments of the present disclosure, a recombinant DNA molecule, vector or construct is provided comprising a transcribable DNA sequence encoding a non-coding RNA molecule that binds or hybridizes to a target mRNA in a plant cell, wherein the target mRNA molecule encodes a GA20 or GA3 oxidase gene, and wherein the transcribable DNA sequence is operably linked to a constitutive or tissue-specific or tissue-preferred promoter. In addition to targeting a mature mRNA sequence, a non-coding RNA molecule may instead target an intronic sequence of a GA oxidase gene or mRNA transcript, or a GA oxidase mRNA sequence overlapping coding and non-coding sequences. According to other embodiments, a recombinant DNA molecule, vector or construct is provided comprising a transcribable DNA sequence encoding a non-coding RNA (precursor) molecule that is cleaved or processed into a mature non-coding RNA molecule that binds or hybridizes to a target mRNA in a plant cell, wherein the target mRNA molecule encodes a GA20 or GA3 oxidase protein, and wherein the transcribable DNA sequence is operably linked to a constitutive or tissue-specific or tissue preferred promoter. For purposes of the present disclosure, a “non-coding RNA molecule” is an RNA molecule that does not encode a protein. Non-limiting examples of a non-coding RNA molecule include a microRNA (miRNA), a miRNA precursor, a small interfering RNA (siRNA), a siRNA precursor, a small RNA (18-26 nt in length) and precursors encoding the same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracr-RNA), a guide RNA (gRNA), and a single-guide RNA (sgRNA).

According to embodiments of the present disclosure, suitable tissue-specific or tissue preferred promoters for expression of a GA20 oxidase or GA3 oxidase suppression element may include those promoters that drive or cause expression of its associated suppression element or sequence at least in the vascular and/or leaf tissue(s) of a corn plant, or possibly other tissues in the case of GA3 oxidase. Expression of the GA oxidase suppression element or construct with a tissue-specific or tissue-preferred promoter may also occur in other tissues of the corn plant outside of the vascular and leaf tissues but active GA levels in the developing reproductive tissues of the plant (particularly in the female reproductive organ or car) are preferably not significantly reduced or impacted (relative to wild-type or control plants), such that development of the female organ or car may proceed normally in the transgenic plant without off-types in the car and a loss in yield potential.

Any vascular promoters known in the art may potentially be used as the tissue-specific or tissue-preferred promoter. Examples of vascular promoters include the RTBV promoter (see, e.g., SEQ ID NO: 65), a known sucrose synthase gene promoter, such as a corn sucrose synthase-1 (Sus1 or Sh1) promoter (see, e.g., SEQ ID NO: 67), a corn Sh1 gene paralog promoter, a barley sucrose synthase promoter (Ss1) promoter, a rice sucrose synthase-1 (RSs1) promoter (see, e.g., SEQ ID NO: 68), or a rice sucrose synthase-2 (RSs2) promoter see, e.g., SEQ ID NO: 69), a known sucrose transporter gene promoter, such as a rice sucrose transporter promoter (SUT1) (see, e.g., SEQ ID NO: 70), or various known viral promoters, such as a Commelina yellow mottle virus (CoYMV) promoter, a wheat dwarf geminivirus (WDV) large intergenic region (LIR) promoter, a maize streak geminivirus (MSV) coat protein (CP) promoter, or a rice yellow stripe 1 (YS1)-like or OsYSL2 promoter (SEQ ID NO: 71), and any functional sequence portion or truncation of any of the foregoing promoters with a similar pattern of expression, such as a truncated RTBV promoter (see, e.g., SEQ ID NO: 66).

Any leaf promoters known in the art may potentially be used as the tissue-specific or tissue-preferred promoter. Examples of leaf promoters include a corn pyruvate phosphate dikinase or PPDK promoter (see, e.g., SEQ ID NO: 72), a corn fructose 1,6 bisphosphate aldolase or FDA promoter (see, e.g., SEQ ID NO: 73), and a rice Nadh-Gogat promoter (see, e.g., SEQ ID NO: 74), and any functional sequence portion or truncation of any of the foregoing promoters with a similar pattern of expression. Other examples of leaf promoters from monocot plant genes include a ribulose biphosphate carboxylase (RuBisCO) or RuBisCO small subunit (RBCS) promoter, a chlorophyll a/b binding protein gene promoter, a phosphoenolpyruvate carboxylase (PEPC) promoter, and a Myb gene promoter, and any functional sequence portion or truncation of any of these promoters with a similar pattern of expression.

Any constitutive promoters known in the art may potentially be used. Examples of constitutive promoters that may be used in corn or maize plants include, for example, various actin gene promoters, such as a rice Actin 1 promoter (see, e.g., U.S. Pat. No. 5,641,876; see also SEQ ID NO: 75 or SEQ ID NO: 76) and a rice Actin 2 promoter (see, e.g., U.S. Pat. No. 6,429,357; see also, e.g., SEQ ID NO: 77 or SEQ ID NO: 78), a CaMV 35S or 19S promoter (see, e.g., U.S. Pat. No. 5,352,605; see also, e.g., SEQ ID NO: 79 for CaMV 35S), a maize ubiquitin promoter (see, e.g., U.S. Pat. No. 5,510,474), a Coix lacryma-jobi polyubiquitin promoter (see, e.g., SEQ ID NO: 80), a rice or maize Gos2 promoter (see, e.g., Pater et al., The Plant Journal, 2 (6): 837-44 (1992)); see also, e.g., SEQ ID NO: 81 for the rice Gos2 promoter), a FMV 35S promoter (see, e.g., U.S. Pat. No. 6,372,211), a dual enhanced CMV promoter (see, e.g., U.S. Pat. No. 5,322,938), a MMV promoter (see, e.g., U.S. Pat. No. 6,420,547; see also, e.g., SEO ID NO: 82), a PCLSV promoter (see, e.g., U.S. Pat. No. 5,850,019; see also, e.g., SEQ ID NO: 83), an Emu promoter (see, e.g., Last et al., Theor. Appl. Genet. 81:581 (1991); and Mcelroy et al., Mol. Gen. Genet. 231:150 (1991)), a tubulin promoter, an octopine synthase (ocs) promoter, a mannopine synthase (mas) promoter, or a plant alcohol dehydrogenase (e.g., maize Adh1) promoter, any other promoters including viral promoters known or later-identified in the art to provide constitutive expression in a corn plant, any other constitutive promoters known in the art that may be used in corn plants, and any functional sequence portion or truncation of any of the foregoing promoters.

Any other constitutive, vascular and/or leaf promoters known in the art may also be used, including promoter sequences from related genes (e.g., sucrose synthase, sucrose transporter, and viral gene promoter sequences) from the same or different plant species or virus that have a similar pattern of expression. Further provided are promoter sequences with a high degree of homology to any of the foregoing. For example, a vascular promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 65, 66, 67, 68, 69, 70, and 71, any functional sequence portion or truncation thereof, and/or any sequence complementary to any of the foregoing sequences; a leaf promoter may comprise, for example, a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 72, 73, and 74, any functional sequence portion or truncation thereof, and/or any sequence complementary to any of the foregoing sequences; and a constitutive promoter may comprise a DNA sequence that is at least at least 70%, at least 80%, at least 85%, at least 90%, at least 95%, at least 96%, at least 97%, at least 98%, at least 99%, at least 99.5% or 100% identical to one or more of SEQ ID NOs: 75, 76, 77, 78, 79, 80, 81, 82, and 83, any functional sequence portion or truncation thereof, and/or any sequence complementary to any of the foregoing sequences. Examples of vascular and/or leaf promoters may further include other known, engineered and/or later-identified promoter sequences shown to have a pattern of expression in vascular and/or leaf tissue(s) of a corn plant. Furthermore, any known or later-identified constitutive promoter may also be used for expression of a GA20 oxidase or GA3 oxidase suppression element. Common examples of constitutive promoters are provided below.

As understood in the art, the term “promoter” may generally refer to a DNA sequence that contains an RNA polymerase binding site, transcription start site, and/or TATA box and assists or promotes the transcription and expression of an associated transcribable polynucleotide sequence and/or gene (or transgene). A promoter may be synthetic or artificial and/or engineered, varied or derived from a known or naturally occurring promoter sequence. A promoter may be a chimeric promoter comprising a combination of two or more heterologous sequences. A promoter of the present disclosure may thus include variants of promoter sequences that are similar in composition, but not identical to, other promoter sequence(s) known or provided herein. A promoter may be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or nearly all tissues of the plant are referred to as “constitutive” promoters. However, the expression level with a “constitutive promoter” is not necessarily uniform across different tissue types and cells. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential or predominant expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. A tissue-specific or tissue-preferred promoter may also be defined in terms of the specific or preferred tissue(s) in which it drives expression of its associated transcribable DNA sequence or suppression element. For example, a promoter that causes specific expression in vascular tissues may be referred to as a “vascular specific promoter”, whereas a promoter that causes preferential or predominant expression in vascular tissues may be referred to as a “vascular-preferred promoter”. Likewise, a promoter that causes specific expression in leaf tissues may be referred to as a “leaf-specific promoter”, whereas a promoter that causes preferential or predominant expression in leaf tissues may be referred to as a “leaf preferred promoter”. An “inducible” promoter is a promoter that initiates transcription in response to an environmental stimulus such as cold, drought or light, or other stimuli, such as wounding or chemical application. A promoter may also be classified in terms of its origin, such as being heterologous, homologous, chimeric, synthetic, etc. A “heterologous” promoter is a promoter sequence having a different origin relative to its associated transcribable sequence, coding sequence, or gene (or transgene), and/or not naturally occurring in the plant species to be transformed, as defined above.

According to present embodiments, a plant cell transformed with a construct or molecule comprising a transcribable DNA sequence for suppression of an endogenous GA oxidase gene, or with a construct used for genome editing, may include any plant cell that is competent for transformation as understood in the art based on the method of transformation, such as a meristem cell, an embryonic cell, a callus cell, etc. As used herein, a “transgenic plant cell” simply refers to any plant cell that is transformed with a stably integrated recombinant DNA molecule, construct, or sequence. A transgenic plant cell may include an originally transformed plant cell, a transgenic plant cell of a regenerated or developed R₀, plant, a transgenic plant cell cultured from another transgenic plant cell, or a transgenic plant cell from any progeny plant or offspring of the transformed R₀ plant, including cell(s) of a plant seed or embryo, or a cultured plant cell, callus cell, etc.

Several of the GA oxidases in corn plants consist of a family of related GA oxidase genes. For example, corn has a family of at least nine GA20 oxidase genes that includes GA20 oxidase_1, GA20 oxidase_2, GA20 oxidase_3, GA20 oxidase_4, GA20 oxidase_5, GA20 oxidase_6, GA20 oxidase_7, GA20 oxidase_8, and GA20 oxidase_9. However, there are three known or potential GA3 oxidases in corn, GA3 oxidase_1, GA3 oxidase_2 and GA3 oxidase_3. The DNA and protein sequences by SEQ ID NOs for each of these GA20 oxidase genes are provided in Table 1, and the DNA and protein sequences by SEQ ID NOs for each of these GA3 oxidase genes are provided in Table 2.

TABLE 1
DNA and protein sequences by sequence identifier
for GA20 oxidase genes in corn.

| GA20 oxidase | | Coding Sequence | |
|---|---|---|---|
| Gene | cDNA | (CDS) | Protein |
| GA20 oxidase_1 | SEQ ID NO: 1 | SEQ ID NO: 2 | SEQ ID NO: 3 |
| GA20 oxidase_2 | SEQ ID NO: 4 | SEQ ID NO: 5 | SEQ ID NO: 6 |
| GA20 oxidase_3 | SEQ ID NO: 7 | SEQ ID NO: 8 | SEQ ID NO: 9 |
| GA20 oxidase_4 | SEQ ID NO: 10 | SEQ ID NO: 11 | SEQ ID NO: 12 |
| GA20 oxidase_5 | SEQ ID NO: 13 | SEQ ID NO: 14 | SEQ ID NO: 15 |
| GA20 oxidase_6 | SEQ ID NO: 16 | SEQ ID NO: 17 | SEQ ID NO: 18 |
| GA20 oxidase_7 | SEQ ID NO: 19 | SEQ ID NO: 20 | SEQ ID NO: 21 |
| GA20 oxidase_8 | SEQ ID NO: 22 | SEQ ID NO: 23 | SEQ ID NO: 24 |
| GA20 oxidase_9 | SEQ ID NO: 25 | SEQ ID NO: 26 | SEQ ID NO: 27 |

TABLE 2
DNA and protein sequences by sequence identifier
for GA3 oxidase genes in corn.

| GA3 oxidase | | Coding Sequence | |
|---|---|---|---|
| Gene | cDNA | (CDS) | Protein |
| GA3 oxidase_1 | SEQ ID NO: 28 | SEQ ID NO: 29 | SEQ ID NO: 30 |
| GA3 oxidase_2 | SEQ ID NO: 31 | SEQ ID NO: 32 | SEQ ID NO: 33 |
| GA3 oxidase_3 | SEQ ID NO: 87 | SEQ ID NO: 88 | SEQ ID NO: 89 |

The genomic DNA sequence of GA20 oxidase_3 is provided in SEQ ID NO: 34, and the genomic DNA sequence of GA20 oxidase_5 is provided in SEQ ID NO: 35. For the GA20 oxidase_3 gene, SEQ ID NO: 34 provides 3000 nucleotides upstream of the GA20 oxidase_3 5′-UTR; nucleotides 3001-3096 correspond to the 5′-UTR; nucleotides 3097-3665 correspond to the first exon; nucleotides 3666-3775 correspond to the first intron; nucleotides 3776-4097 correspond to the second exon; nucleotides 4098-5314 correspond to the second intron; nucleotides 5315-5584 correspond to the third exon; and nucleotides 5585-5800 correspond to the 3′-UTR. SEQ ID NO: 34 also provides 3000 nucleotides downstream of the end of the 3′-UTR (nucleotides 5801-8800). For the GA20 oxidase_5 gene, SEQ ID NO: 35 provides 3000 nucleotides upstream of the GA20 oxidase_5 start codon (nucleotides 1-3000); nucleotides 3001-3791 correspond to the first exon; nucleotides 3792-3906 correspond to the first intron; nucleotides 3907-4475 correspond to the second exon; nucleotides 4476-5197 correspond to the second intron; nucleotides 5198-5473 correspond to the third exon; and nucleotides 5474-5859 correspond to the 3′-UTR. SEQ ID NO: 35 also provides 3000 nucleotides downstream of the end of the 3′-UTR (nucleotides 5860-8859).

The genomic DNA sequence of GA3 oxidase_1 is provided in SEQ ID NO: 36 and 84, and the genomic DNA sequence of GA3 oxidase_2 is provided in SEQ ID NO: 37 and 85, and the genomic DNA sequence of GA3 oxidase_3 is provided in SEQ ID NO: 86. While SEQ ID NOs: 36 and 37 provide 5′-UTR, exon, intron and 3′-UTR sequences for the GA3 oxidase_1 and GA3 oxidase_2 genes, respectively, SEQ ID NOs: 84 and 85 further provide upstream and downstream genomic sequences and additional 5′ and 3′ UTR sequences for the GA3 oxidase_1 and GA3 oxidase_2 genes, respectively. For the GA3 oxidase_1 gene, nucleotides 1-29 of SEQ ID NO: 36 correspond to the 5′-UTR; nucleotides 30-514 of SEQ ID NO: 36 correspond to the first exon; nucleotides 515-879 of SEQ ID NO: 36 correspond to the first intron; nucleotides 880-1038 of SEQ ID NO: 36 correspond to the second exon; nucleotides 1039-1158 of SEQ ID NO: 36 correspond to the second intron; nucleotides 1159-1663 of SEQ ID NO: 36 correspond to the third exon; and nucleotides 1664-1788 of SEQ ID NO: 36 correspond to the 3′-UTR. Alternatively for the GA3 oxidase_1 gene, SEQ ID NO: 84 provides 3000 nucleotides upstream of the GA3 oxidase_1 5′-UTR (nucleotides 1-3000); nucleotides 3001-3161 of SEQ ID NO: 84 correspond to the 5′-UTR; nucleotides 3162-3646 of SEQ ID NO: 84 correspond to the first exon; nucleotides 3647-4011 of SEQ ID NO: 168 correspond to the first intron; nucleotides 4012-4170 of SEQ ID NO: 84 correspond to the second exon; nucleotides 4171-4290 of SEQ ID NO: 84 correspond to the second intron; nucleotides 4291-4795 of SEQ ID NO: 84 correspond to the third exon; and nucleotides 4796-5406 of SEQ ID NO: A correspond to the 3′-UTR. SEQ ID NO: 84 also provides 3000 nucleotides downstream of the end of the 3′-UTR (nucleotides 5407-8406). For the GA3 oxidase_2 gene, nucleotides 1-38 of SEQ ID NO: 37 correspond to the 5′-UTR; nucleotides 39-532 of SEQ ID NO: 37 correspond to the first exon; nucleotides 533-692 of SEQ ID NO: 37 correspond to the first intron; nucleotides 693-851 of SEQ ID NO: 37 correspond to the second exon; nucleotides 852-982 of SEQ ID NO: 37 correspond to the second intron; nucleotides 983-1445 of SEQ ID NO: 37 correspond to the third exon; and nucleotides 1446-1698 of SEQ ID NO: 37 correspond to the 3′-UTR. Alternatively for the GA3 oxidase_2 gene, SEQ ID NO: 85 provides 3000 nucleotides upstream of the GA3 oxidase_2 5′-UTR (nucleotides 1-3000); nucleotides 3001-3056 of SEQ ID NO: 169 correspond to the 5′-UTR; nucleotides 3057-3550 of SEQ ID NO: 85 correspond to the first exon; nucleotides 3551-3710 of SEQ ID NO: 85 correspond to the first intron; nucleotides 3711-3869 of SEQ ID NO: 85 correspond to the second exon; nucleotides 3870-3991 of SEQ ID NO: 85 correspond to the second intron; nucleotides 3992-4463 of SEQ ID NO: 85 correspond to the third exon; and nucleotides 4464-4581 of SEQ ID NO: 85 correspond to the 3′-UTR. SEQ ID NO: 85 also provides 3000 nucleotides downstream of the end of the 3′-UTR (nucleotides 4582-7581). For the GA3 oxidase_3 gene, SEQ ID NO: 86 provides 3000 nucleotides upstream of the GA3 oxidase_3 5′-UTR (nucleotides 1-3000); nucleotides 3001-3130 of SEQ ID NO: 86 correspond to the 5′-UTR; nucleotides 3131-3483 of SEQ ID NO: 86 correspond to the first exon; nucleotides 3484-3582 of SEQ ID NO: 86 correspond to the first intron; nucleotides 3583-3907 of SEQ ID NO: 86 correspond to the second exon; nucleotides 3908-3998 of SEQ ID NO: C correspond to the second intron; nucleotides 3999-4274 of SEQ ID NO: 86 correspond to the third exon; and nucleotides 4275-4332 of SEQ ID NO: 86 correspond to the 3′-UTR. SEQ ID NO: 86 also provides 3000 nucleotides downstream of the end of the 3′-UTR (nucleotides 4333-7332).

In addition to phenotypic observations with targeting the GA20 oxidase_3 and/or GA20 oxidase_5 gene(s), for suppression, a semi-dwarf phenotype is also observed with suppression of the GA20 oxidase_4 gene. The genomic DNA sequence of GA20 oxidase_4 is provided in SEQ ID NO: 38. For the GA20 oxidase_4 gene, SEQ ID NO: 38 provides nucleotides 1-1416 upstream of the 5′-UTR; nucleotides 1417-1543 of SEQ ID NO: 38 correspond to the 5′-UTR; nucleotides 1544-1995 of SEQ ID NO: 38 correspond to the first exon; nucleotides 1996-2083 of SEQ ID NO: 38 correspond to the first intron; nucleotides 2084-2411 of SEQ ID NO: 38 correspond to the second exon; nucleotides 2412-2516 of SEQ ID NO: 38 correspond to the second intron; nucleotides 2517-2852 of SEQ ID NO: 38 correspond to the third exon; nucleotides 2853-3066 of SEQ ID NO: 38 correspond to the 3′-UTR; and nucleotides 3067-4465 of SEQ ID NO: 38 corresponds to genomic sequence downstream of to the 3′-UTR.

According to embodiments of the present disclosure, a recombinant DNA molecule, vector or construct is provided comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule (i) expressed from an endogenous GA oxidase gene and/or (ii) encoding an endogenous GA oxidase protein in the plant, wherein the transcribable DNA sequence is operably linked to a plant-expressible promoter, and wherein the plant is a corn or maize plant.

According to some embodiments, a non-coding RNA molecule targets GA20 oxidase gene(s), such as GA20 oxidase_3 and/or GA20 oxidase_5 gene(s), for suppression and comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or more of SEQ ID NOS: 7, 8, 13 and 14. According to some embodiments, a non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to one or both of SEQ ID NOs: 9 and 15. According to further embodiments, a non-coding RNA molecule may comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% similar to one or both of SEQ ID NOs: 9 and 15. In addition to targeting a mature mRNA sequence (including either or both of the untranslated or exonic sequences), a non-coding RNA molecule may further target the intronic sequences of a GA20 oxidase gene or transcript.

According to some embodiments, a non-coding RNA molecule targets GA3 oxidase gene(s) for suppression and comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or more of SEQ ID NOs: 28, 29, 31, 32, 87 and 88. According to other embodiments, a non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to one or both of SEQ ID NOs: 30, 33 and 89. According to further embodiments, a non-coding RNA molecule may comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% similar to one or both of SEQ ID NOs: 30, 33 and 89. In addition to targeting a mature mRNA sequence (including either or both of the untranslated or exonic sequences), a non-coding RNA molecule may further target the intronic sequences of a GA3 oxidase gene or transcript.

According to some embodiments, a non-coding RNA molecule targets GA20 oxidase_4 gene for suppression and comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of one or both of SEQ ID NOs: 10 and 11. According to other embodiments, a non-coding RNA molecule is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to one or both of SEQ ID NO: 12. According to further embodiments, a non-coding RNA molecule may comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% similar to SEQ ID NOs: 12. In addition to targeting a mature mRNA sequence (including either or both of the untranslated or exonic sequences), a non-coding RNA molecule may further target the intronic sequences of a GA20 oxidase gene or transcript.

In particular embodiments, the maize plant or maize plant part may comprise: i) an endogenous GA20 oxidase_3 gene and a mutant allele of the endogenous GA20 oxidase gene comprising one or more mutations relative to SEQ ID NO: 7, SEQ ID NO: 8, and/or SEQ ID NO: 34; ii) an endogenous GA20 oxidase_4 gene and a mutant allele of the endogenous GA20 oxidase gene comprising one or more mutations relative to SEQ ID NO: 10, SEQ ID NO: 11, and/or SEQ ID NO: 38; iii) an endogenous GA20 oxidase_5 gene and a mutant allele of the endogenous GA20 oxidase gene comprising one or more mutations relative to SEQ ID NO: 13, SEQ ID NO: 14, and/or SEQ ID NO: 35; iv) an endogenous GA3 oxidase_/gene and a mutant allele of the endogenous GA3 oxidase gene comprising one or more mutations relative to SEQ ID NO: 28, SEQ ID NO: 29, SEQ ID NO: 36 and/or SEQ ID NO: 84; v) an endogenous GA3 oxidase_2 gene and a mutant allele of the endogenous GA3 oxidase gene comprising one or more mutations relative to SEQ ID NO: 31, SEQ ID NO: 32, and/or SEQ ID NO: 37 and/or SEQ ID NO: 85; or vi) an endogenous GA3 oxidase_3 gene and a mutant allele of the endogenous GA3 oxidase gene comprising one or more mutations relative to SEQ ID NO: 86, SEQ ID NO: 87, and/or SEQ ID NO: 88. Alternatively, the maize plant or maize plant part may comprise a mutant of the endogenous GA20 oxidase_3 gene and a mutant allele of the endogenous GA20 oxidase_5 gene. For example, the maize plant or maize plant part may be homozygous for a mutant allele of the endogenous GA20 oxidase_3 gene and heterozygous for a mutant allele of the endogenous GA20 oxidase_5 gene. Alternatively, the maize plant or maize plant part may be heterozygous for a mutant allele of the endogenous GA20 oxidase_3 gene and homozygous for a mutant allele of the endogenous GA20 oxidase_5 gene. The maize plant or maize plant part may be heteroallelic for two mutant alleles of the endogenous GA20 oxidase_3 gene comprising a first mutant allele of the endogenous GA20 oxidase_3 gene and a second mutant allele of the endogenous GA20 oxidase_3 gene, and heterozygous for a mutant allele of the endogenous GA20 oxidase_5 gene. Alternatively, the maize plant or maize plant part may be heteroallelic for two mutant alleles of the endogenous GA20 oxidase_5 gene comprising a first mutant allele of the endogenous GA20 oxidase_5 gene and a second mutant allele of the endogenous GA20 oxidase_5 gene, and heterozygous for a mutant allele of the endogenous GA20 oxidase_3 gene. Alternatively, the maize plant or maize plant part may have an expression level and/or activity of an mRNA and/or protein encoded by the mutant allele of the endogenous GA20 oxidase gene reduced relative to the expression level and/or activity of the mRNA and/or protein encoded by a wild-type allele of the same GA20 oxidase gene. Still further, the maize plant or maize plant part may have an expression level and/or activity of the mRNA and/or protein encoded by the mutant allele of the endogenous GA3 oxidase gene reduced relative to the expression level and/or activity of the mRNA and/or protein encoded by a wild-type allele of the same GA3 oxidase gene

According to many embodiments, the non-coding RNA molecule encoded by the transcribable DNA sequence of the recombinant DNA molecule, vector or construct may be a precursor miRNA or siRNA that is processed or cleaved in a plant cell to form a mature miRNA or siRNA that targets a GA20 oxidase or GA3 oxidase gene.

According to embodiments of the present disclosure, GA levels may be reduced in the stalk or stem of a corn or maize plant by targeting only a limited subset of genes within a GA oxidase family for suppression. Without being bound by theory, it is proposed that targeting of a limited number of genes within a GA oxidase family for suppression may produce the short stature phenotype and resistance to lodging in transgenic plants, but without off types in the reproductive or ear tissues of the plant due to differential expression among GA oxidase genes, sufficient compensation for the suppressed GA oxidase gene(s) by other GA oxidase gene(s) in those reproductive tissues, and/or incomplete suppression of the targeted GA oxidase gene(s). Thus, not only may off-types be avoided by limiting expression or suppression of GA oxidase gene(s) with a tissue-specific or tissue preferred promoter, it is proposed that a limited subset of GA oxidase genes (e.g., a limited number of GA20 oxidase genes) may be targeted for suppression, such that the other GA oxidase genes within the same gene family (e.g., other GA20 oxidase genes) may compensate for loss of expression of the suppressed GA oxidase gene(s) in those tissues. Incomplete suppression of the targeted GA oxidase gene(s) may also allow for a sufficient level of expression of the targeted GA oxidase gene(s) in one or more tissues to avoid off-types or undesirable traits in the plant that would negatively affect crop yield, such as reproductive off-types or excessive shortening of plant height. Unlike complete loss-of-function mutations in a gene, suppression may allow for partial activity of the targeted gene to persist. Since the different GA20 oxidase genes have different patterns of expression in plants, targeting of a limited subset of GA20 oxidase genes for suppression may allow for modification of certain traits while avoiding off-types previously associated with GA mutants in corn or maize plants. In other words, the growth, developmental and reproductive traits or off-types previously associated with GA mutants in corn may be decoupled by targeting only a limited number or subset (i.e., one or more, but not all) of the GA20 or GA3 oxidase genes and/or by incomplete suppression of a targeted GA oxidase gene. By transgenically targeting a subset of one or more endogenous GA3 or GA20 oxidase genes for suppression within a plant, a more pervasive pattern of expression (e.g., with a constitutive promoter) may be used to produce semi-dwarf plants without significant reproductive off-types and/or other undesirable traits in the plant, even with expression of the transgenic construct in reproductive tissue(s). Suppression elements and constructs that selectively target the GA20 oxidase 3 and/or GA20 oxidase_5genes may be operably linked to a vascular, leaf and/or constitutive promoter.

With a suppression construct that only targets a limited subset of GA20 oxidase genes, such as the GA20 oxidase_3, GA20 oxidase_4, and/or GA20 oxidase_5 gene(s), or which targets the GA3 oxidase_1, GA3 oxidase_2 and/or GA3 oxidase_3 gene(s), restricting the pattern of expression of the suppression element may be less crucial for obtaining normal reproductive development of the corn or maize plant and avoidance of off-types in the female organ or car due to compensation, etc., from the other GA20 and/or GA3 oxidase genes. Therefore, expression of a suppression construct and element, selectively or preferentially targeting, for instance, the GA20 oxidase_3 and/or GA20 oxidase_5 gene(s), the GA20 oxidase_4 gene, and/or the GA3 oxidase_1, GA3 oxidase_2 and/or GA3 oxidase_3 gene(s) in corn, may be driven by a variety of different plant-expressible promoter types including constitutive and tissue-specific or tissue-preferred promoters, such as a vascular or leaf promoter, which may include, for example, the RTBV promoter introduced above (e.g., a promoter comprising the RTBV (SEQ ID NO: 65) or truncated RTBV (SEQ ID NO: 66) sequence), and any other promoters that drive expression in tissues encompassing much or all of the vascular and/or leaf tissue(s) of a plant. Any known or later-identified constitutive promoter with a sufficiently high level of expression may also be used for expression of a suppression construct targeting a subset of GA20 and/or GA3 oxidase genes in corn, particularly the GA20 oxidase_3 and/or GA20 oxidase_5 gene(s), the GA20 oxidase_4 gene, and/or the GA3 oxidase_1, GA3 oxidase_2 and/or GA3 oxidase_3 gene(s).

A sufficient level of expression of a transcribable DNA sequence encoding a non-coding RNA molecule targeting a GA oxidase gene for suppression may be necessary to produce a short stature, semi-dwarf phenotype that resists lodging, since lower levels of expression may be insufficient to lower active GA levels in the plant to a sufficient extent to cause a significant phenotype. Thus, tissue-specific and tissue-preferred promoters that drive, etc., a moderate or strong level of expression of their associated transcribable DNA sequence in active GA-producing tissue(s) of a plant may be preferred. Furthermore, such tissue-specific and tissue-preferred should drive, etc., expression of their associated transcribable DNA sequence during one or more vegetative stage(s) of plant development when the plant is growing and/or elongating including one or more of the following vegetative stage(s): V_{E}, V1, V2, V3, V4, V5, V6, V7, V8, V9, V10, V11, V12, V13, V14, Vn, V_{T}, such as expression at least during V3-V12, V4-V12, V5-V12, V6-V12, V7-V12, V8-V12, V3-V14, V5-V14, V6-V14, V7-V14, V8-V14, V9-V14, V10-V14, etc., or during any other range of vegetative stages when growth and/or elongation of the plant is occurring.

According to many embodiments, the plant-expressible promoter may preferably drive expression constitutively or in at least a portion of the vascular and/or leaf tissues of the plant. Different promoters driving expression of a suppression element targeting the endogenous GA20 oxidase_3 and/or GA20 oxidase_5 gene(s), the GA20 oxidase_4 gene, the GA3 oxidase_1, GA3 oxidase_2 and/or GA3 oxidase_3 gene(s) in corn, may be effective at reducing plant height and increasing lodging resistance to varying degrees depending on their particular pattern and strength of expression in the plant. However, some tissue-specific and tissue-preferred promoters driving expression of a GA20 or GA3 oxidase suppression element in a plant may not produce a significant short stature or anti-lodging phenotypes due to the spatial temporal pattern of expression of the promoter during plant development, and/or the amount or strength of expression of the promoter being too low or weak. Furthermore, some suppression constructs may only reduce and not eliminate expression of the targeted GA20 or GA3 oxidase gene(s) when expressed in a plant, and thus depending on the pattern and strength of expression with a given promoter, the pattern and level of expression of the GA20 or GA3 oxidase suppression construct with such a promoter may not be sufficient to produce an observable plant height and lodging resistance phenotype in plants.

According to present embodiments, a recombinant DNA molecule, vector or construct for suppression of one or more endogenous GA20 or GA3 oxidase gene(s) in a plant is provided comprising a transcribable DNA sequence encoding a non-coding RNA molecule, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule expressed from an endogenous GA oxidase gene and encoding an endogenous GA oxidase protein in the plant, wherein the transcribable DNA sequence is operably linked to a plant-expressible promoter, and wherein the plant is a corn plant. As stated above, in addition to targeting a mature mRNA sequence, a non-coding RNA molecule may further target the intronic sequence(s) of a GA oxidase gene or transcript. According to some embodiments, a non-coding RNA molecule may target a GA20 oxidase 3 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7 or SEQ ID NO: 8. According to some embodiments, a non-coding RNA molecule targeting a GA20 oxidase 3 gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEO ID NO: 7 or SEO ID NO: 8. According to some embodiments, a non-coding RNA molecule may target a GA20 oxidase gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 9.

According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein that is at least 80%, at least 85%, at least 90%, at least 95%, at least 96%, at least 97%, at least 98%, at least 99%, at least 99.5%, or 100% similar to SEQ ID NO: 9.

As mentioned above, a non-coding RNA molecule may target an intron sequence of a GA oxidase gene instead of, or in addition to, an exonic, 5′-UTR or 3′-UTR of the GA oxidase gene. Thus, a non-coding RNA molecule targeting the GA20 oxidase 3 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 34, and/or of nucleotides 3666-3775 or 4098-5314 of SEQ ID NO: 34. It is important to note that the sequences provided herein for the GA20 oxidase_3 gene may vary across the diversity of corn plants, lines and germplasms due to polymorphisms and/or the presence of different alleles of the gene. Furthermore, a GA20 oxidase_3 gene may be expressed as alternatively spliced isoforms that may give rise to different mRNA, cDNA and coding sequences that can affect the design of a suppression construct and non-coding RNA molecule. Thus, a non-coding RNA molecule targeting a GA20 oxidase_3 gene for suppression may be more broadly defined as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 34.

According to embodiments of the present disclosure, a recombinant DNA molecule, vector or construct for suppression of an endogenous GA20 oxidase_5 gene in a plant is provided comprising a transcribable DNA sequence encoding a non-coding RNA molecule, wherein the non-coding RNA molecule targeting the GA20 oxidase_5 gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 13 or SEQ ID NO: 14. According to some embodiments, a non-coding RNA molecule targeting the GA20 oxidase_5 gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 13 or SEQ ID NO: 14. According to some embodiments, a non-coding RNA molecule may target a GA20 oxidase gene for suppression comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 15. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein that is at least 80%, at least 85%, at least 90%, at least 95%, at least 96%, at least 97%, at least 98%, at least 99%, at least 99.5%, or 100% similar to SEQ ID NO: 15.

As mentioned above, a non-coding RNA molecule may target an intron sequence of a GA oxidase gene instead of, or in addition to, an exonic or untranslated region of the mature mRNA of the GA oxidase gene. Thus, a non-coding RNA molecule targeting the GA20 oxidase_5 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 35, and/or of nucleotides 3792-3906 or 4476-5197 of SEQ ID NO: 35. The sequences provided herein for GA20 oxidase_5 may vary across the diversity of corn plants, lines and germplasms due to poly morphisms and/or the presence of different alleles of the gene. Furthermore, a GA20 oxidase_5 gene may be expressed as alternatively spliced isoforms that may give rise to different mRNA, cDNA and coding sequences that can affect the design of a suppression construct and non-coding RNA molecule. Thus, a non-coding RNA molecule targeting a GA20 oxidase_3 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 35.

According to further embodiments, a recombinant DNA molecule, vector or construct for joint suppression of endogenous GA20 oxidase_3 and GA20 oxidase_5 genes in a plant is provided comprising a transcribable DNA sequence encoding a non-coding RNA molecule, wherein the non-coding RNA molecule targeting the GA20 oxidase_3 and GA20 oxidase_5 genes for suppression comprises a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 7 and/or SEO ID NO: 8, and (ii) at least 80% at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 13 and/or SEQ ID NO: 14. According to some of these embodiments, the non-coding RNA molecule jointly targeting the GA20 oxidase_3 and GA20 oxidase_5 genes for suppression may be complementary to at least 19 consecutive nucleotides, but no more than 27 consecutive nucleotides, such as complementary to 19, 20, 21, 22, 23, 24, 25, 26, or 27 consecutive nucleotides, of (i) SEO ID NO: 7 (and/or SEQ ID NO: 8) and (ii) SEQ ID NO: 13 (and/or SEQ ID NO: 14). According to many embodiments, a non-coding RNA molecule jointly targeting the GA20 oxidase_3 and GA20 oxidase_5 genes for suppression comprises a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 9, and (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 15. As mentioned above, the non-coding RNA molecule may target an intron sequence of a GA oxidase gene. Thus, the non-coding RNA molecule may target an intron sequence(s) of one or both of the GA20 oxidase_3 and/or GA20 oxidase_5 gene(s) as identified above.

According to particular embodiments, the non-coding RNA molecule encoded by a transcribable DNA sequence comprises (i) a sequence that is at least 95%, at least 96%, at least 97%, at least 98%, at least 99%, at least 99.5%, or 100% complementary to SEQ ID NO: 39, 41, 43 or 45, and/or (ii) a sequence or suppression element encoding a non-coding RNA molecule comprising a sequence that is at least 95%, at least 96%, at least 97%, at least 98%, at least 99%, at least 99.5%, or 100% identical to SEQ ID NO: 40, 42, 44 or 46. According to some embodiments, the non-coding RNA molecule encoded by a transcribable DNA sequence may comprise a sequence with one or more mismatches, such as 1, 2, 3, 4, 5 or more complementary mismatches, relative to the sequence of a target or recognition site of a targeted GA20 oxidase gene mRNA, such as a sequence that is nearly complementary to SEQ ID NO: 40 but with one or more complementary mismatches relative to SEQ ID NO: 40. According to a particular embodiment, the non-coding RNA molecule encoded by the transcribable DNA sequence comprises a sequence that is 100% identical to SEQ ID NO: 40, which is 100% complementary to a target sequence within the cDNA and coding sequences of the GA20 oxidase_3 (i.e., SEQ ID NOs: 7 and 8 respectively), and/or to a corresponding sequence of a mRNA encoded by an endogenous GA20 oxidase_3 gene. However, the sequence of a non-coding RNA molecule encoded by a transcribable DNA sequence that is 100% identical to SEQ ID NO: 40, 42, 44 or 46 may not be perfectly complementary to a target sequence within the cDNA and coding sequences of the GA20 oxidase_5 gene (i.e., SEQ ID NOs: 13 and 14, respectively), and/or to a corresponding sequence of a mRNA encoded by an endogenous GA20 oxidase_5 gene. For example, the closest complementary match between the non-coding RNA molecule or miRNA sequence in SEQ ID NO: 40 and the cDNA and coding sequences of the GA20 oxidase_5 gene may include one mismatch at the first position of SEQ ID NO: 39 (i.e., the “C” at the first position of SEQ ID NO: 39 is replaced with a “G”; i.e., GTCCATCATGCGGTGCAACTA). However, the non-coding RNA molecule or miRNA sequence in SEQ ID NO: 40 may still bind and hybridize to the mRNA encoded by the endogenous GA20 oxidase_5 gene despite this slight mismatch.

According to embodiments of the present disclosure, a recombinant DNA molecule, vector or construct for suppression of one or more endogenous GA3 oxidase gene(s) in a plant is provided comprising a transcribable DNA sequence encoding a non-coding RNA molecule wherein the non-coding RNA molecule comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule expressed from an endogenous GA3 oxidase gene and encoding an endogenous GA3 oxidase protein in the plant, wherein the transcribable DNA sequence is operably linked to a plant-expressible promoter, and wherein the plant is a corn or maize plant. In addition to targeting a mature mRNA sequence, a non-coding RNA molecule may further target the intronic sequences of a GA3 oxidase gene or transcript.

According to some embodiments, a non-coding RNA molecule may target a GA3 oxidase_1 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 28 or SEQ ID NO: 29. According to some embodiments, a non-coding RNA molecule targeting a GA3 oxidase gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 28 or SEQ ID NO: 29. According to some embodiments, a non-coding RNA molecule targeting a GA3 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 30. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein that is at least 80%, at least 85%, at least 90%, at least 95%, at least 96%, at least 97%, at least 98%, at least 99%, at least 99.5%, or 100% similar to SEQ ID NO: 30.

As mentioned above, a non-coding RNA molecule may target an intron sequence of a GA3 oxidase gene instead of, or in addition to, an exonic, 5′-UTR or 3′-UTR of the GA oxidase gene. Thus, a non-coding RNA molecule targeting the GA3 oxidase_1 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 36 and/or 84, and/or of nucleotides 515-879 or 1039-1158 of SEQ ID NO: 36, and/or of nucleotides 3647-4011 or 4171-4290 of SEQ ID NO: 84. The sequences provided herein for GA3 oxidase_1 may vary across the diversity of corn plants, lines and germ plasms due to polymorphisms and/or the presence of different alleles of the gene. Furthermore, a GA3 oxidase_1 gene may be expressed as alternatively spliced isoforms that may that can affect the design of a suppression construct and non-coding RNA molecule. Thus, a non-coding RNA molecule targeting a GA3 oxidase_1 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 36 and/or 84.

According to some embodiments, a non-coding RNA molecule may target a GA3 oxidase_2 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 31 or SEQ ID NO: 32. According to some embodiments, a non-coding RNA molecule targeting the GA3 oxidase gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 31 or SEQ ID NO: 32. According to some embodiments, a non-coding RNA molecule targeting the GA3 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 33. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein that is at least 80%, at least 85%, at least 90%, at least 95%, at least 96%, at least 97%, at least 98%, at least 99%, at least 99.5%, or 100% similar to SEQ ID NO: 33.

As mentioned above, a non-coding RNA molecule may target an intron sequence of a GA3 oxidase gene instead of, or in addition to, an exonic, 5′-UTR or 3′-UTR of the GA3 oxidase gene. Thus, a non-coding RNA molecule targeting the GA3 oxidase_2 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 37 and/or 85, and/or of nucleotides 533-692 or 852-982 of SEQ ID NO: 37, and/or of nucleotides 3551-3710 or 3870-3991 of SEQ ID NO: 85. The sequences provided herein for GA3 oxidase_2 may vary across the diversity of corn plants, lines and germ plasms due to polymorphisms and/or the presence of different alleles of the gene. Furthermore, a GA3 oxidase_2 gene may be expressed as alternatively spliced isoforms that may give rise to different mRNA, cDNA and coding sequences that can affect the design of a suppression construct and non-coding RNA molecule. Thus, a non-coding RNA molecule targeting a GA3 oxidase_2 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 37 and/or 85.

According to some embodiments, a non-coding RNA molecule may target a GA3 oxidase_3 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 87 or SEQ ID NO: 88. According to some embodiments, a non-coding RNA molecule targeting the GA3 oxidase gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEQ ID NO: 87 or SEQ ID NO: 88. According to some embodiments, a non-coding RNA molecule targeting the GA3 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 89. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein that is at least 80%, at least 85%, at least 90%, at least 95%, at least 96%, at least 97%, at least 98%, at least 99%, at least 99.5%, or 100% similar to SEQ ID NO: 89.

As mentioned above, a non-coding RNA molecule may target an intron sequence of a GA3 oxidase gene instead of, or in addition to, an exonic, 5′ UTR or 3′ UTR of the GA3 oxidase gene. Thus, a non-coding RNA molecule targeting the GA3 oxidase_3 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 86 and/or of nucleotides 3484-3582 or 3908-3998 of SEQ ID NO: 86. The sequences provided herein for GA3 oxidase_3 may vary across the diversity of corn plants, lines and germplasms due to polymorphisms and/or the presence of different alleles of the gene. Furthermore, a GA3 oxidase_3 gene may be expressed as alternatively spliced isoforms that may give rise to different mRNA, cDNA and coding sequences that can affect the design of a suppression construct and non-coding RNA molecule. Thus, a non-coding RNA molecule targeting a GA3 oxidase_3 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 86.

According to particular embodiments, a non-coding RNA molecule encoded by a transcribable DNA sequence for targeting a GA3 oxidase gene comprises (i) a sequence that is at least 95%, at least 96%, at least 97%, at least 98%, at least 99%, at least 99.5%, or 100% complementary to SEQ ID NO: 57 or 59, and/or (ii) a sequence or suppression element encoding a non-coding RNA molecule comprising a sequence that is at least 95%, at least 96%, at least 97%, at least 98%, at least 99%, at least 99.5%, or 100% identical to SEQ ID NO: 58 or 60. According to some embodiments, the non-coding RNA molecule encoded by a transcribable DNA sequence may comprise a sequence with one or more mismatches, such as 1, 2, 3, 4, 5 or more complementary mismatches, relative to the sequence of a target or recognition site of a targeted GA3 oxidase gene mRNA, such as a sequence that is nearly complementary to SEQ ID NO: 57 or 59 but with one or more complementary mismatches relative to SEQ ID NO: 57 or 59. According to a particular embodiment, the non-coding RNA molecule encoded by the transcribable DNA sequence comprises a sequence that is 100% identical to SEQ ID NO: 58 or 60, which is 100% complementary to a target sequence within the cDNA and coding sequences of a GA3 oxidase_1 or GA3 oxidase_2 gene in corn (i.e., SEQ ID NOs: 28, 29, 31 and/or 32), and/or to a corresponding sequence of a mRNA encoded by an endogenous GA3 oxidase_1 or GA3 oxidase_2 gene.

According to some embodiments, a non-coding RNA molecule may target a GA20 oxidase_4 gene for suppression and comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 10 or SEQ ID NO: 11. According to some embodiments, a non-coding RNA molecule targeting a GA20 oxidase_4 gene for suppression may be complementary to at least 19 consecutive nucleotides, but no more than 27 consecutive nucleotides, such as complementary to 19, 20, 21, 22, 23, 24, 25, 26, or 27 consecutive nucleotides, of SEO ID NO: 10 or SEO ID NO: 11. According to some embodiments, a non-coding RNA molecule targeting the GA20 oxidase gene for suppression comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 12. According to further embodiments, a non-coding RNA molecule may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein that is at least 80%, at least 85%, at least 90%, at least 95%, at least 96%, at least 97%, at least 98%, at least 99%, at least 99.5%, or 100% similar to SEQ ID NO: 12.

As mentioned above, a non-coding RNA molecule may target an intron sequence of a GA20 oxidase gene instead of, or in addition to, an exonic, 5′-UTR or 3′-UTR of the GA20 oxidase gene. Thus, a non-coding RNA molecule targeting a GA20 oxidase_4 gene for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 38, and/or of nucleotides 1996-2083 or 2412-2516 of SEQ ID NO: 38. The sequences provided herein for GA20 oxidase_4 may vary across the diversity of corn plants, lines and germplasms due to polymorphisms and/or the presence of different alleles of the gene. Furthermore, a GA20 oxidase_4 gene may be expressed as alternatively spliced isoforms that may give rise to different mRNA, cDNA and coding sequences that can affect the design of a suppression construct and non-coding RNA molecule. Thus, a non-coding RNA molecule targeting a GA20 oxidase_4 gene for suppression may be defined more broadly as comprising a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of SEQ ID NO: 38.

According to particular embodiments, a non-coding RNA molecule encoded by a transcribable DNA sequence for targeting a GA20 oxidase_4 gene comprises (i) a sequence that is at least 95%, at least 96%, at least 97%, at least 98%, at least 99%, at least 99.5%, or 100% complementary to SEQ ID NO: 61, and/or (ii) a sequence or suppression element encoding a non-coding RNA molecule comprising a sequence that is at least 95%, at least 96%, at least 97%, at least 98%, at least 99%, at least 99.5%, or 100% identical to SEQ ID NO: 62. According to some embodiments, the non-coding RNA molecule encoded by a transcribable DNA sequence may comprise a sequence with one or more mismatches, such as 1, 2, 3, 4, 5 or more complementary mismatches, relative to the sequence of a target or recognition site of a targeted GA20 oxidase gene mRNA, such as a sequence that is nearly complementary to SEQ ID NO: 61 but with one or more complementary mismatches relative to SEQ ID NO: 61. According to a particular embodiment, the non-coding RNA molecule encoded by the transcribable DNA sequence comprises a sequence that is 100% identical to SEQ ID NO: 62, which is 100% complementary to a target sequence within the cDNA and coding sequences of a GA20 oxidase_4 gene in corn (i.e., SEQ ID NO: 10 or 11), and/or to a corresponding sequence of a mRNA encoded by an endogenous GA20 oxidase_4 gene.

According to embodiments of the present disclosure, a recombinant DNA construct is provided comprising a transcribable DNA sequence encoding a non-coding RNA molecule targeting an endogenous GA20 oxidase_3 and/or the GA20 oxidase_5 gene(s) for suppression, wherein the transcribable DNA sequence is operably linked to a constitutive, tissue-specific or tissue-preferred promoter, and wherein the transcribable DNA sequence causes the expression level of an endogenous GA20 oxidase_3 and/or the GA20 oxidase_5 gene(s) to become reduced or lowered in one or more tissue(s) of a plant transformed with the transcribable DNA sequence. Such a non-coding RNA molecule encoded by the transcribable DNA sequence may comprise a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 9, and/or (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA20 oxidase protein in the plant that is at least 80%, at least 85%, at least 90%, at least 95%, at least 96%, at least 97%, at least 98%, at least 99%, at least 99.5%, or 100% identical to SEQ ID NO: 15.

According to embodiments of the present disclosure, a recombinant DNA construct is provided comprising a transcribable DNA sequence encoding a non-coding RNA molecule targeting an endogenous GA3 oxidase_1, GA3 oxidase_2 and/or GA3 oxidase_3 gene(s) for suppression, wherein the transcribable DNA sequence is operably linked to a constitutive, tissue-specific or tissue-preferred promoter, and wherein the transcribable DNA sequence causes the expression level of an endogenous GA3 oxidase_1, GA3 oxidase_2 and/or GA3 oxidase_3 gene(s) to become reduced or lowered in one or more tissue(s) of a plant transformed with the transcribable DNA sequence. Such a non-coding RNA molecule encoded by the transcribable DNA sequence may comprise a sequence that is (i) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 30, (ii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 33, and/or (iii) at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA3 oxidase protein in the plant that is at least 80%, at least 85%, at least 90%, at least 95%, at least 96%, at least 97%, at least 98%, at least 99%, at least 99.5%, or 100% identical to SEQ ID NO: 89.

In some embodiments, a maize plant herein (or plant part thereof) comprises a mutant allele at the GA20 oxidase_3 locus and a mutant allele at the GA20 oxidase_5 locus. In some embodiments, the plant or plant part herein may comprise either a mutant allele at the GA20 oxidase_3 locus or a mutant allele at the GA20 oxidase_5 locus. In some embodiments, the maize plant herein (or plant part thereof) may comprise one or two mutant alleles at the GA20 oxidase_3 locus, which may be homozygous, heterozygous, and/or heteroallelic (as described herein). In some embodiments, the maize plant herein may comprise one or two mutant alleles at the GA20 oxidase_5 locus, which may be homozygous, heterozygous, and/or heteroallelic (as described herein). In an aspect, the present disclosure provides a modified corn plant, or plant part thereof, comprising a mutant allele at GA20 oxidase_3 locus and a mutant allele at GA20 oxidase_5 locus, wherein at least one of the GA20 oxidase_3 and GA20 oxidase_5 loci comprises homozygous mutant alleles. In another aspect, the present disclosure provides a modified corn plant, or plant part thereof, comprising a first homozygous mutation in one of GA20 oxidase_3 and GA20 oxidase_5 genes and further comprising a second heterozygous or homozygous mutation in the other one of the GA20 oxidase_3 and GA20 oxidase_5 genes.

In some embodiments, the maize plant herein (or plant part thereof) may be homozygous for a mutant allele at the GA20 oxidase_3 locus. If both alleles at a locus are mutant alleles, then the plant is described as being homozygous for the mutant alleles. In some embodiments, the maize plant may be heterozygous for a mutant allele at the GA20 oxidase_3 locus. If one allele at a locus on one chromosome of a plant is a mutant allele and the other corresponding allele on the homologous chromosome of the plant is wild-type, then the plant is described as being heterozygous for the mutant allele. In some embodiments, for diploid organisms such as corn, the maize plant may be heteroallelic at the GA20 oxidase_3 locus and may comprise a first mutant allele on one chromosome at the GA20 oxidase_3 locus and a second mutant allele on a second homologous chromosome at the GA20 oxidase_3 locus. In connection with the latter, where the maize plant is heteroallelic at the GA20 oxidase_3 locus, for instance, the maize plant may include two different mutant GA20 oxidase_3 alleles at the same locus. A plant homozygous for mutant alleles at a locus may comprise the same mutant allele or different mutant alleles if heteroallelic or biallelic.

In another embodiment, the maize plant herein (or plant part thereof) may be homozygous for a mutant allele at the GA20 oxidase_5 locus. In some embodiments, the maize plant may be heterozygous for a mutant allele at the GA20 oxidase_5 locus. In some embodiments, for diploid organisms such as corn, the maize plant may be heteroallelic at the GA20 oxidase_5 locus and may comprise a first mutant allele on one chromosome at the GA20 oxidase_5 locus and a second mutant allele on a second homologous chromosome at the GA20 oxidase_5 locus. In connection with the latter, where the maize plant is heteroallelic at the GA20 oxidase_5 locus, for instance, the maize plant may include two different mutant GA20 oxidase_5 alleles at the same locus.

A mutant allele of the endogenous GA20 oxidase_3 gene of the maize plant (or plant part thereof) may comprise one or more mutation(s) or edit(s), such as an insertion, deletion or substitution of one or more nucleotides, or any combination thereof, in the endogenous GA20 oxidase_3 gene relative to SEQ ID NO: 7, 8 and/or 34. Such mutation(s) or edit(s) of a mutant allele of the GA20 oxidase_3 gene may be present in one or more exon(s), intron(s), upstream or regulatory region(s), and/or untranslated region(s) of the GA20 oxidase_3 gene. A mutant allele of the endogenous GA20 oxidase_5 gene of the maize plant (or plant part thereof) may comprise one or more mutation(s) or edit(s), such as an insertion, deletion or substitution of one or more nucleotides, or any combination thereof, in the endogenous GA20 oxidase_5 gene relative to SEQ ID NO: 13, 14 and/or 35. Such mutation(s) or edit(s) of a mutant allele of the GA20 oxidase_5 gene may be present in one or more exon(s), intron(s), upstream or regulatory region(s), and/or untranslated region(s) of the GA20 oxidase_5 gene. A mutant allele of the endogenous GA20 oxidase_4 gene of the maize plant (or plant part thereof) may comprise one or more mutation(s) or edit(s), such as an insertion, deletion or substitution of one or more nucleotides, or any combination thereof, in the endogenous GA20 oxidase_4 gene relative to SEQ ID NO: 10, 11 and/or 38. Such mutation(s) or edit(s) of a mutant allele of the GA20 oxidase_4 gene may be present in one or more exon(s), intron(s), upstream or regulatory region(s), and/or untranslated region(s) of the GA20 oxidase_4 gene. A mutant allele of the endogenous GA3 oxidase_1 gene of the maize plant (or plant part thereof) may comprise one or more mutation(s) or edit(s), such as an insertion, deletion or substitution of one or more nucleotides, or any combination thereof, in the endogenous GA3 oxidase_1 gene relative to SEQ ID NO: 28, 29, 36 and/or 84. Such mutation(s) or edit(s) of a mutant allele of the GA3 oxidase_1 gene may be present in one or more exon(s), intron(s), upstream or regulatory region(s), and/or untranslated region(s) of the GA3 oxidase_1 gene. A mutant allele of the endogenous GA3 oxidase_2 gene of the maize plant (or plant part thereof) may comprise one or more mutation(s) or edit(s), such as an insertion, deletion or substitution of one or more nucleotides, or any combination thereof, in the endogenous GA3 oxidase_2 gene relative to SEQ ID NO: 31, 32, 37 and/or 85. Such mutation(s) or edit(s) of a mutant allele of the GA3 oxidase_2 gene may be present in one or more exon(s), intron(s), upstream or regulatory region(s), and/or untranslated region(s) of the GA3 oxidase_2 gene. A mutant allele of the endogenous GA3 oxidase_3 gene of the maize plant (or plant part thereof) may comprise one or more mutation(s) or edit(s), such as an insertion, deletion or substitution of one or more nucleotides, or any combination thereof, in the endogenous GA3 oxidase_3 gene relative to SEQ ID NO: 86, 87 and/or 88. Such mutation(s) or edit(s) of a mutant allele of the GA3 oxidase_3 gene may be present in one or more exon(s), intron(s), upstream or regulatory region(s), and/or untranslated region(s) of the GA3 oxidase_3 gene.

In some embodiments, a maize plant herein (or plant part thereof) comprises a mutant allele of the endogenous bm3 gene. In connection therewith with, in various ones of such embodiments the maize plant (or plant part thereof) includes the mutant allele of the endogenous bm3 gene in combination with a mutant allele of the endogenous br2 or GA oxidase gene or with a transgene encoding a RNA molecule targeting a GA oxidase gene for suppression (e.g., as a stack, etc.).

A mutant allele of the endogenous bm3 gene of the maize plant generally refers to a mutation in the COMT gene of the maize plant, which encodes caffeic acid O-methyltransferase, an enzyme involved in lignin biosynthesis (and which may be characterized by a brown pigmentation in the leaf midrib at the v3 to v10 stage and lower lignin content in corn plant tissue). Sec, International PCT Pat. Appl. Publ. No. WO 2020/117837 (which is incorporated herein by reference in its entirety). Such mutant allele may result in a loss of gene function, gain of gene function, no change in gene function, or other changes in gene expression in the maize plant.

A mutant allele of the endogenous bm3 gene of the maize plant (or plant part thereof) may comprise an insertion, deletion or substitution of one or more nucleotides, or any combination thereof, in the endogenous bm3 gene. A mutant allele of the endogenous bm3 gene of the maize plant (or plant part thereof) may comprise one or more mutation(s) or edit(s), such as an insertion, deletion or substitution of one or more nucleotides, or any combination thereof, in the endogenous bm3 gene relative to SEQ ID NO: 94, 95 and/or 96. SEQ ID NO: 94 and 95 are the genomic wild-type sequences of the bm3 gene locus of the 01DKD2 and LH244 corn germplasms, respectively. SEQ ID NO: 96 is the genomic wild-type sequence of the bm3 gene locus of another corn germplasm. Such mutation(s) or edit(s) of a mutant allele of the bm3 gene may be present in one or more exon(s), intron(s), upstream or regulatory region(s), and/or untranslated region(s) of the bm3 gene. For example, a mutant allele of the endogenous bm3 gene may be the bm3-1 allele, bm3-2 allele, or bm3-3 allele. See, e.g., Sattler S. et al, Plant Science 178:229-238 (2010) and International PCT Pat. Appl. Publ. No. WO 2020/117837 (both of which are incorporated herein by reference in its entirety). The bm3-1 mutant allele may refer to a mutation in which a long terminal repeat (LTR) retrotransposon is inserted into the second exon of COMT. And, the mutant bm3-2 allele and the mutant bm3-3 allele may each refer to separate deletions within the second exon of COMT. In all instances, though, the COMT activity of the mutant plant is significantly reduced, if not completely abrogated. As described herein, desired mutant alleles of the bm3 gene may be selected based on an observable phenotype or using a selection agent with a selectable marker, a screenable marker, or a molecular technique.

A mutant allele of the endogenous bm3 gene may occur in or be introduced into the maize plant (or plant part thereof) through introgression, such as from an existing line, or a mutant allele of the endogenous bm3 gene may be a mutagenized or edited allele of the endogenous bm3 gene (e.g., via targeted genome editing, etc.). See, e.g., International PCT Pat. Appl. Publ. No. WO 2020/117837 (describing various methods for genetically modifying maize plants with respect to the bm3 gene, and which is incorporated herein by reference in its entirety). In the latter, where the mutant allele of the endogenous bm3 gene is an edited allele of the endogenous bm3 gene, the edited allele of the bm3 gene may then be introduced into the maize plant (e.g., into a short stature, dwarf, semi-dwarf, or brachytic maize plant already comprising a mutant allele of the endogenous br2 gene (as generally described above), etc.). In connection therewith, one or more edited allele(s) of the endogenous bm3 gene may be synthesized via genome editing techniques that utilize genome modification enzymes such as, for example, ZFNs, engineered or native meganucleases, TALENs, RNA-guided endonucleases (e.g., making use of clustered regularly interspaced short palindromic repeats (CRISPR) technology, etc.), etc., and then introduced into the maize plant. Examples of edited mutant bm3 alleles are discussed, for example, in International PCT Pat. Appl. Publ. No. WO 2020/117837 (which is incorporated herein by reference).

In some embodiments, the maize plant herein (or plant part thereof) may be homozygous for a mutant allele of the endogenous bm3 gene. If both alleles at a locus are mutant allele(s), then the plant is described as being homozygous for the mutant allele(s). In some embodiments, the maize plant may be heterozygous for a mutant allele of the endogenous bm3 gene. If one allele at a locus on one chromosome of a plant is a mutant allele and the other corresponding allele on the homologous chromosome of the plant is wild-type, then the plant is described as being heterozygous for the mutant allele. In some embodiments, for diploid organisms such as corn or maize, the maize plant may be heteroallelic for the endogenous bm3 gene and may comprise a first mutant allele on one chromosome of the endogenous bm3 gene and a second mutant allele on a second homologous chromosome of the endogenous bm3 gene. In connection with the latter, where the maize plant is heteroallelic for the endogenous bm3 gene, for instance, the maize plant may include two different mutant bm3 gene alleles at the same locus. A plant homozygous for mutant alleles at a locus may comprise the same mutant allele or different mutant alleles if heteroallelic.

In one example embodiment, trait associated markers were developed to select two bm3 gene mutant alleles from donor lines. The bm3 gene mutant allele markers included brown midrib 3 (bm3 gene) mutation from MGCSC-408E, Cgbm304B01 and brown midrib 3 (bm3 gene) mutation from MGCSC-415E, Cgbm304B02. For each bm3 gene mutant allele, the trait associated marker's presence at chromosome 4 at genetic position 81.2 indicated presence of the desired mutant allele. In connection therewith, each bm3 gene allele that the marker was not present at this position was considered a wild-type.

According to some embodiments, a corn or maize plant, plant part, etc., may comprise a mutation or transgene other than a mutation in an endogenous bm3 gene to give rise to improved silage characteristics, which may also be combined with a short stature trait or phenotype as provided herein. Several different genes have been identified in corn or maize that when mutated can cause a brown midrib phenotype, namely bm1, bm2, bm3, bm4, and bm5. Much of the description herein is focused on the bm3 gene, but a corn or maize plant, plant part, etc., may alternatively or additionally comprise a mutation(s) or mutant allele(s) of a bm1, bm2, bm4, or bm5 gene(s), which may be homozygous, heterozygous, or heteroallelic for the mutation(s) or mutant allele(s). Sec, e.g., Sattler, S. E. et al., Plant Science 178:229-238 (2010), and Ali, F. et al., Plant Breeding 129:724-726 (2010), the entire contents and disclosures of which are incorporated herein by reference.

In some embodiments, the maize plant herein (or plant part thereof) may comprise one or two mutant alleles of the endogenous br2 or GA oxidase gene, which may be homozygous, heterozygous, and/or heteroallelic (as described herein). In some embodiments, the maize plant herein may comprise one or two mutant alleles of the endogenous bm3 gene, which may be homozygous, heterozygous, and/or heteroallelic (as described herein).

From the above, the maize plant (or plant part thereof) comprising both a mutant allele of the endogenous br2 gene, a mutant allele of an endogenous GA oxidase gene, or a transgene targeting an endogenous GA oxidase gene for suppression, and a mutant allele of the endogenous bm3 gene may be grown, created, achieved, synthesized, etc. (broadly, provided or produced) in various different manners (or by various different methods or processes, etc.). For example, in some embodiments, a method of producing the maize plant may include making the maize plant from one or more parental maize plants by traditional crop modification techniques or processes (e.g., by selective breeding, crossbreeding, backcrossing, introgression, etc. using one or more maize plant(s) from one or more elite line(s); etc.). In other embodiments, a method of producing the maize plant may include genetically modifying a maize plant (e.g., a maize plant from an elite line, etc.) via one or more of the techniques described above (e.g., to comprise either or both a mutant allele of the endogenous br2 gene, a mutant allele of an endogenous GA oxidase gene, or a transgene targeting an endogenous GA oxidase gene for suppression, and a mutant allele of the endogenous bm3 gene (depending on the genotype of the maize plant), etc.) to thereby produce the maize plant comprising both a mutant allele(s) of the endogenous br2 gene and a mutant allele(s) of the endogenous bm3 gene.

In one example embodiment, a maize plant (or plant part thereof) from an elite line of maize plants already comprising a mutant allele of the endogenous br2 gene (e.g., comprising one or more of the br2-23, br2-7081, br2-7861, br2-qph1, br2-qpa1, br2-NC238 or br2-1005 mutant alleles, etc. as described herein) may be crossed with a maize plant from an elite line of maize plants already comprising a mutant allele of the endogenous bm3 gene (e.g., comprising one or more of the bm3-1, bm3-2 or bm3-3 mutant alleles, etc. as described herein). In doing so, a hybrid maize plant, for example, may be produced by crossing the two maize plants from the parental elite lines. Specifically, this may include crossing a maize plant from a parental elite line containing at least one copy of a mutant allele of the br2 gene with a maize plant from a parental elite line containing at least one copy of a mutant allele of the bm3 gene.

In another example embodiment, a maize plant (or plant part thereof) from an elite line of plants already comprising a mutant allele of the endogenous br2 gene or an endogenous GA oxidase may undergo conversion (e.g., targeted genome editing or otherwise as described herein, etc.) with one or more mutant alleles of the endogenous bm3 gene (e.g., one or more of the bm3-1, bm3-2 or bm3-3 mutant alleles, etc. as described herein) to thereby produce a maize plant comprising both a mutant allele of the endogenous br2 gene and a mutant allele of the endogenous bm3 gene. Alternatively, a maize plant from an elite line of plants already comprising a mutant allele of the endogenous bm3 gene may undergo conversion (e.g., targeted genome editing or otherwise as described herein, etc.) with one or more mutant alleles of the endogenous br2 gene (e.g., one or more of the br2-23, br2-7081, br2-7861, br2-qph1, br2-qpa1, br2-NC238 or br2-1005 mutant alleles, etc. as described herein) or an endogenous GA oxidase gene to thereby produce a maize plant comprising both a mutant allele of the endogenous br2 gene and a mutant allele of the endogenous bm3 gene.

In still other example embodiments, the maize plant (or plant part thereof) comprising both a mutant allele of the endogenous br2 gene, a mutant allele of an endogenous GA oxidase gene, or a transgene targeting an endogenous GA oxidase gene for suppression, and a mutant allele of the endogenous bm3 gene may be produced from a maize plant of a parental elite line, by way of the maize plant undergoing multiple conversions with multiple mutant alleles. For example, a maize plant of a parental elite line not comprising either a mutant allele of the endogenous br2 gene, a mutant allele of an endogenous GA oxidase gene, or a transgene targeting an endogenous GA oxidase gene for suppression, or a mutant allele of the endogenous bm3 gene may undergo conversion with one or more mutant alleles of the br2 gene, a mutant allele of an endogenous GA oxidase gene, or a transgene targeting an endogenous GA oxidase gene for suppression, and then subsequently undergo conversion with one or more mutant alleles of the bm3 gene. Alternatively, the maize plant of the parental elite line not comprising either a mutant allele of the endogenous br2 gene, a mutant allele of an endogenous GA oxidase gene, or a transgene targeting an endogenous GA oxidase gene for suppression, or a mutant allele of the endogenous bm3 gene may undergo conversion with one or more mutant alleles of the bm3 gene, and then subsequently undergo conversion with one or more mutant alleles of the br2 gene, a mutant allele of an endogenous GA oxidase gene, or a transgene targeting an endogenous GA oxidase gene for suppression. In some examples, such conversions may take place (or may occur or may be performed) substantially simultaneously.

The above examples of growing, creating, achieving, synthesizing, etc. (broadly, providing or producing) the maize plant (or plant part thereof) comprising both a mutant allele of the endogenous br2 gene, a mutant allele of an endogenous GA oxidase gene, or a transgene targeting an endogenous GA oxidase gene for suppression, and a mutant allele of the endogenous bm3 gene are provided for illustration only. It should be appreciated that other ways of growing, creating, achieving, synthesizing, etc. the maize plant are within the scope of the present disclosure, as may be known and implemented by those having ordinary skill in the art.

As described herein, the description herein of the maize plant comprising a mutant allele of the endogenous br2 gene and/or a mutant allele of the endogenous bm3 gene is also applicable to one or more parts of the maize plant (e.g., a maize plant part, etc.), including, for example, a stem of the maize plant, a leaf of the maize plant, a seed of the maize plant, combinations thereof, etc. As such, consistent with the above description, a maize plant seed is thus also provided herein, where the maize plant seed may have a mutant allele of the endogenous br2 gene, a mutant allele of the endogenous bm3 gene, or both a mutant allele of the endogenous br2 gene and a mutant allele of the endogenous bm3 gene.

While reference herein is made to the bm3 gene, it should be appreciated that the current description also applies, more generally, to a maize plant (or plant part thereof) and/or population of maize plants with any Brown midrib (BMR) mutation(s). For instance, the current description may also be applicable to other BMR mutations such as, for example, mutations of the bm1 gene, bm2 gene, bm4 gene, bm5 gene, bm6 gene, etc. BMR mutations may be recognized as mutations that exhibit a reddish-brown pigmentation in the leaf midrib, mutations that alter lignin content and digestibility, etc. BMR mutations may also be discovered as naturally occurring mutants or generated via genetic modifications. As previously stated, a mutant allele of the endogenous bm3 gene of the maize plant generally refers to a mutation in the COMT gene of the maize plant, which encodes caffeic acid O-methyltransferase, an enzyme involved in lignin biosynthesis. In another example, bm1 is caused by a mutation of a cinnamyl alcohol dehydrogenase (CAD) gene, and a mutant allele of the endogenous bm1 gene may be bm1-E1 and bm1-E2. Sec, e.g., Xiong W. et al, Front. Plant Sci. 11:594798 (2020). Still other BMR mutations include mutations at other genomic locations.

In some example embodiments, the maize plant described herein (e.g., comprising a mutant allele of the endogenous br2 gene, a mutant allele of an endogenous GA oxidase gene, or a transgene targeting an endogenous GA oxidase gene for suppression, and/or a mutant allele of the endogenous bm3 gene, etc.) may be grown in one or more field(s) (or other growing space), in combination with multiple other of the same maize plants (e.g., in the same field, in different fields, etc.) (e.g., as part of a population of maize plants, etc.). As such, a population of the maize plants may be provided in the field(s). In particular, for example, seeds (e.g., comprising both a mutant allele of the endogenous br2 gene, a mutant allele of an endogenous GA oxidase gene, or a transgene targeting an endogenous GA oxidase gene for suppression, and a mutant allele of the endogenous bm3 gene, etc.) may be planted in the field(s), as desired, and the maize plants may then be grown from the seeds. In doing so, the maize plant seeds may be planted and grown in one or more rows in the field(s) (e.g., parallel rows, etc.).

For instance, the maize plant seeds may be planted (e.g., via a farm implement corn planter, etc.) (and grown) in multiple rows (e.g., generally parallel rows (e.g., in single rows, double rows, split rows, etc.), etc.) or otherwise (e.g., in arrangements other than in rows, etc.) with any desired spacing, etc. In one example (and without limitation), the seeds may be planted in rows with average spacing between adjacent rows of the maize plant seeds (and growing maize plants) of about 80 cm or less, about 70 cm or less, about 60 cm or less, about 50 cm or less, about 40 cm or less, about 35 cm or less, or about 30 cm or less, about 25 cm or less, or about 76 cm, about 51 cm, about 38 cm, about 30 cm, about 25 cm, etc. Additionally, or alternatively, the maize plant seeds may be planted in the rows (e.g., in the field(s), etc.) at a planting density of about 27,000 seeds/Ha (about 10,931 seeds/acre) or more, about 67,000 seeds/Ha (about 27,126 seeds/acre) or more, about 100,000 seeds/Ha (about 40,486 seeds/acre) or more, about 133,000 seeds/Ha (about 53,846 seeds/acre) or more, about 167,000 seeds/Ha (about 67,476 seeds/acre) or more, about 200,000 seeds/Ha (about 80,971 seeds/acre) or more, about 233,000 seeds/Ha (about 94,332 seeds/acre) or more, about 267,000 seeds/Ha (about 108,097 seeds/acre) or more, about 333,000 seeds/Ha (about 134,953 seeds/acre) or more, about 400,000 seeds/Ha (about 161,943 seeds/acre) or more, etc. Additionally, or alternatively, the maize plants growing from the planted seeds (e.g., planted, growing or grown as (or as part of) a population of maize plants, etc.) may be present (e.g., in the field(s), etc.) at a growing density of about 20,000 plants/Ha (about 8,098 plants/acre) or more, about 50,000 plants/Ha (about 20,243 plants/acre) or more, about 75,000 plants/Ha (about 30,364 plants/acre) or more, about 100,000 plants/Ha (about 40,486 plants/acre) or more, about 125,000 plants/Ha (about 50,607 plants/acre) or more, about 150,000 plants/Ha (about 60,729 plants/acre) or more, about 175,000 plants/Ha (about 70,850 plants/acre) or more, about 200,000 plants/Ha (about 80,972 plants/acre) or more, about 250,000 plants/Ha (about 101,215 plants/acre) or more, about 300,000 plants/Ha (about 121,457 plants/acre) or more, etc.

In connection therewith, in some example embodiments, companion crop plants may also be grown in the field(s) in combination with (together with, etc.) the maize plants (e.g., in the same field(s), in a different field(s), etc.) (e.g., as part of a population of companion crop plants planted and grown with a population of maize plants herein within a growing space, etc.), thereby resulting in intercropped plants (e.g., a population of intercropped plants comprising both the maize plants comprising a mutant allele of the endogenous br2 gene, a mutant allele of an endogenous GA oxidase gene, or a transgene targeting an endogenous GA oxidase gene for suppression, and/or a mutant allele of the endogenous bm3 gene and the companion crop plants, etc.). The companion or intercropped plants or crop plants may include any desired and/or suitable plants within the scope of the present disclosure, including, for example (and without limitation), wheat plants, barley plants, oat plants, alfalfa plants, rye plants, clover plants, grass, triticale, cereal plants, legume, bean plants (e.g., Tarbais beans, Preisgewinner beans, etc.), pea plants (e.g., cowpeas, etc.), soybean plants, sunflower plants, other oil containing crop plants, etc.

For instance, companion crop plant seeds may be planted in the field(s) with the maize plant seeds (e.g., comprising both a mutant allele of the endogenous br2 gene, a mutant allele of an endogenous GA oxidase gene, or a transgene targeting an endogenous GA oxidase gene for suppression, and a mutant allele of the endogenous bm3 gene, etc.), as desired, and the maize plants and companion crop plants may then be grown generally together from the seeds (in the field(s)). In doing so, the maize plant seeds may be planted and grown in one or more rows in the field(s) (e.g., parallel rows, etc.) (e.g., in single rows, double rows, split rows, etc.), and the companion crop plant seeds may be planted and grown in one or more rows generally between or generally adjacent rows of the maize plant seeds (e.g., in single rows, double rows, split rows, etc.). In one example, the maize plant seeds and the companion crop plant seeds may be planted in generally alternating rows, such that the companion crop plant seeds are generally planted between adjacent rows of the maize plant seeds (e.g., defining a general pattern of maize-companion crop-maize, etc.). In another example, the maize plant seeds and the companion crop plant seeds may be planted such that two rows of the companion crop plant seeds are disposed (or located, etc.) between adjacent rows of the maize plant seeds (e.g., defining a general pattern of maize-companion crop-companion crop-maize, etc.). In still another example, the maize plant seeds and the companion crop plant seeds may be planted such that the companion crop plant seeds are disposed (or located, etc.) between every two adjacent rows of the maize plant seeds (e.g., defining a general pattern of maize-maize-companion crop-maize-maize, etc.). In another example, the population of companion crop plants herein may be planted and grown in a row or plurality of parallel rows, and each row of companion crop plants may be planted between two adjacent rows of the population of maize plants. In connection therewith, the maize plant seeds and the companion crop plant seeds may be planted (and grown) in the multiple rows the field(s) (e.g., in any desired pattern, etc.) (or in arrangements or patterns other than rows, etc.) with any desired average spacing between adjacent rows of the seeds/plants, including, for example (and without limitation), of about 80 cm or less, about 70 cm or less, about 60 cm or less, about 50 cm or less, about 40 cm or less, about 35 cm or less, about 30 cm or less, about 25 cm or less, about 76 cm, about 51 cm, about 38 cm, about 30 cm, etc. It should be appreciated that the maize plant seeds and/or the companion crop plant seeds may be planted in other patterns (e.g., where maize plant seeds and the companion crop plant seeds are planted together in a same row (or rows), where different patterns of maize plants and companion crop plants are provided than described above, etc.) and/or at other spacings within the scope of the present disclosure.

Further, the companion crop plant seeds may be planted in the rows at a planting density of about 13,000 seeds/Ha (about 5,263 seeds/acre) or more, about 27,000 seeds/Ha (about 10,931 seeds/acre) or more, about 40,000 seeds/Ha (about 16,194 seeds/acre) or more, about 53,000 plants/Ha (about 21,457 plants/acre) or more, about 67,000 seeds/Ha (about 27,126 seeds/acre) or more, about 100,000 seeds/Ha (about 40,486 seeds/acre) or more, about 133,000 seeds/Ha (about 53,846 seeds/acre) or more, about 167,000 seeds/Ha (about 67,476 seeds/acre) or more, about 200,000 seeds/Ha (about 80,971 seeds/acre) or more, about 233,000 seeds/Ha (about 94,332 seeds/acre) or more, about 267,000 seeds/Ha (about 108,097 seeds/acre) or more, etc. Additionally, or alternatively, the companion crop plants growing from the planted companion crop plant seeds (e.g., planted, growing or grown as (or as part of) a population of companion crop plants, etc.) may be present at a growing density of about 10,000 plants/Ha (about 4,049 plants/acre) or more, about 20,000 plants/Ha (about 8,098 plants/acre) or more, about 30,000 plants/Ha (about 12,146 plants/acre) or more, about 40,000 plants/Ha (about 16,194 plants/acre) or more, about 50,000 plants/Ha (about 20,243 plants/acre) or more, about 75,000 plants/Ha (about 30,364 plants/acre) or more, about 100,000 plants/Ha (about 40,486 plants/acre) or more, about 125,000 plants/Ha (about 50,607 plants/acre) or more, about 150,000 plants/Ha (about 60,729 plants/acre) or more, about 175,000 plants/Ha (about 70,850 plants/acre) or more, about 200,000 plants/Ha (about 80,972 plants/acre) or more, etc.

Moreover, in some example embodiments, the maize plant seeds and the companion crop plant seeds, together (e.g., the intercropped seeds, etc.), may be planted in the rows at a planting density of about 67,000 seeds/Ha (about 27,126 seeds/acre) or more, about 100,000 seeds/Ha (about 40,486 seeds/acre) or more, about 133,000 seeds/Ha (about 53,846 seeds/acre) or more, about 167,000 seeds/Ha (about 67,476 seeds/acre) or more, about 200,000 seeds/Ha (about 80,971 seeds/acre) or more, about 233,000 seeds/Ha (about 94,332 seeds/acre) or more, about 267,000 seeds/Ha (about 108,097 seeds/acre) or more, about 333,000 seeds/Ha (about 134,953 seeds/acre) or more, about 400,000 seeds/Ha (about 161,943 seeds/acre) or more, about 466,000 seeds/Ha (about 188,664 seeds/acre) or more, about 533,000 seeds/Ha (about 215,924 sees/acre) or more, about 600,000 seeds/Ha (about 242,914 seeds/acre) or more, about 667,000 seeds/Ha (about 270,040 seeds/acre) or more, etc. Additionally, or alternatively, the maize plants and the companion crop plants (e.g., the intercropped plants, etc.) growing from the planted seeds (e.g., planted, growing or grown as (or as part of) a population of maize plants and companion crop plants, etc.) may be present at a growing density of about 50,000 plants/Ha (about 20,243 plants/acre) or more, about 75,000 plants/Ha (about 30,364 plants/acre) or more, about 100,000 plants/Ha (about 40,486 plants/acre) or more, about 125,000 plants/Ha (about 50,607 plants/acre) or more, about 150,000 plants/Ha (about 60,729 plants/acre) or more, about 175,000 plants/Ha (about 70,850 plants/acre) or more, about 200,000 plants/Ha (about 80,972 plants/acre) or more, about 250,000 plants/Ha (about 101,215 plants/acre) or more, about 300,000 plants/Ha (about 121,457 plants/acre) or more, about 350,000 plants/Ha (about 141,700 plants/acre) or more, about 400,000 plants/Ha (about 161,943 plants/acre) or more, about 450,000 plants/Ha (about 182,186 plants/acre) or more, about 500,000 plants/Ha or more (about 202,429 plants/Ha), etc.

In connection with the above, and as generally described, in some embodiments of the present disclosure, the maize plant described herein (e.g., the maize plant comprising a mutant allele of the endogenous br2 gene, a mutant allele of an endogenous GA oxidase gene, or a transgene targeting an endogenous GA oxidase gene for suppression, the maize plant comprising a mutant allele of the endogenous br2 gene, a mutant allele of an endogenous GA oxidase gene, or a transgene targeting an endogenous GA oxidase gene for suppression and a mutant allele of the bm3 gene, the maize plant having one or more of the traits described herein, etc.) may be included in the population of maize plants (or maize plant population, or more generally, plant population) (e.g., as grown in the field(s), etc.). In some examples, then, at least one of the maize plants in the population may have a mutant allele of the endogenous br2 gene, a mutant allele of an endogenous GA oxidase gene, or a transgene targeting an endogenous GA oxidase gene for suppression, may have both a mutant allele of the endogenous br2 gene, a mutant allele of an endogenous GA oxidase gene, or a transgene targeting an endogenous GA oxidase gene for suppression, and/or a mutant allele of the bm3 gene, and/or may have one or more of the traits described herein. In other examples, multiple of the maize plants in the population may have a mutant allele of the endogenous br2 gene, a mutant allele of an endogenous GA oxidase gene, or a transgene targeting an endogenous GA oxidase gene for suppression, may have both a mutant allele of the endogenous br2 gene, a mutant allele of an endogenous GA oxidase gene, or a transgene targeting an endogenous GA oxidase gene for suppression, and a mutant allele of the bm3 gene, and/or may have one or more of the traits described herein. In still other examples, at least half (e.g., a plurality, etc.) of the maize plants in the population may have a mutant allele of the endogenous br2 gene, a mutant allele of an endogenous GA oxidase gene, or a transgene targeting an endogenous GA oxidase gene for suppression, may have both a mutant allele of the endogenous br2 gene, a mutant allele of an endogenous GA oxidase gene, or a transgene targeting an endogenous GA oxidase gene for suppression and a mutant allele of the bm3 gene, and/or may have one or more of the traits described herein. In further examples, all of the maize plants in the population may have a mutant allele of the endogenous br2 gene, a mutant allele of an endogenous GA oxidase gene, or a transgene targeting an endogenous GA oxidase gene for suppression, may have both a mutant allele of the endogenous br2 gene, a mutant allele of an endogenous GA oxidase gene, or a transgene targeting an endogenous GA oxidase gene for suppression and a mutant allele of the bm3 gene, and/or may have one or more of the traits described herein. In some embodiments, the maize plants of the population herein may comprise one or two mutant alleles of the endogenous br2 gene, a mutant allele of an endogenous GA oxidase gene, or a transgene targeting an endogenous GA oxidase gene for suppression, which may be homozygous, heterozygous, and/or heteroallelic for the endogenous br2 or GA oxidase gene or the transgene targeting an endogenous GA oxidase gene for suppression, and may comprise a first mutant allele of the endogenous br2 or GA oxidase gene and a second mutant allele of the endogenous br2 or GA oxidase gene. In some embodiments, the maize plants of the population herein may comprise one or two mutant alleles of the endogenous bm3 gene, which may be homozygous, heterozygous, and/or heteroallelic for the endogenous bm3 gene and may comprise a first mutant allele of the endogenous bm3 gene and a second mutant allele of the endogenous bm3 gene.

The population of maize plants may be included together in a field or other growing space, or they may be spread apart (e.g., in multiple different fields, etc.). In connection therewith, the population of the maize plants may include any desired number of the maize plants (comprising a mutant allele of the endogenous br2 gene, a mutant allele of an endogenous GA oxidase gene, or a transgene targeting an endogenous GA oxidase gene for suppression, and/or a mutant allele of the endogenous bm3 gene). For example, the population may include one maize plant, or it may include multiple maize plants (e.g., about 2 maize plants or more, about 5 maize plants or more, about 10 maize plants or more, about 20 maize plants or more, about 50 maize plants or more, about 75 maize plants or more, about 100 maize plants or more, about 150 maize plants or more, about 200 maize plants or more, about 500 maize plants or more, about 1,000 maize plants or more, about 5,000 maize plants or more, about 10,000 maize plants or more, about 50,000 maize plants or more, about 100,000 maize plants or more, about 200,000 maize plants or more, about 300,000 maize plants or more, about 500,000 maize plants or more, etc.).

Further, in some embodiments of the present disclosure, a maize plant seed (e.g., a seed comprising a mutant allele of the endogenous br2 gene, a mutant allele of an endogenous GA oxidase gene, or a transgene targeting an endogenous GA oxidase gene for suppression, a seed comprising a mutant allele of the endogenous br2 gene, a mutant allele of an endogenous GA oxidase gene, or a transgene targeting an endogenous GA oxidase gene for suppression and a mutant allele of the bm3 gene, etc.) may be included in a population of seeds (or maize plant seed population, or more generally, seed population). As such, in some examples, at least one of the maize plant seeds in the population may comprise a mutant allele of the endogenous br2 gene, a mutant allele of an endogenous GA oxidase gene, or a transgene targeting an endogenous GA oxidase gene for suppression, may comprise both a mutant allele of the endogenous br2 gene, a mutant allele of an endogenous GA oxidase gene, or a transgene targeting an endogenous GA oxidase gene for suppression and a mutant allele of the bm3 gene, and/or may have one or more of the traits described herein. In other examples, multiple of the maize plant seeds in the population may comprise a mutant allele of the endogenous br2 gene, a mutant allele of an endogenous GA oxidase gene, or a transgene targeting an endogenous GA oxidase gene for suppression, may comprise both a mutant allele of the endogenous br2 gene, a mutant allele of an endogenous GA oxidase gene, or a transgene targeting an endogenous GA oxidase gene for suppression and a mutant allele of the bm3 gene, and/or may have one or more of the traits described herein. In still other examples, at least half (e.g., a plurality, etc.) of the maize plant seeds in the population may comprise a mutant allele of the endogenous br2 gene, a mutant allele of an endogenous GA oxidase gene, or a transgene targeting an endogenous GA oxidase gene for suppression, may comprise both a mutant allele of the endogenous br2 gene, a mutant allele of an endogenous GA oxidase gene, or a transgene targeting an endogenous GA oxidase gene for suppression and a mutant allele of the bm3 gene, and/or may have one or more of the traits described herein. In further examples, all of the maize plant seeds in the population may comprise a mutant allele of the endogenous br2 gene, a mutant allele of an endogenous GA oxidase gene, or a transgene targeting an endogenous GA oxidase gene for suppression, may comprise both a mutant allele of the endogenous br2 gene, a mutant allele of an endogenous GA oxidase gene, or a transgene targeting an endogenous GA oxidase gene for suppression and a mutant allele of the bm3 gene, and/or may have one or more of the traits described herein.

The population of maize plant seeds may be included together, for example, in common storage, in a common container, etc., or they may be spread apart (e.g., in multiple different storage areas, containers, etc.). In connection therewith, the population of the maize plant seeds may include any desired number of the maize plant seeds (comprising a mutant allele of the endogenous br2 gene and/or a mutant allele of the endogenous bm3 gene). For example, the population may include one maize plant seed, or it may include multiple maize plant seeds (e.g., about 2 maize plant seeds or more, about 5 maize plant seeds or more, about 10 maize plant seeds or more, about 20 maize plant seeds or more, about 50 maize plant seeds or more, about 75 maize plant seeds or more, about 100 maize plant seeds or more, about 150 maize plant seeds or more, about 200 maize plant seeds or more, about 500 maize plant seeds or more, about 1,000 maize plant seeds or more, about 5,000 maize plant seeds or more, about 10,000 maize plant seeds or more, about 50,000 maize plant seeds or more, about 100,000 maize plant seeds or more, about 200,000 maize plant seeds or more, about 300,000 maize plant seeds or more, about 500,000 maize plant seeds or more, etc.).

Further, the companion crop plants (and/or plant seeds) described herein may be included in a population of companion crop plants (and/or plant seeds). The population of maize plants herein (e.g., the maize plant comprising a mutant allele of the endogenous br2 gene, a mutant allele of an endogenous GA oxidase gene, or a transgene targeting an endogenous GA oxidase gene for suppression, the maize plant (and/or plant seeds) comprising a mutant allele of the endogenous br2 gene, a mutant allele of an endogenous GA oxidase gene, or a transgene targeting an endogenous GA oxidase gene for suppression and a mutant allele of the bm3 gene, the maize plant having one or more of the traits described herein, etc.) and the population of companion crop plants (and/or plant seeds) may be included together in a population of intercropped plants (or intercropped plant population, or more generally, plant population) (e.g., as planted or grown in the field(s), etc.).

In some example embodiments, the maize plant described herein (e.g., the maize plant comprising a mutant allele of the endogenous br2 gene, a mutant allele of an endogenous GA oxidase gene, or a transgene targeting an endogenous GA oxidase gene for suppression, the maize plant comprising a mutant allele of the endogenous bm3 gene, the maize plant comprising a mutant allele of the endogenous br2 gene, a mutant allele of an endogenous GA oxidase gene, or a transgene targeting an endogenous GA oxidase gene for suppression and a mutant allele of the bm3 gene, the maize plant having one or more of the traits described herein, etc.), or population of maize plants described herein, may be used as or may be included in biomass configured for use, for example, as a silage product, in biomass configured for use in energy production (e.g., ethanol production, etc.), etc. In connection with use as a silage product (or in producing or making a silage product (e.g., as a method of producing silage, etc.)), the maize plant(s) may be harvested and the above-ground biomass (e.g., era, stalks, leaves, cars, etc.) from the harvested plant(s) may be used to produce the silage product. In addition, in some example embodiments, the silage product may also include plants (e.g., above-ground biomass, etc.) from one or more of the companion crop(s) herein (e.g., wheat plants, barley plants, oat plants, alfalfa plants, rye plants, clover plants, grass, triticale, cereal plants, legume, bean plants, pea plants, soybean plants, sunflower plants, etc.).

In connection therewith, an example method of producing a silage product may be as follows. The silage product may be produced by initially planting a plurality of maize plant seeds (e.g., comprising both a mutant allele of the endogenous br2 gene, a mutant allele of an endogenous GA oxidase gene, or a transgene targeting an endogenous GA oxidase gene for suppression and/or a mutant allele of the endogenous bm3 gene, etc.) in a growing space (e.g., in a field, etc.). In addition, in some examples, a plurality of companion crop plant seeds may also be planted with the maize plant seeds in the growing space (although this is not required in all embodiments). For instance, as generally described above, the maize plant seeds may be planted in the growing space in rows (e.g., generally parallel rows, etc.) at desired spacing(s) and/or at desired density(ies). And, the companion crop plant seeds may similarly be planted in the growing space, generally between rows of the maize plant seeds, at desired spacing(s) and/or at desired density(ies). The planted maize plant seeds and companion crop plant seeds (e.g., the planted intercropped plant seeds, etc.) may then be grown to produce a plurality of maize plants and a plurality of companion crop plants (e.g., having the spacing(s) and/or density(ies) described above) within the growing space (e.g., a plurality of intercropped plants, etc.).

At a desired time, the maize plants and the companion crop plants (e.g., the intercropped plants, etc.) grown from the seeds may be harvested from the growing space (e.g., via one or more forage harvesters, etc.). In particular, an above-ground biomass of the plants may be harvested for use in producing the silage product. In one example, the maize plants and the companion crop plants may be harvested when a moisture content (e.g., an average moisture content, etc.) of the maize plants, the companion crop plants, or a combination thereof achieves a desired moisture content (e.g., between about 50% and about 80%, between about 55% and about 75%, between about 60% and about 70%, between about 65% and about 70%, etc.). In other examples, the maize plants and the companion crop plants may be harvested at other times, as desired or required, etc.

Once the maize plants and the companion crop plants are harvested, the corresponding above-ground biomass is processed to produce (or form) the silage product. In one example, this may include chopping, cutting, shredding, etc. the above-ground biomass into pieces. The pieces may then be collected and stored (e.g., layered, stacked, etc.) in an airtight or generally airtight manner (e.g., in a silo, etc.). The pieces may be stored for a time period from about 1 day to about 2 years, from about 1 week to about 2 years, from about 1 week to about 1 year, from about 2 weeks to about 6 months, from about 2 weeks to about 3 months, etc. In connection with such storage, the pieces may undergo fermentation (e.g., naturally, with aid of an inoculant, etc.) for a similar time period (e.g., for a time period from about 1 week to about 2 years, from about 1 week to about 1 year, from about 1 week to about 6 months, from about 2 weeks to about 6 months, from about 2 weeks to about 3 months, etc.). And, after the pieces of the biomass are stored and/or fermented for a desired time, the resulting silage product may be available to be fed to one or more livestock animals (e.g., cattle, horses, etc.). The method herein may then include feeding the silage product to the one or more livestock animals.

Alternatively, once the maize plants and the companion crop plants are harvested (as described above), the corresponding above-ground biomass may be processed (e.g., collected, harvested, chopped, cut, shredded, stored, and/or fermented, etc.) for subsequent use in producing energy. For example, the resulting biomass may be burned (either directly or after desired processing) for heating buildings and water, for industrial process heat, and/or for generating electricity via steam turbines, etc. Alternatively, the biomass may be thermo-chemically converted to produce one or more fuels (e.g., via pyrolysis and gasification, etc.). Or further, the biomass may be biologically converted to produce one or more liquid biofuels, for example, via fermentation, etc. (e.g., ethanol, etc.). In this latter example, the produced one or more fuels may then be burned for energy (e.g., to produce electricity, etc.), etc.

As described above, the maize plant(s) (and/or plant part(s) thereof) described herein (e.g., comprising a mutant allele of the endogenous br2 gene, a mutant allele of an endogenous GA oxidase gene, or a transgene targeting an endogenous GA oxidase gene for suppression, comprising a mutant allele of the endogenous br2 gene, a mutant allele of an endogenous GA oxidase gene, or a transgene targeting an endogenous GA oxidase gene for suppression and a mutant allele of the endogenous bm3 gene, etc.) (including the population(s) thereof described herein), as well as the silage product(s) (broadly, biomass) produced therefrom, may have one or more trait(s), characteristic(s), etc. that make it (them) suitable for grain and forage (e.g., silage, etc.) production, energy production, etc. For instance, in some example embodiments, the maize plant(s) (or parts thereof) and/or the silage product(s) (broadly, biomass) produced therefrom, as described herein, may exhibit (or may have) one or more of following traits (as described more below): reduced lignin content, improved fiber digestibility, reduced acid detergent fiber (ADF), increased starch content, increased neutral detergent fiber digestibility (NDFD) (also termed, cell wall digestibility (DCW)), improved nitrogen use efficiency (NUE), reduced root lodging (e.g., within the population of maize plants in the field(s), etc.), increased biomass (e.g., increased dry matter biomass (DMB), etc.), improved or increased protein content (and/or N-stover), increased milk output, increased silage yield, increased grain yield, increased stem cross-section area, improved standability, and/or shorter height as compared, for example, to a wild-type or control maize plant (e.g., a control plant not having a mutant allele of the endogenous br2 gene, a mutant allele of an endogenous GA oxidase gene, or a transgene targeting an endogenous GA oxidase gene for suppression, a control plant not having a mutant allele of the endogenous bm3 gene, a control plant not having both a mutant allele of the endogenous br2 gene, a mutant allele of an endogenous GA oxidase gene, or a transgene targeting an endogenous GA oxidase gene for suppression and a mutant allele of the endogenous bm3 gene, etc.) or plant part thereof and/or a silage product including such a wild-type or control maize plant or plant part.

In some example embodiments, a majority of maize plants in a population of maize plants (e.g., maize hybrid plants, etc.) may comprise a mutant allele of the endogenous br2 gene, a mutant allele of an endogenous GA oxidase gene, or a transgene targeting an endogenous GA oxidase gene for suppression, and the population of maize plants may have one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In some example embodiments, each (or all) of the maize plants in the population of maize plants (e.g., maize hybrid plants, etc.) comprises a mutant allele of the endogenous br2 gene, a mutant allele of an endogenous GA oxidase gene, or a transgene targeting an endogenous GA oxidase gene for suppression and/or a mutant allele of the endogenous bm3 gene, and the population of maize plants has one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In some of the above embodiments, the maize plants in the population of maize plants may be used for more than one purpose (e.g., may be used as a dual purpose (DP), product). In some of the above embodiments, a majority to all of the maize plants of the population may be homozygous for the mutant allele of the endogenous br2 or GA oxidase gene. In some of the above embodiments, a majority to all of the maize plants of the population may be heteroallelic for the endogenous br2 or GA oxidase gene and comprise a first mutant allele of the endogenous bm3 gene and a second mutant allele of the endogenous br2 or GA oxidase gene.

In some example embodiments, a majority of maize plants in a population of maize plants (e.g., maize hybrid plants, etc.) may comprise a mutant allele of the endogenous br2 gene, a mutant allele of an endogenous GA oxidase gene, or a transgene targeting an endogenous GA oxidase gene for suppression and/or a mutant allele of the endogenous bm3 gene, and the population of maize plants may have one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In some example embodiments, each (or all) of the maize plants in the population of maize plants (e.g., maize hybrid plants, etc.) comprises a mutant allele of the endogenous bm3 gene, and the population of maize plants has one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In some of the above embodiments, the maize plants in the population of maize plants may be used for more than one purpose (e.g., may be used as a dual purpose (DP), product). In some of the above embodiments, a majority to all of the maize plants of the population may be homozygous for the mutant allele of the endogenous bm3 gene. In some of the above embodiments, a majority to all of the maize plants of the population may be heteroallelic for the endogenous bm3 gene and comprise a first mutant allele of the endogenous bm3 gene and a second mutant allele of the endogenous bm3 gene.

In some example embodiments, a majority of maize plants in a population of maize plants (e.g., maize hybrid plants, etc.) may comprise a mutant allele of the endogenous br2 gene, a mutant allele of an endogenous GA oxidase gene, or a transgene targeting an endogenous GA oxidase gene for suppression and a mutant allele of the endogenous bm3 gene, and the population of maize plants may have one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In some example embodiments, each (or all) of the maize plants in the population of maize plants (e.g., maize hybrid plants, etc.) comprises a mutant allele of the endogenous br2 gene and a mutant allele of the endogenous bm3 gene, and the population of maize plants has one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants. In some of the above embodiments, the maize plants in the population of maize plants may be used for more than one purpose (e.g., may be used as a dual purpose (DP), product). In some of the above embodiments, a majority to all of the maize plants of the population may be homozygous for the mutant allele of the endogenous br2 gene and/or may be homozygous for the mutant allele of the endogenous bm3 gene. In some of the above embodiments, a majority to all of the maize plants of the population may be heteroallelic for the endogenous br2 gene and comprise a first mutant allele of the endogenous bm3 gene and a second mutant allele of the endogenous br2 gene and/or may be heteroallelic for the endogenous bm3 gene and comprise a first mutant allele of the endogenous bm3 gene and a second mutant allele of the endogenous bm3 gene.

For example, in some embodiments, the maize plant(s) (or parts thereof), populations of maize plants, and/or the silage product(s) produced therefrom (as described herein) may exhibit (or may have) a generally reduced lignin content, for example, as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression or a mutant allele of the endogenous bm3 gene, etc.) or plant part and/or a silage product including such a wild-type or control maize plant or plant part. Generally, lignin polymers may limit digestibility of the fiber in the corn plant. As such, the reduced lignin content provided for herein may result in maize plant(s) and/or silage product(s) with fiber that is more digestible (e.g., for animals, etc.), for example, as compared to a wild-type or control maize plant or plant part and/or a silage product of such a wild-type or control maize plant or plant part.

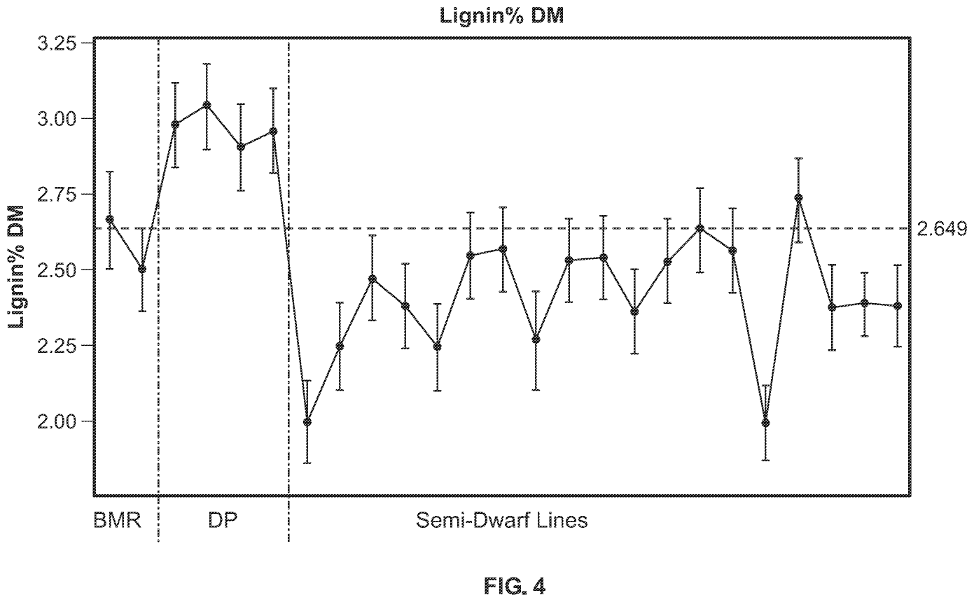

The maize plant(s) herein (or parts thereof), populations of maize plants, and/or the silage product(s) produced therefrom (as described herein) may comprise a lignin content of about 6% or less, about 5.5% or less, about 5% or less, about 4.5% or less, about 4% or less, about 3.5% or less, or about 3% or less, or between about 1.5% and about 6%, or between about 2% and about 6%, or between about 1.5% and about 5%, or between about 2% and about 5%, or between about 1.5% and about 4%, or between about 2% and about 4%, or between about 1.5% and about 3%, or between about 2% and about 3% (see, e.g., FIGS. 4 and 11-12, etc.). Such lignin content of the maize plant(s) (or parts thereof) and/or the silage product(s) produced therefrom may be reduced by about 1%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, or more, as compared to a wild-type or control maize plant or plant part and/or a silage product of a wild-type or control maize plant or plant part. Further, in one particular example embodiment, such lignin content of the maize plant(s) (or parts thereof) and/or the silage product(s) produced therefrom may be reduced by about 1%, about 1.5%, about 2%, about 2.5%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, or more, as compared to a control maize plant having a native br3 mutant allele, or plant part and/or a silage product of the control maize plant or plant part having a native br3 mutant allele.

In some example embodiments, the maize plant(s) (or parts thereof), populations of maize plants, and/or the silage product(s) produced therefrom (as described herein) may exhibit (or may have) a generally reduced ADF, for example, as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression or a mutant allele of the endogenous bm3 gene, etc.) or plant part thereof and/or a silage product including such a wild-type or control maize plant or plant part. In general, ADF is a percentage of plant material in a forage that is difficult or not digestible by an animal (e.g., including cellulose, lignin, silica, pectin fiber, etc.). To that point, plants/silage products with generally lower ADF may be more digestible and may have more energy content. Herein, the maize plant(s) (or parts thereof) and/or the silage product(s) produced therefrom may comprise an ADF of about 50% or less, about 45% or less, about 40% or less, about 35% or less, about 30% or less, about 25% or less, about 20% or less, about 15% or less, between about 15% and about 40%, between about 20% and about 40%, between about 25% and about 50%, between about 25% and about 45%, between about 25% and about 40%, between about 20% and about 30%, etc.

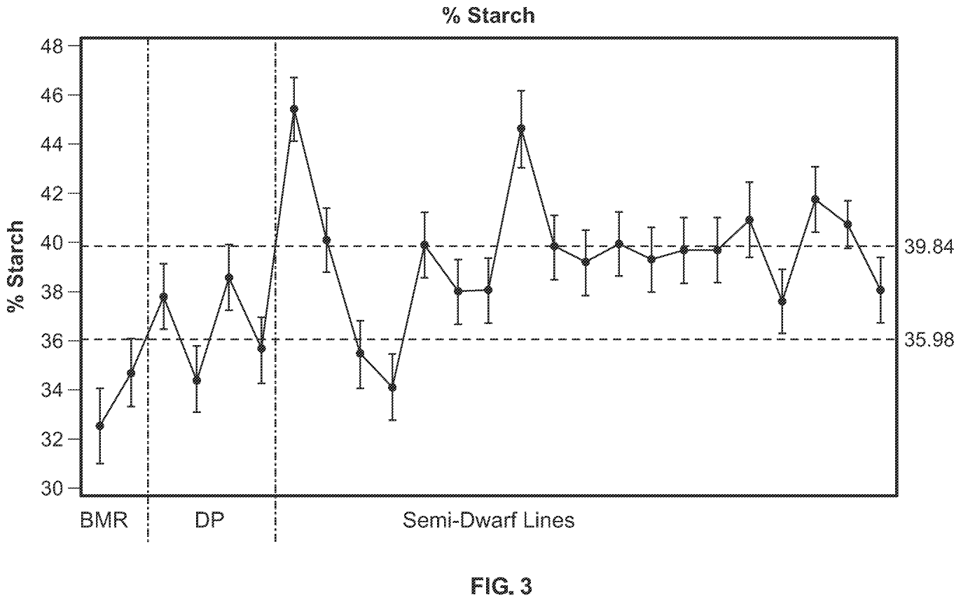

In some example embodiments, the maize plant(s) (or parts thereof), populations of maize plants, and/or the silage product(s) produced therefrom (as described herein) may exhibit (or may have) a generally increased starch content, for example, as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression or a mutant allele of the endogenous bm3 gene, etc.) or plant part thereof and/or a silage product including such a wild-type or control maize plant or plant part. For instance, the maize plant(s) (or parts thereof) herein and/or the silage product(s) produced therefrom may comprise a starch content (e.g., by weight, etc.) of about 15% or more, about 20% or more, about 30% or more, about 40% or more, about 50% or more, about 60% or more, between about 15% and about 70%, between about 25% and about 70%, between about 15% and about 60%, between about 15% and about 50%, between about 25% and about 50%, between about 30% and about 50%, between about 15% and about 40%, between about 30% and about 40%, between about 15% and about 30%, etc. (see, e.g., FIG. 3, etc.). Such starch content of the maize plant(s) (or parts thereof) and/or the silage product(s) produced therefrom may be increased by about 1% or more, about 2% or more, about 5% or more, about 10% or more, about 15% or more, about 20% or more, about 25% or more, about 30% or more as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression, a control plant not having a mutant allele of the endogenous bm3 gene, a control plant not having both a mutant allele of the endogenous br2 gene, a mutant allele of an endogenous GA oxidase gene, or a transgene targeting an endogenous GA oxidase gene for suppression, and a mutant allele of the endogenous bm3 gene, etc.) or plant part thereof and/or a silage product of a wild-type or control maize plant or plant part or as compared to a control maize plant or plant part and/or a silage product of the control maize plant or plant part.

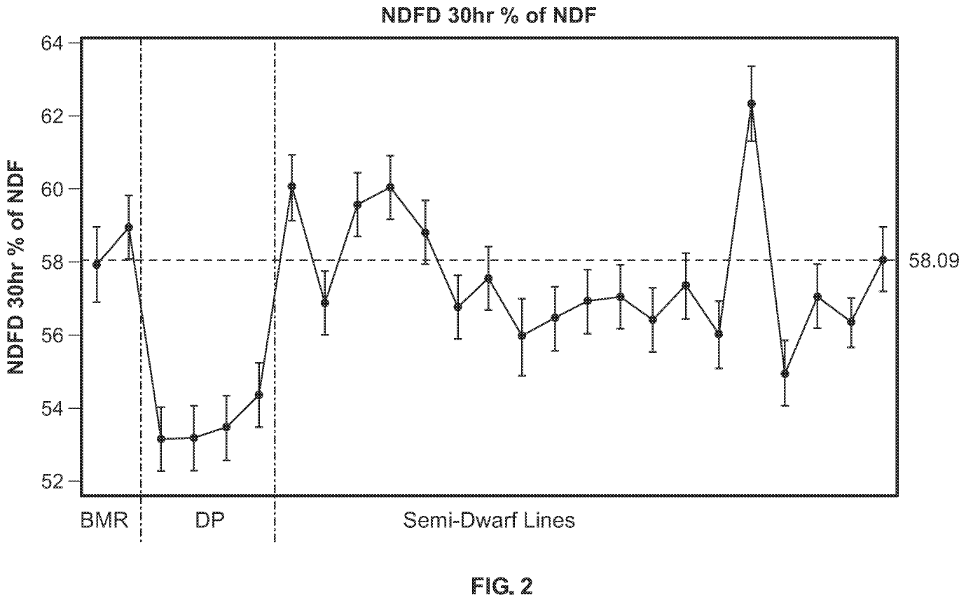

In some example embodiments, the maize plant(s) (or parts thereof), populations of maize plants, and/or the silage product(s) produced therefrom (as described herein) may exhibit (or may have) a generally increased NDFD for example, as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression or a mutant allele of the endogenous bm3 gene, etc.) or plant part thereof and/or a silage product including such a wild-type or control maize plant or plant part. Generally, NDF (neutral detergent fiber) refers to a measure of a fraction of feed that is not soluble in neutral detergent solution. To that end, NDF levels in forage generally increase as the plant matures. NDFD, then, generally refers to the percentage of NDF that is digestible (e.g., such that a relatively higher percentage generally indicates more digestible NDF (or an improvement therein), etc.). NDFD may be determined in vitro, for example, by incubating a ground feed sample in rumen fluid and measuring (e.g., at start and end of desired time period, for example, 30 hours, 48 hours, etc.) its disappearance to simulate the amount and rate of digestion that would occur in the rumen. See, e.g., Hoffman, P. et al., Using NDF Digestibility in Ration Formulation, Focus on Forage Vol. 6, No. 3 (2004). The maize plant(s) (or parts thereof) herein and/or the silage product(s) produced therefrom may comprise a NDFD (30 hour) of about 40% or more, about 45% or more, about 50% or more, about 55% or more, about 60% or more, about 65% or more, about 70% or more, about 75% or more, about 78% or more, about 80% or more, between about 20% and about 80%, between about 40% and about 80%, between about 50% and about 75%, between about 50% and about 70%, between about 55% and about 65%, between about 55% and about 60%, etc. (see, e.g., FIGS. 2 and 13-14, etc.). Such NDFD of the maize plant(s) (or parts thereof) and/or the silage product(s) produced therefrom may be increased by about 1% or more, about 2% or more, about 3% or more, about 5% or more, about 10% or more, about 15% or more, etc. as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression, a control plant not having a mutant allele of the endogenous bm3 gene, a control plant not having both a mutant allele of the endogenous br2 gene, a mutant allele of an endogenous GA oxidase gene, or a transgene targeting an endogenous GA oxidase gene for suppression, and a mutant allele of the endogenous bm3 gene, etc.) or plant part thereof and/or a silage product of such a wild-type or control maize plant or plant part.

In some example embodiments, the maize plant(s) (or parts thereof), populations of maize plants, and/or the silage product(s) produced therefrom (as described herein) may exhibit (or may have) a generally increased protein content, for example, as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression or a mutant allele of the endogenous bm3 gene, etc.) or plant part thereof and/or a silage product of a wild-type or control maize plant or plant part. In general, protein content is generally related to nitrogen content whereby higher nitrogen uptake may lead to higher protein. To that end, in one example, protein content may be determined via the Kjeldahl method as the nitrogen update multiplied by a factor of 6.25 (see, e.g., Machre, H. et al., “Protein Determination—Method Matters,” Foods 7, 5 (2018), etc.). The maize plant(s) (or parts thereof) herein and/or the silage product(s) produced therefrom may comprise a protein content of about 2% or more, about 4% or more, about 5% or more, about 6% or more, about 8% or more, about 10% or more, about 12% or more, about 14% or more, about 15% or more, about 16% or more, about 18% or more, about 20% or more, between about 2% and about 20%, between about 6% and about 20%, between about 10% and about 20%, etc. Such protein content of the maize plant(s) (or parts thereof) and/or the silage product(s) produced therefrom may be increased by about 2%, about 4%, about 6%, about 8%, about 10%, about 12%, or more as compared to a wild-type or control maize plant or plant part and/or a silage product of a wild-type or control maize plant or plant part.

In some example embodiments, the maize plant(s) (or parts thereof), populations of maize plants, and/or the silage product(s) produced therefrom (as described herein) may exhibit (or may have) a generally improved NUE, for example, as compared to a wild-type or control maize plant (e.g., a control plant not having a mutant allele of the endogenous br2 gene, a mutant allele of an endogenous GA oxidase gene, or a transgene targeting an endogenous GA oxidase gene for suppression or a mutant allele of the endogenous bm3 gene, etc.) or plant part thereof and/or a silage product including such a wild-type or control maize plant or plant part. NUE generally represents ability of the maize plant(s) to utilize nitrogen. As described above, protein content is generally related to nitrogen content whereby higher nitrogen uptake may lead to higher protein. The maize plant(s) (or parts thereof) and/or the silage product(s) produced therefrom may comprise a NUE of about 25% or more, about 30% or more, about 40% or more, about 50% or more, about 60% or more, about 70% or more, etc., as compared to a wild-type or control maize plant or plant part and/or a silage product of a wild-type or control maize plant or plant part.

In some example embodiments, the maize plant(s) (or parts thereof) and/or populations of maize plants, such as, but not limited to, a companion cropping system, may exhibit (or may have, provide or enable) improved weed management, for example, as compared to wild-type or control maize plants (e.g., a control maize plants not having a mutant allele of the endogenous br2 gene, a mutant allele of an endogenous GA oxidase gene, or a transgene targeting an endogenous GA oxidase gene for suppression or a mutant allele of the endogenous bm3 gene), or plant parts thereof. Further provided are silage product(s) (as described herein) produced from such maize plant(s) (or parts thereof), populations of maize plants, and/or companion cropping system. A better or improved weed control (e.g., weed suppression) may be achieved by increasing plant density, reducing row spacing, and/or intercropping plants (e.g., with a companion crop), which may be planted between maize plants and/or rows or maize plants.

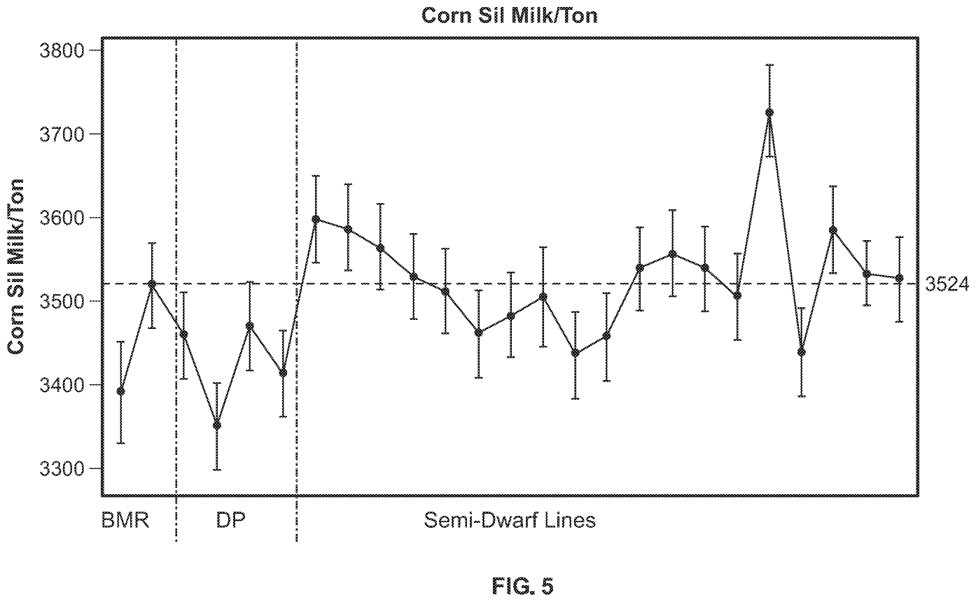

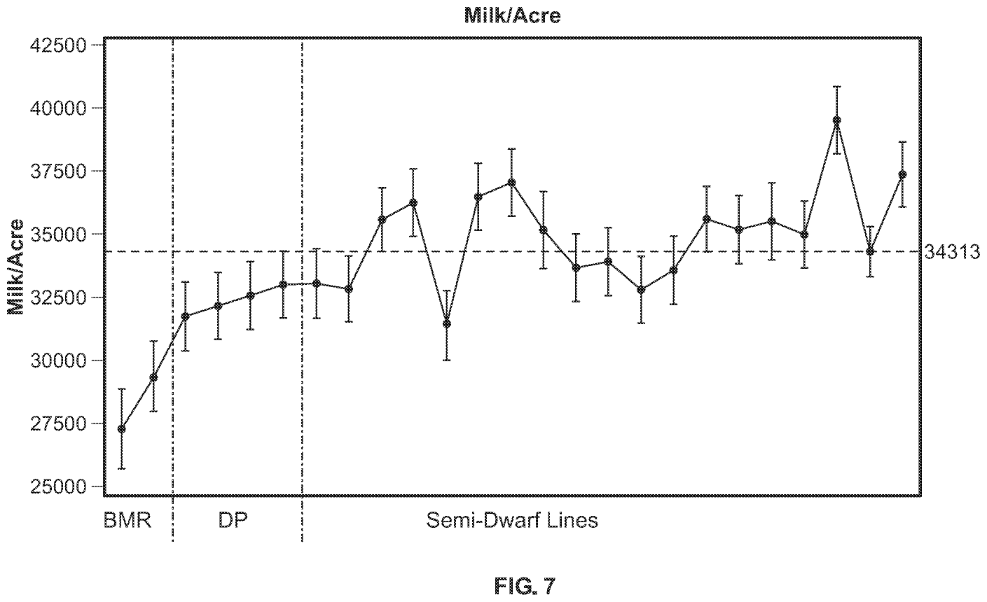

In some example embodiments, the maize plant(s) (or parts thereof), populations of maize plants, and/or the silage product(s) produced therefrom (as described herein) may exhibit (or may have) a generally improved milk output per acre and/or per ton of silage (dry matter). For example, the maize plant(s) (or parts thereof) and/or the silage product(s) produced therefrom may comprise a milk yield per acre output of about 20,000 lbs/acre or more, about 25,000 lbs/acre or more, about 30,000 lbs/acre or more, about 35,000 lbs/acre or more, about 40,000 lbs/acre or more, about 45,000 lbs/acre or more, about 50,000 lbs/acre, about 55,000 lbs/acre, about 60,000 lbs/acre or more, about 65,000 lbs/acre or more, etc. (see, e.g., FIG. 7, etc.). Additionally, or alternatively, the maize plant(s) herein (or parts thereof) and/or the silage product(s) produced therefrom may comprise a milk yield per ton output of about 1,500 lbs/ton or more, about 2,000 lbs/ton or more, about 2,500 lbs/ton or more, 3,000 lbs/ton or more, about 3,200 lbs/ton or more, about 3,400 lbs/ton or more, about 3,600 lbs/ton or more, about 3,800 lbs/ton or more, or about 4,000 lbs/ton or more, etc. (see, e.g., FIG. 5, etc.).

In some example embodiments, the maize plant(s) (or parts thereof), populations of maize plants, and/or the silage product(s) produced therefrom (as described herein) may exhibit (or may have) a generally improved DMB. In connection therewith, DMB generally includes or refers to the above-ground plant biomass, and moisture content is measured to determine the biomass-dry matter content (e.g., via one or more sensors or by taking samples which are then dried and weighed, etc.). The maize plant(s) herein (or parts thereof) and/or the silage product(s) produced therefrom may comprise a DMB of about 0.5 kg/m² or more, about 1 kg/m² or more, about 1.5 kg/m² or more, about 2 kg/m² or more, about 2.5 kg/m² or more, between about 0.5 kg/m² and about 2 kg/m², etc.

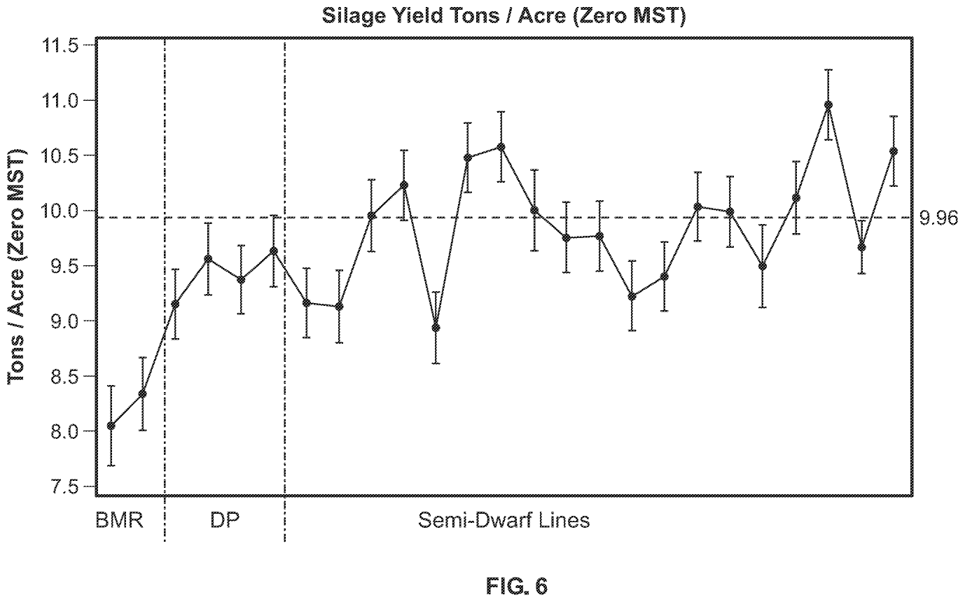

In some example embodiments, the maize plant(s) (or parts thereof), populations of maize plants, and/or the silage product(s) produced therefrom (as described herein) may exhibit (or may have) a generally improved silage yield. For example, the maize plant(s) herein (or parts thereof), populations of maize plants, and/or the silage product(s) produced therefrom may comprise (or produce) a silage yield of about 5 tons/Ha (about 2 tons/acre) or more, about 7 tons/Ha (about 2.8 tons/acre) or more, about 10 tons/Ha (about 4 tons/acre) or more, about 12 tons/Ha (4.9 tons/acre) or more, about 15 tons/Ha (6.1 tons/acre) or more, about 20 tons/Ha (8 tons/acre) or more, about 25 tons/Ha (about 10.1 tons/acre) or more, about 30 tons/Ha (about 12 tons/acre) or more, between about 7 tons/Ha and about 30 tons/Ha, ‘between about 7 tons/Ha (about 2.8 tons/acre) and about 12 tons/Ha (4.9 tons/acre), etc. (see, e.g., FIG. 6, etc.).

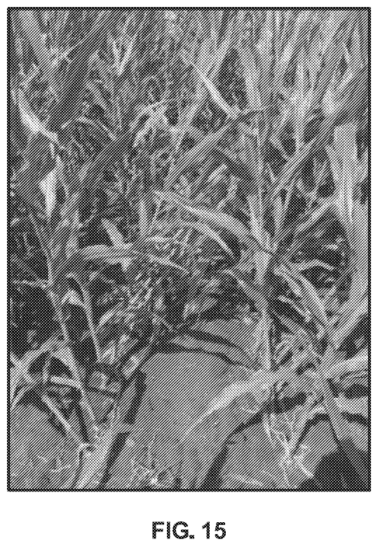

In some example embodiments, the maize plant(s) (or parts thereof), populations of maize plants, and/or the silage product(s) produced therefrom (as described herein) may exhibit (or may have) a generally improved (e.g., increased, etc.) resistance to stalk and root lodging and/or green snap (see, e.g., FIG. 15, etc.), for example, due to the shorter stalks and lower car placement associated therewith, as compared to a wild-type or control maize plant or plant part and/or a silage product of a wild-type or control maize plant or plant part.

In some embodiments, the maize plant(s) (or plant part thereof) and/or populations of maize plants (as described herein) may have a reduced average plant height (or shorter average plant height) as compared to a population of wild-type or control maize plants (e.g., including a wild-type or control maize plant not having a mutated br2 gene allele and/or not having a mutated bm3 gene allele, etc.), for example, about 10% or more, about 15% or more, about 20% or more, about 30% or more, about 40% or more, about 50% or more, about 60% or more, about 70% or more, between about 10% and about 70%, etc. shorter than the wild-type or control maize plant.

As described above, the description herein of the maize plant having a mutant allele of the endogenous br2 gene, a mutant allele of an endogenous GA oxidase gene, or a transgene targeting an endogenous GA oxidase gene for suppression, and/or a mutant allele of the endogenous bm3 gene is also applicable to one or more parts of the maize plant (e.g., a maize plant part, etc.), including, for example, a stem of the maize plant, a leaf of the maize plant, a seed of the maize plant, combinations thereof, etc. As such, the various compositions, traits, characteristics, etc., described herein for the maize plant (having a mutant allele of the endogenous br2 gene, a mutant allele of an endogenous GA oxidase gene, or a transgene targeting an endogenous GA oxidase gene for suppression, and/or a mutant allele of the endogenous bm3 gene) should be understood (as applicable or as appropriate) to also apply to a plant part thereof (e.g., to a maize plant seed (e.g., a seed of a maize plant herein, a seed produced as described herein (e.g., via genetic modification, etc.), etc.), etc.). For instance, consistent with the above description, a maize plant seed (having one or more of the various compositions, traits, characteristics, etc. described herein for the maize plant) is thus also provided herein, where the maize plant seed has both a mutant allele of the endogenous br2 gene, a mutant allele of an endogenous GA oxidase gene, or a transgene targeting an endogenous GA oxidase gene for suppression, and a mutant allele of the endogenous bm3 gene.

In addition, the maize plant(s) (or plant part thereof) and/or populations of maize plants described above may be grown in a growing space (e.g., a field, a greenhouse, or another controlled environment, etc.). As such, at least one example embodiment of the present disclosure includes a growing space (e.g., a field, a greenhouse, or another controlled environment, etc.) with the maize plant(s) and/or population of maize plants, described above, present and growing (or grown) in the growing space.

In some embodiments, a method for producing maize plant seeds herein may include crossing a first maize plant to a second maize plant, where either: (i) the first plant is a female plant and second plant is a male plant; or (ii) the second plant is the female plant and first plant is the male plant. In one example, then, the first maize plant may comprise a mutant allele of the endogenous br2 gene, a mutant allele of an endogenous GA oxidase gene, or a transgene targeting an endogenous GA oxidase gene for suppression, and a mutant allele of the endogenous bm3 gene. In another example, the first maize plant may comprise a mutant allele of the endogenous br2 gene, a mutant allele of an endogenous GA oxidase gene, or a transgene targeting an endogenous GA oxidase gene for suppression, and the second maize plant may comprise a mutant allele of the endogenous bm3 gene. In still another example, the first maize plant may comprise a mutant allele of the endogenous bm3 gene, and the second maize plant may comprise a mutant allele of the endogenous br2 gene, a mutant allele of an endogenous GA oxidase gene, or a transgene targeting an endogenous GA oxidase gene for suppression. In a further example, the second maize plant may comprise a mutant allele of the endogenous br2 gene, a mutant allele of an endogenous GA oxidase gene, or a transgene targeting an endogenous GA oxidase gene for suppression, and a mutant allele of the endogenous bm3 gene. In still further examples, the first maize plant may comprise either a mutant allele of the endogenous br2 gene, a mutant allele of an endogenous GA oxidase gene, or a transgene targeting an endogenous GA oxidase gene for suppression, or a mutant allele of the endogenous bm3 gene, and the second maize plant may not contain either a mutant allele of the endogenous br2 gene, a mutant allele of an endogenous GA oxidase gene, or a transgene targeting an endogenous GA oxidase gene for suppression, or a mutant allele of the endogenous bm3 gene.

The method may then include harvesting one or more maize plant progeny seeds (e.g., maize hybrid plant seeds, etc.) from the female maize plant, wherein the maize plant progeny seeds comprise either a mutant allele of the endogenous br2 gene, a mutant allele of an endogenous GA oxidase gene, or a transgene targeting an endogenous GA oxidase gene for suppression, a mutant allele of the endogenous bm3 gene, or a mutant allele of both the endogenous br2 gene, a mutant allele of an endogenous GA oxidase gene, or a transgene targeting an endogenous GA oxidase gene for suppression, and a mutant allele of the endogenous bm3 gene. In some example embodiments, the maize plant progeny seeds may be homozygous for a mutant allele of the endogenous bm3 gene. In some embodiments, the maize plant progeny seeds may be heterozygous for a mutant allele of the endogenous bm3 gene. Alternatively, the maize plant progeny seeds may be heteroallelic for an endogenous bm3 gene and may comprise a first mutant allele on one chromosome of the endogenous bm3 gene and a second mutant allele on a second homologous chromosome of the endogenous bm3 gene. Additionally, the maize plant progeny seeds may comprise one or two mutant alleles of the endogenous bm3 gene, which may be homozygous, heterozygous, and/or heteroallelic (as described herein). The maize plant progeny seeds may also be homozygous for a mutant allele of the endogenous br2 gene, a mutant allele of an endogenous GA oxidase gene, or a transgene targeting an endogenous GA oxidase gene for suppression. In some embodiments, the maize plant progeny seeds may be heterozygous for a mutant allele of the endogenous br2 gene, a mutant allele of an endogenous GA oxidase gene, or a transgene targeting an endogenous GA oxidase gene for suppression. The maize plant progeny seeds may be heteroallelic for the endogenous br2 or GA oxidase gene and may comprise a first mutant allele on one chromosome of the endogenous br2 or GA oxidase gene and a second mutant allele on a second homologous chromosome of the endogenous br2 or GA oxidase gene. Additionally, the maize plant progeny seeds may comprise one or two mutant alleles of the endogenous br2 or GA oxidase gene, which may be homozygous, heterozygous, and/or heteroallelic (as described herein).

In some embodiments, the maize plant progeny seeds may be collected, accumulated, bulked, etc. to produce a population (e.g., a plurality, etc.) of the maize plant progeny seeds. In doing so, the seeds in the population may include a mutant allele of the endogenous br2 gene, a mutant allele of an endogenous GA oxidase gene, or a transgene targeting an endogenous GA oxidase gene for suppression, or both of a mutant allele of the endogenous br2 gene, a mutant allele of an endogenous GA oxidase gene, or a transgene targeting an endogenous GA oxidase gene for suppression, and a mutant allele of the endogenous bm3 gene. For instance, in some examples, a majority of the seeds in the population may include a mutant allele of the endogenous br2 gene, a mutant allele of an endogenous GA oxidase gene, or a transgene targeting an endogenous GA oxidase gene for suppression, or both of a mutant allele of the endogenous br2 gene, a mutant allele of an endogenous GA oxidase gene, or a transgene targeting an endogenous GA oxidase gene for suppression, and a mutant allele of the endogenous bm3 gene. In some examples, each of the seeds in the population may include a mutant allele of the endogenous br2 gene, a mutant allele of an endogenous GA oxidase gene, or a transgene targeting an endogenous GA oxidase gene for suppression, or both of a mutant allele of both the endogenous br2 gene, a mutant allele of an endogenous GA oxidase gene, or a transgene targeting an endogenous GA oxidase gene for suppression, and a mutant allele of the endogenous bm3 gene. In one particular example, a majority of the maize plant progeny seeds in the population of seeds comprises a mutant allele of an endogenous br2 gene, a mutant allele of an endogenous GA oxidase gene, or a transgene targeting an endogenous GA oxidase gene for suppression, and/or a mutant allele of an endogenous bm3 gene. In another particular example, each of the maize plant progeny seeds in the population of seeds comprises a mutant allele of an endogenous br2 gene, a mutant allele of an endogenous GA oxidase gene, or a transgene targeting an endogenous GA oxidase gene for suppression, and a mutant allele of an endogenous bm3 gene.

Further, the method may include selecting one or more progeny maize plant progeny seeds that are homozygous, heterozygous and/or heteroallelic for a mutant allele of the endogenous bm3 gene and/or homozygous, heterozygous and/or heteroallelic for a mutant allele of the endogenous br2 gene, a mutant allele of an endogenous GA oxidase gene, or a transgene targeting an endogenous GA oxidase gene for suppression. And, the method may then include planting the selected one or more maize plant progeny seeds in a growing space (e.g., a field, a greenhouse, or another controlled environment, etc.) and growing one or more maize progeny plants (e.g., maize hybrid plants, etc.) from the maize plant progeny seeds.

In some example embodiments, in connection with producing maize plant seeds herein, the maize plant parent seeds may be homozygous for a mutant allele of the endogenous bm3 gene, or the maize plant parent seeds may be heterozygous for a mutant allele of the endogenous bm3 gene. Alternatively, the maize plant parent seeds may be heteroallelic for the endogenous bm3 gene and may comprise a first mutant allele (e.g., on one chromosome, etc.) of the endogenous bm3 gene and a second mutant allele (e.g., at the same locus on a second homologous chromosome, etc.) of the endogenous bm3 gene. In addition, or alternatively, the maize plant parent seeds may be homozygous for a mutant allele of the endogenous br2 gene, a mutant allele of an endogenous GA oxidase gene, or a transgene targeting an endogenous GA oxidase gene for suppression, or the maize plant parent seeds may be heterozygous for a mutant allele of the endogenous br2 gene, a mutant allele of an endogenous GA oxidase gene, or a transgene targeting an endogenous GA oxidase gene for suppression. Further, the maize plant parent seeds may be heteroallelic for the endogenous br2 or GA oxidase gene and may comprise a first mutant allele (e.g., on one chromosome, etc.) of the endogenous br2 or GA oxidase gene and a second mutant allele (e.g., at the same locus on a second homologous chromosome, etc.) of the endogenous br2 or GA oxidase gene.

In connection therewith, the method for producing the maize plant seeds may then further, or additionally, include selecting (e.g. via selection techniques such as markers, etc. described herein) one or more parent maize plant seeds that are homozygous, heterozygous, and/or heteroallelic for a mutant allele of the endogenous bm3 gene and/or homozygous, heterozygous and/or heteroallelic for a mutant allele of the endogenous br2 gene, a mutant allele of an endogenous GA oxidase gene, or a transgene targeting an endogenous GA oxidase gene for suppression to cross with another maize plant. Parent maize plants may be selected from a previous cross and/or may come from a line of multiple generations of plant crosses.

As discussed above, the plants, plant parts, and/or seeds described above may be used as silage, grain, or biofuels, such as in the production of ethanol. In some embodiments, the plants, plant parts, and/or seeds, or a population of the plants, plant parts, and/or seeds, may be used for one or more than one purpose (e.g., used as a dual purpose (DP), product). For example, the harvested plants or plant parts may be subsequently separated, and a portion thereof used as silage and a portion thereof used a grain. Alternatively, the harvested plants or plant parts may be subsequently separated, and a portion thereof used as silage and a portion thereof used for energy production (e.g., to produce ethanol), etc.

It should be appreciated that the plants, plant parts, and/or seeds described herein may comprise any combination of a mutant allele of the endogenous br2 gene, a mutant allele of an endogenous GA oxidase gene, or a transgene targeting an endogenous GA oxidase gene for suppression, and/or a mutant allele of the endogenous bm3 gene. For example, the plants, plant parts, and/or seeds may include a mutant allele of the endogenous br2 gene, a mutant allele of an endogenous GA oxidase gene, or a transgene targeting an endogenous GA oxidase gene for suppression, and may not include a mutant allele of the endogenous bm3 gene. In another example, the plants, plant parts, and/or seeds may include a mutant allele of the endogenous bm3 gene and may not include a mutant allele of the endogenous br2 gene, a mutant allele of an endogenous GA oxidase gene, or a transgene targeting an endogenous GA oxidase gene for suppression. In yet another example, the plants, plant parts, and/or seeds may include both a mutant allele of the endogenous br2 gene, a mutant allele of an endogenous GA oxidase gene, or a transgene targeting an endogenous GA oxidase gene for suppression, and a mutant allele of the endogenous bm3 gene.

In some example embodiments, a maize plant or maize plant part comprises a mutant allele of the endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter, wherein the maize plant or maize plant part has one or more traits selected from the group consisting of reduced lignin content, improved fiber digestibility, reduced ADF, increased starch content, increased NDFD or DCW, increased biomass, increased protein content (and/or N-stover), increased milk output, increased grain yield, and increased stem cross-section area, as compared to a wild-type or control maize plant or plant part. In connection therewith, the mutant allele of the endogenous br2 gene comprises an insertion, deletion, and/or substitution of one or more nucleotides, or any combination thereof, in the endogenous br2 gene, or the mutant allele of the endogenous GA20 oxidase gene comprises an insertion, deletion, and/or substitution of one or more nucleotides, or any combination thereof, in the endogenous GA20 oxidase gene, or the mutant allele of the endogenous GA3 oxidase gene comprises an insertion, deletion, and/or substitution of one or more nucleotides, or any combination thereof, in the endogenous GA3 oxidase gene.

In addition, in connection with the above maize plant or maize plant part, the mutant allele of the endogenous br2 gene is selected from the group consisting of the br2-23 allele, br2-7081 allele, br2-7861 allele, br2-qph1 allele, br2-qpa1 allele, br2-NC238 allele, and br2-1005 allele. Additionally, or alternatively, the mutant allele of the endogenous br2 gene is an edited allele of the endogenous br2 gene; and/or the maize plant or maize plant part is homozygous for the mutant allele of the endogenous br2 gene; and/or the maize plant or maize plant part is heteroallelic for the endogenous br2 gene and comprises a first mutant allele of the endogenous br2 gene and a second mutant allele of the endogenous br2 gene; and/or the maize plant or maize plant part is a maize hybrid plant or a maize plant part thereof.

### EXAMPLES

The following examples are exemplary in nature. Variations of the following examples are possible without departing from the scope of the disclosure.

### Example 1: Brachytic Trait Improves Silage Quality

Nineteen semi-dwarf (SD) lines (e.g., maize plants with a mutant allele of the endogenous br2 gene, etc.) were each planted in 20-inch row plots at a density of 55,000 plants per acre. Four elite commercial dual purpose (DP) lines and two commercial brown mid-rib (BMR) lines (e.g., maize plants with a mutant allele of the endogenous bm3 gene, etc.) were each planted in 20-inch row plots at a density of 40,000 plants per acre as controls. Each line plot was replicated four times. When plants reached full maturity, each plot was harvested and sent to Dairyland Laboratories in Arcadia, WI for silage quality analysis. Key silage traits measured included NDFD, percent starch, lignin, milk per ton, tons per acre and milk per acre. NDFD is measured as percent digestion after 30 hours (e.g., NDFD 30 hr, or NDFD30, or NDF30, etc.). Lignin is measured as percent dry matter (DM) digestibility. Protocols necessary for silage quality analysis are adopted by industry through The Association of Official Analytical Chemists (Baur, F. J. et al., The Association of Official Analytical Chemists (AOAC). J Am Oil Chem. Soc. 54, 171-172 (1977)) and performed by Dairyland Laboratories in accordance with these protocols.

Most of the SD products tested had significantly higher NDFD 30 hr, compared to dual purpose (DP) products (see, FIG. 2), while approximately one-half of the SD products tested had a NDFD 30 hr measurement similar to BMR products. Most of the SD products had percent starch higher than BMR products but similar to DP products (see, FIG. 3). Most SD products have lignin percentages based on dry matter (lignin % DM) significantly lower than elite DP products but similar to BMR products (see, FIG. 4). Four SD products had milk per ton significantly higher than elite DP products, but over half of the SD products tested had milk per ton that trended higher than both BMR and elite DP products (see, FIG. 5). Silage dry matter yield and milk per acre for most SD products were significantly higher than BMR products (see, FIGS. 6 and 7). Five of the SD products had milk per acre higher than elite DP products (see, FIG. 7).

Across key silage traits, there is a significant genetic variation that differentiates SD, elite dual purpose and BMR products. Within the population of SD products tested, there is genetic variation for key silage traits. The SD products tested showed an overall improvement in silage quality, both in energy content and energy availability.

### Example 2: Brachytic Trait Improves Cell Wall Digestibility

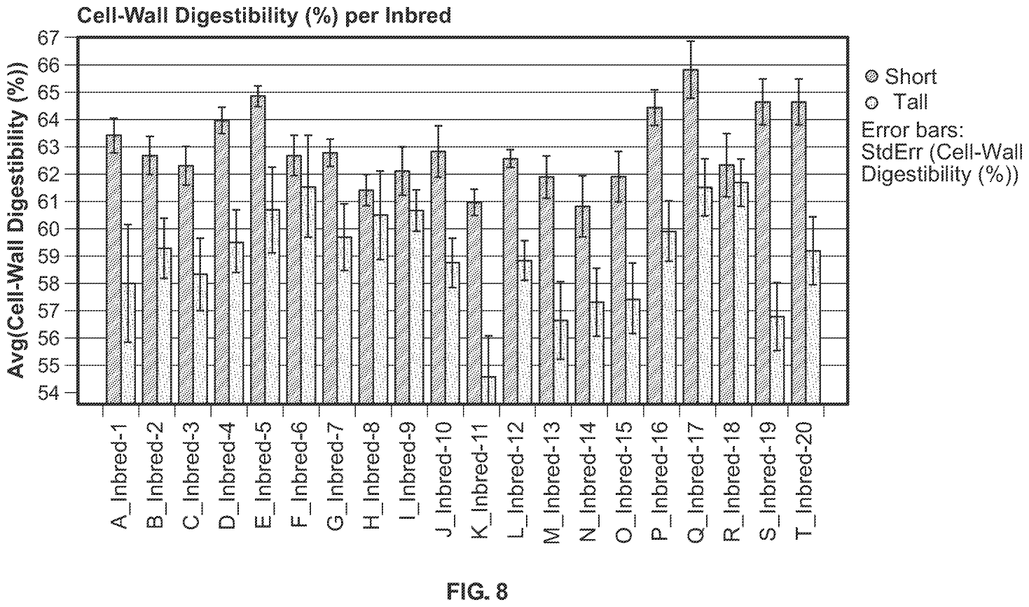

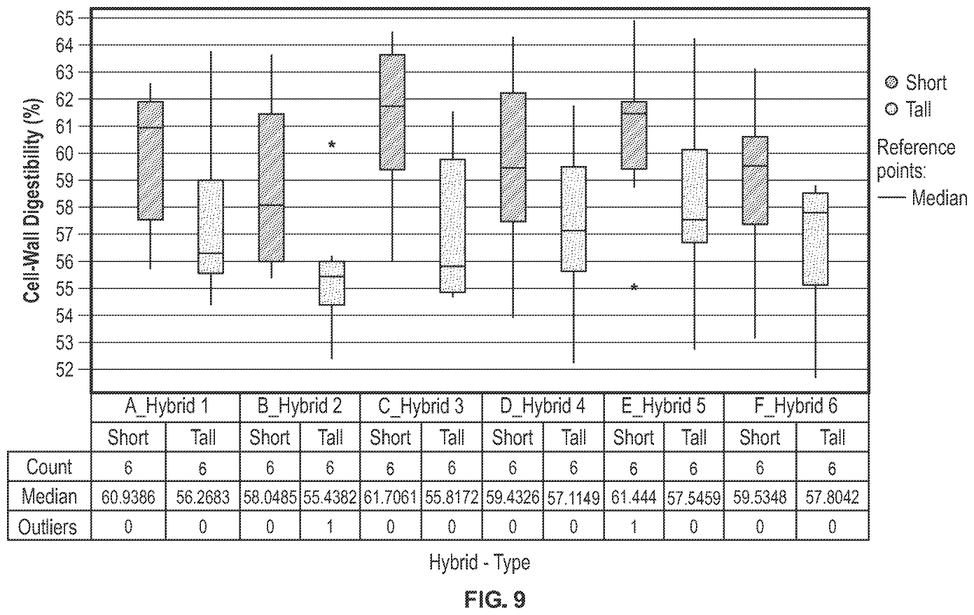

Twenty isogenic inbred lines (conventional and SD lines) were planted at 3 growing locations with 2 replicates per location. When plants reached full maturity, each plot was harvested, and silage quality analysis was performed. The SD lines had a significant increase in cell wall digestibility (DCW) (see, FIG. 8). No effect on NDF was observed between conventional and SD lines. A lower ADL content was observed in the SD lines. These silage quality metrics demonstrate an improvement in cell wall digestibility in SD lines compared to conventional lines. Six isogenic hybrids (conventional and SD lines) were also subject to silage quality analysis. The hybrid SD lines showed a significant increase in DCW (see, FIG. 9).

To further study the improvement in cell wall digestibility, three internodes per field replication were collected from one growing location. A 150 μm stem cross-section was obtained from the internode below the car from each stalk for FASGA Staining. The FASGA staining was performed by INRAE (National Research Institute for Agriculture, Food and Environment in France). Staining protocol was performed in accordance with Legland et al. (Histological quantification of maize stem sections from FASGA-stained images. Plant Methods 13, 84 (2017)). Because lignin is acidic, lignified tissues are stained in red even if this stain is not completely specific for only lignin. FASGA thus stains lignified tissues in red, whereas non-lignified or poorly lignified tissues appear as blue. Quantification of colors in the FASGA stained sections were performed with standard imaging software.

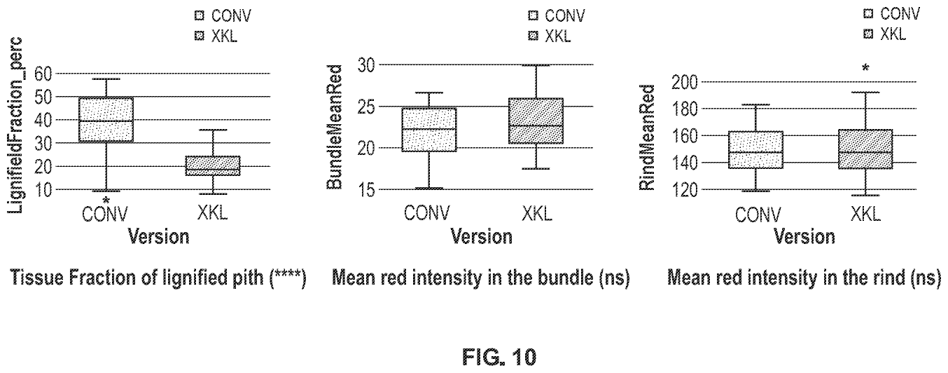

Cross-sections of the SD hybrids demonstrated an overall increase in stem area by 0.32 cm² as compared to the conventional hybrid. Histology of the cross-sections of the SD hybrids displayed a significant increase in the bundle fraction and an increase in bundle number as compared to conventional hybrids. There was no observed difference for rind fraction or bundle intensity. The SD hybrids demonstrated a difference in lignin distribution as compared to conventional lines. There was a decrease in tissue fraction of lignified pith (see, FIG. 10). These results demonstrate that SD hybrids have increased cell wall digestibility by observing a lower level of lignin, particularly in the pith, as compared to isogenic conventional hybrids.

### Example 3: Reduction of Lignin Contents

To evaluate the effect of bm3 mutation on silage quality in short and tall stature corn hybrids, 18 parental elite lines (LN01 through LN18) went through BC3F3 conversions with bm3 mutation (see, Morrow et al. (1997) Mol. Breeding. 3:351-7; Vignols et al. (1995) Plant Cell 7:407-16). The bm3 donor lines 408E and 415E were obtained from the maize genetic stock center and used for conversions. Of the 18 lines with bm3 mutation conversion, 9 lines (LN03 through LN06, and LN08 through LN12) were also subject to conversion with short stature br2 mutation (see, e.g., PCT/US2016/029492, the entire content and disclosure of which are incorporated herein by reference). Tall stature refers to the normal corn plant height of the hybrids, which is tall as compared with plants of short stature br2 mutation.

Corn hybrids were produced by crossing the parental elite lines, and grouped into hybrids with bm3 mutation (BMR) and without bm3 mutation (wild-type hybrids, or WT). The hybrids were also divided into groups of short and tall statures, based on the presence or absence of br2 mutation respectively.

The corn hybrids were grown in 2 consecutive growing seasons, under standard agronomic practice (SAP), in replicated microplots, with 10 to 12 replications per hybrid. Each microplot consisted of 12 plants in a single row. Short and tall stature hybrids were grown in separate blocks. Above-ground biomass was harvested at physiological maturity R6 stage, and properly mixed and grounded for silage quality analysis by Dairyland Laboratories, Inc. (217 E Main St, Arcadia, WI 54612). In Growing Season-1 (or a first growing season) corn ears were excluded from above-ground biomass.

Lignin content analysis was conducted according to Fiber (Acid Detergent) and Lignin in Animal Feed: AOAC Official Method 973.18 (1996).

Tables 3 and 4 show lignin content level analysis results for two consecutive growing seasons. In each of the short and tall stature groups, Base Hybrid indicates the two elite parental lines of the hybrid, Type indicates either presence of bm3 mutation (BMR), or absence (wild-type, or WT). Median lignin content percentage and standard error (SE) are shown in the tables.

TABLE 3
Isogenic hybrid comparisons for Lignin content
level, of Growing Season-1

| Stature | Base Hybrid | Type | % Lignin | Lignin SE |
|---|---|---|---|---|
| Short | LN08 + LN10 | BMR | 3.88 | 0.14 |
| | | WT | 4.42 | 0.09 |
| | LN04 + LN10 | BMR | 3.6 | 0.13 |
| | | WT | 4.84 | 0.15 |
| Tall | LN07 + LN13 | BMR | 3.45 | 0.12 |
| | | WT | 4.34 | 0.12 |
| | LN07 + LN15 | BMR | 3.82 | 0.11 |
| | | WT | 4.91 | 0.13 |
| | LN16 + LN13 | BMR | 3.8 | 0.11 |
| | | WT | 4.76 | 0.11 |
| | LN16 + LN15 | BMR | 4.08 | 0.12 |
| | | WT | 5 | 0.12 |

TABLE 4
Isogenic hybrid comparisons for Lignin
content level, of Growing Season-2

| Stature | Base Hybrid | Type | % Lignin | Lignin SE |
|---|---|---|---|---|
| Short | LN05 + LN09 | BMR | 2.4 | 0.085 |
| | | WT | 2.6 | 0.085 |
| | LN05 + LN10 | BMR | 2.2 | 0.095 |
| | | WT | 2.9 | 0.089 |
| | LN05 + LN11 | BMR | 2.1 | 0.085 |
| | | WT | 2.6 | 0.085 |
| | LN06 + LN09 | BMR | 2.5 | 0.085 |
| | | WT | 2.7 | 0.089 |
| | LN08 + LN03 | BMR | 2.5 | 0.085 |
| | | WT | 2.9 | 0.085 |
| | LN08 + LN10 | BMR | 2.3 | 0.089 |
| | | WT | 2.6 | 0.085 |
| | LN08 + LN11 | BMR | 2.2 | 0.085 |
| | | WT | 2.6 | 0.095 |
| | LN12 + LN09 | BMR | 2.3 | 0.085 |
| | | WT | 2.8 | 0.085 |
| | LN12 + LN10 | BMR | 2.3 | 0.085 |
| | | WT | 2.8 | 0.089 |
| | LN12 + LN11 | BMR | 2.2 | 0.085 |
| | | WT | 2.6 | 0.085 |
| Tall | LN01 + LN02 | BMR | 2.2 | 0.085 |
| | | WT | 2.6 | 0.085 |
| | LN01 + LN14 | BMR | 2.1 | 0.085 |
| | | WT | 2.6 | 0.089 |
| | LN07 + LN13 | BMR | 2.3 | 0.085 |
| | | WT | 2.8 | 0.089 |
| | LN07 + LN15 | BMR | 2.5 | 0.085 |
| | | WT | 2.7 | 0.085 |
| | LN16 + LN13 | BMR | 2.3 | 0.089 |
| | | WT | 2.7 | 0.085 |
| | LN16 + LN15 | BMR | 2.5 | 0.085 |
| | | WT | 3.1 | 0.085 |
| | LN17 + LN02 | BMR | 2.3 | 0.06 |
| | | WT | 2.6 | 0.062 |
| | LN17 + LN14 | BMR | 2.5 | 0.089 |
| | | WT | 2.7 | 0.085 |

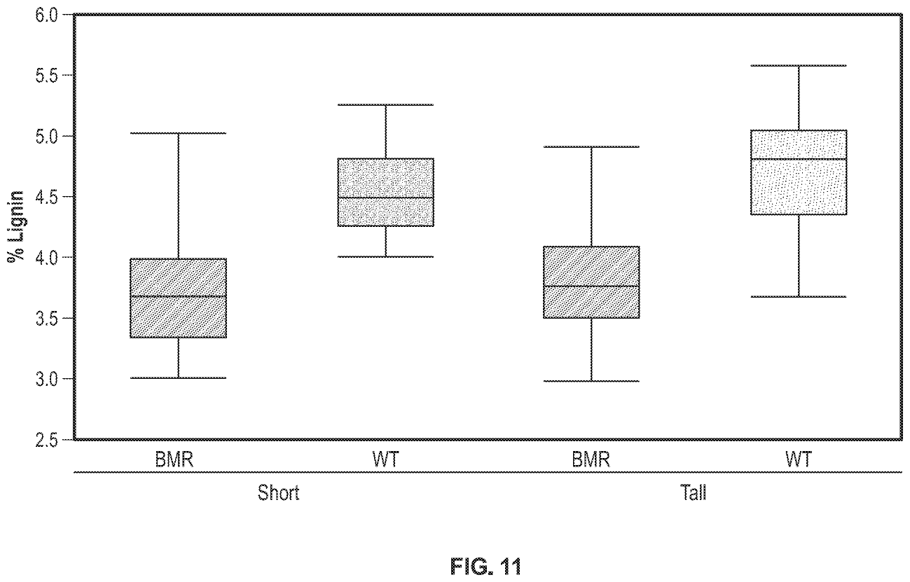

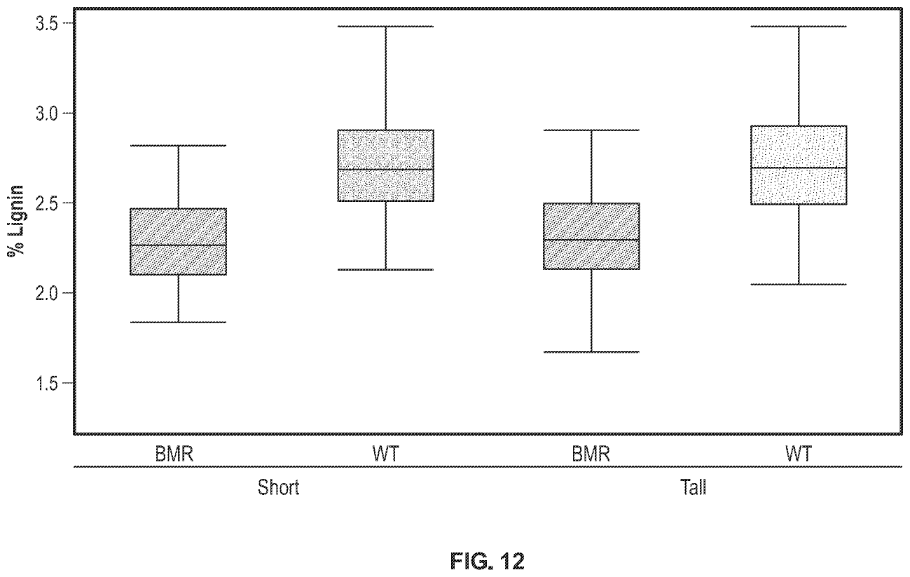

FIGS. 11 and 12 show in box plot of pooled isogenic hybrid comparisons of lignin content, of Growing Season-1 (first growing season) and Growing Season-2 (second growing season) respectively. A box plot provides a compact view of a distribution of values. The box extends from the 25^{th} percentile to the 75^{th} percentile where the distance between the 75^{th} and 25^{th} percentiles is the interquartile range (IQR). The median is marked within the box. For outlier box plots, whiskers extend to the last point that is within 1.5*IQR from the ends of the box.

To evaluate the statistical differences of the 4 groups of hybrids, mean separation tests were done suing Least Square Means (Lsmeans) Differences student's t pairwise comparison. Results are summarized in Tables 5 and 6, of Growing Season-1 and Growing Season-2 respectively. Lignin content levels of different statistical grouping letters are significantly different. “Tall” refers to plants with a non-mutant br2 gene and “short” refers to plants with a mutant allele of the endogenous br2 gene. “WT” refers to plants with a non-mutant bm3 gene. “BMR” refers to plants with a mutant allele of the endogenous bm3 gene.

TABLE 5
Grouping of different lignin content levels
by hybrids, of Growing Season-1

| Hybrid Group | Statistical Grouping | Least Sq Mean | Std Error |
|---|---|---|---|
| Tall, WT | A | 4.7 | 0.07 |
| Short, WT | A | 4.5 | 0.09 |
| Tall, BMR | B | 3.8 | 0.06 |
| Short, BMR | B | 3.7 | 0.11 |

α = 0.05
t = 1.97897

TABLE 6
Grouping of different lignin content levels
by hybrids, of Growing Season-2

| Hybrid Group | Statistical Grouping | Least Sq Mean | Std Error |
|---|---|---|---|
| Tall, WT | A | 2.73 | 0.031 |
| Short, WT | A | 2.72 | 0.03 |
| Tall, BMR | B | 2.32 | 0.031 |
| Short, BMR | B | 2.3 | 0.029 |

α = 0.05
t = 1.96652

As shown in Tables 5 and 6, the lignin content levels are significantly lower for BMR mutant hybrids (e.g., plants having both mutant alleles of the br2 gene and mutant alleles of the bm3 gene) as compared with the hybrids without BMR mutant (Group A vs B).

### Example 4: Improved Digestibility

Following the hybrid corn growth and sampling in each of the two consecutive growing seasons, as described in Example 3, digestibility analysis (NDF30, Neutral Detergent Fiber after 30-hour incubating) was conducted according to Amylase-Treated Neutral Detergent Fiber in Feeds AOAC Official Method 2002.04 2005.

Tables 7 and 8 show NDF30 digestibility results for two consecutive growing seasons. In each of the short (e.g., plants having a mutant allele for the br2 gene) and tall (e.g., plants having non-mutant or wild-type alleles for the br2 gene) stature groups, Base Hybrid indicates the two elite parental lines of the hybrid, Type indicates either presence of bm3 mutation (BMR), or absence (wild-type, or WT). Median NDF30 digestibility and standard error (SE) are shown in the tables.

TABLE 7
Isogenic hybrid comparisons for NDF30 digestibility,
of Growing Season-1

| | | | % Digestibility | |
|---|---|---|---|---|
| Stature | Base Hybrid | Type | (NDF30) | NDF30 SE |
| Short | LN08 + LN10 | BMR | 64.27 | 0.79 |
| | | WT | 58.24 | 0.53 |
| | LN04 + LN10 | BMR | 64.46 | 0.75 |
| | | WT | 55.89 | 0.85 |
| Tall | LN07 + LN13 | BMR | 58.44 | 0.67 |
| | | WT | 54.65 | 0.67 |
| | LN07 + LN15 | BMR | 62.63 | 0.65 |
| | | WT | 54.25 | 0.71 |
| | LN16 + LN13 | BMR | 60.71 | 0.65 |
| | | WT | 51.78 | 0.67 |
| | LN16 + LN15 | BMR | 59.02 | 0.67 |
| | | WT | 51.63 | 0.67 |

TABLE 8
Isogenic hybrid comparisons for NDF30 digestibility,
of Growing Season-2

| | | | % Digestibility | |
|---|---|---|---|---|
| Stature | Base Hybrid | Type | (NDF30) | NDF30 SE |
| Short | LN05 + LN09 | BMR | 66.4 | 0.759 |
| | | WT | 61.3 | 0.759 |
| | LN05 + LN10 | BMR | 67.6 | 0.848 |
| | | WT | 59.6 | 0.8 |
| | LN05 + LN11 | BMR | 72.5 | 0.759 |
| | | WT | 66 | 0.759 |
| | LN06 + LN09 | BMR | 65.9 | 0.759 |
| | | WT | 61.3 | 0.8 |
| | LN08 + LN03 | BMR | 66.8 | 0.759 |
| | | WT | 61.9 | 0.759 |
| | LN08 + LN10 | BMR | 67.2 | 0.8 |
| | | WT | 62.4 | 0.759 |
| | LN08 + LN11 | BMR | 71.3 | 0.759 |
| | | WT | 65.4 | 0.848 |
| | LN12 + LN09 | BMR | 67.3 | 0.759 |
| | | WT | 61.1 | 0.759 |
| | LN12 + LN10 | BMR | 68.9 | 0.759 |
| | | WT | 61.6 | 0.8 |
| | LN12 + LN11 | BMR | 73 | 0.759 |
| | | WT | 64.6 | 0.759 |
| Tall | LN01 + LN02 | BMR | 67.7 | 0.759 |
| | | WT | 61.1 | 0.759 |
| | LN01 + LN14 | BMR | 69.5 | 0.759 |
| | | WT | 60.7 | 0.8 |
| | LN07 + LN13 | BMR | 67.2 | 0.759 |
| | | WT | 61 | 0.8 |
| | LN07 + LN15 | BMR | 64.3 | 0.759 |
| | | WT | 61.3 | 0.759 |
| | LN16 + LN13 | BMR | 67.6 | 0.8 |
| | | WT | 60.4 | 0.759 |
| | LN16 + LN15 | BMR | 66.4 | 0.759 |
| | | WT | 57.1 | 0.759 |
| | LN17 + LN02 | BMR | 67 | 0.537 |
| | | WT | 60.7 | 0.55 |
| | LN17 + LN14 | BMR | 65.3 | 0.8 |
| | | WT | 61 | 0.759 |

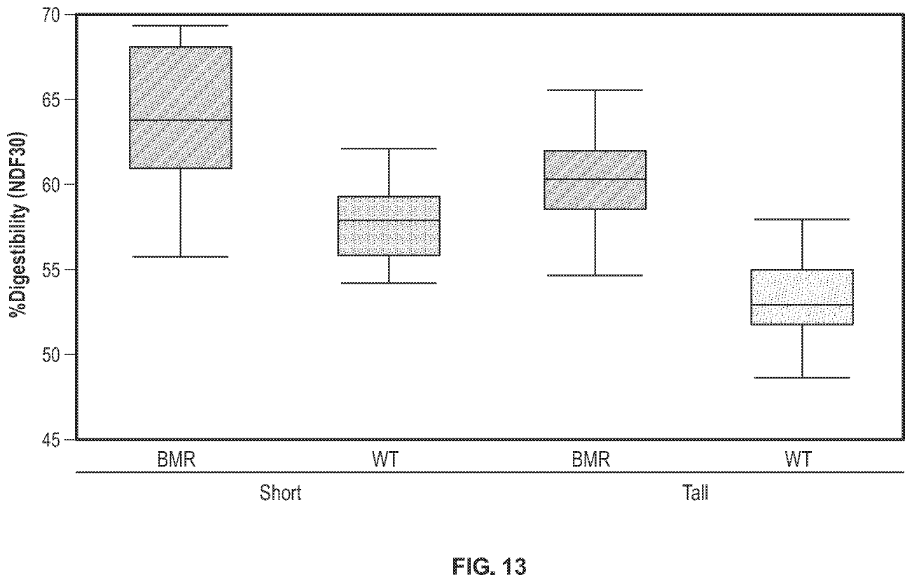

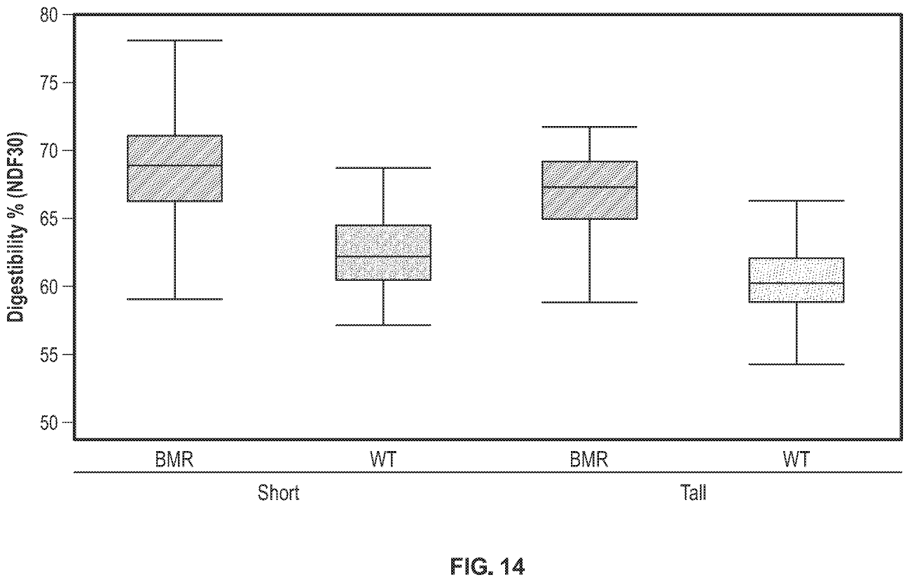

FIGS. 13 and 14 show in box plot of pooled isogenic hybrid comparisons of NDF30 digestibility, of Growing Season-1 and Growing Season-2 respectively. A box plot provides a compact view of a distribution of values. The box extends from the 25th percentile to the 75th percentile where the distance between the 75th and 25th percentiles is the interquartile range (IQR). The median is marked within the box. For outlier box plots, whiskers extend to the last point that is within 1.5*IQR from the ends of the box.

To evaluate the statistical differences of the 4 groups of hybrids, mean separation tests were done suing Least Square Means (Lsmeans) Differences student's t pairwise comparison. Results are summarized in Tables 9 and 10, of Growing Season-1 and Growing Season-2 respectively. NDF30 digestibility levels of different statistical grouping letters are significantly different. “Short,BMR”, “Tall,BMR”, “Short, WT”, and “Tall, WT” are defined as above.

TABLE 9
Grouping of different lignin content levels by hybrids,
of Growing Season-1

| Hybrid Group | Statistical Grouping | Least Sq Mean | Std Error |
|---|---|---|---|
| Short, BMR | A | 64.4 | 0.62 |
| Tall, BMR | B | 60.3 | 0.38 |
| Short, WT | C | 57.6 | 0.51 |
| Tall, WT | D | 53.1 | 0.39 |

α = 0.05
t = 1.97897

TABLE 10
Grouping of different lignin content levels by hybrids,
of Growing Season-2

| Hybrid Group | Statistical Grouping | Least Sq Mean | Std Error |
|---|---|---|---|
| Short, BMR | A | 68.7 | 0.3 |
| Tall, BMR | B | 66.9 | 0.32 |
| Short, WT | C | 62.5 | 0.3 |
| Tall, WT | D | 60.4 | 0.32 |

α = 0.05
t = 1.96652

As shown in Tables 9 and 10, the NDF30 digestibility level is significantly higher for BMR mutant hybrids as compared with the hybrids without BMR mutant (Groups A and B vs Groups C and D). Furthermore, the NDF30 digestibility levels are significantly higher for BMR mutant hybrids of shorter stature, as compared with BMR mutant hybrids of tall stature (Group A vs Group B). This demonstrates the synergistic effect in improving digestibility, when BMR mutant is combined with short stature.

### Example 5: Improved Lodging Resistance

Brown midrib corn has always been considered to be more prone to lodging due to its lower lignin content. It was reported that mean lodging rate of 36.3% was observed in commercial PIONEER® BMR corn varieties (see, e.g., www.uvm.edu/sites/default/files/Northwest-Crops-and-Soils-Program/2014-ResearchReports/2014_BMR_Population_Report.pdf 2014 Brown Mid-Rib Corn Population Trial, by Dr. Heather Darby of University of Vermont Extension). It was suggested that corn planting population density can be lowered to allow for less stress on each individual plant, thus reducing lodging.

In Growing Season-1 as described in Example 3, corn plant lodging was observed in the microplot field of BMR (e.g., plants having a mutant allele in the bm3 gene) and non-BMR (WT) hybrids, of short and tall stature. FIG. 15 shows representative lodging in the field. The observation results are summarized in Table 11, with the groups of pooled isogenic hybrids. A microplot is considered as with lodging, if one or more plants lodged. Average percent of plants lodged was 31.7%, in microplots with lodging. “Short, BMR”, “Tall, BMR”, “Short, WT”, and “Tall, WT” are defined as above.

TABLE 11
Observation of corn plant lodging, grouped by pooled
isogenic hybrids, of Growing Season-1

| | | Number of Plots | % of Plots with |
|---|---|---|---|
| Group | Total Plots | with Lodging | Lodging |
| Short, BMR | 21 | 0 | 0 |
| Short, WT | 22 | 0 | 0 |
| Tall, BMR | 48 | 11 | 22.9% |
| Tall, WT | 44 | 0 | 0 |

In Growing Season-1 as described in Example 3, corn plant lodging was observed in the microplot field of BMR and non-BMR (WT) hybrids, of short and tall stature. FIG. 15 shows representative lodging in the field. The observation results are summarized in Table 11, with the groups of pooled isogenic hybrids. A microplot is considered as with lodging, if one or more plants lodged. Average percent of plants lodged was 31.7%, in microplots with lodging.

As shown in Table 11, tall stature corn plants with BMR mutation experienced significant lodging, while short stature plants with BMR mutation had no observed lodging. Recall that tall stature plants refer to plants of normal height of the hybrids, without presence of the br2 mutation. BMR mutation in combination with short stature seems to meet the long felt yet unsolved needs, to significantly reduce plant lodging.

### Example 6: Improved Silage Traits of Corn with Mutant Br2 and Bm3 Genes

In this example, maize plant(s) (or parts thereof) and/or the silage product(s) produced therefrom, comprising a mutant allele of the endogenous br2 gene and/or a mutant allele of the endogenous bm3 gene as described herein, exhibited (or included) one or more of the following characteristics in Table 12.

TABLE 12

| Category | Avg | Min | Max | Median | StdDev |
|---|---|---|---|---|---|
| Acid Detergent Fiber (ADF) (%) | 21.89 | 15.82 | 30.67 | 21.83 | 2.21 |
| Ash (%) | 4.86 | 3.46 | 7.99 | 4.79 | 0.58 |
| Crude Protein (%) | 7.92 | 5.57 | 10.73 | 7.99 | 0.74 |
| Dry Matter (DM) (%) | 33.18 | 24.31 | 45.91 | 32.53 | 3.46 |
| Moisture (%) | 66.82 | 54.09 | 75.69 | 67.47 | 3.46 |
| Neutral Detergent Fiber (NDF) | 39.11 | 28.26 | 52.18 | 39.01 | 3.76 |
| (%) | | | | | |
| Neutral Detergent Fiber (NDF)- | 37.93 | 27.22 | 50.72 | 37.81 | 3.7 |
| Organic Matter (%) | | | | | |
| Non-Fiber Carbohydrates (NFC) | 47.4 | 31.79 | 57.52 | 47.38 | 3.38 |
| (%) | | | | | |
| Starch (%) | 33.59 | 15.69 | 47.89 | 33.42 | 5.19 |
| Total Digestible Nutrients (TDN) | 76.32 | 70.07 | 78 | 76.63 | 1.54 |
| (%) | | | | | |
| Milk/Ton (lbs/ton) | 3636.97 | 3137 | 3975 | 3644.5 | 133.95 |
| Net Energy for Lactation (NEL) | 0.75 | 0.67 | 0.79 | 0.75 | 0.02 |
| (Mcal/lb) | | | | | |
| In Vitro Starch Digestibility | 67.04 | 60.41 | 72.23 | 66.78 | 1.98 |
| (IVSD) 7 hr (4 mm) % of Starch | | | | | |
| Lignin Percent DM | 2.85 | 1.74 | 4.82 | 2.84 | 0.36 |
| Milk/Acre (lbs/acre) | 64184.36 | 28215.37 | 103517.8 | 66977.8 | 13844.26 |
| NDF Rate (k_{d}) (%) | 6.38 | 4.96 | 8.08 | 6.31 | 0.67 |
| NDFD 24 hr % of NDF | 59.32 | 47.32 | 69.99 | 59.43 | 4.12 |
| NDFD 30 hr % of NDF | 64.09 | 53.13 | 74.16 | 64.05 | 3.91 |
| NDFD 48 hr % of NDF | 67.88 | 57.94 | 76.93 | 67.79 | 3.4 |
| Net Energy for Gain (NEG) | 0.55 | 0.47 | 0.6 | 0.55 | 0.02 |
| (Mcal/lb) | | | | | |
| Sugar (%) | 3.24 | 0.89 | 6.87 | 3.14 | 1.13 |
| Tonnage (tons/Ha) | 26.61 | 8.74 | 39.04 | 28.53 | 7.33 |
| Tons per acre DM | 8.92 | 2.85 | 14.81 | 9.25 | 2.9 |
| Total Fatty Acids (TFA) | 2.11 | 1.26 | 3.15 | 2.08 | 0.31 |
| Total Volatile Fatty Acids (VFA) | 2.71 | 0.02 | 5.67 | 2.81 | 0.9 |
| uNDF 120 hr % DM | 9.68 | 7.01 | 14.36 | 9.68 | 1.17 |
| uNDF 240 hr % DM | 8.82 | 6.22 | 13.27 | 8.79 | 1.11 |
| uNDF 24 hr % DM | 15.34 | 12.06 | 21.42 | 15.29 | 1.4 |
| uNDF 30 hr % DM | 13.55 | 10.2 | 19.65 | 13.48 | 1.46 |
| uNDF 48 hr % DM | 12.12 | 9.12 | 16.89 | 12.07 | 1.3 |

### Example 7: Improved Silage Traits of Transgenic Short Stature Corn

Hybrid corn plants comprising a transgene conveying a short stature phenotype (transgenic short stature corn or tSSC plants) and a mutant allele of a brown midrib 3 (bmr3 or bm3) gene produced through genome editing were prepared for field testing as follows: the brown mid-rib 3 (bmr3 or bm3) gene edited corn line (e-bmr3; with 2808 bp deletion of the entire coding region, see, e.g., PCT Application Pub. No. WO 2020/117837, which is incorporated herein by reference) was crossed to a transgenic short stature corn line containing a transgene targeting the GA20 oxidase_3 and GA20 oxidase_5 genes for suppressin (tSSC; see, e.g., PCT Application Pub. No. WO 2018/035354, which is incorporated herein by reference) to generate F1 seed. F1 progeny plants were selfed (e.g., seeds were produced without the plants being pollinated, etc.) and planted to generate segregating seed. Plants homozygous for both the tSSC and e-bmr3 genotypes were selected to produce R2 seed (tSSC/tSSC: e-bmr3/e-bmr3). Hybrids of tSSC/e-bmr3 were generated by crossing the double homozygous R2 plants to a different parental line comprising a different mutant allele of the brown midrib 3 gene (bmr3) to generate hybrid corn seeds and plants having the following genotype at the two loci: tSSC/WT: e-bmr3/bmr3, which may also be referred to in this example as tSSC/e-bmr3. Hybrid short stature corn plants comprising the tSSC trait and heterozygous for the mutant allele of the bm3 gene locus, for use as a control, were generated by crossing homozygous tSSC corn plants to corn plants comprising the native bmr3 mutant allele having the following genotype: tSSC/WT: bmr3/WT, which may also be referred to in this example as simply tSSC.

By crossing the gene edited bmr3 line (e-bmr3) to the native bmr3 mutant line (bmr3), normal stature (tall) hybrid corn plants comprising the edited bmr3 allele (heteroallelic with the native mutant bmr3 gene), but without the tSSC transgene were generated, which may be referred to in this example as simply e-bmr3). By crossing an inbred wild-type line devoid of the tSSC transgene to the native bmr3 mutant line (bmr3), normal stature (tall) wild-type control plants were also generated, which may be referred to in this example as WT.

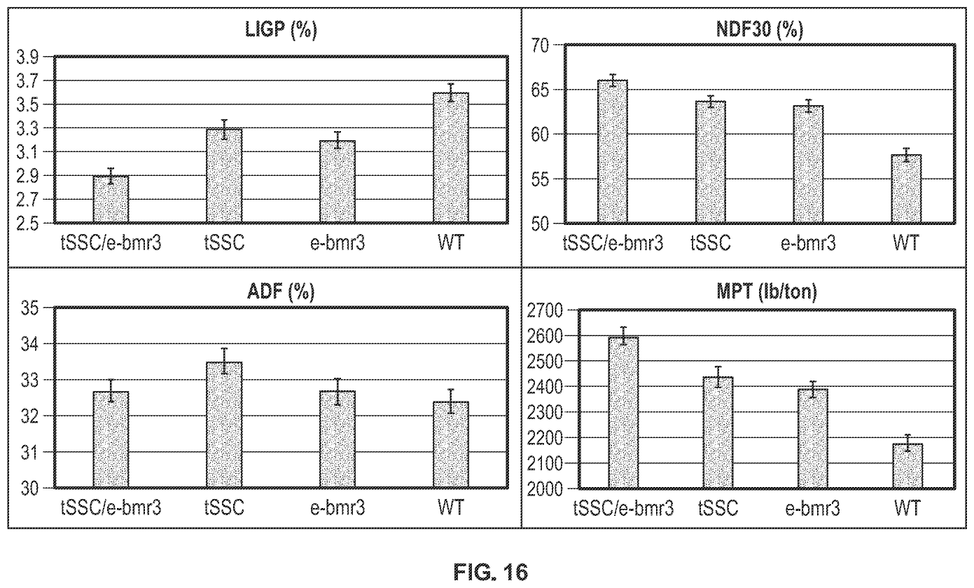

Hybrid tSSC/e-bmr3 corn plants were planted along with hybrid tSSC corn plants in 30″ row plots replicated 16 times. Normal (tall) stature e-bmr3 and WT plants were also planted in the same arrangement. When plants reached full maturity, each plot was harvested and sent to DAIRYLAND LABORATORIES® in Arcadia, WI for silage quality analysis. Key silage traits measured included digestibility as NDF30 (Neutral Detergent Fiber after 30-hour incubating), Lignin content in percentage (LIGP), Acid Detergent Fiber content in percentage (ADF), and milk content in pound per ton of biomass (MPT). Improved silage quality is marked by increased digestibility, reduced lignin content, increased acid detergent fiber content, and/or increased milk content. Protocols for silage quality analysis adopted by industry through The Association of Official Analytical Chemists (see, e.g., Baur, F. J. et al., The Association of Official Analytical Chemists (AOAC). J Am Oil Chem. Soc. 54, 171-172 (1977)) were performed for these studies. The field trial for this experiment was conducted in one growing season, and results are summarized in Table 13 below and FIG. 16. Mean trait measurements were averaged over 8 to 10 samples, and standard errors (StdErr) are provided.

As shown in Table 13 and FIG. 16, transgenic short stature corn plants without the homozygous bmr3 mutant alleles (tSSC plants) demonstrated significantly improved silage traits in this experiment relative to normal (tall) stature wild-type control plants (WT), including reduced lignin content (LIGP), increased digestibility (NDF30), and increased milk content (MPT). Corn plants homozygous mutant bmr3 alleles (e-bmr3; with the gene edited bmr3 and native bmr3 mutant alleles) also demonstrated significantly improved silage traits in this experiment over the WT control with either the normal or short stature (tSSC) trait. Short stature corn plants comprising homozygous bmr3 mutant alleles (tSSC/e-bmr3) demonstrated one or more further improved silage traits in this experiment over WT, e-bmr3 and tSSC plants, with perhaps the exception of Acid Detergent Fiber content (ADF). It is not clear if the compositional silage benefits with the tSSC trait are due to changes in above ground biomass of the shorter plants which alter the relative proportions of different tissue types (e.g., stalk vs. leaves) between taller and shorter stature corn plants.

TABLE 13
Silage quality traits of short and normal stature corn hybrids,
with or without homozygous bmr3 mutant alleles.

| | | LIGP | LIGP- | NDF30 | NDF- | ADF | ADF- | MPT | MPT- |
|---|---|---|---|---|---|---|---|---|---|
| Stature | Type | (%) | StdErr | (%) | StdErr | (%) | StdErr | (lb/ton) | StdErr |
| Short | tSSC/ | 2.9 | 0.064 | 66.1 | 0.61 | 32.7 | 0.32 | 2587.8 | 35.06 |
| | e-bmr3 | | | | | | | | |
| | tSSC | 3.3 | 0.069 | 63.7 | 0.66 | 33.5 | 0.35 | 2432.6 | 37.48 |
| Normal | e-bmr3 | 3.2 | 0.070 | 63.2 | 0.71 | 32.7 | 0.34 | 2382.3 | 30.77 |
| | WT | 3.6 | 0.070 | 57.7 | 0.71 | 32.4 | 0.34 | 2175.3 | 30.77 |

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having.” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on.” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” as well as the phrase “at least one of” includes any and all combinations of one or more of the associated listed items.

The term “about” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. For example, the terms “generally,” “about.” and “substantially.” may be used herein to mean within manufacturing tolerances. Or for example, the term “about” as used herein when modifying a quantity of an ingredient or reactant of the invention or employed refers to variation in the numerical quantity that can happen through typical measuring and handling procedures used, for example, when making concentrates or solutions in the real world through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about,” the claims include equivalents to the quantities.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer.” “beneath,” “below,” “lower,” “above,” “upper.” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A maize plant or maize plant part comprising:
a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and
a mutant allele of an endogenous brown midrib 3 (bm3) gene.

2. The maize plant or maize plant part of claim 1, wherein the mutant allele of the endogenous bm3 gene comprises an insertion, deletion, or substitution of one or more nucleotides, or any combination thereof, in the endogenous bm3 gene; and/or
wherein the mutant allele of the endogenous br2 gene comprises an insertion, deletion, or substitution of one or more nucleotides, or any combination thereof, in the endogenous br2 gene.

3. The maize plant or maize plant part of claim 1, wherein the mutant allele of the endogenous bm3 gene is a bm3-1 allele, bm3-2 allele, or bm3-3 allele; and/or
wherein the mutant allele of the endogenous br2 gene is selected from the group consisting of a br2-23 allele, br2-7081 allele, br2-7861 allele, br2-qph1 allele, br2-qpa1 allele, br2-NC238 allele, and br2-1005 allele.

4. The maize plant or maize plant part of claim 1, wherein the mutant allele of the endogenous bm3 gene is an edited allele of the endogenous bm3 gene; and/or
wherein the mutant allele of the endogenous br2 gene is an edited allele of the endogenous br2 gene.

5. The maize plant or maize plant part of claim 1, wherein the mutant allele of the endogenous bm3 gene comprises one or more mutations relative to SEQ ID NO: 94, SEQ ID NO: 95 and/or SEQ ID NO: 96; and/or
wherein the mutant allele of the endogenous br2 gene comprises one or more mutations relative to SEQ ID NO: 90, SEQ ID NO: 91 and/or SEQ ID NO: 92.

6. The maize plant or maize plant part of claim 1, wherein the expression level and/or activity of the mRNA and/or protein encoded by the mutant allele of the endogenous bm3 gene is reduced in the maize plant or maize plant part relative to the expression level and/or activity of the mRNA and/or protein encoded by a wild-type bm3 gene allele; and/or
wherein the expression level and/or activity of the mRNA and/or protein encoded by the mutant allele of the endogenous br2 gene is reduced in the maize plant or maize plant part relative to the expression level and/or activity of the mRNA and/or protein encoded by a wild-type br2 gene allele.

7. The maize plant or maize plant part of claim 1, wherein the maize plant or maize plant part is homozygous for the mutant allele of the endogenous bm3 gene; and/or
wherein the maize plant or maize plant part is homozygous for the mutant allele of the endogenous br2 gene.

8. The maize plant or maize plant part of claim 1, wherein the maize plant or maize plant part is heteroallelic for the endogenous bm3 gene and comprises a first mutant allele of the endogenous bm3 gene and a second mutant allele of the endogenous bm3 gene; and/or
wherein the maize plant or maize plant part is heteroallelic for the endogenous br2 gene and comprises a first mutant allele of the endogenous br2 gene and a second mutant allele of the endogenous br2 gene.

9.-15. (canceled)

16. The maize plant or maize plant part of claim 1, wherein the non-coding RNA molecule comprises a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA molecule encoding an endogenous GA oxidase protein in maize plant or maize plant part, the endogenous GA oxidase protein being at least 80%, at least 85%, at least 90%, at least 95%, at least 96%, at least 97%, at least 98%, at least 99%, at least 99.5%, or 100% identical to SEQ ID NO: 9, 12, 15, 30, 33 or 89; and/or
wherein the non-coding RNA molecule comprises a sequence that is (i) at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a first mRNA molecule encoding a first endogenous GA20 oxidase protein in the maize plant or maize plant part, the first endogenous GA20 oxidase protein being at least 80%, at least 85%, at least 90%, at least 95%, at least 96%, at least 97%, at least 98%, at least 99%, at least 99.5%, or 100% identical to SEQ ID NO: 9; and/or (ii) at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of a second mRNA molecule encoding a second endogenous GA20 oxidase protein in the maize plant or maize plant part, the second endogenous GA20 oxidase protein being at least 80%, at least 85%, at least 90%, at least 95%, at least 96%, at least 97%, at least 98%, at least 99%, at least 99.5%, or 100% identical to SEQ ID NO: 15.

17. (canceled)

18. The maize plant or maize plant part of claim 1, wherein the plant-expressible promoter is a vascular promoter, RTBV promoter, a leaf promoter, or a constitutive promoter.

19.-21. (canceled)

22. The maize plant or maize plant part of claim 1, wherein the non-coding RNA molecule encoded by the transcribable DNA sequence is a precursor miRNA or siRNA that is processed or cleaved in a plant cell to form a mature miRNA or siRNA.

23. The maize plant or maize plant part of claim 1,
wherein the endogenous GA20 oxidase gene is the endogenous GA20 oxidase_3 gene, and wherein the mutant allele of the endogenous GA20 oxidase gene comprises one or more mutations relative to SEQ ID NO: 7, SEQ ID NO: 8, and/or SEQ ID NO: 34, or
wherein the endogenous GA20 oxidase gene is the endogenous GA20 oxidase_4 gene, and wherein the mutant allele of the endogenous GA20 oxidase gene comprises one or more mutations relative to SEQ ID NO: 10, SEQ ID NO: 11, and/or SEQ ID NO: 38, or
wherein the endogenous GA20 oxidase gene is the endogenous GA20 oxidase_5 gene, and wherein the mutant allele of the endogenous GA20 oxidase gene comprises one or more mutations relative to SEQ ID NO: 13, SEQ ID NO: 14, and/or SEQ ID NO: 35, or
wherein the endogenous GA3 oxidase gene is the endogenous GA3 oxidase_1 gene, and wherein the mutant allele of the endogenous GA3 oxidase gene comprises one or more mutations relative to SEQ ID NO: 28, SEQ ID NO: 29, SEQ ID NO: 36 and/or SEQ ID NO: 84, or
wherein the endogenous GA3 oxidase gene is the endogenous GA3 oxidase_2 gene, and wherein the mutant allele of the endogenous GA3 oxidase gene comprises one or more mutations relative to SEQ ID NO: 31, SEQ ID NO: 32, and/or SEQ ID NO: 37 and/or SEQ ID NO: 85, or
wherein the endogenous GA3 oxidase gene is the endogenous GA3 oxidase_3 gene, and wherein the mutant allele of the endogenous GA3 oxidase gene comprises one or more mutations relative to SEQ ID NO: 86, SEQ ID NO: 87, and/or SEQ ID NO: 88.

24. The maize plant or maize plant part of claim 1, comprising a mutant allele of the endogenous GA20 oxidase_3 gene and a mutant allele of the endogenous GA20 oxidase_5 gene; and
wherein:
the maize plant or maize plant part is homozygous for a mutant allele of the endogenous GA20 oxidase_3 gene and heterozygous for a mutant allele of the endogenous GA20 oxidase_5 gene; or
the maize plant or maize plant part is heterozygous for a mutant allele of the endogenous GA20 oxidase_3 gene and homozygous for a mutant allele of the endogenous GA20 oxidase_5 gene; or
the maize plant or maize plant part is heteroallelic for two mutant alleles of the endogenous GA20 oxidase_3 gene comprising a first mutant allele of the endogenous GA20 oxidase_3 gene and a second mutant allele of the endogenous GA20 oxidase_3 gene, and heterozygous for a mutant allele of the endogenous GA20 oxidase_5 gene; or
the maize plant or maize plant part is heteroallelic for two mutant alleles of the endogenous GA20 oxidase_5 gene comprising a first mutant allele of the endogenous GA20 oxidase_5 gene and a second mutant allele of the endogenous GA20 oxidase_5 gene, and heterozygous for a mutant allele of the endogenous GA20 oxidase_3 gene.

25.-28. (canceled)

29. The maize plant or maize plant part of claim 1, wherein the expression level and/or activity of the mRNA and/or protein encoded by the mutant allele of the endogenous GA20 oxidase gene is reduced in the maize plant or maize plant part relative to the expression level and/or activity of the mRNA and/or protein encoded by a wild-type allele of the same GA20 oxidase gene; or
wherein the expression level and/or activity of the mRNA and/or protein encoded by the mutant allele of the endogenous GA3 oxidase gene is reduced in the maize plant or maize plant part relative to the expression level and/or activity of the mRNA and/or protein encoded by a wild-type allele of the same GA3 oxidase gene.

30. The maize plant or maize plant part of claim 1, wherein the maize plant or maize plant part has one or more traits selected from the group including reduced lignin content, improved fiber digestibility, reduced acid detergent fiber (ADF), increased starch content, increased neutral detergent fiber digestibility (NDFD) or cell wall digestibility (DCW), reduced root lodging, increased biomass, increased protein content (and/or N-stover), increased milk output, increased grain yield, and increased stem cross-section area, as compared to a wild-type or control maize plant or plant part.

31. The maize plant or maize plant part of claim 1, wherein the endogenous bm3 gene locus comprises SEQ ID NO: 94, 95 or 96; and/or
wherein the endogenous br2 gene locus comprises SEQ ID NO: 90, 91 or 92.

32. The maize plant or maize plant part of claim 1, wherein the maize plant or maize plant part is a maize hybrid plant or a maize plant part thereof.

33. The maize plant of claim 1, wherein the maize plant has a shorter plant height as compared to the wild-type or control maize plant.

34. The maize plant of claim 1, wherein the maize plant has a plant height that is about 10% or more shorter than the wild-type or control maize plant;
wherein the maize plant or maize plant part comprises an increased protein content over the wild-type or control maize plant or maize plant part of about 2% or more;
wherein the maize plant or maize plant part comprises a reduced lignin content over the wild-type or control maize plant or plant part of about 1% or more;
wherein the maize plant or maize plant part comprises an increased starch content over the wild-type or control maize plant or maize plant part of about 5% or more; and/or
wherein the maize plant or maize plant part comprises an increased NDFD over the wild-type or control maize plant or plant part of about 1% or more.

35. The maize plant of claim 1, wherein the maize plant has increased resistance to root lodging and/or green snap, as compared to the wild-type or control maize plant.

36. The maize plant or maize plant part of claim 1, wherein the maize plant or maize plant part comprises a protein content of about 2% or more;
wherein the maize plant or maize plant part comprises a lignin content of about 6% or less;
wherein the maize plant or maize plant part comprises a starch content of about 20% or more;
wherein the maize plant or maize plant part comprises a NDFD of about 50% or more; and/or
wherein the maize plant or maize plant part comprises an ADF of about 50% or less.

37.-49. (canceled)

50. The maize plant part of claim 1, wherein the maize plant part is a maize plant seed.

51.-52. (canceled)

53. A maize plant seed comprising:
a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and
a mutant allele of an endogenous brown midrib 3 (bm3) gene.

54. A plurality of maize plant seeds wherein a majority of the maize plant seeds in the plurality of the maize plant seeds comprises
a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and
a mutant allele of an endogenous brown midrib 3 (bm3) gene.

55. The plurality of maize plant seeds of claim 54, wherein each of the maize plant seeds in the plurality of the maize plant seeds comprises the mutant allele of the endogenous br2 gene, the mutant allele of an endogenous GA20 oxidase gene, or the mutant allele of the endogenous GA3 oxidase gene, or the transgene comprising the transcribable DNA sequence; and the mutant allele of the endogenous bm3 gene.

56. A plurality of maize plant seeds, wherein a majority of the maize plant seeds in the plurality of maize plant seeds comprise a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or a mutant allele of an endogenous brown midrib 3 (bm3) gene.

57. A population of maize plants, wherein a majority of the maize plants of the population comprises a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or optionally a mutant allele of an endogenous brown midrib 3 (bm3) gene, and wherein the population of maize plants has one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants.

58. The population of maize plants of claim 57, wherein the population of wild-type or control maize plants includes a population of wild-type dual purpose control maize plants.

59. The population of maize plants of claim 57, wherein at least a majority of the maize plants of the population further comprises a mutant allele of an endogenous bm3 gene; and/or
wherein at least a majority of the maize plants of the population are a maize hybrid plant.

60. The population of maize plants of claim 57, wherein each of the maize plants of the population further comprises a mutant allele of an endogenous bm3 gene.

61. The population of maize plants of claim 57, wherein the maize plants of the population are homozygous for the mutant allele of the endogenous bm3 gene;
wherein the maize plants of the population are heteroallelic for the endogenous bm3 gene and comprise a first mutant allele of the endogenous bm3 gene and a second mutant allele of the endogenous bm3 gene;
wherein the maize plants of the population are homozygous or heterozygous for the mutant allele of the endogenous br2 gene, the mutant allele of an endogenous GA20 oxidase gene, the mutant allele of an endogenous GA3 oxidase gene, or the transgene comprising the transcribable DNA sequence; and/or
wherein the maize plants of the population are heteroallelic for the endogenous br2 gene and comprise a first mutant allele of the endogenous br2 gene, the endogenous GA20 oxidase gene, or the endogenous GA3 oxidase gene, and a second mutant allele of the endogenous br2 gene, the endogenous GA20 oxidase gene, or the endogenous GA3 oxidase gene.

62.-67. (canceled)

68. The population of maize plants of claim 57, wherein the population of maize plants has an average plant height of about 10% or more shorter than a population of wild-type or control maize plants; and/or
wherein the population of maize plants has increased resistance to lodging and/or green snap, as compared to a population of wild-type or control maize plants.

69.-71. (canceled)

72. The population of maize plants of claim 57, wherein the population of maize plants are grown in a field at a growing or planting density of about 20,000 plants/Ha or more;
wherein the population of maize plants are planted at a planting density of about 27,000 seeds/Ha or more;
wherein the population of maize plants comprises a milk per ton output of about 3,000 lbs/ton or more, about 3,200 lbs/ton or more, about 3,400 lbs/ton or more, about 3,600 lbs/ton or more, about 3,800 lbs/ton or more, or about 4,000 lbs/ton or more; and/or
wherein the population of maize plants are planted and grown in a plurality of parallel rows, wherein the average spacing between adjacent rows is about 80 cm or less, about 70 cm or less, about 60 cm or less, about 50 cm or less, about 40 cm or less, about 35 cm or less, or about 30 cm or less, or about 76 cm, about 51 cm, about 38 cm, or about 30 cm.

73.-75. (canceled)

76. A population of intercropped silage plants comprising the population of maize plants of claim 57 and a population of one or more companion crop plants wherein the population of companion crop plants includes one or more of wheat, barley, oat, alfalfa, rye, clover, grass, triticale, cereal, legume, bean, pea, or soybean.

77. The population of intercropped silage plants of claim 76, wherein the population of intercropped silage plants has a dry matter biomass (DMB) of between about 0.5 kg/m² and about 2 kg/m²; and/or
wherein the population of intercropped silage plants has a silage yield of greater than or equal to about 5 tons/acre.

78. The population of intercropped silage plants of claim 76, wherein the population of maize plants are planted and grown in a plurality of parallel rows, and wherein the population of companion crop plants are planted and grown between adjacent rows of the population of maize plants;
wherein the population of maize plants are planted and grown in a plurality of parallel rows, wherein the average spacing between adjacent rows of maize plants is about 80 cm or less; and/or
wherein the population of companion crop plants are planted and grown in a row or plurality of parallel rows, and wherein each row of companion crop plants is planted between two adjacent rows of the population of maize plants.

79.-81. (canceled)

82. The population of intercropped silage plants of claim 76, wherein the population of intercropped silage plants are grown at a growing density of about 50,000 plants/Ha or more; and/or
wherein the population of intercropped silage plants are planted at a planting density of about 67,000 seeds/Ha or more.

83. (canceled)

84. The population of intercropped silage plants of claim 76, wherein the population of companion crop plants are grown at a growing density of about 10,000 plants/Ha or more; and/or
wherein the population of companion crop plants are planted at a planting density of about 13,000 seeds/Ha or more.

85.-86. (canceled)

87. The population of maize plants of claim 57, wherein the endogenous br2 gene comprises SEQ ID NO: 90, and wherein SEQ ID NO: 90 comprises one or more genetic mutations.

88. (canceled)

89. A silage product comprising at least a portion of the population of maize plants of claim 57, or an above ground biomass of at least a portion of the population.

90. The silage product of claim 89, wherein the silage product has one or more characteristics selected from reduced lignin content, improved fiber digestibility, reduced ADF, increased starch content, increased NDFD or DCW, increased biomass, increased protein content (and/or N-stover), increased milk output, increased silage yield, increased grain yield, or increased stem cross-section area, as compared to a silage product comprising a wild-type or control maize plant or maize plant part.

91. The silage product of claim 90, wherein the silage product comprises a protein content of about 4% or more;
wherein the silage product comprises a lignin content of about 6% or less;
wherein the silage product comprises a starch content of about 20% or more;
wherein the silage product comprises a NDFD of about 50% or more; and/or
wherein the silage product comprises an ADF of about 50% or less.

92. (canceled)

93. The silage product of claim 90, wherein the silage product comprises an increased protein content over a silage product including the wild-type or control maize plant or maize plant part of about 2% or more;
wherein the silage product comprises a reduced lignin content over a silage product including the wild-type or control maize plant or plant part of about 1% or more;
wherein the silage product comprises an increased starch content over a silage product including the wild-type or control maize plant or maize plant part of about 5% or more; and/or
wherein the silage product comprises an increased NDFD over a silage product including the wild-type or control maize plant or plant part of about 1% or more.

94.-104. (canceled)

105. A method for producing silage, the method comprising:
harvesting an above-ground biomass of the population of maize plants of claim 57; and
chopping the above-ground biomass into pieces to form a silage product.

106. The method of claim 105, further comprising:
planting in a field a plurality of maize plant seeds or a plurality of intercropped silage plant seeds; and
growing the population of maize plants or the population of intercropped silage plants from the maize plant seeds or the intercropped silage plant seeds.

107. A method for producing silage, the method comprising:
planting in a field a plurality of maize plant seeds of claim 61;
growing a plurality of maize plants from the plurality of maize plant seeds;
harvesting an above-ground biomass of the population of maize plants; and
chopping the above-ground biomass into pieces to form a silage product.

108. The method of claim 107, further comprising:
planting a plurality of companion crop plant seeds with the plurality of maize plant seeds.

109. The method of claim 105, further comprising:
storing the silage product for a time period of about 1 day to about 2 years;
fermenting the silage product for a time period of about 1 day to about 2 years; and/or
feeding the silage product to one or more livestock animals.

110.-111. (canceled)

112. The method of claim 105, wherein the above-ground biomass of the population of maize plants or the population of intercropped silage plants is harvested when the moisture content of the population of maize plants or the population of intercropped silage plants is between about 50% and about 80%.

113. A method for producing maize plant seeds, comprising:
crossing a first maize plant to a second maize plant, wherein either:
(a) the first maize plant comprises a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter;
and a mutant allele of an endogenous brown midrib 3 (bm3) gene;
(b) the first maize plant comprises a mutant allele of an endogenous br2 gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter, and the second maize plant comprises a mutant allele of an endogenous bm3 gene;
(c) the first maize plant comprises a mutant allele of the endogenous bm3 gene, and the second maize plant comprises a mutant allele of an endogenous br2 gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; or
(d) the second maize plant comprises a mutant allele of an endogenous br2 gene, a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and a mutant allele of an endogenous bm3 gene;
wherein either (i) the first plant is the female plant, and second plant is the male plant; or (ii) the second plant is the female plant, and first plant is the male plant; and
harvesting one or more maize plant progeny seeds from the female maize plant, wherein the maize plant progeny seeds comprise the mutant allele of an endogenous br2 gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and the mutant allele of an endogenous bm3 gene.

114. The method of claim 113, wherein the maize plant progeny seeds are homozygous for the mutant allele of the endogenous bm3 gene.

115. The method of claim 114, wherein the maize plant progeny seeds are heteroallelic for the endogenous bm3 gene and comprises a first mutant allele of the endogenous bm3 gene and a second mutant allele of the endogenous bm3 gene.

116. The method of claim 113, wherein the maize plant progeny seeds are homozygous or heterozygous for the mutant allele of the endogenous br2 gene, or the mutant allele of the endogenous GA20 oxidase gene, or the mutant allele of the endogenous GA3 oxidase gene, or the transgene comprising the transcribable DNA sequence; and/or
wherein the maize plant progeny seeds are heteroallelic for the endogenous br2 gene, the mutant allele of the endogenous GA20 oxidase gene, or the mutant allele of the endogenous GA3 oxidase gene, and comprises a first mutant allele of the endogenous br2 gene, the mutant allele of the endogenous GA20 oxidase gene, or the mutant allele of the endogenous GA3 oxidase gene, and a second mutant allele of the endogenous br2 gene, the mutant allele of the endogenous GA20 oxidase gene, or the mutant allele of the endogenous GA3 oxidase gene.

117. (canceled)

118. The method of claim 113, further comprising:
selecting one or more progeny maize plant progeny seeds that are homozygous or heteroallelic for the mutant allele of the endogenous bm3 gene and/or homozygous or heteroallelic for the mutant allele of the endogenous br2 gene, the mutant allele of the endogenous GA20 oxidase gene, or the mutant allele of the endogenous GA3 oxidase gene.

119. The method of claim 113, further comprising:
planting the one or more maize plant progeny seeds in a field, greenhouse, or controlled environment; and
growing one or more maize progeny plants from the maize plant progeny seeds.

120.-121. (canceled)

122. A population of maize plant seeds; wherein the at least one maize plant seed of the population comprises a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or a mutant allele of an endogenous brown midrib 3 (bm3) gene.

123. The population of seeds of claim 122, comprising a plurality of maize plant seeds; wherein at least a majority of the maize plant seeds comprises the mutant allele of the endogenous br2 gene, the mutant allele of an endogenous GA20 oxidase gene, the mutant allele of an endogenous GA3 oxidase gene, or the transgene comprising the transcribable DNA sequence; and/or a mutant allele of the endogenous bm3 gene; and/or
wherein at least a majority of the maize plant seeds comprises a mutant allele of the endogenous br2 gene and a mutant allele of the endogenous bm3 gene.

124.-126. (canceled)

127. The population of seeds of claim 122, wherein the maize plant seed(s) is/are homozygous for the mutant allele of the endogenous br2 gene, the mutant allele of an endogenous GA20 oxidase gene, the mutant allele of an endogenous GA3 oxidase gene, or the transgene comprising the transcribable DNA sequence;
wherein the maize plant seed(s) is/are heteroallelic for the endogenous br2 gene, the endogenous GA20 oxidase gene, or the endogenous GA3 oxidase gene, and comprise(s) a first mutant allele of the endogenous br2 gene, the endogenous GA20 oxidase gene, or the endogenous GA3 oxidase gene and a second mutant allele of the endogenous br2 gene, the endogenous GA20 oxidase gene, or the endogenous GA3 oxidase gene;
wherein the maize plant seed(s) is/are homozygous for the mutant allele of the endogenous bm3 gene; and/or
wherein the maize plant seed(s) is/are heteroallelic for the endogenous bm3 gene and comprise(s) a first mutant allele of the endogenous bm3 gene and a second mutant allele of the endogenous bm3 gene.

128.-131. (canceled)

132. A method for producing silage, the method comprising:
planting in a field a population of seeds, wherein at least one maize plant seed of the population comprises a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or a mutant allele of an endogenous brown midrib 3 (bm3) gene;
growing plants from the seeds, wherein a plurality of the growing plants include maize plants comprising a mutant allele of an endogenous br2 gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and/or a mutant allele of an endogenous bm3 gene;
harvesting an above-ground biomass of the plants; and
chopping the above-ground biomass into pieces to form a silage product.

133. The method of claim 132, wherein the growing plants further include companion crop plants.

134. The method of claim 132, further comprising storing the silage product for a time period of about 1 day to about 2 years;
fermenting the silage product for a time period of about 1 day to about 2 years; and/or
feeding the silage product to one or more livestock animals.

135.-136. (canceled)

137. A method comprising using, the population of maize plants of claim 57 to produce a silage product.

138.-152. (canceled)

153. A population of maize plants, wherein at least a majority of the maize plants of the population comprises a mutant allele of an endogenous brachytic 2 (br2) gene, or a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and a mutant allele of an endogenous brown midrib gene.

154. (canceled)

155. The population of maize plants of claim 153, wherein the mutant allele of the endogenous brown midrib gene incudes a mutant allele of an endogenous brown midrib 3 (bm3) gene.

156. The population of maize plants of claim 155, wherein the maize plants of the population are homozygous for the mutant allele of the endogenous bm3 gene; and/or
wherein the maize plants of the population are heteroallelic for the endogenous bm3 gene and comprise a first mutant allele of the endogenous bm3 gene and a second mutant allele of the endogenous bm3 gene.

157. (canceled)

158. The population of maize plants of claim 153, wherein the maize plants of the population are homozygous or heterozygous for the mutant allele of the endogenous br2 gene, or the mutant allele of the endogenous GA20 oxidase gene, or the mutant allele of the endogenous GA3 oxidase gene, or the transgene comprising the transcribable DNA sequence; and/or
wherein the maize plants of the population are heteroallelic for the endogenous br2 gene, the endogenous GA20 oxidase gene, or the endogenous GA3 oxidase gene, and comprise a first mutant allele of the endogenous br2 gene, the endogenous GA20 oxidase gene, or the endogenous GA3 oxidase gene, and a second mutant allele of the endogenous br2 gene, the endogenous GA20 oxidase gene, or the endogenous GA3 oxidase gene.

159. (canceled)

160. The population of maize plants of claim 153, wherein the population of maize plants has one or more traits selected from reduced lignin content, improved fiber digestibility, reduced ADF, increased starch content, increased NDFD, increased biomass, increased protein content (and/or N-stover), increased milk per acre output, and increased silage yield, as compared to a population of wild-type or control maize plants.

161. A population of seeds comprising maize plant seeds, wherein at least a majority of the maize plant seeds of the population comprises a mutant allele of an endogenous brachytic 2 (br2) gene, a mutant allele of an endogenous GA20 oxidase gene, or a mutant allele of an endogenous GA3 oxidase gene, or a transgene comprising a transcribable DNA sequence encoding a non-coding RNA molecule comprising a sequence that is complementary to a mRNA molecule encoding an endogenous GA20 oxidase protein or a GA3 oxidase protein, wherein the transcribable DNA sequence is operably linked to a plant expressible promoter; and a mutant allele of an endogenous brown midrib gene.

162. (canceled)

163. The population of maize plant seeds of claim 161, wherein the mutant allele of the endogenous brown midrib gene incudes a mutant allele of an endogenous brown midrib 3 (bm3) gene.

164. The population of maize plant seeds of claim 163, wherein the maize plant seeds comprising the mutant allele of the endogenous bm3 gene are homozygous for the mutant allele of the endogenous bm3 gene; and/or
wherein the maize plant seeds comprising the mutant allele of the endogenous bm3 gene of the population are heteroallelic for the endogenous bm3 gene and comprise a first mutant allele of the endogenous bm3 gene and a second mutant allele of the endogenous bm3 gene.

165. (canceled)

166. The population of maize plant seeds of claim 161, wherein the maize plant seeds comprising the mutant allele of the endogenous br2 gene, the endogenous GA20 oxidase gene, or the endogenous GA3 oxidase gene are homozygous for the mutant allele of the endogenous br2 gene, the endogenous GA20 oxidase gene, or the endogenous GA3 oxidase gene; and/or
wherein the maize plant seeds comprising the mutant allele of the endogenous br2 gene, the endogenous GA20 oxidase gene, or the endogenous GA3 oxidase gene are heteroallelic for the endogenous br2 gene, the endogenous GA20 oxidase gene, or the endogenous GA3 oxidase gene and comprise a first mutant allele of the endogenous br2 gene the endogenous GA20 oxidase gene, or the endogenous GA3 oxidase gene and a second mutant allele of the endogenous br2 gene, the endogenous GA20 oxidase gene, or the endogenous GA3 oxidase gene.

167.-168. (canceled)


SEQUENCE LISTING (96 sequences)

SEQ ID NO: 1 - DNA, 1741 bp, Zea mays
  source 1..1741: mol_type = genomic DNA
gacggtagttttcatctaaagtttattcttcgtcacatgggatggccgtttgcttgtttgttgcttccgggaggcggtggtgaattgaagcagatcgacaagcatggctgcccactggtctcgatcgatcggcctgccatgccatgccatgccactagagtccgtcctgactggccgcccgttcccccgtataaaaaggcaggcaggcaggcagagcggggacgagcaagcaagcagttgcagttgcagcggcctcctcctctgcttcctccctcctcctcctcaccatggtgctggctgcgcacgatccccctccccttgtgttcgacgctgcccgcctgagcggcctctccgacatcccgcagcagttcatctggccggcggacgagagccccaccccggactccgccgaggagctggccgtgccgctcatcgacctctccggggacgccgccgaggtggtccggcaggtccggcgcgcctgcgacctgcacggcttcttccaggtggtggggcacggcatcgacgcggcgctgacggcggaggcccaccgctgcatggacgccttcttcacgctgccgctcccggacaagcagcgcgcgcagcgccgccagggggacagctgcggctacgccagcagcttcacgggccggttcgcgtccaagctgccctggaaggagacgctgtcgttccgctacaccgacgacgacgacggcgacaagtccaaggacgtcgtggcgtcctacttcgtggacaagctgggcgaggggtaccggcaccacggggaggtgtacgggcgctactgctctgagatgagccgtctgtcgctggagctcatggaggtgctaggcgagagcctgggcgtgggccggcgccacttccggcgcttcttccaggggaacgactccatcatgcgcctcaactactacccgccgtgccagcggccctacgacacgctgggcacggggccgcattgcgaccccacgtcgctcaccatcctgcaccaggacgacgtgggcggactccaggtgttcgacgccgccacgctcgcgtggcgctccatcaggccccgcccgggcgccttcgtcgtcaacatcggcgacaccttcatggcgctctccaacgggcgctacaggagctgcctccaccgcgccgtcgtcaacagccgggtggcacgccgctcgctcgccttcttcctgtgcccggagatggacaaggtggtcaggccgcccaaggagctggtggacgacgccaacccgagggcgtacccggacttcacgtggaggacgctgctggacttcaccatgaggcactacaggtcggacatgaggacgctcgaggccttctccaactggctcagcaccagtagcaatggcggacagcacctgctggagaagaagtaggcatgctatttgggtatggaagatggtggatgtaagcaaacaaagccaaattaagcagagtaggttaattaaggttggctgatgatccatttagggaaggagctgatctccctgactccctcctccaattttctcaaccaaatttatatagtataataataataataaaatagcaagtaatagttgtatcgtattattattaattaatttattagctggtaggcaagtagtattaaataccatttgtagtacgatgggcgtatttctattttggcgttttgctctgtgttttttgacgtttcctttggatttggggggacctcagatcagctcggcct

SEQ ID NO: 2 - DNA, 1116 bp, Zea mays
  source 1..1116: mol_type = genomic DNA
atggtgctggctgcgcacgatccccctccccttgtgttcgacgctgcccgcctgagcggcctctccgacatcccgcagcagttcatctggccggcggacgagagccccaccccggactccgccgaggagctggccgtgccgctcatcgacctctccggggacgccgccgaggtggtccggcaggtccggcgcgcctgcgacctgcacggcttcttccaggtggtggggcacggcatcgacgcggcgctgacggcggaggcccaccgctgcatggacgccttcttcacgctgccgctcccggacaagcagcgcgcgcagcgccgccagggggacagctgcggctacgccagcagcttcacgggccggttcgcgtccaagctgccctggaaggagacgctgtcgttccgctacaccgacgacgacgacggcgacaagtccaaggacgtcgtggcgtcctacttcgtggacaagctgggcgaggggtaccggcaccacggggaggtgtacgggcgctactgctctgagatgagccgtctgtcgctggagctcatggaggtgctaggcgagagcctgggcgtgggccggcgccacttccggcgcttcttccaggggaacgactccatcatgcgcctcaactactacccgccgtgccagcggccctacgacacgctgggcacggggccgcattgcgaccccacgtcgctcaccatcctgcaccaggacgacgtgggcggactccaggtgttcgacgccgccacgctcgcgtggcgctccatcaggccccgcccgggcgccttcgtcgtcaacatcggcgacaccttcatggcgctctccaacgggcgctacaggagctgcctccaccgcgccgtcgtcaacagccgggtggcacgccgctcgctcgccttcttcctgtgcccggagatggacaaggtggtcaggccgcccaaggagctggtggacgacgccaacccgagggcgtacccggacttcacgtggaggacgctgctggacttcaccatgaggcactacaggtcggacatgaggacgctcgaggccttctccaactggctcagcaccagtagcaatggcggacagcacctgctggagaagaagtag

SEQ ID NO: 3 - protein, 371 aa, Zea mays
MVLAAHDPPPLVFDAARLSGLSDIPQQFIWPADESPTPDSAEELAVPLIDLSGDAAEVVRQVRRACDLHGFFQVVGHGIDAALTAEAHRCMDAFFTLPLPDKQRAQRRQGDSCGYASSFTGRFASKLPWKETLSFRYTDDDDGDKSKDVVASYFVDKLGEGYRHHGEVYGRYCSEMSRLSLELMEVLGESLGVGRRHFRRFFQGNDSIMRLNYYPPCQRPYDTLGTGPHCDPTSLTILHQDDVGGLQVFDAATLAWRSIRPRPGAFVVNIGDTFMALSNGRYRSCLHRAVVNSRVARRSLAFFLCPEMDKVVRPPKELVDDANPRAYPDFTWRTLLDFTMRHYRSDMRTLEAFSNWLSTSSNGGQHLLEKK

SEQ ID NO: 4 - DNA, 1517 bp, Zea mays
  source 1..1517: mol_type = genomic DNA
caggaataaaataagcctccgcccggcttcgttgcatccacgcacgcagcaagcgatcggatttcgccagcatggcggcggcggccgtggtgttcgacgccgaggcgctgagccgggaggagcacatcccggcgcagttcgtgtggcccaccgaggagcgggcgccggcgggcggcgtggaggaggtcgccatccccgtggtcgacctcggcgagttcctccgccgcggggtgctcccgcgcggcgtggcggaggcgtgcgagcgccacggcgtcttccaggtggtgaaccacggcgtgggcgccgcgctgctcgccgaggcctaccgctgttgcgacgccttttacgcgctcccgctcgcggacaagcagcgcgcgcagcgccggcacggggagaaccacggctacgccagcagcttcacgggccgcttccactgctgcctgccgtggaaggagacgctgtccttcaactgccccgccggtgccgggactgcgcgcgccgtcgtcggctacttcgtcgacgtcctcggcgaggactaccgccacatgggggaggtgtaccaggagtactgcgacgcgatgacgcgtctggcgctggacgtgacggaggtgctggcggcagcgctggggctggaccgcggcgcactgcgcggcttcttcgagggcggcgactccgtcatgcggctgaaccactacccggcgtgccggcagccgcacctgacgctggggacgggcccgcaccgggacccgacgtcgctgacgctgctgcaccaggacgacgtgggcgggctgcaggtgcgcgccggcggcgggccgtggcgcgcggtgcggccccgcgcggacgcgttcgtggtcaacattggcgacaccttcgccgcgctcaccgacgggcgtcacaccagctgcctgcaccgcgccgtggtgaccggcggcggctcccgccggtcgctcgccttcttcctcaacccgccgctggaccgcgtcgtccgcccgccgggcgcgctcctccaggagaacaagcaggcgggccgcccgcgcgcgttcccggacttcacgtggcgcgagttcctcgagttcacgcagaagcactaccggtcggacgcgggcaccatggacgccttcgtgtcgtggatcgcgggaggccgccgccaccatggcggacaggaggagggcaactgagatcgatgcatctctagctgtaggcagcagcgcagcagctaccaagaataatggccggcgacggagatgcagctacgacgcacaaataaattgagtgtttgtggtacaataaggacgaggacgatcaatggcgacctgtaaccggtgcagttttagttaatctttcatggcgatatggcattaaccaatcgttggtgtaaaatgcgtgcatgctttgcatgccaatgttggccatgtgatggcacagcgtgagtgtagctcacccaccgtgacaacgtgctaatttcgtgtggtcctagataccaaggtcgtctaatgaacttgatggattgatgattt

SEQ ID NO: 5 - DNA, 1107 bp, Zea mays
  source 1..1107: mol_type = genomic DNA
atggcggcggcggccgtggtgttcgacgccgaggcgctgagccgggaggagcacatcccggcgcagttcgtgtggcccaccgaggagcgggcgccggcgggcggcgtggaggaggtcgccatccccgtggtcgacctcggcgagttcctccgccgcggggtgctcccgcgcggcgtggcggaggcgtgcgagcgccacggcgtcttccaggtggtgaaccacggcgtgggcgccgcgctgctcgccgaggcctaccgctgttgcgacgccttttacgcgctcccgctcgcggacaagcagcgcgcgcagcgccggcacggggagaaccacggctacgccagcagcttcacgggccgcttccactgctgcctgccgtggaaggagacgctgtccttcaactgccccgccggtgccgggactgcgcgcgccgtcgtcggctacttcgtcgacgtcctcggcgaggactaccgccacatgggggaggtgtaccaggagtactgcgacgcgatgacgcgtctggcgctggacgtgacggaggtgctggcggcagcgctggggctggaccgcggcgcactgcgcggcttcttcgagggcggcgactccgtcatgcggctgaaccactacccggcgtgccggcagccgcacctgacgctggggacgggcccgcaccgggacccgacgtcgctgacgctgctgcaccaggacgacgtgggcgggctgcaggtgcgcgccggcggcgggccgtggcgcgcggtgcggccccgcgcggacgcgttcgtggtcaacattggcgacaccttcgccgcgctcaccgacgggcgtcacaccagctgcctgcaccgcgccgtggtgaccggcggcggctcccgccggtcgctcgccttcttcctcaacccgccgctggaccgcgtcgtccgcccgccgggcgcgctcctccaggagaacaagcaggcgggccgcccgcgcgcgttcccggacttcacgtggcgcgagttcctcgagttcacgcagaagcactaccggtcggacgcgggcaccatggacgccttcgtgtcgtggatcgcgggaggccgccgccaccatggcggacaggaggagggcaactga

SEQ ID NO: 6 - protein, 368 aa, Zea mays
MAAAAVVFDAEALSREEHIPAQFVWPTEERAPAGGVEEVAIPVVDLGEFLRRGVLPRGVAEACERHGVFQVVNHGVGAALLAEAYRCCDAFYALPLADKQRAQRRHGENHGYASSFTGRFHCCLPWKETLSFNCPAGAGTARAVVGYFVDVLGEDYRHMGEVYQEYCDAMTRLALDVTEVLAAALGLDRGALRGFFEGGDSVMRLNHYPACRQPHLTLGTGPHRDPTSLTLLHQDDVGGLQVRAGGGPWRAVRPRADAFVVNIGDTFAALTDGRHTSCLHRAVVTGGGSRRSLAFFLNPPLDRVVRPPGALLQENKQAGRPRAFPDFTWREFLEFTQKHYRSDAGTMDAFVSWIAGGRRHHGGQEEGN

SEQ ID NO: 7 - DNA, 1522 bp, Zea mays
  source 1..1522: mol_type = genomic DNA
gcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaacgagccggccgtctctttcgccggggcccgcgcggggttcgcccacgtggtgatcaggtggcagacatgtggcccacgggccccgcgccgccttccccatttttggacgaccctactgctactactactagtgtacatatgcaaaaaaatacatatatatataggtactttctctaatatttttatatataagcaaggcggcctggtgttcttttctttgttttgtcgacaactgtttgatcccatcctatggacgatggatagttcaatgtttgtac

SEQ ID NO: 8 - DNA, 1161 bp, Zea mays
  source 1..1161: mol_type = genomic DNA
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 9 - protein, 386 aa, Zea mays
MDASPTPPLPLRAPTPSIDLPAGKDRADAAANKAAAVFDLRREPKIPEPFLWPHEEARPTSAAELEVPVVDVGVLRNGDGAGLRRAAAQVAAACATHGFFQVCGHGVDAALGRAALDGASDFFRLPLAEKQRARRVPGTVSGYTSAHADRFASKLPWKETLSFGFHDGAAAPVVVDYFTGTLGQDFEPVGRVYQRYCEEMKELSLTIMELLELSLGVERGYYREFFEDSRSIMRCNYYPPCPVPERTLGTGPHCDPTALTILLQDDVGGLEVLVDGEWRPVRPVPGAMVINIGDTFMALSNGRYKSCLHRAVVNRRQERQSLAFFLCPREDRVVRPPASAAPRQYPDFTWADLMRFTQRHYRADTRTLDAFTRWLSHGPAAAAPCT

SEQ ID NO: 10 - DNA, 1457 bp, Zea mays
  source 1..1457: mol_type = genomic DNA
taatcacctcatcacaggtccccccagcctcactctcgcgccggctcaaggtacattgcgtgtcctagccaagacacgcagctcatctcagcctcacacgcacagcaagagcgaggcgtgattcgccatgggcggcctcactatggaccaggccttcgtgcaggcccccgagcaccgccccaagcccatcgtcaccgaggccaccggcatccctctcatcgacctctcgcctctggccgccagcggcggcgccgtggacgcgctggccgccgaggtgggcgcggcgagccgggactggggcttcttcgtggtcgtgggccacggcgtgcccgcagagaccgtggcgcgcgcgacggaggcgcagcgagcgttcttcgcgctgccggcagagcggaaggccgccgtgcggaggaacgaggcggagccgctcgggtactacgagtcggagcacaccaagaacgtgagggactggaaggaggtgtacgacctcgtgccgcgcgagccgccgccgccggcagccgtggccgacggcgagcttgtgttcgataacaagtggccccaggatctaccgggcttcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccttgcccgagccccgacctggccctcggcgtggggcggcacaaggacgccggcgccctgaccatcctgtaccaggacgacgtcggggggctcgacgtccggcggcgctccgacggcgagtgggtccgcgtcaggcccgtgcccgactcgttcatcatcaacgtcggcgacctcatccaggtacgagagcgcggagcaccgggtgtcggtgaactcggcgagggagaggttctccatgccctacttcttcaacccggcgacctacaccatggtggagccggtggaggagctggtgagcaaggacgatccgcccaggtacgacgcctacaactggggcgacttcttcagcaccaggaagaacagcaacttcaagaagctcaacgtggagaacattcagatcgcgcatttcaagaagagcctcgtcctcgcctaactactgctactgctaggatccatgccattgccatgtcgtcttcagattcagagcacgccatgtcgtcgctagcttcgtggtagaacaaataatgatgtgcgtgctgtgtgtaagcatggatatggatgtgaatatgtaatatgatgagcactcctactttggtatgtttgggaataacagacttgtgttggtctggttcattatttgtaagaaaatcaaaaagagttagtagggcaggaggctaaccacagtcatgctgcaccacatccctggtggaaagctggccgggttacgcta

SEQ ID NO: 11 - DNA, 1116 bp, Zea mays
  source 1..1116: mol_type = genomic DNA
atgggcggcctcactatggaccaggccttcgtgcaggcccccgagcaccgccccaagcccatcgtcaccgaggccaccggcatccctctcatcgacctctcgcctctggccgccagcggcggcgccgtggacgcgctggccgccgaggtgggcgcggcgagccgggactggggcttcttcgtggtcgtgggccacggcgtgcccgcagagaccgtggcgcgcgcgacggaggcgcagcgagcgttcttcgcgctgccggcagagcggaaggccgccgtgcggaggaacgaggcggagccgctcgggtactacgagtcggagcacaccaagaacgtgagggactggaaggaggtgtacgacctcgtgccgcgcgagccgccgccgccggcagccgtggccgacggcgagcttgtgttcgataacaagtggccccaggatctaccgggcttcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccttgcccgagccccgacctggccctcggcgtggggcggcacaaggacgccggcgccctgaccatcctgtaccaggacgacgtcggggggctcgacgtccggcggcgctccgacggcgagtgggtccgcgtcaggcccgtgcccgactcgttcatcatcaacgtcggcgacctcatccaggtacgagagcgcggagcaccgggtgtcggtgaactcggcgagggagaggttctccatgccctacttcttcaacccggcgacctacaccatggtggagccggtggaggagctggtgagcaaggacgatccgcccaggtacgacgcctacaactggggcgacttcttcagcaccaggaagaacagcaacttcaagaagctcaacgtggagaacattcagatcgcgcatttcaagaagagcctcgtcctcgcctaactactgctactgctaggatccatgccattgccatgtcgtcttcagattcagagcacgccatgtcgtcgctagcttcgtggtag

SEQ ID NO: 12 - protein, 371 aa, Zea mays
MGGLTMDQAFVQAPEHRPKPIVTEATGIPLIDLSPLAASGGAVDALAAEVGAASRDWGFFVVVGHGVPAETVARATEAQRAFFALPAERKAAVRRNEAEPLGYYESEHTKNVRDWKEVYDLVPREPPPPAAVADGELVFDNKWPQDLPGFREALEEYAKAMEELAFKLLELIARSLKLRPDRLHGFFKDQTTFIRLNHYPPCPSPDLALGVGRHKDAGALTILYQDDVGGLDVRRRSDGEWVRVRPVPDSFIINVGDLIQVRERGAPGVGELGEGEVLHALLLQPGDLHHGGAGGGAGEQGRSAQVRRLQLGRLLQHQEEQQLQEAQRGEHSDRAFQEEPRPRLTTATARIHAIAMSSSDSEHAMSSLASW

SEQ ID NO: 13 - DNA, 1733 bp, Zea mays
  source 1..1733: mol_type = genomic DNA
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaa

SEQ ID NO: 14 - DNA, 1392 bp, Zea mays
  source 1..1392: mol_type = genomic DNA
atgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctag

SEQ ID NO: 15 - protein, 463 aa, Zea mays
MRPRLPPNVPSLPSSLSLLANSLSSPVTNTPTRPDSFPAYLQLAHLMVSQERQEPAVPSSSSSSAKRAATSMDASPAPPLLLRAPTPSPSIDLPAGKDKADAAASKAGAAVFDLRREPKIPAPFLWPQEEARPSSAAELEVPMVDVGVLRNGDRAGLRRAAAQVAAACATHGFFQVCGHGVDAALGRAALDGASDFFRLPLAEKQRARRVPGTVSGYTSAHADRFAAKLPWKETLSFGYHDGAASPVVVDYFVGTLGQDFEPMGWVYQRYCEEMKELSLTIMELLELSLGVELRGYYREFFEDSRSIMRCNYYPPCPEPERTLGTGPHCDPTALTILLQDDVGGLEVLVDGEWRPVRPVPGAMVINIGDTFMALSNGRYKSCLHRAVVNQRRARRSLAFFLCPREDRVVRPPASAAPRRYPDFTWADLMRFTQRHYRADTRTLDAFTRWLSHGPAQAAAPPCT

SEQ ID NO: 16 - DNA, 1510 bp, Zea mays
  source 1..1510: mol_type = genomic DNA
aaagagcgcgcgacggcggcccctgggagagccatgcgagactggaggcggaaccgcgcacgacaccaagctgccgcgccggactgctgcacgcaagcgcagcgcaggaccgaccgacctccgtaggcacgcacggcgccggcggcatggcggagcacctcctgtcgacggccgtgcacgacacgctgccggggagctacgtgcggccggagccggagcgcccgcgcctcgcggaggtcgtgaccggcgcgcgcatccccgtcgtggacctgggcagccccgaccgcggcgcggtcgtggccgccgtcggcgacgcctgccgctcgcacggcttcttccaggtcgtcaaccacgggatacacgccgccctggtcgcggcggtgatggccgcggggcgcggcttcttccggctgccccccgaggagaaggccaagctctactccgacgaccccgccaggaagatccggctgtccaccagcttcaacgtgcgcaaggagacggtgcacaactggcgcgactacctccgcctgcactgccatcccctcgacgagttcctgcccgattggccgtccaacccgcccgatttcaaggagaccatgggcacctactgcaaggaggtccgggagctcgggttcaggctgtacgccgcgatctcggagagcctgggcctagaggcgagctacatgaaggaagcgctgggggagcaggagcagcacatggcggtcaacttctacccgccgtgcccggagccggagctcacctacggcctcccggcgcacaccgaccccaacgcgctcaccatcctgctcatggacccggacgtcgccggcctgcaggtgctccacgccggccagtgggtcgccgtcaacccgcagcccggcgcgctcatcatcaacatcggcgaccagctgcaggcgctgagcaacgggcagtaccggagcgtgtggcaccgcgcggtggtgaactcggaccgggagcgcatgtccgtggcgtcgttcctgtgcccgtgcaaccacgtcgtgctcggccccgcgcggaagctcgtcaccgaggacaccccggccgtgtacaggaactacacgtacgacaagtactacgccaagttctggagcaggaacctggaccaggagcactgcctcgagctcttcagaacctagcgaatcggatacggatggatggatacattacatacgcgccctctgtttttctccatgacgttagaagaacacgttctgcaatgtttgtccattcaaggtggtatcaatcaaggctgtggtcgttgcaattcttccgctccatatacatgattaaatgctttgaaagaaaaagaaaaaaaagaaacacaagtattatggcactactagtgtttttaggaacaaggaaagaggggttgcccctgctggctatatatattaaatataaataaaggtaaggctgtagacattggtgaataagagaaagtatttgagtttctctattgtcactccagaacagactcctttgcctcgat

SEQ ID NO: 17 - DNA, 1011 bp, Zea mays
  source 1..1011: mol_type = genomic DNA
atggcggagcacctcctgtcgacggccgtgcacgacacgctgccggggagctacgtgcggccggagccggagcgcccgcgcctcgcggaggtcgtgaccggcgcgcgcatccccgtcgtggacctgggcagccccgaccgcggcgcggtcgtggccgccgtcggcgacgcctgccgctcgcacggcttcttccaggtcgtcaaccacgggatacacgccgccctggtcgcggcggtgatggccgcggggcgcggcttcttccggctgccccccgaggagaaggccaagctctactccgacgaccccgccaggaagatccggctgtccaccagcttcaacgtgcgcaaggagacggtgcacaactggcgcgactacctccgcctgcactgccatcccctcgacgagttcctgcccgattggccgtccaacccgcccgatttcaaggagaccatgggcacctactgcaaggaggtccgggagctcgggttcaggctgtacgccgcgatctcggagagcctgggcctagaggcgagctacatgaaggaagcgctgggggagcaggagcagcacatggcggtcaacttctacccgccgtgcccggagccggagctcacctacggcctcccggcgcacaccgaccccaacgcgctcaccatcctgctcatggacccggacgtcgccggcctgcaggtgctccacgccggccagtgggtcgccgtcaacccgcagcccggcgcgctcatcatcaacatcggcgaccagctgcaggcgctgagcaacgggcagtaccggagcgtgtggcaccgcgcggtggtgaactcggaccgggagcgcatgtccgtggcgtcgttcctgtgcccgtgcaaccacgtcgtgctcggccccgcgcggaagctcgtcaccgaggacaccccggccgtgtacaggaactacacgtacgacaagtactacgccaagttctggagcaggaacctggaccaggagcactgcctcgagctcttcagaacctag

SEQ ID NO: 18 - protein, 336 aa, Zea mays
MAEHLLSTAVHDTLPGSYVRPEPERPRLAEVVTGARIPVVDLGSPDRGAVVAAVGDACRSHGFFQVVNHGIHAALVAAVMAAGRGFFRLPPEEKAKLYSDDPARKIRLSTSFNVRKETVHNWRDYLRLHCHPLDEFLPDWPSNPPDFKETMGTYCKEVRELGFRLYAAISESLGLEASYMKEALGEQEQHMAVNFYPPCPEPELTYGLPAHTDPNALTILLMDPDVAGLQVLHAGQWVAVNPQPGALIINIGDQLQALSNGQYRSVWHRAVVNSDRERMSVASFLCPCNHVVLGPARKLVTEDTPAVYRNYTYDKYYAKFWSRNLDQEHCLELFRT

SEQ ID NO: 19 - DNA, 1387 bp, Zea mays
  source 1..1387: mol_type = genomic DNA
gttttctttttgaacgtaactgacagaagctatctgcctagctacggcgtgtcggttgcttgtctcaccaaagcagcgacatggaagcctgacagctcgtcgcgtcgcgccatttccacccaacaaagcggcggcgccagcacgcactgcttctgcttgtgcgtgctcctccgttccgggcacgcctctaaagtctatacagcctcgaatccatcccggccgccgctcctgggggatactacagcgagccgaagcggggatggcggagatccctgtgatcgacctgcgcgtcgccggctcggcggccgaggagtccgcgcggctgcgggccgcgtgcgagcgcctgggctgcttccgggtgaccggccacggcgtgccctcggtgctcctggcagagatgaaggccgccgtgcgcgcgctcttcgacctccccgacgacgccaagcgccgcaacgccgacgtcatcaccggcagcggctacgtcgcccccagcccgaccaacccgctctacgaggccttcgggctcctcgacgccgccgtgcccaccgacgtcgacgccttttgcgcgctcctcgacgcgccgcccaacatcagggagaccgtcaaggcctacgcggagaagatgcacgatgtgatcgttggcgtcgcccgcgagctggcgtctagcctggggctagtcgaggagcactcgttccaggactggccgtgccagttccgcatcaacaggtacaactacacgcgggagacggtgggctcctccggcgtgcagacccacacggactcgggcttcctcaccgtgctccatgaggacgagtgtgtcggcggcctcgaggtcctggacccgggcaccggcgagttcgtgcccgtggaccccgtcgcgggctcctttctcgtaaacatcggcgacgtcggcacggcgtggagcaacgggaggctgcacaacgtgaagcaccgggtgcggtgcgtcgcacccgtgccgcgcatctccatcgccatgttcctgctcgcacccaaggacgacagcgtgagcgcaccggcggcgttcgtggacgcggaccacccgcgcaggtacaaggtgttcaactacaacgactatcggaggctcagactgtccaccggcgagcacgcaggcgaggcgctcgcacggatggcggcgtgacgtggctggagctgcaaattggattggaagccgagacaagccgttagttatttaccatgcccgtgcgttcaccgcacacaatcatattcaaaagccataaaataaaaaataattttaatatcagtcaacatatggtttaaatatcatatggagtacaatattccgaatttttttttgtaatttagtctgtcttttgaaaaaaatgcacatctagacctccggatgact

SEQ ID NO: 20 - DNA, 900 bp, Zea mays
  source 1..900: mol_type = genomic DNA
atggcggagatccctgtgatcgacctgcgcgtcgccggctcggcggccgaggagtccgcgcggctgcgggccgcgtgcgagcgcctgggctgcttccgggtgaccggccacggcgtgccctcggtgctcctggcagagatgaaggccgccgtgcgcgcgctcttcgacctccccgacgacgccaagcgccgcaacgccgacgtcatcaccggcagcggctacgtcgcccccagcccgaccaacccgctctacgaggccttcgggctcctcgacgccgccgtgcccaccgacgtcgacgccttttgcgcgctcctcgacgcgccgcccaacatcagggagaccgtcaaggcctacgcggagaagatgcacgatgtgatcgttggcgtcgcccgcgagctggcgtctagcctggggctagtcgaggagcactcgttccaggactggccgtgccagttccgcatcaacaggtacaactacacgcgggagacggtgggctcctccggcgtgcagacccacacggactcgggcttcctcaccgtgctccatgaggacgagtgtgtcggcggcctcgaggtcctggacccgggcaccggcgagttcgtgcccgtggaccccgtcgcgggctcctttctcgtaaacatcggcgacgtcggcacggcgtggagcaacgggaggctgcacaacgtgaagcaccgggtgcggtgcgtcgcacccgtgccgcgcatctccatcgccatgttcctgctcgcacccaaggacgacagcgtgagcgcaccggcggcgttcgtggacgcggaccacccgcgcaggtacaaggtgttcaactacaacgactatcggaggctcagactgtccaccggcgagcacgcaggcgaggcgctcgcacggatggcggcgtga

SEQ ID NO: 21 - protein, 299 aa, Zea mays
MAEIPVIDLRVAGSAAEESARLRAACERLGCFRVTGHGVPSVLLAEMKAAVRALFDLPDDAKRRNADVITGSGYVAPSPTNPLYEAFGLLDAAVPTDVDAFCALLDAPPNIRETVKAYAEKMHDVIVGVARELASSLGLVEEHSFQDWPCQFRINRYNYTRETVGSSGVQTHTDSGFLTVLHEDECVGGLEVLDPGTGEFVPVDPVAGSFLVNIGDVGTAWSNGRLHNVKHRVRCVAPVPRISIAMFLLAPKDDSVSAPAAFVDADHPRRYKVFNYNDYRRLRLSTGEHAGEALARMAA

SEQ ID NO: 22 - DNA, 1496 bp, Zea mays
  source 1..1496: mol_type = genomic DNA
gtcggtctcttgtctcaccaaaccggcgacatggtacatggaggccagcccgtcgcttggcgccacaagtctcggtgccgtccgtccgacaagcggcgccagcgcacgctggctgctcgtgcacgcctctaaatacggccccggacccgccaccaagcgaaggccaatcccgtccgccgccccccaccaaccacgaaccacgcaagcgaacccggccggcgcggggcagcggcgatggcggagatcccggtgatcgacctgcgcctcgccggctcgtcgcccgacgagtcggcgcggctgcgcgacgcgtgcgagcgcctgggctgctttcgggtgaccggccacggcgcgcccgcggggctcctggccgacatgaaggccgccgtgcgcgcgctcttcgacctccccgacgacgccaagcgccgcaacgccgacgtcatccccggcagcggctacgtcgcgccctgccccgccaacccgctctacgaggccttcgggctcctcgacgccgccgcgcccgccgacgtcgacgccttctgcgcgcgcctcgacgcgccgcccaaagtcagggagaccgtcaagacctacgcggagaagatgcacgacgtgatcgtcggcgtcgccggcgagctggccaccagcctggggctgggcctggaggagcactcgttccaggactggccgtgccagttccgcatcaacaggtacaactacacgcaggagacggtgggctcctccggcgtgcagacccacacggactcgggcttcctcaccgtgctccaggaggacgagtgcgtcggcggcctcgaggtgctggaccccgccgccggtgagttcgtgcccgtggaccccgtcgccggctccttcctcgtcaacatcggcgacgtcggcacggcgtggagcaacgggaggctccacaacgtgaagcaccgggtgcggtgcgtcgcgcccgtgccgcgcatctccatcgccatgttcctgctggcgcccaaggacgaccgcgtgagcgccccggaggcgttggtcgacgcgggccacccgcgtcggtacaagccgttcaactacgacgactaccggaggctccggctgtccaccggcgagcgcgcaggcgaggcgctcgcgcggatggcggcgtgatgtcgtcacgcacgtgcaagccgttaattataggctcgcgcatgcatacgcctacacgagaggttgtctcgttaagccgttctattaaaatgtgtgggggagaaagatgactaccgtggtgccatgtggattgctatcgggtctgatcaataaaatcttgcaacacttgcacgtgcgattccatatcctagcacgggtgggcgccacgctagtaggtagagaccggagcggccaaaaaatggctacagcaccagtaggtgaactctcaagcaacactggctatcccacttctgacgttgtctctctcatcactatgtatgaccagcgaatgaagtgtttaaaaatctgacgccgtgaaa

SEQ ID NO: 23 - DNA, 903 bp, Zea mays
  source 1..903: mol_type = genomic DNA
atggcggagatcccggtgatcgacctgcgcctcgccggctcgtcgcccgacgagtcggcgcggctgcgcgacgcgtgcgagcgcctgggctgctttcgggtgaccggccacggcgcgcccgcggggctcctggccgacatgaaggccgccgtgcgcgcgctcttcgacctccccgacgacgccaagcgccgcaacgccgacgtcatccccggcagcggctacgtcgcgccctgccccgccaacccgctctacgaggccttcgggctcctcgacgccgccgcgcccgccgacgtcgacgccttctgcgcgcgcctcgacgcgccgcccaaagtcagggagaccgtcaagacctacgcggagaagatgcacgacgtgatcgtcggcgtcgccggcgagctggccaccagcctggggctgggcctggaggagcactcgttccaggactggccgtgccagttccgcatcaacaggtacaactacacgcaggagacggtgggctcctccggcgtgcagacccacacggactcgggcttcctcaccgtgctccaggaggacgagtgcgtcggcggcctcgaggtgctggaccccgccgccggtgagttcgtgcccgtggaccccgtcgccggctccttcctcgtcaacatcggcgacgtcggcacggcgtggagcaacgggaggctccacaacgtgaagcaccgggtgcggtgcgtcgcgcccgtgccgcgcatctccatcgccatgttcctgctggcgcccaaggacgaccgcgtgagcgccccggaggcgttggtcgacgcgggccacccgcgtcggtacaagccgttcaactacgacgactaccggaggctccggctgtccaccggcgagcgcgcaggcgaggcgctcgcgcggatggcggcgtga

SEQ ID NO: 24 - protein, 300 aa, Zea mays
MAEIPVIDLRLAGSSPDESARLRDACERLGCFRVTGHGAPAGLLADMKAAVRALFDLPDDAKRRNADVIPGSGYVAPCPANPLYEAFGLLDAAAPADVDAFCARLDAPPKVRETVKTYAEKMHDVIVGVAGELATSLGLGLEEHSFQDWPCQFRINRYNYTQETVGSSGVQTHTDSGFLTVLQEDECVGGLEVLDPAAGEFVPVDPVAGSFLVNIGDVGTAWSNGRLHNVKHRVRCVAPVPRISIAMFLLAPKDDRVSAPEALVDAGHPRRYKPFNYDDYRRLRLSTGERAGEALARMAA

SEQ ID NO: 25 - DNA, 1614 bp, Zea mays
  source 1..1614: mol_type = genomic DNA
accacacgaattgcacatctccacagctcacgattccaacactagctacatatatatgtagctttctaggctactatatacactcaccaccaagtgtgaagtgtgtatatatagtgacagctactgcaatatatacatacgcgtcacctatatattagccaagctagctatatgagcttggttgcggcgccaatggcgatcgtcgacgtggccaacgcccagctgcagcaagcagcagcagcagctgccaagaaagacgaggacggccatgagcagcaggagtcgtcctacgactacggcgcgctgatgaaaggcgtgaggcacctgtcggacagcggcattaccaggctgcccgacaggtacgtcctgcccgcgtccgaccgccccggcgtccttgccgtctcgtcgtccgtggcgggcagcggcagggtcaagctccctgtcgtcaacctcgccggcctccgcgacccctgccagcgcgccgccgtgctggccacgctcgacgccgcgtgccgggagtacggcttctttcaggtggtaaaccacgggttcgggagcgacgtgagcggcgggatgctggacgtggcgcagcgcttcttcgagctgccgctggccgagcgagcgcggcacatgtcggcggacgtgcgggcgccggtgcgctacggcaccagcttcaaccaggccaaggacgacgtgctctgctggcgcgacttcctcaagctcgtctgccagccgctgcaggcggtgctcccgtactggccccagcagccggcggacctcagggacgtggccaccaggtacgccacggcgagccaccggctgttcatggaggtcatggaggcggcgctggaggccctgggcatccccacggccggcggcgtgctcggggagctggcagcgtcgtcgtcgcacatgatgacggtgaactgctacccggcgtgcccgcagcctgagctcacgctggggatgccctcgcactcggactacggcctcttcacgttcgtcctgcaggaccacgtcgagggcctccaggtcatgcacgacggccgctggctcaccatcgaccccatcccgggatcgttcgtcgtcaacgtcggcgaccacctagagatctacagcaacgggcggtacaagagcgcgctgcaccgggtgcacgtgaactccacgcggccgcgcatctcggtggcgtcgttccacagcctgccggcggagcgagtgatcgggccggcgccggagctggtggacgacgaggccggcaacccgcggcggtacatggacaccgacttcgctaccttcctcgcctacctcgcatccgcggacggcaagaacaagaccttcctccagtcaaggaagctgcctgctgctgctcctccatgcctctagctaactagatagctgcttattaatctgacagaataaaattaatcagttcagcgcacaattccacaagcgaaaacaaacctggatttgttttaattagctctgcccttcattattacattcaagctagctcttggtcaacgcatgcacacaagcttgagcattgactggtcccttttcaatcggttgcattgtactccctccgtaccaaaattggttgtcgctatagtattt

SEQ ID NO: 26 - DNA, 1212 bp, Zea mays
  source 1..1212: mol_type = genomic DNA
atgagcttggttgcggcgccaatggcgatcgtcgacgtggccaacgcccagctgcagcaagcagcagcagcagctgccaagaaagacgaggacggccatgagcagcaggagtcgtcctacgactacggcgcgctgatgaaaggcgtgaggcacctgtcggacagcggcattaccaggctgcccgacaggtacgtcctgcccgcgtccgaccgccccggcgtccttgccgtctcgtcgtccgtggcgggcagcggcagggtcaagctccctgtcgtcaacctcgccggcctccgcgacccctgccagcgcgccgccgtgctggccacgctcgacgccgcgtgccgggagtacggcttctttcaggtggtaaaccacgggttcgggagcgacgtgagcggcgggatgctggacgtggcgcagcgcttcttcgagctgccgctggccgagcgagcgcggcacatgtcggcggacgtgcgggcgccggtgcgctacggcaccagcttcaaccaggccaaggacgacgtgctctgctggcgcgacttcctcaagctcgtctgccagccgctgcaggcggtgctcccgtactggccccagcagccggcggacctcagggacgtggccaccaggtacgccacggcgagccaccggctgttcatggaggtcatggaggcggcgctggaggccctgggcatccccacggccggcggcgtgctcggggagctggcagcgtcgtcgtcgcacatgatgacggtgaactgctacccggcgtgcccgcagcctgagctcacgctggggatgccctcgcactcggactacggcctcttcacgttcgtcctgcaggaccacgtcgagggcctccaggtcatgcacgacggccgctggctcaccatcgaccccatcccgggatcgttcgtcgtcaacgtcggcgaccacctagagatctacagcaacgggcggtacaagagcgcgctgcaccgggtgcacgtgaactccacgcggccgcgcatctcggtggcgtcgttccacagcctgccggcggagcgagtgatcgggccggcgccggagctggtggacgacgaggccggcaacccgcggcggtacatggacaccgacttcgctaccttcctcgcctacctcgcatccgcggacggcaagaacaagaccttcctccagtcaaggaagctgcctgctgctgctcctccatgcctctag

SEQ ID NO: 27 - protein, 403 aa, Zea mays
MSLVAAPMAIVDVANAQLQQAAAAAAKKDEDGHEQQESSYDYGALMKGVRHLSDSGITRLPDRYVLPASDRPGVLAVSSSVAGSGRVKLPVVNLAGLRDPCQRAAVLATLDAACREYGFFQVVNHGFGSDVSGGMLDVAQRFFELPLAERARHMSADVRAPVRYGTSFNQAKDDVLCWRDFLKLVCQPLQAVLPYWPQQPADLRDVATRYATASHRLFMEVMEAALEALGIPTAGGVLGELAASSSHMMTVNCYPACPQPELTLGMPSHSDYGLFTFVLQDHVEGLQVMHDGRWLTIDPIPGSFVVNVGDHLEIYSNGRYKSALHRVHVNSTRPRISVASFHSLPAERVIGPAPELVDDEAGNPRRYMDTDFATFLAYLASADGKNKTFLQSRKLPAAAPPCL

SEQ ID NO: 28 - DNA, 1863 bp, Zea mays
  source 1..1863: mol_type = genomic DNA
tgccaccataccactagtgcaaggtcctagatttacacttggtgctacaccttgcttcgcccccttccttccttccttccttccttccctccttccttggtctctaggcagctagcagtgtggtgctgctgccggccgcctattggccgcctgggactgggatccattaattactgcgcgcgcgcggctaaccaaccaatcccagcgtgcgtaatctattgcccacatgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctgtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtagccgagaccgatcgccggagactgatgctgatgatgatgcatatatacatgagagaaatcgtcgagtagactagccgattgcaaaagcaaccccagctgccgaaacctggcatatcgatcccattctctgctgcgcacatgtatgcatgcatgcgcttcgtccgttcgactcgtgtgtgcttgcttgcttgcgcgtgcagcagaactaattccgttccgcagctagctgctctgctctgctctgctggaatgtaattaagtagtagtatatggtagtagagaaaagattagctaggcgatcgatatagatgacgggccggggaagaagacgaattaattaagatcgatcgacgacgacgagctgtgcgtggctggctgtgttcttctctagcctagttacagaggccggctgctgctgcttccaatcgggctgcttgtcgctactgacgatcgttagtggatccattaactaatctggaattctggatt

SEQ ID NO: 29 - DNA, 1149 bp, Zea mays
  source 1..1149: mol_type = genomic DNA
atgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctgtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtag

SEQ ID NO: 30 - protein, 382 aa, Zea mays
MPTPSHLNKNPRYLDFRAARRVPESHAWPGLHDHPVVDGGAPGPDAVPVVDLGAADPAPAPAAAVARAAEQWGAFLLTGHGVPADLLARVEDRIATMFALPADDKMRAVRGPGDACGYGSPPISSFFSKCMWSEGYTFSPASLRADLRKLWPKAGDDYTSFCDVMEEFHKHMRALADKLLELFLMALGLTDEQASAVEAERRIAETMTATMHLNWYPRCPDPRRALGLIAHTDSGFFTFVMQSLVPGLQLFRHAPDRWVAVPAVPGAFVVNVGDLFHILTNGRFHSVYHRAVVNRDLDRISLGYFLGPPPHAKVAPLREAVPPGRAPAYRAVTWPEYMGVRKKAFTTGASALKMVALAAAADLDDDGDAAVVHQQQQLVVSS

SEQ ID NO: 31 - DNA, 1439 bp, Zea mays
  source 1..1439: mol_type = genomic DNA
gacctccattttgattatctctatcctgtacgtgccgagagtccttcaaagccgacgacgagacgacgatgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctgcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaagccctaaggaactactagctgaatccataaactaataaagaattcgtgaataagggcgttggaagactggacacaacacaagagagttgctatatatcgtatttctgaaatttaaggcaaatatcttagttaaaaaactggtatatttaaatagacaatatatatctaaaataaagatagttcaccatttttacggtcgaacaatgataaagttatatattgtctgaatagtaacaaattaaagatttccaggag

SEQ ID NO: 32 - DNA, 1116 bp, Zea mays
  source 1..1116: mol_type = genomic DNA
atgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctgcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaa

SEQ ID NO: 33 - protein, 371 aa, Zea mays
MQSSSSSASTPAAASGLVFDLGSAAGVPETHAWPGVNEYPSVESAGRDVVPVVDMGVACPDATRALARAADEWGVFLLVGHGVPREVAARAEEQVARLFVLPAPDKARAGRRPGEPTATGYGRPPLALRFSKLMWSEGYTFRAATVREEFRRVWPDGGDDYLRFCDVMEEYDREMRALGGRLLDLFFMALGLTDVQFATGETERRIRETWTATMHPILCPEPERAIGLTAHTDSGFITLIMQSPVPGLQLLRRGPDRWVTVPAPPGALIVMLGDLFQVLTNGRFRSPIHRAVVSRERERISVPYFLCPPEDMTVAPLASALLPGRKAVFRAVTWPEYMEVKHKVFGTDAPALEMLQLQVDEEEQGERAATT

SEQ ID NO: 34 - DNA, 8800 bp, Zea mays
  source 1..8800: mol_type = genomic DNA
taaatttgtgatccttgtgaagttgttatatcatgaattgtgaacttgttgcatttgtgatcttttgtcaactttgttgtattgtgaagtttgatatgtttaccgatcgtattttagatttcgatcgttaccggtgtattttccgcaccaaacttttgtttccgatgttttcgaaataccgatatcgtttccgtttctatagttacccttttcaattttatttccgattaaaaatatgaaaacggtaatggttttagtgtttatcgaccgttttcatctctaatcatccctgccggtgaagtttaatttttcccttggctaaagagatgcaagctgctgtaaaatacgttaaaacaggcaaggcagccccagcagccagcatcgcgtgcccgtctatgtacatcagtggatacgtagcatctctagtgagtaatataacgattgcatttggctggaggacgtatgttatataagtatgtcatttaccagttgcattagtatcttccctaactcctataataactctcttcgtggaatggacgtagacgtatgctatataagtattaaaaaatagttttttaagctggtgtcctcaattttgctattgttctcgtttttatctttagttgtgtcacaaatttaatccgtacaacaaatcaaaaataccatacccttcttatattaattttctaacataacatttgtttagatattttcagtcgtgaaaatacaattctaattctaacgtcgtagtatcaaatcaaaccatccagaatttgaccaagcttaattataaaaaatataaaatttatgatactgaatagatagcattagatttgttatataatatatttttataaaataccatttttatggtataaatattggtactcctttactttaaactatagatagttttgactaaggatgcaactagaattgcatcctcttttcactgcaccttcattagttttaatatttatttagatgggcccttgcaaactgtagatatcatctcttgcaacattctttctatagcaccacgaaaatgtattgcggctttgaaattataattgaattagttgtatcatttctttcaccgatgcgttaaattcaaaattaagtgttatatttcttcataatttgttaaatatatagaccctataatccaccattatttactataatagcatacattaacattggttttagcctacactacgacactcgaggcattgaattttcctctatcaaagaattatatgtgtagtagtattgttcttgacaaaaagggggattaaaattaaactaccaatattgatacttatcttatcacatccatgaatacaatcaacactcttacaaaagataagatacaagattaaaaagtaccatgataatacattaagattattagcaatgcattaaattaaataaatgtgcaagtgaatcatgattttagttttatctattttacttttaaaatatgatattctctgactacttctaagcataaatgtgattctaagtcatgaccgatcgtgcttattcagaaaaatgaaggagacacagatttctataaaaaaaggttgtcatgggactattgggtcaaccatcttattcatttgggaaaataagtttagaacacatcaacccattttagatgttgagtttggccctaatggtccattgaccttacttttgtgggttgacatagaccatctatcccaagttattgttgtgtcacattccctgatatcatgaatctatattttagctttccgttttcatatttttagtcgttacatattttttatccgcgtactagattaaaactctagttgttgcaatacattttgttcatttttttctatttcttctttactaacaacatattctagttcctagctacattcttaagtaccatagtgctataaacattttttatcctacattattccacttaagaaattgaattttctgcataaaaaaattatatgtccagtagtgttgtcttataaaagcataaagtgattaaaattaaaaccattattgatatcttatttttcaaaaaaaaatataagcttatagaaagtgaattaatttcatggtaaattaatatagtttaaattgaattattagtgttattactatgtttattatcaatgaaacatttttcatggttgatataacttagtgttacttattttagtattttttatataattctagttaacttttagtttttgatttaaaaaaacgagaattgtgtccttttgtggagtgagtataaagaaagtaatatctgttcatcataatttggttttttaaggtacgtgaaacttgctttatatttggactcaagctatgtctaaatacatagtaaaaaagcaatatttctagaaaagacaaaacatcttataatttagaatcaaggaaatatatagattttatgtgcagtgagaagccatttacaatggaacgttcaacgttgggccaatagatattttgcgatatgatgatgggcatatttttgcatggttgtccctccactagctatagtttgatgatacgatacgctgcacacaccattgggttgtaccatgttagtgtagcaacagtagaaacccaattgtggccgtgaaccatgataatactaggtagagtgctagctagaggtttcaggctattgatgcgtgaattaaactttctgttgtgttgcgaggaaacgagtattgtgaaatatttgaaacggttttttttgtgaaagatttgaaacggtatttttgttgtgaaataaagatcaaggctaaataaattcaaactaataaaacatattaattgacggcctgaagcccccgcccccatggccccatgccatagcatcaggtcccacatgacatgaggccgcgcctccctctatgttggctccctgccttcgccgttgtcgtcgctcccgaactccctctcctcccctgttacaaatacccccacccgcccggacagcttccctgcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgacggcgcggggggtttttcacaaaacagactcggcaagctacggaggttgcgggagggttcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaacgagccggccgtctctttcgccggggcccgcgcggggttcgcccacgtggtgatcaggtggcagacatgtggcccacgggccccgcgccgccttccccatttttggacgaccctactgctactactactagtgtacatatgcaaaaaaatacatatatatataggtactttctctaatatttttatatataagcaaggcggcctggtgttcttttctttgttttgtcgacaactgtttgatcccatcctatggacgatggatagttcaatgtttgtacgtacgtaactactctctatagactagaatgggctcatgaaactggaccgatcgacacggacgtcacgtgcgtctggtaccggtagtgcaacgggtgccgaatgtttgctgggcccggacgagaatcgcttctcctcgtcctcggtcctcaccctgaacgaacgaataaggaaaatgctgcaccgaaagctccagacgtttccgaattccaaattccaaaaccccaaatcttcttgcttcacatcagtcttacccggttcatctgtgacaaaaaaaaaatagtgctagtttaggaactcaggtcgagattgaaggcaattgtggaggaatttaccctataatccttatgagaatttgagttcccaaactaactgagttggagcattcaacatttccctaaattttgtgcacatgtttctttgctatttatctttggacatgggacgatgggagacgcagatttaggggacccttcaattcagaacttcaggtgcacaaaccgaggttggcttgcctgcattcttgtttcggacatgcccaactaggccactactcactaccttcatctgagataccaattgctgacctaaatgacaagtatacacttacatttcagtgatagctgcaacaaaaaaaaaaatcttaccgcattttatctctgcattctgcatgccgcatcctgaacattacgtatctttcccggtgctctgttgcgttctcacgcagttgatggcatgcagtcttgcgccaccgaatccagtgtactggtcgtggtgacttgtcgcacagacagcagcccggcagcaccaagcgtgtcactgtaaactgttgggcgttaaacaacaacttgcacaacagctcaaatatggcatatgctatccgacaaactgaacaaggtgcccaattgatctgaatgtacctgtgatttccagcactatcgtacagcaacgttgtcaaaacaagtggggtggggttggggacagattttttcgataaagaagcttttataaaaaataacaatgatacaaatcctgggttatagatgtacagaaagcacgaagcacgaaagtccagtccaaagcacgtttttttgcctggtactagcccgatccggccggcacgaataagcgggtcgagctcggacgggaagctaagcacgacggactagcccgacacgacccgtttacctctaatcccgttaaacccgcttttttgcactaaaccgtgcttaccgaaccgtttagcccgttttttggcctgatttttcgtgcttaacgggccaggctcggacaaggaaacaagcccgcgggcttagacgatccggcctggttttttaaccgtgcctagcgggtcgagcccaaaataggtcgggcttcactgggcccgagccgggcgacccgtttggccatctctaacctgagtacaacttccgacttctgcaaaatacatacagccaaataaaagaaaagtaaaaagatctaaaaaatctcagctagaaacaatcaatccgaagactagatcgcctcccataagtctggaaaaaagagaccatggccacttgcaccaagatgtgtgcattattctacggttacaccaaagatttttgttctttaagggctagtttgggaaccataattttcaaagggatttctattttcctaaggaaaattagttcatttttccataagaaattagaaatccattggaaaattgtggttctcaaactagccctaagcgtcgaaaagaaccatatgcatatctagagacaaaattcctctaatttctattcaggcttcagcacatatacttcacgtgcttgcgtcaagttccttgggccgccacatggacttatggacttctcgacgcagcgaaagccgtcgttgcccttggtgtagctaggtcatccgcacctcccactggccagtggccactgcagggacttggccatgggcttgtttggttcagcttttttctgaccagcttttctgaaaatctggctgtgtgaagaatctggctgtgagagaatctgagtatcattacgattacgtgtggatgaagataaagttgttcatagggctcaggatctagaaagtgacggattcctactattacaatgactcaaccgattatgtgtttatgttgattttggatgatttttgccccaacaaattttatagaagctggctgaaaagctgagcgtttggcagtccacaacagtttttggtggccagaagctgccagaagccgatacaaacagggtccatgctttccatttcgtttaccgtgtacgcggtgtccctcacaatcaatcagtttaccttgtggctccaacacacatcaacctcggcacaacaacactgtgaatcattttcggcggtccatattattttcgacggttcatccctggccgccgaaaattgtctgttattttcggcggcttgacctagccgtcgaaaataggctgctattttcggcggccaaatccgagccgccgaaaataaggcttttaaaaaccgtcggctccttcttcttctctgttctttctctcctctcccaaagcccgccgccgctcacccgccgctcgctcgccgggctgcccgccactccgccgtcgtcgtcgagccaccgcatcgagaggtaaattttttttgcgtgttttattccttattttcggcggttgatatttaggcgccgccaaaattagtgtatatttgtaattgtgtttgtttaattactatttgtaattagtattattgtttaattcgatttcattaatgtattagtggtatatgtgatttagggattaggggcatattgtatttaggcattaatttcatattaatatgtggtattattatattattggttttaatagtacattatattggtacgtagaatagttgcattattagtgtttgtggtacttagtttgacttgat

SEQ ID NO: 35 - DNA, 8859 bp, Zea mays
  source 1..8859: mol_type = genomic DNA
cctattttgtgtctaatactcttcttatattaattgtttggtcaaactttagataaatttgactaatgatgcaattaaaactgcatcacctttactaaggtactgctttatatgtttcgacaaaattttcaattattctctatgtgttttaatctttgcgctacacctccattgatttaaatactcatttattttaaaccataacttaaattatatcggatctttgcatcctttctatggcaccatacatgaatcgatattttggctgcaaatttttaatcatgttagttttagcattttttcatatccatgtgttaagtttgaatcatgtgttgtttttatataatttattgaaaatatagatcctaaacttcactaatacttacaacaatagcatcatcatgtgttttaatccacgccacaacactcaaggcattgaattttcttctaccaaagagttgtatgtgtgtattgttctttaaaaaatagagtgattataattaaactaccagtattcatatgtaaaatgtatagacatctaaaataaaatttgcaaaaaacattgttgcagactttcaatataattaagaatgggttttagggtcatgatatatggtttgttaaagaaacttgtttttttttgcaattgataaactataaaatacattttcactattgtgtgcatatgtacttggtatacatagtggcatatatcatttttgtttactttgaggtttgaattatctatgttaaaattggataacatagatacattggtgtgcgtcctttggcccatttacttgactgaggagcaatactataaagtaaaacatatttggatattttatcttaaactcctagcataatattgatttaattatgaacaaatatatgtttaggtgatagtttcatgggtggtaaactatataagaaggcttaccatgatctttgcaaactctaggctatgaaagagttccatgatttgtcttagaagcatagacaaaacagtgataatgatctaaatcacacttatggcactgatgaccatatatgcaaagctaaatgcatgttaagttgtattatatcatatgtttacaatgactatcgcatataacgaggaatacattgtctatatagatagctattactgtagtagtgccaaatgttggacaacatgaatcataatcttcaaacctagagaaattgtagtcagtcgtacacatatcgtctagtaagttgtctatactttttatttattgtatcaaattttattgttatcttgcttgcttgtttgtttgtaccatagacacaatatggtcaaaaagtggtcaatcgattcgaagaagattgcaattgacgagtgctaacagttgatccttttgttgtgcacgctagcggagtagcatgaaaagagtaaaatatgaaattagcgttctaaactgtttgtgctataggtacttcgtatttaatggagtgactaactataggaaggtgagagctcagaagtcagcaccctcacacagagttctagagttagtggtcatcgaaccacgacaaactacatgatgagcagaagaggcaacatcaagactatgatcaatagtttcgggtcaatgaatgacatcgtgatgagtatttatctaactatatagaacaacaacacatgatgttttaagtaagttcaactgatcttctattgctatctttaagtatttaacgtagcgaataatgttttatctatttcattcataaataatgttgtgacaaaaggggataaccatcacttttaccatgttctagataccacaaccatctccaccatcataatgggttcttcattggtgcttggacctcaaataatcatatctatagccaacttagctcaattctaataaaattaggcaacttggcttcattgtagcaaaaatagccaacttagctcaattttatctaaacttagctaatctagcacaacttagatcaatattaggaaaaactaatcaatctaatctagctcaactatagcgaaagatagatattgtagcataacttagtagatctatctcaaattttagcaaaaactaatcaatttagataaactctataaaattttaatcattatgacttatttccaactaattgtaacttgcatgatttttatgttccttctttataattagcaacacctaaagacacgaatgatgaggggtctaacgcattcattaaccagttgttaaataatactctaggtagatgataagaactctaattattctatgaatctaagctaaaagatgtttaatatttaagtattggtgtttattatgttatttagaacgattcatgttacttaaagatttgttatgatttttaaatatgattatgataatttatgtggtgtggattaacttgtgaacatatgtgatgtagatgaatatgtatgttgtggatggaaccatatgaatatatatacacactcatatactattcgttggtgtaggtaaagcttcatccatcggtaattactaaatggtcttcagtcattaccactaggtgaagcttcacacgaccgataattattgaagaacgctcattaatttccggtaatggcttattggccttcactagtcggtgaaaattagctatttttataccaataaaaattagctaatatatgtaaaccaggtctaatttttatgggcctcttaccgaccaaaattgattagattattgttacaatagttttagtcaaaagctagctatgctataaaaattttgaattaaagtgagtttcgtaataaaaattgcatacttttaaaataaaataattaaaaaacagtttttagaaatacaatcaaacaccttatgctataaaaaaattgtaatgtacctacaaatatataatactttactttaaaataggcctgtgccttctcggctctatatgggctgcctccaacgaagcgccatggccatgggctccactgtgtcgggtcccacatgaggccgcgcctccctccaaatgttccctccctgccttcgtctttgtcgttgctcgcaaactccctgtcctcccctgttacaaatacccccacccgcccggacagcttccctgcatacttgcagctcgcacatctcatggtgtcgcaggaacgacaagagccagctgtgcctagcagcagcagcagcagcgccaagcgcgcagccacgtccatggacgccagcccggccccgccgctcctcctccgcgcccccactcccagccccagcattgacctccccgctggcaaggacaaggccgacgcggcggccagcaaggccggcgcggccgtgttcgacctgcgccgggagcccaagatccccgcgccattcctgtggccgcaggaagaggcgcggccgtcctcggccgcggagctggaggtgccgatggtggacgtgggcgtgctgcgcaatggcgaccgcgcggggctgcggcgcgccgcggcgcaggtggccgcggcgtgcgcgacgcacgggttcttccaggtgtgcgggcacggcgtggacgcggcgctggggcgcgccgcgctggacggcgccagcgacttcttccggctgccgctcgccgagaagcagcgcgcccggcgcgtccccggcaccgtgtccgggtacacgagcgcgcacgccgaccggttcgcggccaagctcccctggaaggagaccctgtcgttcggctaccacgacggcgccgcgtcgcctgtcgtcgtggactacttcgtcggcaccctcggccaggatttcgagccaatggggtaagtaaggtagtaagaaggagcgccggtttacatttaccgcacgtcggcgtgcggtcgagtcgggactcgggagacgtatgaacccccgtcccgtcccatgcatgtgtggcaggtgggtgtaccagaggtactgcgaggagatgaaggagctgtcgctgacgatcatggagctgctggagctgagcctgggcgtggagctgcgcggctactaccgggagttcttcgaggacagccggtccatcatgcggtgcaactactacccgccgtgcccggagccggagcgcacgctgggcacgggcccgcactgcgaccccacggcgctcaccatcctcctgcaggacgacgtgggcgggctggaggtgctggtggacggtgagtggcgccccgtccggcccgtcccgggcgccatggtcatcaacatcggcgacaccttcatggtaacgaaacgaaagcgctcgctcctctgttttccttggccgctcttgtcctgtgtgtatattcagttgagctctctctgtgctgttatttcccgaatcctagtggacctaaacgggcaggttattacagcacgcacacgtaggcatgtcatgtagctagtacatacatagcgatgccgatgcaaatgcaatagagacatgcgttcgagttggttcctatctcggcgggctacggcaggtacacgcggccgcggcgcgctctctctagtctatccgcggccgcgcccaggccgatcgaggcttccgggggagagttgcgacaagagaacggaccgagggggtcggctagcggtagcaagttccctgttggtttgtggcgttggagcgttgcggagaggcttgcgcggcggcggggacgtcgacggggacgtggcggggagacgatacgatgggtgccgggcagggcaacgctttcggcgggtggccgtgtccaggtgcgcgcggccttgtcggtttccccctctcggtgtccatggccgagaaatgggtcgacgaccgagaccgacgctcggtgcggcgcccatcccgtctgatccgccgcgccacgcgagcggccctatgcgatgccgcacgggcgcggagggccgtcgcgcggagtataatgtatagtatatagtacaaggttggttggagtcgggttgggttggatcgggtcaccggtacgtggtggctgctgttgcccccgccgtttccgcttgcacttttgtcgcggtttcgctggcgatccggcacgcggcgcccacaccacgccggggctccaaacagctcgggcccttggccgtgtgggtggcaggcacttgcacgcgtccggttgtcgcggcctggcccgccgccgggcgcaccgcaacaatgagacagcccgacacgatgattcttgtgcactgtgctaacccgcatgccatgcaggcgctgtcgaacgggaggtacaagagctgcctgcaccgcgcggtggtgaaccagcggcgggcgcggcggtcgctggccttcttcctgtgcccgcgcgaggaccgggtggtgcgcccgccggccagtgctgcgccgcggcgctacccggacttcacctgggccgacctcatgcgcttcacgcagcgccactaccgcgccgacacccgcacgctggacgccttcacccgctggctctcccacggcccggcccaggcggcggcgcctccctgcacctagcgagccgggccaaggccgtctctttcgccccacgtgcgcgcccagctgggcaggtggccagacacgcggcccgcgggccccgcgccgccttgccattttttgacgctggccctactgctgtgctactagtgtacatatgcaagagtacatatatatatatatatatacgtattttctatatattatatataaaagcaaggcggcccggtgcccttctcttgttttgtccacaactgtttgatcccattattctatggaccatggatacttcaatgtttgtactaagaccgtgaacgtgggattcttttccttcctctgtgttttttctgagaaaaattaaactgatttctgtgaaatttctttgttttaacaagaaaacagaaaaattacatgaggaaaacgctccatttatttcaacaagaaaaaaatacatgaaacagaaggagaaaaaacgtgttcgttctatcattttcacacgagaaaaaaaaacatagaaaacagaaaaactccccgcgttcagatgagctcaagaaaatggaacgacacggacgtcacccgcgtcttgtagcagtgggcgcacgggtgccgaatgtttgctgggcccccaagagaatcgcttctcctcacgctgaatgaatgaatcaacgagggaaacgctgcaccctgagttccagacgtttccgaattccaaacgtttttgtggcgtgcgtccatggggcgcccccaaacttcggacgtttccggcgctccaacaaatcttctcgcttcacacgtcaccgtcgtcccggattcatttgcctcgtcgctccaccattcgctgctctcctctccacgtactcttaccctgacctttgggaaagaactgaacattcgagatgcacaacagttcaaatataacatatgcagcacaagatcgttcgactgctatccgacaagccaacaacgtgcccagtagaactgaatgtacctgtgatttccagcactaacttacagcaacgttgtgaaaaaacaaaaacgaaaacaaacggcagaaaaaacagatgtattgttctacagttacaccaaatattttctggtcctttcagcaccaacaagagccatacgcatatctagaagacaaaattcctctaatttcacccctacgtggtagcagttcctcctcaacacagttcacgtgctagcgtcgagttctttgggccgccacatcgacttctcgacgcagagcaggccctcgctgcccttggtgtaggtcatccgcacctcccactgcacggacttggccatgctctccagctcatttatcgtgtccgcggtgtccctcacgatcagcttgccctgtggcctcagtacacggtcgacctcggcgaaaactgcagccagtttgcatctgtaaacaggcaacacagatttttagtatctaaaacactgcaggcaaacgccacaggttttagtcgcaagaagcaataaaagcatgcaaacaatgctacgtgtacgtatcaaaggaacatgtcaaaactcgttgcatgaacgatcattgatgtttccttgctgaactagtcacatcagtctgcttcaacttctgggtttcactagtagatataccagaagggtagaataatgtgaagagcaagaaatacagacctctttctgagctttgagaacagatggtccgcgtgcagaaggtcatacgttcttgggtaagtgctgaaagactcgcaccagtcatggtacatgccaaacaaaccgcgctcgtagatgatgggcagcgtgtctggtgaatcgatcggcacgatattcatgacccagaccttttggtccctcagagctgcagcaaaactgccatgcaacaatgtaaagcattagtcaagaagaaggtgtacagtgcatttctccttgtcaacagtcttcagtaacaaaaaaaaagtgttatgcttgactgaatctttcaaagaaatatgcttgatgacttatggtggacaagttgcctgttatagtgttatgttttaattaactatgtgccagcttgggtaactagtagttatgtagtgtgatctgaattaccaaaatataaataaataaataaacatgcccaagaaactacgaaaaccatttacttaccctccatagacagctctcatgtccatgacatttctcactttggaccagtcaattcccatgccattcacatacgatttacttacaacccgtttccagtgggcattatctgcctcaaaatcttcatttgcaggctttccatagacaccaaccttggaaccatcaatccagaaaggggtcttctcaagcctttgcggccataactctggccattttgatcctcggacttttgagccaccaggcagtttgtgcatgcatgcttccaacggtacattcctgcaaatcaaaaggctgtgtaagcaaagcagagaagcacttttctccattgaaaatatactcttctcaaagaaccgaaaccataccaagcagcatctgcatcatcagattccttgcacaatggcgggctgttttcagatcttttctcatagcaaatattgtccattggtttctgatatatgaccataccaacttggtttaacttatccttagtcttgttgaccatcttccagcacatggactttgtcaaagtagacatggctgaaaagggtatgtggccacatgttatgttagaaataaaattcaattttgaacagttggtccatagcatgtattttgaacaaatgcaatccttctccatccatgaaagaagttgacccttcatacttaggattattcagtactttcactcatgtctgctgaatttgttctcttggtagttgctatacaagaaagggggaagtacagagtagctaaacttatacaagctatagtctgatatttgtatgaaacataaattttggtatggatgtcttattaaaatgggaggttgtataatatttttctagcctacctcaacttgcttgagactaaaaggctttgttgttgttgttgaggctgtatggtgctttgactttacaaatcaagttatcagctaccctacttatggatatacacctctcataaaatgatggtaagaagtttcgatatgtcacattaacataagaacttcattcagttagggtacaacgaagttaagtagttacggaaataccattccaaatctcaacatcctctgggagcttttggtaaacaggagtggcagaccagacaaagtaaccaccagggcgtaacaagcggttcaattccagcaaaagcatgccacctaaaagtagcgagccagcaataagattcagttctatagcaaatcaataaatgaaaggaggacatgtcaatatgtaaccagcaggacaaaccttcgatgtgccaaggga

SEQ ID NO: 36 - DNA, 1788 bp, Zea mays
  source 1..1788: mol_type = genomic DNA
aatcccagcgtgcgtaatctattgcccacatgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctggtacgttgcgttgcgtgcttgtgtgcgcgcacacctgccgaccgcggccacaccgtacgcaacccacgcgtacgtacgtgcgctagctacctgcttcgctcgcttcgctcctctcgcctcgccatgcatatgcacgtacggccgtacaggtacagcagcaggtcacacgcacgaacgcacgcacgcaccagcaccgatatgatacatcatcgacgtgtcgtccccccgtctaaggccatgcatgcatgcaagcacgcctagctagcccttttggcttgctagctgacgaggggagctaggacgagcatacttactgtgcgcgtcatgctcaattgctcacactatactactacttgttactacagtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtgggtatatattattgtctgtcatgttgtcgtcgtcgtacgcgttgcggttgggtgtacatgtatataacacaaacaacaaaaaactaacgccgtgccgacgacgacgacgatcatcaggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtagccgagaccgatcgccggagactgatgctgatgatgatgcatatatacatgagagaaatcgtcgagtagactagccgattgcaaaagcaaccccagctgccgaaacctggcatatcgatcccattc

SEQ ID NO: 37 - DNA, 1698 bp, Zea mays
  source 1..1698: mol_type = genomic DNA
cgtgccgagagtccttcaaagccgacgacgagacgacgatgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctggtacgtacgagcgccatgtcacgtgcttgtgctttcatgcctcgtaccgtcgtcgtgctgtacgtgttatgtttatcggccggtacgtcacgcgtgctacactggttaacgacgtgagcgtgcccacgttgactgcatgcatgtgcatgcgcgcgcccagcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtacgtacgtcaaaaacgaatatctgaccaatgcaaacgtttttctgcaatgccagtcatccactcatcctgtacgtacctctggactctgcttgtccatctactgatgacacgtatggtaggtaccccaggtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaagccctaaggaactactagctgaatccataaactaataaagaattcgtgaataagggcgttggaagactggacacaacacaagagagttgctatatatcgtatttctgaaatttaaggcaaatatcttagttaaaaaactggtatatttaaatagacaatatatatctaaaataaagatagttcaccatttttacggtcgaacaatgataaagttatatattgtctgaatagtaacaaattaaagatttccagg

SEQ ID NO: 38 - DNA, 4095 bp, Zea mays
  source 1..4095: mol_type = genomic DNA
cggtctaagtgaccgtttgagagaggaaaagggttgaaagagacccggtctttgtgaccacctcaacggggagtaggtttataagaaccgaacctcggtaaaacgaatcaccgtgtcatccgccttatttgcttgtgatttgttttcgccctctctttcggactcgtttatatttctaacgctaaccccgacttgtagttgtgcttaaagtttgtaaatttcagattcgccctattcaccccctctaggcgactttcatataaatattgggagaaatatgaaaaacaaatgaaggtcgaacgagtcagagacaccataaaaaagaggtcgtcttaactagggtgctaaacctcaacattgtagtagatcttagtactgagtttgacatctttgacaccaacaagatggtgatacgttactttctacgttaacttgggtaggtatatcgactatagtggcctataacactaggctatgtaatatgatattgtgttgagtctttataaacatgattttttttaaaaaaaagagctaaaataaaaaatagaaatcgacggtacgatgcaagttcttctcaagacaaccaaacgcacccttgcccctttattgaaattgaagtatgtgctttatcaaatgtttaaatactaattataagtattaaatataatttaattataatactaattatatagataaagactaaataacaagacaaatttattaaatataattaattcattattaacaaatacttaatgtagcacgatcgaatcatggactaattagtcttgatagactcgtcttaccatttaatcataattagttttgtatactgtttataatatttctaactagctagtattaaacttttgatgtaacctaactaaagtttagtcacgccaatacataaggactcggatcgttcgatcacccatgacatcacgtatactaagagcatctccaaaagctctccagaagtctcccctaaatctatttttttgggaaaaacacaaaaacatgtctccaacagttcccttaaagcgcccccaactttttcatagcccttaaaactccctcatttgtagctacaaatgaggggttttttgggctccccagaaacaaactgttgatttaagggatctgttggagaaaggattaaaatttaccctcacttattatttagatgtcccttaaaactgattttgaggagtcgttttatgtagagctcttggagatgctctaacacaccgagcacaaccgcatcatcaatcaaaacaacccaaagtttgttcggtacaagtcatcagcctgtgtacacacatcagcctcggccccgggagaagcgctagcaaacaaggttcacctaaaaatccatccagattcattgaatccaaccagcacaaacgtcccatttattaatcacctcatcacaggtccccccagcctcactctcgcgccggctcaaggtacattgcgtgtcctagccaagacacgcagctcatctcagcctcacacgcacagcaagagcgaggcgtgattcgccatgggcggcctcactatggaccaggccttcgtgcaggcccccgagcaccgccccaagcccatcgtcaccgaggccaccggcatccctctcatcgacctctcgcctctggccgccagcggcggcgccgtggacgcgctggccgccgaggtgggcgcggcgagccgggactggggcttcttcgtggtcgtgggccacggcgtgcccgcagagaccgtggcgcgcgcgacggaggcgcagcgagcgttcttcgcgctgccggcagagcggaaggccgccgtgcggaggaacgaggcggagccgctcgggtactacgagtcggagcacaccaagaacgtgagggactggaaggaggtgtacgacctcgtgccgcgcgagccgccgccgccggcagccgtggccgacggcgagcttgtgttcgataacaagtggccccaggatctaccgggcttcaggtgacgaaattaactatatatccctttcgatcatagttgcgttaataaattaagggaatcgtgagcgtacgtacgtaagtttccgcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccttgcccgagccccgacctggccctcggcgtggggcggcacaaggacgccggcgccctgaccatcctgtaccaggacgacgtcggggggctcgacgtccggcggcgctccgacggcgagtgggtccgcgtcaggcccgtgcccgactcgttcatcatcaacgtcggcgacctcatccaggtacgtgcccacctgatgaactgagctgaacgtaggttgcatgcactgcatgtgtataggcttctcagatcgcttcgtgtggcgtaaggtgtggagcaacgacaggtacgagagcgcggagcaccgggtgtcggtgaactcggcgagggagaggttctccatgccctacttcttcaacccggcgacctacaccatggtggagccggtggaggagctggtgagcaaggacgatccgcccaggtacgacgcctacaactggggcgacttcttcagcaccaggaagaacagcaacttcaagaagctcaacgtggagaacattcagatcgcgcatttcaagaagagcctcgtcctcgcctaactactgctactgctaggatccatgccattgccatgtcgtcttcagattcagagcacgccatgtcgtcgctagcttcgtggtagaacaaataatgatgtgcgtgctgtgtgtaagcatggatatggatgtgaatatgtaatatgatgagcactcctactttggtatgtttgggaataacagacttgtgttggtctggttcattatttgtaagaaaatcaaaaagagttagtagggcaggaggctaaccacagtcatgctgcaccacatccctggtggaaagctggccgggttacgctacgctcgtgcagccagattactgcagggccgggatatgcttccggtggaaggaaggggacggtggctgaggaccatggggctggagcctgggagagaggtcgagctagaagaaagggggagagagaagacgcacaacgaagatgggtcagccagggatttcgacccaagggggagctagtggattttgggagaaaacagaaaagagaaaagagaaaagaagaaaaatttgttggtgtgaacacaaggttgatttgtcttttcttatttggattgatgatgagtcgtggactaaccgacccgtgagctattgtgtcgtataatcatgtctctcggtttctggtgtgcaggtttgaagcacagagacggtggtcgacgcaaaggtgaacgtcatgcaggttcgtgccgatggaccgggagcagtgaaagacgagcgttgggacttgaacaagggaccagagtcgccggatgactagccgcagtggctgacgcctggaacacgcatagacgtgaggacgtggtagagcaggtgaaaatcgcctagagggggggggggtgaatagacaaaacctaaaaattataaactttgaacacaaactttacctgaggttaccgttagaacgagtattaatgaaatcggagtgcggaaggcaagttcttcttgctacgagttgcttaatcaatattgataactttgggagtcaactcaaaatgatcacaagcaaaagaactagagagagaggagaggaagaatcaactcgcaaagtaatgatcaacacaaatgaacacaatgatttatttctcgaggtttggttccgaagaacctactccccgttcaggagtccacataggacatgtctctttcaaccctttctctctctcaaatggtcacatagactggttcagttgagagcacctagaggggggtgaataggtgatcttgtaaaatcaaacactaatagccacaaaacttagtttaaagtgttagtacggctaagtagctttgaagcgagttattgtgaacacaacaat

SEQ ID NO: 39 - DNA, 21 bp, synthetic construct
  misc_feature 1..21: note = suppression oligo
  source 1..21: mol_type = other DNA
ctccatcatgcggtgcaacta

SEQ ID NO: 40 - RNA, 21 nt, synthetic construct
  misc_feature 1..21: note = suppression oligo
  source 1..21: mol_type = other RNA
tagttgcaccgcatgatggag

SEQ ID NO: 41 - DNA, 19 bp, synthetic construct
  misc_feature 1..19: note = suppression oligo
  source 1..19: mol_type = other DNA
ggtactgcgaggagatgaa

SEQ ID NO: 42 - RNA, 21 nt, synthetic construct
  misc_feature 1..21: note = suppression oligo
  source 1..21: mol_type = other RNA
ttcatctcctcgcagtaccta

SEQ ID NO: 43 - DNA, 19 bp, synthetic construct
  misc_feature 1..19: note = suppression oligo
  source 1..19: mol_type = other DNA
caggcgccatggtcatcaa

SEQ ID NO: 44 - RNA, 21 nt, synthetic construct
  misc_feature 1..21: note = suppression oligo
  source 1..21: mol_type = other RNA
ttgatgaccatggcgcctgga

SEQ ID NO: 45 - DNA, 19 bp, synthetic construct
  misc_feature 1..19: note = suppression oligo
  source 1..19: mol_type = other DNA
tcatgcggtgcaactacta

SEQ ID NO: 46 - RNA, 21 nt, synthetic construct
  misc_feature 1..21: note = suppression oligo
  source 1..21: mol_type = other RNA
tagtagttgcaccgcatgata

SEQ ID NO: 47 - DNA, 19 bp, synthetic construct
  misc_feature 1..19: note = suppression oligo
  source 1..19: mol_type = other DNA
tcgctcgccttcttcctca

SEQ ID NO: 48 - RNA, 21 nt, synthetic construct
  misc_feature 1..21: note = suppression oligo
  source 1..21: mol_type = other RNA
tgaggaagaaggcgagcgaca

SEQ ID NO: 49 - DNA, 19 bp, synthetic construct
  misc_feature 1..19: note = suppression oligo
  source 1..19: mol_type = other DNA
tccaacgggcggtacaaga

SEQ ID NO: 50 - RNA, 21 nt, synthetic construct
  misc_feature 1..21: note = suppression oligo
  source 1..21: mol_type = other RNA
tcttgtaccgcccgttggacc

SEQ ID NO: 51 - DNA, 19 bp, synthetic construct
  misc_feature 1..19: note = suppression oligo
  source 1..19: mol_type = other DNA
gcatcaacaggtacaacta

SEQ ID NO: 52 - RNA, 21 nt, synthetic construct
  misc_feature 1..21: note = suppression oligo
  source 1..21: mol_type = other RNA
tagttgtacctgttgatgcga

SEQ ID NO: 53 - DNA, 19 bp, synthetic construct
  misc_feature 1..19: note = suppression oligo
  source 1..19: mol_type = other DNA
tggacgatggatagttcaa

SEQ ID NO: 54 - RNA, 21 nt, synthetic construct
  misc_feature 1..21: note = suppression oligo
  source 1..21: mol_type = other RNA
ttgaactatccatcgtccatc

SEQ ID NO: 55 - DNA, 19 bp, synthetic construct
  misc_feature 1..19: note = suppression oligo
  source 1..19: mol_type = other DNA
tggaccatggatacttcaa

SEQ ID NO: 56 - RNA, 21 nt, synthetic construct
  misc_feature 1..21: note = suppression oligo
  source 1..21: mol_type = other RNA
ttgaagtatccatggtccatc

SEQ ID NO: 57 - DNA, 19 bp, synthetic construct
  misc_feature 1..19: note = suppression oligo
  source 1..19: mol_type = other DNA
gcaaggtcctagatttaca

SEQ ID NO: 58 - RNA, 21 nt, synthetic construct
  misc_feature 1..21: note = suppression oligo
  source 1..21: mol_type = other RNA
tgtaaatctaggaccttgcaa

SEQ ID NO: 59 - DNA, 19 bp, synthetic construct
  misc_feature 1..19: note = suppression oligo
  source 1..19: mol_type = other DNA
cagagtacatggaggtcaa

SEQ ID NO: 60 - RNA, 21 nt, synthetic construct
  misc_feature 1..21: note = suppression oligo
  source 1..21: mol_type = other RNA
ttgacctccatgtactctgga

SEQ ID NO: 61 - DNA, 19 bp, synthetic construct
  misc_feature 1..19: note = suppression oligo
  source 1..19: mol_type = other DNA
ccatgccctacttcttcaa

SEQ ID NO: 62 - RNA, 21 nt, synthetic construct
  misc_feature 1..21: note = suppression oligo
  source 1..21: mol_type = other RNA
ttgaagaagtagggcatggaa

SEQ ID NO: 63 - DNA, 19 bp, synthetic construct
  misc_feature 1..19: note = suppression oligo
  source 1..19: mol_type = other DNA
acatggcggtcaacttcta

SEQ ID NO: 64 - RNA, 21 nt, synthetic construct
  misc_feature 1..21: note = suppression oligo
  source 1..21: mol_type = other RNA
tagaagttgaccgccatgtga

SEQ ID NO: 65 - DNA, 726 bp, Rice tungro bacilliform virus
  source 1..726: mol_type = genomic DNA
tcctacaaaagggagtagtaatatttaatgagcttgaaggaggatatcaactctctccaaggtttattggagacctttatgctcatggttttattaaacaaataaacttcacaaccaaggttcctgaagggctaccgccaatcatagcggaaaaacttcaagactataagttccctggatcaaataccgtcttaatagaacgagagattcctcgctggaacttcaatgaaatgaaaagagaaacacagatgaggaccaacttatatatcttcaagaattatcgctgtttctatggctattcaccattaaggccatacgaacctataactcctgaagaatttgggtttgattactacagttgggaaaatatggttgatgaagacgaaggagaagttgtatacatctccaagtatactaagattatcaaagtcactaaagagcatgcatgggcttggccagaacatgatggagacacaatgtcctgcaccacatcaatagaagatgaatggatccatcgtatggacaatgcttaaagaagctttatcaaaagcaactttaagtacgaatcaataaagaaggaccagaagatataaagcgggaacatcttcacatgctaccacatggctagcatctttactttagcatctctattattgtaagagtgtataatgaccagtgtgcccctggactccagtatataaggagcaccagagtagtgtaatagat

SEQ ID NO: 66 - DNA, 165 bp, Rice tungro bacilliform virus
  source 1..165: mol_type = genomic DNA
acgaatcaataaagaaggaccagaagatataaagctggaacatcttcacatgctaccacatggctagcatctttactttagcatctctattattgtaagagtgtataatgaccagtgtgcccctggactccagtatataaggagcaccagagtagtgtaatagat

SEQ ID NO: 67 - DNA, 2000 bp, Zea mays
  source 1..2000: mol_type = genomic DNA
ctgcaatatatacaccaaaagtattataaactgtcatatatatgaccaaaacctttttattttagaaaagtatattaatcatggtatattaatcaaagttgttgttggggctgcaaaaatcatacccttcttccacaagctgttccttgaactgcaggtactcaggaactctcagctcctcaacagcgagctcactgacgttgaccctcacatactcccagacaccaggcctagggcggatggcaagtgcaacccatggggggatgacaatcgcctcctgtaagataatagagctagaatgattaaagaaggtgcacactacaaaaggaacagtgctgtccagcgagatctgaatctgatgcaaacctgagctgccctcaggacatcctcaaaagcaccatccttgagcttctcgcgctcagcctcagggatcgcattgttgtactcggcaatgatctggtggggctgcagcataccctttccaaggtttttcagcctgcgcaaaacgatgtgccaaataacatcagactatgccagatctataaactcatcaaacatatacaatttcaagaaatagtttagacgtatgatcagcagtcagtagcgtgggaacatatgcaacatagcgaagaggcacaacagcaaattcattcgaaaaaatgaaaacaaagattcctctcttttaactgaacttctcgaaacccctttcatgcctacacatccgatctagtcagatgcctatgcgttcatgctgaacagaacgtgtcagaactaagcataaactggttagcaagcattatcgtattcgatagaccctttagtaacaagctatacattgggtaagttcagactccaatcattctgttcagaaacatcgtattgaatataaaactaaagaacacacatgcaggtgcagccagatctaacagcagtttacagtcggtactaaaaaaagcatggtgtatgtatgtatcatcagtatccagtactaggtttcgacaaaatcctggatgctaattaaatactcatcttattagggaacacaggaacattatgtctacagcattgaatgatggccacatcatgctagatctaacaatacataatatgatggaactggtcttaaaaagtcgcattcgctcaaataatacccgtagcaaaataaatgtaaacttgcagacgaagcgggggaaatgagggcagacctggtgaagacggcgacaagctcattggggtgggcagagagtgagtcgccaatgcgctccctgacgctgtggaggcggctcaggacacggtcacctgcaccttcccccattgctgtcctcttcctggatcctcaggcctgcacagcgaaaccgaaacggaagcggaagcttcagtcagcagagaaaactgaaaccgaaaaacggttcagatccgttgacataaaagctgcgatgacatcctaaaactaaaacccctccagcaagacataaacccaactgccaacaaccagtcttttaagtctcgacacacccttgacgctgcgccacgaaactatattgcaggcaagaaaccaacagaacctaactctggaaggggggaaagaaacggcagacaggagcaagacccaaaaaaaaacgactcagatcctggtactatagtcctagtacctagaccagaaagaagaaacaaccaatacaacaagaggcatacaagaactgaatcgatgaactgaaacgcttcagaggaccgaggaatggcggagaagggaggcgcctatttatacagatctgacgagagaaccgaacaaaaacacatcgatgggaaccatggagaagaaaagggctggccgcatggcaccaatggcctcggcctccaaaaagccgttgaatccaaagcaggcgaggacgaagcgtgacgcggcagggtacttctctagaaaagcacggcatcagcaaggtgggggggctggggttccttattgcaggcaatcacgaggtgattagcacaaacggaag

SEQ ID NO: 68 - DNA, 2000 bp, Oryza sativa
  source 1..2000: mol_type = genomic DNA
ctgaaatatacatcagagatattacaatgacatatatatcgataaaagaaaaataataaaattaagttttaaattttaagaatatatgtttttagtatcccaattatgcagatttcatacccttcttccacaagctgttccttgaactgcaagtactcggggactgtcagcaactcaacagcgagctcgctcacattgaccctcacatactcccagacaccgggcctcgggcggatggcaagggcaacccatggggagataacaatcccctcctgcatgataaaaaacaattacaagttaagttagagcaagcggtagagtaaagatggatctctgtgatgcaatgaaatctgaatctgattcaaacctgtgcactcctcaggacatcctcaaaagcaccgtccttcagcttctcacgatcagcctcagagattgcgttgttgtactcagcaatgatctggtgggcctgaagcattccctttccgaggttaaccagcctgcgcaaataacagtgtcaacaaaaatatcaggccagatctatcaactcagcctataaatatctcaataagataattttagcacttgagcatttgcgcataataagaaaatttgctattagccacttaaaaagaccatatatgatctgtttgcattgagatgaattaaaaatttcattgtagatatgaaatgattagttttgaccatttaattggacttaatgaaatatgcgcgataatcagatctacgcgctcgcgccaatagatctagtaagatgtaggttttttattttttttgtgaaactttgctaccacaacaagcatctgtaccagtgcagaattcattacttgtattcagtttgtaaaccgtatatataatataaataacatgcacatgcagtcagatctagcactaccagtccacagtaatccaaaactacatttgtatatttcatcattattcagtagtactaggtttgtacaaaatcttggctgcagaaggccgcacttaaatattcattctaatcagaaacttaaaaaaaaagtgactacaaaatgattgcatccaattcagtaaatatgagccattcctggccagatctaacaatctcaacaacaaagatcctatatgaacatctccttctaaaagaaaatacagtaacatctgaaggcagtagactagaaaccaacaaaatctaatgctgggaaatcactaaatcagcacgaacctggtgaagacggcgacgagctcattggggtgggcggagagggagtcgccgatgcgctccctgacgctgtggaggcggctcaggacgcggtcgccggcagcttcccccattgctctcctcttcctcttggctcctcaagcctgcgtgcacaaccaaccaccatcatcagatacatccagacccagtcaacacaatcactccaggaaaaaaaaaagtcaagccataaaccccaaccaaaaaccacgcctttgacaaacactggaagaaaaagaaaatcgcagctttttcacaagcaatctagaagaaaagaaaaagaaaagactacatagcagctataattgactgagaagcatacaggaatcaaacaatggagaaggggagggaggaagaacaatgatgctccaggctgaggaccgaggaactgggtgaagcggggtaggcgcgtatttatgcagatctgaggagagaaaccaccaaaacaatccgatggtttcaacgaaaaagatcgtcgcttcttgctgcaccagctcacccatagccgttgagatcgaagctaagctagcagcagcaaagctggaacgaagagtgacgcattcaagctcctctcctctcctctcctctcctccggagcacgaggccagcatgggatggattggggtttcttgttggccatggcaaaggaggaggtcattaacgttgacacggcgtaatttaattaaatcttatcttaaaatatgatttaagtggtagtaacaaggaagattaatactatgaa

SEQ ID NO: 69 - DNA, 2000 bp, Oryza sativa
  source 1..2000: mol_type = genomic DNA
gctgtttaagaaaaacagaagtaaaattcagtcactgttattttgcttcagttataatctgcaaatcgtcgttctggtacttactgtccatcaacaagctgttccttgaatgccaagtactcagaaacactcagctcttccactgccaactcacttacattcacccgaatgtagtcccagacaccaggccttggcctgatggccagtgcaacccagggcggcagcacaatggcttcctacatacagtcaaggaagtaagttagaaagactggtatttgactttgagttgactatcataaccatcctggctcattgccaaatttacctgagcagcccggagaatgtcttcaaagggagcatatttctctttgtcagcttcgatcaaggcatcgaactccgcaagcagctggtgacgctggagcattccctttccctggttaacatacctgcatagagtgatatttaagaaatagaaccaatgcttagatctcacatcctttctgcggctgaactatgttaatggcactaccacataaacctgatttttacttcttatttttaagaccacatgatctgtacttaatctagctatgaacaaacaatatttcaacatcatctaagattcatgactcaagacaaaaatgttagagctcatcacagattattatagataccatcattaaaactaaagagatgcataaccttgtcagctaagaatttgtaacatactaacatgttatcgtttcacatctgggttgactaagaactaaccaactgtatggataaaatcattgaaaactcaaaacaattagtagcaggttccaagaagacacaagatattatattgagatcttcacctagagaagagtgcaatcaactcattgggatgagacgagaaggtggcaccgaggcgttcgcggagactgtggaggcgagctagcttggcagccatgactcaatttcaggaactgcaaagaaaggttacacttagcaacacgtaccaaaaccactcacttgcacaagaataattagtcaacagccatcactaagcattgcaagactatctctgaacaggaaagccatgctaaatcaacactaataacatcacacaaaagcattggaagatcaaaacataactaaaaacagctgtttcatctacacaactgaaagcatctatggtttacgaagcagagtgcgagtactgattcaaaataaatcaacctgaaccaatatactctgacaatgttttcaaagggataaaagaaccagctttatcaaatggatttgttgggttttagtaagtatcattgagataccgatggcatatctcaaactttgcaaaattataatggcatggttccaaattaccctttagtattagcaccagttagatcctaattcctaaatccgataggacagagcgaaagatccctggagatatgaagatttggctacagattaagcagagccaacatgaagttccgaatattatgaatccgcaagcggggagatcaaagagaagaatacggaaggtcgcgactccatgaaagaatccaaccaaaaacccaaagatttttctcagttcaaaaaaaaaaaacccttcatttttggttcgccatccaccgacaggcaccaagacattcctcaggaagcaaaaaagattaagcagaacaagtgataagcaagacacagtatcaacggactacgagtcgagaaaatcactgaggcgcgattcttactgcaccaagtaaaaaaaaatttgggggcaaaaagaactctgcaatggggcggagcaacgtggcagcaaaactaaaggtcgaggatttgaggttttttgccggttttcctcgaaaccccgaatccgctcatagtaaacccactaaactgcagcagaaacccccctcttggttcagatttaccgaaagcagtaaacccaagaacatgtcagcaaaaactcctgcaagattcagctgacgacccaccaaagaatcgcaagaaat

SEQ ID NO: 70 - DNA, 2000 bp, Oryza sativa
  source 1..2000: mol_type = genomic DNA
tggcggacgcgccacgcacaaacacaaacctgcacacccctgtgtcagaggaggagaggccaagaaaggaaatcgagtggaggaagtgaggagcggcggagacgtaggaggaggagggggagatggaaatggaaagccgcgcgagagaggaggcgcgtgctggatgggaggaggaggaggaggtggtgggtttgtgtttggagagacgagcgagagaggcgaagcatttaaagggaggaagagggggagagagagagagagagagagagagagagagagagagagagaaaggaggaatataataaagggtggtgcacctgccaactgctatgctcaccaacactttgtacacacccagttacacccccctgcctttattatttccagtgcagtaataacttcaacaattattgaaatgaaaatggaattaatggagttagtatcggattagcgacacgcttgccgagcttctagacggtgcgattatttcagcgggaacgactttctgtaggtgaatttaatagaggagtgttttaaatccactcgacgttgtaatagctggtttaattcgtttgtactgtcgagtagttatccaaaatcaattttggatatttaaaagaaaaaaaaacagatccgaagtattggacctactggcaaataggaattttgctatatataggtgtgcgttcatttataatggagtagcatggagttttattaatccagtaaatgttttcattgatttaattaatataacgaatttcgcttgaggccatatttgttaaacgcttttatctctatcatcattcatcctaccagtaaagagcaccggagatcgcacttcatttaaatatatgtccatgttggataaaccatagtttattatagtgttcttttatatgttttgtggggaatttagattgtttaatatggcatacatatccatccatcattattatattctaacacaactggataagtgttctaaactattgtagaataactttgtagtatgatcgatcttgtggaataaaaaaagtctgacaataacctttcataaaggaatatgaatacccgtaatcaacgcatcaaatcattcacggtgtacgcctagcgaattcgttggcgagtgctcgtgcggccgtgggctcgctgtgatgcatgcatggctctctggctacgtcgagatagcgattagtagcaaaattaagcaagccacttattaattaatctttggagatatcatatgattaaggcattaattcgtacgtactcgtcgtcagcgttttctgcaaagtccactacagttttttctttctttgctgaaaatgctgatgtgttggagatggagtgacgtgcacaacctgccgccacgtggatggttgctggagcctacgtgtcatcttaatttgaacaaaaaaaaaagaggaataatacatcaatacattttcgaatttcagttctgccattgaccagtaatacacatgtcggcctcacattttaccctgatcttagtaacgggtggtcgcctggtcggtcactgaaaaaagttcaggaaattatagtcaaactgaaacgaacatattcactccttaaaaaaactaaatctttttatatatttgtgatattgtaaaatagctacgggataatgatatagatatatatagtgataagggatagatggatcgagatatggagttgtgctttctttaatttccactacttgggctaccatattatggtagttggtatgaaaagatacacagcagtatagtgatgtgatcaatgacatgtatatctcacatgctcccatgttggagtcaaattttgctagactaaaatccaattccaagcagtccctagccaagaacaaacaaaattcagtgaggtcactgctgcaccaaggactgcatgcatgcaggagaagggcattttctcttttttcttttggagactcgattcaattcggtcggtcggtcgcaatggtcagcttaattaaa

SEQ ID NO: 71 - DNA, 2000 bp, Oryza sativa
  source 1..2000: mol_type = genomic DNA
tgtgaaaggtggcggcaccagcttagccgcagcttctctcgtcgtctccctgaaacgagagggaggaagttggtagcgtgatatatttaggcatgtcatctcttgtataagaagtcttatctgtgctaattcacacggttctctaatctctctccattctgtttttgtaaattggttcagtagatagcgtagggttatgcttatatatactccgtgaagtatatatttaaaaattagtcacacgtaaagtactatacatgttttatcgtctaataacaataaaaacactaatcataaaatttttttaaataatacgaatggttaaacgttgaatatgaaccgtgcaaaactatatttattttgtaacagagaaaatatttcacattaattagattgttgttttatggaaggttggagagctgcgccgccgttgcgcagacctaggaggctgcttataagttataatcaatcaattcacggatgccggctgggacgcggcccatcgtccgggaagacgacaactcaacgcaaaaagccgatatgcctccaaattgccattgccacctctacggctgtttatactgctccaaatcaaaagcgtccatggaagaatctagtatttcccgcaaagacgatgatgatatgcaggattggatatatagggggttgttgcatgattgctagaactcccgtttccgaagttgttcgtccatttttaaagctgccaaataggaatttattttgttttcaagtgtaatagagttctgtccagatgagtgaattataatttggttcacattttatttgctaagtttcagtttgaacattctcaaataacttttttcttcactttttaaccgagtaacttagttattttttccgtttggaccacccaacaatttgttgctaagtgcatctcacccgtcaaataattcctttgaatccaaattcaattatatcccaaaaataaaaaacttctgaattccacatcaattcaaaccccaaccattttaatttctctccatattttccatttctctatttttacctttctcttttttccatctatttatttttttccttttctatttctttctttctccttcctttctctgtttccttcttcttctcctcggctaggcccgagccagcccgtgccgcctcgcgccaaccctgtgccgccttacgccgcgcttgcgtgcgctcgcgcccacctcgtgcccaacccgcgcacgccacacgcacacacgaggacgatcgacggacgaatgcaatcatatccccttccttactcagctagaaggctcaagaaccgcaactttgatctcttccaccctctcaaatccgccccaacccctgctgactcaatcgccattaccggaggaaaaatccccgaaaccctattaccggcgccactaacagagctccaaaattcgtcgcataattcgaaaatattctgaaattgaaggtaaaaatggaatctacatgcgaagtactccctttcccctccaatccgtcactggaacgccgccggcgccgcctcccgctgccactgccctgtttggccgccgacagccgcacggcgcgccgctgctccaggccgccctagcttcaaccaccgccacctttggctccgcctccctcctcttatgctcaccaagcccgcctccctcgccggagatcgccggaaccaccgccgccatggccgccaccgcctcctgcttctggccgccgccgccagcctcgccaccggcgcctatgccaccgccgaccacggaaacggagtccctacaccttggggaccacaaaaccggcggcatccctcccaaaaccggcctcctccaccgccggcgttcgtgggattccggccagttctgtgcagagcgagagaagaagaggaaaaatagattttcctattgaaagataaatcagaaaattcctttttcttttcctatcaagttgaccatccgtttgacctcaaaatcaaaatctgagaccta

SEQ ID NO: 72 - DNA, 786 bp, Zea mays
  source 1..786: mol_type = genomic DNA
gacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagagaccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggatt

SEQ ID NO: 73 - DNA, 1160 bp, Zea mays
  source 1..1160: mol_type = genomic DNA
atgtgctggtgccccataaggtaggcacctaggtctgtgtttgaagcatcgacagatttgtaaacatgttcctatgaacctatttctgattgataatttgtcaaaactcatcatttgtcttcatccttgcctgcttgcgttcacgtgacaaagtacgtgtatgtcttcggcctttgctgtgtatgtttcgcattgcttagatgtggtgaaagaacatcagaagatgcattgatggcgtgcttaaaccagtgatgtgctccaggtgttcctgcagtctgcagagatatttactcttgtagtcttgttgacagcacagttgtatgtgatttcttggatgtaatgtaaaccaaatgaaagataggaacagttcgtcctcttccgtatacgaaggtcactgtatcatttgtcgtggcacaagatgatctgcaggcaggactgcaacatggtttcttggactgtcctgaatgcccgttcttgttctttagttgagccagagcagcagcctggtgtcggtgcctgagacctgacgaagcacacggcaaacaaacaagtcgcagcagctagcaggggcgttgccatcgccacaagcccccaagagacccgccgaggaaaagaaaaaaaaactacggccgccgttgccaagccgagcgtgcgaaccgatccacggatgggagatcagagatcacccaccgcaggcgggcggcagtggctggcgaggtgcgtccacagaacctgctgcaggtccctgtccgtcccggcgaccccttttctaggcgagcaactccccatggcagagctgcacgcagcagggcccgtcgttggttgcagctttaaccctttttgttttaaccatacaatgcagagtcgcagaggtgaaacaggacggaaattacagaaaagatggtggtgtgccagcagccccagcatgaagaagatcaggacaaaagaaaagcttgtgattggtgacagcaacaggattggattggagccaagctaggcagtgagaggcaggcagcaagacgcgtcagccactgaaatccagagggcaacctcggcctcacaactcatatccccttgtgctgttgcgcgccgtggttagccaggtgtgctgcaggcctcctccttgtttatatatgggagatgctctcaccctctaaggt

SEQ ID NO: 74 - DNA, 1532 bp, Oryza sativa
  source 1..1532: mol_type = genomic DNA
tagtcctctaatatatgaaattttgatataggtaaagaagggtattgcaaggataagaatgtaaaaagaaataagagtaatccttaccgataatagtattccttctctaccgttaaaagttaaacctgtgcgtgtagcattttaatccaggatctatcgaatccgtccctcgttggcgtgggcgacgaacacgtgcagaagaagctttccccagaaagcacctcaccgcctcgccgtctggcagactggcacgcggggccctaccctcgctgcgcctgggcccgtccgccttctgcacactgtcacgcccccacccgctcgccgcctcgcgcctctctctccgcctccgccgcggccgcccgacgtgatagcgacacgtaggactcgccaaacacaaaaaatccatcgcgatttttggaattttgttacaaaccaaatcccgcattagagatttaatttgatttaatttaattacgtaggagtaccagataaggagatcgagttaaaaaagctaacggcgcggcgtggttatctccgaatcggctgtggctccccgcgtcggcgtcggcgcggcggcggcgcgccggccgaaccctggccgtcggatcgggcgtcgtcctgggccccacgcgccacgggcggctgtcgtttgctcctcggagcggggtgggcccaccatggccaccaccacaggtcgcggtcgcggctgacctggcggtggtcccgtgctcgcggtgttttttttttttcactctctttctctcggtggacagtagcgggggccgcggcccgcgggggcagagattgcaaaaacagcggaaacggaagattgcaaaattgcaactgctttcctgtttttaattcgggatcaaaaagattctttcgtcggggtccccgtgccattgttgtattgcgcgtaggtccttgcttgtaaaagataatctccttaattttttctttgtactactagtgtatatgcagtaagaatataccatgagtaaaatgaaccacaaaactaattacgatataccattctcatgtagacgttctcttttcttttgctagtcatacgtgcatatataaccaaacaaaaaaatgtttgaagtactcctatccaatttattactccagtagacaacaaaagaaaatgtttgaagtaataactgatccatggtacagtagggttgtcgtcaatcttgtgtttctttcattccattgtacttacaatcgtactccagctagcacagcacaatgggcttaagctttggaccccaaattctgatcttgtcggggacccgtacgaaaatactcccgtagagatgcagataccgtcacaacctacaaccaacgaatgttaagaaaacaaagggaaaaaaaaagaggcgaattcggaggagaaaaaacggtggctaaaatatagtgcgggtgtggggacgcgacgcgagcgacgaaagaggagagaggatgggttggcctgcccccccctcccctgtctataaatgcagaggcgccgagtgccctagtcgccgctc

SEQ ID NO: 75 - DNA, 841 bp, Oryza sativa
  source 1..841: mol_type = genomic DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccct

SEQ ID NO: 76 - DNA, 1392 bp, Oryza sativa
  source 1..1392: mol_type = genomic DNA
ctcgaggtcattcatatgcttgagaagagagtcgggatagtccaaaataaaacaaaggtaagattacctggtcaaaagtgaaaacatcagttaaaaggtggtataagtaaaatatcggtaataaaaggtggcccaaagtgaaatttactcttttctactattataaaaattgaggatgttttgtcggtactttgatacgtcatttttgtatgaattggtttttaagtttattcgcgattttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagcttgcccccgttgcagcgatgggtattttttctagtaaaataaaagataaacttagactcaaaacatttacaaaaacaacccctaaagtcctaaagcccaaagtgctatgcacgatccatagcaagcccagcccaacccaacccaacccaacccaccccagtgcagccaactggcaaatagtctccacaccccggcactatcaccgtgagttgtccgcaccaccgcacgtctcgcagccaaaaaaaaaaaaagaaagaaaaaaaagaaaaagaaaaaacagcaggtgggtccgggtcgtgggggccggaaaagcgaggaggatcgcgagcagcgacgaggccggccctccctccgcttccaaagaaacgccccccatcgccactatatacatacccccccctctcctcccatccccccaaccctaccaccaccaccaccaccacctcctcccccctcgctgccggacgacgagctcctcccccctccccctccgccgccgccggtaaccaccccgcgtccctctcctctttctttctccgttttttttttccgtctcgtctcgatctttggccttggtagtttgggggcgagaggcggcttcgtcgcccagatcggtgcgcgggaggggcgggatctcgcggctgggtctcggcgtgcggccggatcctcgcggggaatggggctctcggatgtagatctgatccgccgttgttgggggagatgatggggcgtttaaaatttcgccatgctaaacaagatcaggaagaggggaaaagggcactatggtttatatttttatatatttctgctgctgctcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagatggctgacgccgaggata

SEQ ID NO: 77 - DNA, 743 bp, Oryza sativa
  source 1..743: mol_type = genomic DNA
gaattcccggacctccatgcctacatcaactaatttgattccttgagtttacgtttagtgatatgtctatttttagagcttgttggggcttcggcctcagctctagccagccaaacatgttctaccaagtaccctatgttggcatgatatagtgatgcattataacaataaatgagcgagggattgctggctgaaaaagctatactagctgcatttggttatagttaaccgaactattaattgcgtgtacaacaaaataaaaaaaatgcatgttgcacattctttcattaacattatgttttggtagtgtgaattagaaatttgattgacagtagatcgacaaacatagtttcaatatgcttaagttagttatgactttaacatatcagtctccttgatattttcgttttagattcgtctctctactagtgtgtatgtccaccttccatagcagtgaagggttccattccatccctggtaaaaaaaaatcaaccactactatttatttcctaaaaagcaaaatgataaaatatcatttttttaataaaaataaaaaaattttggggtacataattgatgttgccccttgggattaaccttaaaaaagggcgaattttctagggtttggccaagttttgcaatgcaccaaattattccccttgggccggccgccaccccaaaaaaaaccccaacccccaactttccattgaaggccgggcccccttaaatcctcatccccccaa

SEQ ID NO: 78 - DNA, 144 bp, Oryza sativa
  source 1..144: mol_type = genomic DNA
taaaaaagggcgaattttctagggtttggccaagttttgcaatgcaccaaattattccccttgggccggccgccaccccaaaaaaaaccccaacccccaactttccattgaaggccgggcccccttaaatcctcatccccccaa

SEQ ID NO: 79 - DNA, 612 bp, Cauliflower mosaic virus
  source 1..612: mol_type = genomic DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 80 - DNA, 837 bp, Coix lacryma-jobi
  source 1..837: mol_type = genomic DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 81 - DNA, 947 bp, Oryza sativa
  source 1..947: mol_type = genomic DNA
aatccgaaaagtttctgcaccgttttcacgtcctaactaacaatatagggaacgtgtgctaaatataaaatgagaccttatatatgtagcgctgataactagaactatgtaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagatatttttttttaaaaaaaaatagaatgaagatattctgaacgtatcggcaaagatttaaacatataattatataattttatagtttgtgcattcgttatatcgcacgtcattaaggacatgtcttactccatctcaatttttatttagtaattaaagacaattgacttatttttattatttatcttttttcgattagatgcaaggtacttacgcacacactttgtgctcatgtgcatgtgtgagtgcacctcctcaatacacgttcaactagcgacacatctccaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagag

SEQ ID NO: 82 - DNA, 721 bp, Mirabilis mosaic virus
  source 1..721: mol_type = genomic DNA
tggagattcagaaaaatctccatcaacaaataatccaagtaaggattaatggattgatcaacatccttaccgctatgggtaagattgatgaaaagtcaaaaacaaaaatcaattatgcacaccagcatgtgttgatcaccagctattgtgggacaccaatttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaatcccacaagaatttccttatataaggaacacaaatcagaaggaagagatcaatcgaaatcaaaatcggaatcgaaatcaaaatcggaatcgaaatctctcatctctctctaccttctctctaaaaaacacttagatgtgtgagtaatcacccacttggggttgtaatatgtagtagtaaataagggaaccttagggtataccattgttgtaatattattttcagtatcaataaaataatctttcagtttatcttatattcatttgtgtgacaccgtattcccataaaaccgatcctaatctctcc

SEQ ID NO: 83 - DNA, 352 bp, Peanut chlorotic streak virus
  source 1..352: mol_type = genomic DNA
acagagggatttctctgaagatcatgtttgccagctatgcgaacaatcatcgggagatcttgagccaatcaaagaggagtgatgtagacctaaagcaataatggagccatgacgtaagggcttacgccattacgaaataattaaaggctgatgtgacctgtcggtctctcagaacctttactttttatatttggcgtgtatttttaaatttccacggcaatgacgatgtgacctgtgcatccgctttgcctataaataagttttagtttgtattgatcgacacgatcgagaagacacggccatttggacgatcatttgagagtctaaaagaacgagtcttgtaatatgtttt

SEQ ID NO: 84 - DNA, 8406 bp, Zea mays
  source 1..8406: mol_type = genomic DNA
catcatatggtctgttatattgagaaggatatatttacaagcattgaggatgcaaagaaattggattattttcaaggcatgagaacccgtaggaatctacctcatactagtggtatgtaccaattactaatattattaaatttattcaacattacctttgaagtcgtgtttgtaatatcatttccttcaaagttctggagcaagaaacactgatgttgatgttgacattatacatttgaaatgagctacatcatagcttgtatgtcatctttaacatttatgacaacgttgtttcgctgtacaatttttctttatatgctactattagagtttatggtcgcttcgacctaggtcttagccaatttctgagtccaccactaactacgagaacatttacttgtgtttcttcaattcgccaacatgttgccgcacgtcgttgttaccgtgagttttttccctaccctccatagaatattggagtgagaacaaaaaggttgttttggtttacaagaacgtgtcatgtttctactcgaaaaccatgcggtcagtgcgggtgggctaggtatgtagctgcgccgtcgcgctgtccgtggcgtgcgtgcactcggtgtcggactcggacggcgggcagccctcgggcgccggcgcagaggatcggagaagtgaacccgcagccacgctggcgctacgccccgccacaacggcggctcgccggcggcctcgggatcccccacctaatcagtaatcacgattcacgactcgccggaagcagcacccctggccctcgtgttgggcctgttgtgtccgtgtcgttcgacccacctcgagccaaccagatccttacacgtgtccgtgccgtcacatgttttacttgctcgcagttgcagtgtggtgccgactcttttgctctcgtctcgtagccttaggctgacaataagttttaaaatttacactatcaaatttaaagatcgaatcgaattaaaatcgtcggactttatttttattaattttcaaactaaaattaatttagaaccctaacaaattgtgaagaaatatttagatcctaatctactaccacgtcttcgtaccctccgttagtttctctctttcttccatccatggtggtccatatctactaactagtttacttcactgctattggcaggttgtttacctttttcttttcgaaacgctccctacattccgaattataaaataacttaatattttttaaaaataaattataacagaggctactcaggcattgcacacgcagtggccgagcgagctagaacatgtgtttggatgctgcaccgcgctgttaacagcagccctatccagtaaaatatatggcatgtcatattcagtacacactagaaaataaattccaccagatagttacaaagagtcccttgtacaaaaaaatattcagagccaatgatccattatcttctctcttattagatgtcacgaatcttctcatcttgcttcctgcgtagacgtagtcagtataagaacaagtctttttttgtttggtctctccccacttagttttaatatcgcaacatcatttctctgttcttcctttaaagtgccagcttcgtatgccacacgcgaaacacgtagatctgtttcctcttttgcgcttctgataggccgttacgccacatgagccttaagaaaccagccaatctcaacacgatagaaaatcgcacgaagtatagcactcgagcgtagtaccacatcggtagctgaagaatgacaagcgaccggacgccctataaaaaaagggttgggctccttttcaactcatatccttatcagccttttagctaagataaggtgtaacatttgttcttatcaatttaatatttgataggtgcaccatacattatattaaatttattttttacgcatatcagatatgactgtgtgcagtctgaacacacgatcattgtactagttactactttaggactctgatcaagtgattaatgccgtggaacaggctgctggagatatatgaccgtgactttgctcacgcacacatgcacacattcaccgtaccgtacgtcgatctatatatagcagagacacgcacaccacgatacgtgtcataacaactttgaaagtacgccagaagtcctaatagtggactaaacctgtgcgccacttttttcctatttttggagagaacaggtcgatggagaggaaaacctagctagaagaaaaatcacgagtcgatctactacattatacaaggttgcttattgggggcgctggttggatctgtggcgaatggcgaagcgagaagcgagaagcgacagaatgggtagtagtacgtgaagattccgtggtgggagatacgacaacaccctcgaaactggtccttaattaagcgctgatgatggacggcaataatcgtgttggctcgctttccatgtgtgcgtattaattggggtgccagtgcagtgccaccactctcaggctcccgatcgatcgatcgatccccaaaggagaggagaggagaggagcagtacgtacgaactagatttgtcacttgtgttgccgcagctcgcagcagcagcagcagcagcagctgggtggcctttcgtgctccacatcgctccaaacccaaccccccccctccctccgtctctctcaccacatttcctctcctctctttccttcctcccactgaatttccacaaagcccaccgtctctctctctctctctctctctctctctctctctctctctctctctctctctctctctctctctctctctctctctctctgttgtgtgtgtgtgtgttgtgttctctttctctcagctcagcctctcctgatgggtcatcgatccactacgctccaaaacttctcccctatatataccacgccatgtacttctctgcttcttacacttctctcatgccaccataccactagtgcaaggtcctagatttacacttggtgctacaccttgcttcgcccccttccttccttccttccttccttccctccttccttggtctctaggcagctagcagtgtggtgctgctgccggccgcctattggccgcctgggactgggatccattaattactgcgcgcgcgcggctaaccaaccaatcccagcgtgcgtaatctattgcccacatgccgacgccgtcgcacctcaacaagaacccgcgctacctggacttccgggcggcgcggcgggtgccggagtcgcacgcctggccgggcctgcacgaccaccccgtcgtggacggcggcgcgccgggccccgacgccgtgccggtggtggacctgggcgccgcggacccggcgccggcgccggcggcggcggtggcccgcgccgccgagcaatggggcgcgttcctgctcacgggccacggcgtccccgcggacctgctggcgcgcgtggaggaccggatcgccaccatgttcgcgctgccggccgacgacaagatgcgcgccgtgcgcgggcccggcgacgcctgcggctacggctccccgcccatctcctccttcttctccaagtgcatgtggtccgagggctacaccttctcgccggcctccctccgcgccgacctccgcaagctctggcccaaggccggcgacgactacaccagcttctggtacgttgcgttgcgtgcttgtgtgcgcgcacacctgccgaccgcggccacaccgtacgcaacccacgcgtacgtacgtgcgctagctacctgcttcgctcgcttcgctcctctcgcctcgccatgcatatgcacgtacggccgtacaggtacagcagcaggtcacacgcacgaacgcacgcacgcaccagcaccgatatgatacatcatcgacgtgtcgtccccccgtctaaggccatgcatgcatgcaagcacgcctagctagcccttttggcttgctagctgacgaggggagctaggacgagcatacttactgtgcgcgtcatgctcaattgctcacactatactactacttgttactacagtgatgtgatggaggagttccacaagcacatgcgcgccctcgcggacaagctgctggagctgttcctcatggcgctggggctcaccgacgagcaggccagcgccgtcgaggccgagcggaggatcgccgagacgatgaccgccaccatgcatctcaactggtgggtatatattattgtctgtcatgttgtcgtcgtcgtacgcgttgcggttgggtgtacatgtatataacacaaacaacaaaaaactaacgccgtgccgacgacgacgacgatcatcaggtacccgaggtgcccggacccgcggcgcgcgctggggctgatcgcgcacaccgactcgggcttcttcaccttcgtgatgcagagcctcgtgcccgggctgcagctcttccgccacgccccggaccggtgggtggcggtgccggccgtgccgggcgccttcgtcgtcaacgtgggcgacctcttccacatcctcaccaacggccggttccacagcgtgtaccaccgcgccgtcgtgaaccgggacctcgacaggatctcgctcggctacttcctcggcccgccgccgcacgccaaggtggcgccgctgcgcgaggccgtgccgcccggccgggcccccgcgtaccgcgccgtcacgtggcccgagtacatgggcgtccgcaagaaggccttcaccaccggcgcctccgcgctcaagatggtcgccctcgccgccgccgccgacctcgacgacgacggcgacgccgccgtcgtccatcagcagcagcagctagtcgtctcgtcgtagccgagaccgatcgccggagactgatgctgatgatgatgcatatatacatgagagaaatcgtcgagtagactagccgattgcaaaagcaaccccagctgccgaaacctggcatatcgatcccattctctgctgcgcacatgtatgcatgcatgcgcttcgtccgttcgactcgtgtgtgcttgcttgcttgcgcgtgcagcagaactaattccgttccgcagctagctgctctgctctgctctgctggaatgtaattaagtagtagtatatggtagtagagaaaagattagctaggcgatcgatatagatgacgggccggggaagaagacgaattaattaagatcgatcgacgacgacgagctgtgcgtggctggctgtgttcttctctagcctagttacagaggccggctgctgctgcttccaatcgggctgcttgtcgctactgacgatcgttagtggatccattaactaatctggaattctggattaatgcatgtggtttggcatctggctggcgtaaagatgggggaggccaggccggggtcccctagccagtagccaggctggtcaatgataggtctataacctgatcccgtactgttgtttctctctcggtctgcgctgcggagaaaataataattgatcgatccgccgcccggcctggcgtactctgaactgaacgaaacgatgggtgatgcaagagcgttatttgcaataagctgcagtgagtagcagtggagatgtgtaaaaataaatatatatatatatatatatatatatatatatatatatatatatatatatatatatcttctagttattctaggtcatggatgtgcagtgcagctacggctgttactgtagaggacacacctggctcgtaccgtcgtacacgcgctagccaaggaggaagagacacggcccggctcatgtgtcagcctgcgctactacctacgtactgcatgcgtgcttgttgcctgctctcgcttcgaacaaagctgcatcgtgcatggaatggaacaaaccacaccatgcatgctaccaccaaggcaagatatatgcaagttactgcgagaatctttacgtgtgctccagtaaaattttaaccgttggcgtggcaacatagtattaactgttacagcggtgcttattagcttgtctggaaatgaaaagaagacgccagtgatttcacttctccaaacgggacctgggggtatttacttgtagtaacgtgtacagtaattaataatgcgcgcgggcacaactcacaaggggggaagctagtaccagtaccagtacctgctgggggggtttgcatctgcatgcggccaagtgagccccttttgtgtaatggtgccggcactacttccacgaaggaaaaaaaaggatcggagccaagaactgggccgggatcctgaggagatctgcagtggagctgagacccgggggaggggaggatccgcttttaccttttgaatcctgcctggccacaccccgttctgctgctgctggcgtgatgcaaaccaaggagaagacgccatggcgtgagagacccacccaccccttagccccccaactaacattcagatcaggctgcaacttgcggaacagtgtcagactcggacagtactcggtgctgtcagtactaccaacgcagcagcgccactgcatctgtgcatcatattcttatcctcctccttcataggccatgccaaatcagatcatgtctgggcaggatagctccacgaactaaaaaaaaattgggatctaacattcacctacatctatcgcgtaaaacaacaacaccgatacaataaactgggatccatcgacatacaaacctgagctttcatatgaaccgaatgacaggtctgagatttaataaaaagagagggttcaagtcatctccaacctctagagctagcctcttccttctctctttcatgtctacaaaaaaaaatcatgaggaactccattgtcatacaggtggtagtaggggcttaagccccactcgcccccaccactacagtaaactaataaacatccgacggcctccggcggcggcctacgaggccgtcggagataagattatgtccgacggccacaaagggcgctcggaaatagcgcttatctccgacggcctcgaaggcagccgtcggagataaggttatgtccgacggctggcagcgtagccgtcggaggtaagactttttaatccgcaggaaccggcgcgcggttcatttccacgcgcgcgctcttctcttctcttctctgtctctgcccacgcgccgccgccgtcgactgcgccgccgccgtccgctgcccggcgccgtcgcccactgcgccgccgcgccgccgccgcccctcccactgccccgccgccccgcctcccaaagccccaccgccgtcgacgcagccccgcccacacgcctccccgcccagccgccgtcgccgccggccgccggccgccggtcgccgccccgctcgccccgtcgctcacgtcaacaggggtgccccgtcgctgccggctacatctccgcccgccatattatcaaagtaagtatttttagatttattttgtatatattaatattgttttatagtttctaatgttgttattaaataattagtatgttaaataatgtttatcttattatatccgatggcctaaatttgattatttggcgaaatttgattatatggcctaaatttgattacgtaattaaattaccttgttttagtttatttagtggtgctttagataatttaaattttaaatttccgagggtaattattatgccctcggaaataaggtcattttatatgatttgtcattcataacaatgttatttcgtatgtaatattgtattgtggtttattagtttaattatttaataatacacgatgattctagttaatcatagtgtatcgtagtgtgaacaaacgaaacctaggcgtcacccgtttcggcccaacacaccttacaagcgacgcatgtgaggtatgttgggccggaattgtccttttattggacagcctcggggtgaccgacagagttcgtgtttatgacaaaagcatgtgctcctgcttagtttcggcagcataacccctctgttctccaattgcaccatttttaggcgtgcgattggagaacagcggggttatgctgccgaaatttcgcacgaccacatgtcttgtcataaacacggcctcgtcggtcactcctgggtgggtttaggacctatcctttcctacagatgtaggggcgtgatttcttcgtaaaaacggatggcccgtgcattacttatctaattaagttaacgtctcgtagctagtatggaggagaatcgtcgatggatgtatgaaggttggaagaaaagaggtgctctatcaagtgagtgggtggccaagactgatgcttttctcgaccatgcttttgctcggtcagagactggaaccgatgttaggtgcccttgtagcaagtgtcggaacattaatttccttgacaggaggactatgtcgatacatatttgcaagaacggttatatgccaggctatgaggtgtgggtgcaccacggtgaggacccacctcctcgtattgtatcggaagt

SEQ ID NO: 85 - DNA, 7581 bp, Zea mays
  source 1..7581: mol_type = genomic DNA
gctcctattctgatatttacttcaattcaactcaaaagtgtattttttaattcaatatacatgaataaagaactatacttctaataatctccaatgtcgaagtaaactaaatcataactaataataattaataaaataacagatcaagtgaggaagtacaaattaaaatggtagaaagcaaatagtcattttactttaccaatattttgggattaaaaaaatattacacaaatagacattaaagtacttgcgtatatgacattatagcattgacaaggatcactagtacaaagaaactctataaagacggtttataaactattttcactgacattaagacgatagcttataaaaataatatagttaccaatatatattcaacaattaaatgtattcattaatattatattataaaaatattcaacataaaaagtataaaatattcgacatccataaataaaaaataaaagtatttaaataattcaaaacgataaatggtttgaattaaattacgaaaatactaaatatttatgtaacattagttatttgtagatatactattaaatggtatcaatgcatcattagtaataataaaattaaataattggacattccttatagagagaaaactttttaaataggttcatgatacattgcttcttttgcggatatggatagtcactacaggaaaaactggaaaaaaaacgtcagccagaataagaacgttggccccgacgttcttaaatcgcctaagaacgtcggttcctacagaccggcgtacttaagtctaagaacgttgacctttgctttgccgacgtaccttaagttaagaacgtcagccctgtctggctgacgttcttatatgaattatataacagcacgcgatccttctctttcttgtgttctctcgcgtgcataccctatagcgccgccaccagagcccgccatcgccccaccgcgtgcccgcccgccccactacgcggccgctggagcccgccaccgctagccaccatcgaagcccaccaacgccccaccacatggtcacccaagccaccgcgcgctgccaccggcgcccgccaccgcaccgctgacctcctcctcaccacgccgctgtccttcgcgccaccgcacctgctcgccgctgcacatcgagatcgcgcaggtgattagtttatgcttttattccattatgagtatgaatgcctaatctgaatttttggagcgcaagaaataatgtatgattagtttatgcttttattacattattttgcttgaaaatggtttaatgtatagaaaaaccatgtgttgtattgtgaaatagcgctgaatttaatcatatcgtgtttgagtatgattgtgtatcttggcatatgtatgttaggttaatgacaaatagtcctcgcccccgacgcttggtggtcgccccttccctcgcccgttgccagcacgctcgccggtttgctccgtctaggccgcccttcgtccaccgtcgaggccgagtccacgccggtgcctccactaccactcgcttgggggcccacctccagcaccgtccctacgcttcaaaggtttaaaatcttttgtgtttagattcatcttttgtgttgctgcaattgcaatcgctatttggatatgattgtaggtactgccatttacctcaagcacttgtgtttaaagtaaatagtgatacctaatgtttttcacacatttattttgtcattggcacatttacttacacgtaggacacacacttagaaaaacatatgcgtcacccgttcgggaacgacacatctcacatttacttgtgaggtgcatagtccgaattgtctagcttatgttggacatcctgagaatggtagcggttgtgtgttatttggtctctgactcgtgtacctcccgctcgtgatttcggtagtccggagaacaatggggttatgctgccgaaatcatgagcataaggtacacgagtcagaccaaataacacgcaacggctagcattcttatgtacgatggttccaacagtgacaccctagggcactctgatgcatgggttagggttgctgacgaaatttgtggcacgcgcatttgctggcgagcctcgtgtggcgaaatgcccatgtactaggtgtcgaaacctgatccgactggacaagtttgacctctccattcatatttgcaaatatagctttaagctagacaatctggtgtggcgtgagcatggaaaggtggatgctcctccggagtcgaatatagacgaggtcgtagaccgaatagaggacatgctagatgacatctgacatgagtatccatcactagagaccgactgacacccgtcggtggaagtgcagtagttgctcacgaaggcaccaaagtaactgtacttcatatcgtaatacggcttatggagatgaagtccaagcataacttctcaaataactgctacaacgatatcgtgacaacggatatttatattacacgtttatccgataatgaggttggataatttggatatccatttctattttccatcgctagctataagcctataataagtttaattcaatttgtcctatttcctgcttcttcacctcaagtcctcaaactacatgttgtaaagatgaggaagtcgaacactccacgcggtgcccacctcttttcatgcactcaccaactgatcatttgttcatggttttcatcaccacgcgtgcatcaaaaaaaacctagctgataagctcaagccgcatctcacagaaggttcaagaaattatactgaaatcactgccagcgcccaaacaagtgtcagccgttcttgccatttgcatgccacgcgtgctgcaggcttacgtggcagaaacgttgcacggacatgctggcacagttgtcactcgccccatgaattgcttgccttgtgttgcatgcatggtcgctcacacacatgaatgatttcatgttattcgtcttgtatgctatataactgcagtttgtgttgacctccattttgattatctctatcctgtacgtgccgagagtccttcaaagccgacgacgagacgacgatgcagtcgtcgtcgtcatcagcctcgacgccggctgccgcttccggcctcgtcttcgatctcgggtctgcggcgggcgtgccggagacacacgcgtggccgggggtgaacgagtacccgtcggtggagtccgctggccgcgacgtggtcccggtggtggacatgggggtggcctgcccggacgcgacgcgggcgttggcgcgcgccgcagacgagtggggcgtgtttctgctcgtcggccacggcgtgccccgggaagtggcggcgcgtgccgaggagcaggtcgcgcgcctgttcgtgctcccggctcctgacaaggcccgcgcggggcgccgccccggggagcccacggccaccggctacggcaggccgcccctggcactccgcttctccaagctcatgtggtccgaggggtacacgttccgcgccgccaccgtccgcgaagagttccgccgcgtctggcccgacggcggcgacgactacctccgcttctggtacgtacgagcgccatgtcacgtgcttgtgctttcatgcctcgtaccgtcgtcgtgctgtacgtgttatgtttatcggccggtacgtcacgcgtgctacactggttaacgacgtgagcgtgcccacgttgactgcatgcatgtgcatgcgcgcgcccagcgacgtgatggaggagtacgacagagagatgagggctctcggtggcaggctgctcgacctcttcttcatggcgctcggcctcaccgacgtccagttcgccaccggcgagacggagcggaggatccgcgagacctggacggcgacgatgcacccaatcctgtacgtacgtcaaaaacgaatatctgaccaatgcaaacgtttttctgcaatgccagtcatccactcatcctgtacgtacctctggactctgcttgtccatctactgatgacacgtatggtaggtaccccaggtgtccggaaccggagcgcgccatcgggctgacggcgcacacggactcgggcttcatcacgctcatcatgcagagccccgtgcccgggctgcagctgctccgccgcgggccggaccggtgggtgacggtgccggcgccgccgggcgcgctcatcgtcatgctcggcgacctgttccaggtgctcacgaacggccgcttccggagccctatccaccgcgccgtcgtaagccgagagcgcgagcggatctccgtgccctacttcctctgcccgccggaggacatgacggtggcgccgctcgcgtccgctctgctgccggggaggaaggccgtgttccgggccgtgacgtggccagagtacatggaggtcaagcacaaggtgttcggcacggatgcgccggccctggagatgctgcagctgcaggtggatgaggaagaacaaggtgaaagggccgccaccacctaagccctaaggaactactagctgaatccataaactaataaagaattcgtgaataagggcgttggaagactggacacaacacaagagagttgctatatatcgtatttctgaaatttaaggcaaatatcttagttaaaaaactggtatatttaaatagacaatatatatctaaaataaagatagttcaccatttttacggtcgaacaatgataaagttatatattgtctgaatagtaacaaattaaagatttccaggagatggatgcgattgtttcttgttcatccactcgttcataccatgttgccagagtattacacacagttatgagaagggcgctgctatctcgtatgggatatggtgaggcactcaaacatacaacagtggcttttgggtgctctagtttcttacatagttttcggtgtggttgactatgagatagaggatacaattttgaatggactcacggcgccaagtgacggtgattttgccggaaacttgataaagattttgccaagtgtcacttggtactcgcaaagaaaagtcgccgtcacggcgcaagtgacggtgacaaagactttgccgagtatcctccaacacactcgacaaagactccctctttgtcgagtgcttactatcctagtatcaaacaattcagctcggggacgagttgcctctcgaggaggggagagatgtcacgtccagacgtacacgaggcgtcgggcacaagatgtgcgacgtgcagcggaacgcggcgcgcacccaggtgcgagtgaacatgaaggcaaggaaagtggctaactaggaaatagagatagcttcctattagctcctaaattagtgggctgattggttcctattatttattagatcctaaattagtgggttgattccttcctattattagctcctagattggggggttgttccttcctttttattagttaccaaagtagtgggtctttcctattatgaggccctaaattagtgggctgcgggcatatatatccttgtaaaccacagggaggaggcaagcaataaactatctctttcactactcctattaattctctcaagctcgcggttgaggtgctgatcctcaccggagaagacggatggcagctacgagctacgtagccgtggtccggctagttaggggtttgacgctcttgacaaatgtcgaggaacccactcactactaattcagaatttacaaggtgtatttacatagaacttgctaggctagaccatctgtattttcagaatgtcatatgagaaggtctgttaatcaagtgtgtaaagtctagccagagttcagtttttagtcctcaaactccatctttctgcattgagttacaaagtgaggaggtcaattaagtgtccatcaaagaaaaagaccgtagcgtgtagagaaggaatgataaaatagatcacaaatgctatcagcttttaggatagataaatccactactgattcaaaacggactatacatccatctttaagtaattcaaaacaaatgatgtttatattattttgagagcaagttaaaaaatacaatttattacaataagcacatctattatcatcgaaaacacttaatcagtaacgaagtggattataatgaagtgccttaatcagtaacgaagtggattatagcgaaaactatagcagcaataatgccgcagatgatggcaatgacgccaatcaccgtagccactgttaagatagctgccaaaataggggctaagcatagaagaattgaggatctgacgcagcaactgttaagtacgggaactagatctgttatgatggctgagaggctttgcgggagcttggctttgatacatccgactattggagatatgtggttctggaagaatccccttgcgaattgcaatgcattgcgaagtttgggcgatgcgtgcttttggaaaagccttctaagaagtgcctgttcatggagtaacaggatgatgaagggtgtgaggagttgcggcgaaaccgcgagaagcgggagcacacatgccgcagcggcgatgtgcggatcgattggagcgaaggcaaggtacgccgccagcacgaagacagcgaccacagccaaacatgcaatccggagggagaagcccgcaatgccgaggtgcacaaggcgcgttggacgatccatggtggagaacccagcgaagatgctgcagaacgcggccagtgaagacgaagcgaatgtcagggcgtcagagactatgagtgccctgaatgcacgtcccctcgctcggctgatgagcggaggcggcagaacaacagcatcagcagatagattctgtttcgcaacgttgtagaatgtggctaccgatatgttcagcatgaaaatggcacagacagccgtgagccctgcggacttgctgatagactctgataacttgtcttgatcgagctctcctcgtgccttgtcctcacccttgagcagatcccttcgataggtaccatgatcagatccagacagtctcaagtgatactttatcaatactcgggagttctgcacaacaacaacaacaacaacaacaaaagagtttggttttttatattaacctttgatgaaaattaaggtaattaatcaatggaaagaaaagaattaaagacagaaggaaacaagtgatggtaccagtaagaggtgcagtccaggtttaatgcataagatggagaggtcgagaggtgtgtgcccctccttgttcgcgaaactgaggccgacgctgatgttgctcaccaaactgcagaagatgcgggtattcccagtcttcaccgccagatgcagagcggtattcccatcccaatcggccaagttcagtatccccgccaagctccgttggctgcatacgtggtccacgacgctctccatcttcttctccacagcgaggtggaggaaagtctggcctgaatcgttgcgtgactgcgtgcagccagggcacacctccacaagtttcctgacgatgtcaagccttccatttgccgcagcgacatgtatgggaaaccagccattggagtccttcttcagcgcagcagcagtacaagagcccccctctctcgtgtcgtcgtcgtcggcggcggcggcattcccagctggtgatgta

SEQ ID NO: 86 - DNA, 7332 bp, Zea mays
  source 1..7332: mol_type = genomic DNA
tataattatagatgaatgtaagggacatagatgaaattttacccgggctgcatcctgtaaatagatgaagagtaacaccgtactgttcaggttggattgtaatcactctcttgcatccataccttcgagcaagccgaaggtatcaatgttatattaattcttcctatattcatgtgtgttttatgaatactaatatgaatgaatcattcaatcatatttgccatttttgcgtgttcctatttatctatggaatgatgaagacacgttcttcatgaccttcagccgaagatcattatctccgagaggagataacgcttcgaaggacgaaggtccttaatacttaacaaatgtgttgcctggttcttgactcacaacatttgagaacaagtgaccaacaggttcatagtttgagttggtctttagctgagattcccaggtactcgccttcttgtatgatgtattcggccccttaggttgagaggtgtgccgcgaggacaaaacctggtgaggcgttgtcgcgtgggggtctttagaattgtaagtatgaatgctgccttatggggtaggtgggtcttcggtacgaggatgcttgctgtcgagtgcccctgtttcattgttttcggctgcgacatgtagcgtgtcctctgcgagggctgatttgcctttcttgtcgacgagggtcaccttcgtggcgtcgtccaatgaggtgttggctctggtggtcactttcttcggagccatcgcaggttttggtttacgaagcacgaacgataggcgccaaatgttggaaccaactaacacgtggataggtgggcccagacactcaggaaatggcagagctcaagtgcacaacgaagttgtaggtagggaactgtagcgtcttgttatgtgtgtccatagatgtaaagaatgatgatatttataggtaactggacgggtaggtggtcccaagtaccattatacccccggttacctactgcattcctagaatattcctcgcaacggggtcattacaatgcggttaggtactgtaacctaaacataccctaaagtatacccctcgaaggggcttacaagtgggcccgaaccgtcgtagggtgaagcctggccccacgaaaagctcgcgaggcctttgcccataagtcagacccttgacttcgtcgggacttggactccgctatcgagggaggagttgagccttcaggacacgtggcgacggttgctgcgagtcgctctcttctcgcgtggcccttcgcgcagccttcttcgacagccatggtcgaggactccgtcgctgacgcggggtgaagtcctccaccgttattgatcctggactcaaatatggttaattatagccggatagggctgatcgaggtcgtgttcgaccttcgttctaacagtgtgtagataccaaaagtaatcttaatcctcatataacgtcttgatgtaattgtctaatattaataaagcttgctttatgaaaaaaacacgctgttgcaaggagagtcgtatcaaaaagctattttatgcttccgtctacagagacaaatgcgtgtccgtgacatccattaggccccgtttgtttcctttcatttcgaggaattggaatcttactaatggaatatgctatttttttagaatatgacattccaccactttccaaagttatcatataagcctacctcaaattcatggggtgggaaatggaaattgattctatagatttacatgttatttttccgatgtacaacttatagcacattcttatacttgcgcctgtataacataaatgtagtatataactatctctctcatatgatttaggataatatacaaatatattatatatataaatatataaacttaattagttttgtctaaattataattattaaaatggaattcaattccaacgaaacaaacgggtccttatattaattcaacactatagtaattcttgattacatgtggcctaccacaacaattatggaaatctttgtcaagtccccagtgtggtcagttcagcactttgtcgacctggatgcaatgcaactagcaaaacaccatctgatgaaagtcttcaagataagaatcttgcactacttttctcccccctgcttttactggatatagagtgtcgtgcaccaattctaaggctatgcgcagcgcatcccgccagccgcctctccccttggtttaggccctgtttgggaacaaagtttttgaaaacacagtttttgaaatactacaatatactttagtcataacaatactgcagtttacaataccacaattttggaaactgaggtccagacctaagtttagaataccttaaaacaactatagtatttgcaatacttcagttttgaaaacagagattttagccaacttgctaaacacaattctgtatataatactgcagtattcttccaaaactgtgaaaaaacttcactcccaaacacccccttagtgtgtgtagagcaacattgtatactgcagcactcaccgcttatactgcagcactcaccgctataagcctataactgctacagtacaaacgtgatgcacagtgacccaccatatgcagggtaacgctagccgcttctctggcgtggttttgtatacacgtgagtatttattagatatagaaaaattaataatagataaaaagtaggtatagtaaatgatattttatcggtgtagtggggtatatagggagaatatagggggagataaaaaaataagagatgaaatgttaatgatgtgacgcgaaaaagtagtatagaggaaaaaaaattagagaaaatcactgcggatagcctaaaatgtactgaatttttgtttccttgtgctgctattgcactttgcgttttttcttcttatgaaaaaaaagagcccataaacaaaagcaaaaagttctctgtcttgctcctgccccaaggtacgaaggtaaacgacgagcacgatttggtcgcccgagctagcggcctcagccctcacatgacgaagacgaactagccgcacctgctgcgctgacttccgcagccactacaccccacgcgaagcacagcaagatttcactagcagaagccagatcgagtagaggcgggatccggcggaggcgcgaggcatggagatcgcgacggttgacctccgcggggtggtgccggggggcgccgggtgggaggccgcccgcgcggcggtgaccgcgtccatggtggcgcacggctgcgtcgtcgtggcgcacgacgcggtcggcgcggacctccggcaggcgctgttctcccgcgcgctgccggagctcttcgcgctccctctcgaggccaagcagcggacggtgtcgcccaatggggagttcaggggctacatcggccagcgccccggcatggactgggagagcctccgcgtcggaaagcccacggacgccgccagcgtcagcggcttcgctgagacgctctggccggagggaaacccagagttctggtgacccatccttccttccttggccacctttgaatctaactggctgccatctttagttcttctgttcgattgctgatctaactggccacgcacgcgcagtgacacgatcgtggcgttcgccgagaacatgatggagctggaggggacggtggagacgctggtcctggaaggcctcggcgcccgcgggcagagcatccgcgcccacttcggctcgctcgaccacgccgtccggctgtcgcactacggcgtgccgccggacacggagagcagcatgtcgatgcagccgcactacgacgacagcgtggtgaccgcgatcgtgcagcacgaggtggaagggctcgagatgcacgccggggacgggcgctgggtcgccgtccctgccgaagtcggcaccctcaccttcgtcgccggcgagcagttcagggtacgagcacggtccgatccgattgactcctctccctgaatcctgacggtgaccagccatatatattgacattgccgctgcgtacgtgcaggtcgtgacgaacgggcgcgtgccggcgtgcctccaccgcgtgaggacgcccagcaaccgcgagcggttctcggtgctgttcggccgccggcagaaggacggcgtggcggtgcgcgcgatggaggacctcgtcgacgcggagcacccgctggcgtacaacccgctgcgccatgaggagtactccaggtggcgtgtcacacccggttttagaaggcaaaccgaatgcgaaccatgtacgtgccaggatcagttattcacgtacacagcagttacataatatggacatcatcacacagtgctcaaaatagtattaaaaggggaaatagtcgattacatcatacgtctgagacgtccatatagttcttacaataaatcaaagtgcggaaaagaaacgtagataacacggccttcacaggcagccgactgggggttgccgctaacccacacctagaactcgtcgtaatcttggaactcctggaagtctccttccacagcttcatcttcgcctgagcagtggttgcaatgctgacaacctggggggggtttggtgtgtagagcaagggtgagtacacatcaacatactcagcaagtatcctgtttggctgtagtggactagctttatgtggggataagtcaagcagttgcttttagttggtcagattattatttactagtagaaagccaagttttagcattacccaagttattaacccaatgtatcctttccaaacggaaagaataccacttaccaataccataatcataaccggaaacatcaatctcataaccacctgtaccacaatgtctctgatcaagtatcactaatctctggagctcccttggccgctcataaccgcgagcacggctgatatatcagtttcataacactctgcagaggttgtgcactttacccacaagccgtgattccctctctggcccgggcttgcaagacccttattcactcccgaggtgaatggccagggattcactacgaagcctttacaaagattccccggggctgtagccacccgttaggtttcctaaatgcactgcactcctccccaaggggcgaacccaaacttggcagagcgagccgcatacaccgagccccattgacggcacgacggctaagtgaactacaccccggatcctctaattattcagctaagggcaccccattccaccctcatggttgcactgttttcccgggcggtcatccatagaacaggtccttacggagaggcactcgagaaaccgctcgagtccccttgaaaaccacaagtataatcataaagaagaacgggaaaacagcgtatcatagataatcacatcatgttcattgattagagttgagcaatagcatcagactaagtagtaataatccgacccaaataggtaaacaaggacatggataacaaaagctagtcaatccttaggtataaatgtgtaatgcgggaggtgaattaaagaatgaataggacatagataggtcaaaagacacttgcctccaccaaacgactgctgctcaggggcttctcctgcgaattcctcgggctcttcgaccggatcgttctctatgcgagcgcaaacatacatacatacatccacatatttaatacaaaagaacagtacaccatacaagataacaaataaagcgaatatgcatcaagtatgacattcgataacgcatttgttatggttagaaagaaacgggaaaggtctcgcaggggggttaaatcttatgcactaatgacacaattggtttttaacaaaataattctgttatacatatttatatatactaatggaaacctaatcacttttagttgatcaacattgcacagggtaaacaattaactacgtgaatcaatagcataacggaattttaaattaaacttcctattttcccatagcatgaacagtgattagcctacttaaaatacagtaattatgatcacagaaagtaaataaaataaaataaaaaaaaaataaaaaaaacagaggggggggcggttgaaccggccctagggcggttgaaccggcccgggggcaggcggccgagctggggcggccgagcgcggggggcggccgagctgggggggggggcggctgagccggggccaagcggctgaaccggcccgggacaggggggggggcgcgaccaggggccagggcgggcggcgggcgcggccagggggccgagcggcgcgggcgcggccagggggccgagcggcgcggggctgggggcggccgggcggccgggcggcgcggccaagggccagcggccaaggggagaaggaggggagggggaaggagagaggagggagaagggggaggggagggggctcaccggcgaggggggtggccgacggcgagggcgcgcagccggggtcggggcagcggcgacggctagggcagggaggtgggtgggggcggcggcttgggtggggagggagagagggagagaatgagggagagggagaggggatttggggaaaaagaggggggggggtgggggcggctgggcctgctttgggcccaagaggggggcgcaagggggcggctgggccggccggggtcggcccagggcgcgggagagagagagagaggggagagagaaagagagagagaaaagagaaagaaagatcttcttctcattttcgaaatccgatctttctacatgaatgcatttgcactttcaaagcaatcaaaagaaatgcaagggcggcatggtgcatcaaacaacataaagcatttagggtttttatacgtacgggaattccaaaccggatcccgctagaactttggaaaaggtcaaggtttagcgaggggaaaaagaaaaggaaaaggtaacgcccgaattttggcgagtaaagaaaagaaaaaattcaactgcaaaattcggggcgttacaaacctatcccccttaaaagaatctcgccctcgagattcagggctggctagcaaagagctccgggtacttagccatcagatcatcttcacgctcccaggttgcttcttcctcagagtggtgactccatctgactttgcacattctgacggtcttccttcgggtgactctatctgcaacctcaaggatctgagctggcttctcaacataggtcaagtcctcctggacttccagaccttccactggcaactgctcttctggcacacgcaagcacttcttcaactgagacacatgaaagacatcatgcacagcagacaaattctctggcaaactgagctgataagccacttctcctcgtctcgcaagaatctgatacggaccaatgtagcggggtgctagcttgcctttgactccgaatcttctg

SEQ ID NO: 87 - DNA, 1142 bp, Zea mays
  source 1..1142: mol_type = genomic DNA
atgacgaagacgaactagccgcacctgctgcgctgacttccgcagccactacaccccacgcgaagcacagcaagatttcactagcagaagccagatcgagtagaggcgggatccggcggaggcgcgaggcatggagatcgcgacggttgacctccgcggggtggtgccggggggcgccgggtgggaggccgcccgcgcggcggtgaccgcgtccatggtggcgcacggctgcgtcgtcgtggcgcacgacgcggtcggcgcggacctccggcaggcgctgttctcccgcgcgctgccggagctcttcgcgctccctctcgaggccaagcagcggacggtgtcgcccaatggggagttcaggggctacatcggccagcgccccggcatggactgggagagcctccgcgtcggaaagcccacggacgccgccagcgtcagcggcttcgctgagacgctctggccggagggaaacccagagttctgtgacacgatcgtggcgttcgccgagaacatgatggagctggaggggacggtggagacgctggtcctggaaggcctcggcgcccgcgggcagagcatccgcgcccacttcggctcgctcgaccacgccgtccggctgtcgcactacggcgtgccgccggacacggagagcagcatgtcgatgcagccgcactacgacgacagcgtggtgaccgcgatcgtgcagcacgaggtggaagggctcgagatgcacgccggggacgggcgctgggtcgccgtccctgccgaagtcggcaccctcaccttcgtcgccggcgagcagttcagggtcgtgacgaacgggcgcgtgccggcgtgcctccaccgcgtgaggacgcccagcaaccgcgagcggttctcggtgctgttcggccgccggcagaaggacggcgtggcggtgcgcgcgatggaggacctcgtcgacgcggagcacccgctggcgtacaacccgctgcgccatgaggagtactccaggtggcgtgtcacacccggttttagaaggcaaaccgaatgcgaaccatgtacgtgccaggatcagttattcacgtacacagcagttacataatatggacatcatcacacagtgctcaaaatagtattaaaaggggaaatagtcgattaca

SEQ ID NO: 88 - DNA, 954 bp, Zea mays
  source 1..954: mol_type = genomic DNA
atggagatcgcgacggttgacctccgcggggtggtgccggggggcgccgggtgggaggccgcccgcgcggcggtgaccgcgtccatggtggcgcacggctgcgtcgtcgtggcgcacgacgcggtcggcgcggacctccggcaggcgctgttctcccgcgcgctgccggagctcttcgcgctccctctcgaggccaagcagcggacggtgtcgcccaatggggagttcaggggctacatcggccagcgccccggcatggactgggagagcctccgcgtcggaaagcccacggacgccgccagcgtcagcggcttcgctgagacgctctggccggagggaaacccagagttctgtgacacgatcgtggcgttcgccgagaacatgatggagctggaggggacggtggagacgctggtcctggaaggcctcggcgcccgcgggcagagcatccgcgcccacttcggctcgctcgaccacgccgtccggctgtcgcactacggcgtgccgccggacacggagagcagcatgtcgatgcagccgcactacgacgacagcgtggtgaccgcgatcgtgcagcacgaggtggaagggctcgagatgcacgccggggacgggcgctgggtcgccgtccctgccgaagtcggcaccctcaccttcgtcgccggcgagcagttcagggtcgtgacgaacgggcgcgtgccggcgtgcctccaccgcgtgaggacgcccagcaaccgcgagcggttctcggtgctgttcggccgccggcagaaggacggcgtggcggtgcgcgcgatggaggacctcgtcgacgcggagcacccgctggcgtacaacccgctgcgccatgaggagtactccaggtggcgtgtcacacccggttttagaaggcaaaccgaatgcgaaccatgtacgtgccaggatcagttattcacgtacacagcagttacataa

SEQ ID NO: 89 - protein, 317 aa, Zea mays
MEIATVDLRGVVPGGAGWEAARAAVTASMVAHGCVVVAHDAVGADLRQALFSRALPELFALPLEAKQRTVSPNGEFRGYIGQRPGMDWESLRVGKPTDAASVSGFAETLWPEGNPEFCDTIVAFAENMMELEGTVETLVLEGLGARGQSIRAHFGSLDHAVRLSHYGVPPDTESSMSMQPHYDDSVVTAIVQHEVEGLEMHAGDGRWVAVPAEVGTLTFVAGEQFRVVTNGRVPACLHRVRTPSNRERFSVLFGRRQKDGVAVRAMEDLVDAEHPLAYNPLRHEEYSRWRVTPGFRRQTECEPCTCQDQLFTYTAVT

SEQ ID NO: 90 - DNA, 8667 bp, Zea mays
  source 1..8667: mol_type = genomic DNA
aataagttgagtcggggtagatctcaaggctacataaaatagttttttttctagaatggatgcatttgttttaagagaaaaatgatgcacttggaatgcatcaagcgaagggatgcaagaatgttggaaaaacacatgaacccgtatcgcgagatgcttatttagccattcttttatcacagtggatgcatatgcaacaaaaccaaaacagatggttagcgagtgacagtatatagagatctaaagattccgacacttcatcggtaaaaaaagcagcattaaccgagtgaatggaagaaaaacgaatttctcatattacacaacaggttttcttaaaaaaaaaaacgttacctcggtatttatattaagaagagactaaaatatggtcccgtcgagaaaatttctaaacactagtcttcatcactagtgagccgtcaccatctagtttgcaacggtccagtcagagtgcaggacataactcaggactcaagagcagacggagcacggtgagagaattttttttaatcaagcctaaaattcacctccggacaaattgaacctggaacgggtgctactcataaatattaaccatgagaccttttcgccgcagcaggttttctatcggcagttagattttagtgacgatgaaaatgatagaacgcaacgtgccgcatgcatccattcccattcgttttccacagtacatgtaggagtactgtgcaagtagggtccgtacattcagtctctctcaccagttggactcttctactgctacaaagacatgagctgcctggaatgggaaccggaggagcgagcgagcctgacggtctcacacacacacagtcacactcccaagccaattattataagaggcgagatgagcaactccagctcttaaccaatccactcctcctccctctccacctcctctgctttgctctgccactctgctgaggtggggggcagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcaggtacgctatccctcctcctcctgccgccccagcttgtgtgcgtcgcgaattggcggtcaatttggattggatgacaaatcacgtcggtcagccaatcgccgtggctacaaacgagatgttcaaatcgttcgccccgctcgcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgcagtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctccggcgccagcggcatcgcggagcaggcgctcgcgcagatacggatcgtgcaggcgttcgtcggcgaggagcgcgagatgcgggcctactcggcggcgctggccgtggcgcagaggatcggctaccgcagcggcttcgccaagggtctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactgtaaggcccaccacaccactctctccttctcctgctcctcggcccgcccgtcgtcattgctgctgacggtgtctgtggatcgcgtgcagggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtttcgctcatcgagaggttctacgaccccagcgcaggtatactacctagtactgttactacttttagcgcattaatctgaggatgtccagttcgcttgcttgccaatcgccattgccatcgcaacaatacattgccaactgccattgctgggtagactagtactgtagtacagtagcagttagaagaagcctccactgtacattgcattgccaaacaaaagtgaattgtgcagtaactctgtaccaccacattgacatggaaatgaagtgaatgcttggagcatgcagagctggccggcctcatgggctgctgctacctgctagctagccaaccagaaccagccatcctctttcttgcttttctttttactttctttggtcgtggctgtttgtggtcatacatacattcacgcgcagagcagaagagctagctaagctaggtgggtgtgcctgcaacgcgggacaaagaaaactatttgttgcctggcaagatgctactgttgcctagcacatgcctgccattgaccgactgctcagtgagaagtggttcagttgtgctgttgacagtatagatagatatatatatagtagccctgtatattttctttttcagacaagaaaaaaagaagaacgagatgaagtctgcaattcggttttggcagggcaaatcctgctggacgggcacgacctcaggtcgctgaagctgcgatggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggtccgtcccgtatagctagctcactagctgcactgccacttctctcgcttgctccccaccgttgctgcctgttgctctccaatccacttgtcggtgtctggaccacacgtgcctgcttgcctagctgctccacatctgctttccctgtccaaccttatgcaactcactcagtcactctatactatatcaaatacatttctagagtttaaagcttatcttagaataaatgcatctttagctacgagacaacctaacttcagttgttgttgttttttttactttctctcttctcaaaaatacattgattacgtctttacagcgatctttttattccaaacctaaaaatgcatgcactcactctaaaagcgcaaagggagcatcttttttccccatcatctgcacgcagccttttcttttcctcatgtcacgagggactgaaggtgtgtatgcagcgtcaagtcatccatccgttccactccactcactcatgcgtcgcgcactctgcgctcgtgcctgcccggggctaaagctttagtagctagcctcagatcagatactgttcgtgtttgttaggccgcggcagcggcacatgagctcatgacagccggcagcaccaccaccaacaccatggaagaggggtcggggtccatcacatagacatagatgcctgttgtagactagggacgggagggcaattgttaggcgcctgttgccatcgcatttgctgctgtgggttgccaacaagtaacatgccaggatgctttgctatcacgcacaggacaggaggagaggtcctttttctcgacacaagatctactaaactagcacttgctgatgagtgcataggatgaatggatgatgaacatctagagtgagagagagaaaatatgttaataataataaagagtagtaatagtagtagtaggattaagaatcaacctggggtacgtaggaagaggtacaatccctaggaatctagagtatgagaagtatgggaggagttgtgagtgaaacggaacaaattccgagttggtattttgtcgggaatgtcaagttgatttttgatcctagtgcaagcaagaattatcaatcactcagactcagcctgtctgtgtctgtccaccccagctcttgctactctacttactactgtgctactagtggtagggtaggtatcttccataaactgttattataaactgtcatctgagaaagagagccagtcaaacccatgctgctgcttatttttaatcactgtcaaatggcaggcaggcaggcagtctggttagttaataacatctgggaagggtttaatcaaaccaaatcaaatcatacgaaatctagaggccacatgggatggggccatatgtactgtactagcataagtagcggctagattttattagaacacggactcacactcccataactataactgactttgatcatgattccttgccaagcaatgctcgcatgcccatgcatgcatcatccctggtcaaactcaaacactctccaccgtcagggaataagacttattattttattaacaattccatttttatttattaattacggctggacgaggagtactagtttatttgatgagagacatggcagtccaagtcaaactcgtttgtctgaccatggcggtgatggccggttgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggcgacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgggccgggaagacgctggcgctggtggggccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggagggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtaggacggaatggatggatggatgggtttggttcctcgagagattgatgggtgaggaagctgaagctccggatcaaatggtggtactccatgatcgcaacaatgaggggaaaaaaagaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatccgcatcatcatctacctcccaaggcaccccagattgtattaatgtacttagttagcctgttttatatatacttataagtaccaaatagcagaattttactccttatctgcagtagcacgaaagaagaaacggcaagctacacctatctctggaagcaaaagaatctccccattctctctcttcctacgaggacaccccatccttttcagacagacagacagacggaccaaactgcaccacccaagaaccgatcgagagcagaggtagcattctctgcactgtagagtgtagaccatactacgctgctctcgagacatgatcccgggaatatctaccgaattgtttgtcagtggcatagccgcatagctctcagctgcctaatgaattgtttggtgccaaccgcgctgcaggcttgcagacgacccgggaagggtcagggattgcacaactactagcatcagtggagagcgaaaggccagaacggcaggcttgcaggaaaacagggaccgtgagtttgacctggccttggggtggggagtcagtcagtcacatgcgatgcgatgcagtggaggaacccatcttccaacgcacgcggcacgattgatttcagggttgctttcgccgctgccaatccaccgatccctggacactggtgcgttccttcgtcgcacatgacggtgcgtcgccccaaccgcacaggactgggagaagggacggattgggg

SEQ ID NO: 91 - DNA, 4548 bp, Zea mays
  source 1..4548: mol_type = genomic DNA
ggtggggggcagaggagctccccctccctcctctcccctcctcgccatgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgcagtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctccggcgccagcggcatcgcggagcaggcgctcgcgcagatacggatcgtgcaggcgttcgtcggcgaggagcgcgagatgcgggcctactcggcggcgctggccgtggcgcagaggatcggctaccgcagcggcttcgccaagggtctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtttcgctcatcgagaggttctacgaccccagcgcagggcaaatcctgctggacgggcacgacctcaggtcgctgaagctgcgatggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggcgacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgggccgggaagacgctggcgctggtggggccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggagggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtaggacggaatggatggatggatgggtttggttcctcgagagattgatgggtgaggaagctgaagctccggatcaaatggtggtactccatgatcgcaacaatgaggggaaaaaaagaaaggagaaaatacggtggttcatatgattgtacaatttgacgatctgtttgagtcggggttttaggatgatgtaaaccttcactcgccttttttttactcttgtttctcatccgcatcagtatcatctatctacatacagtgtcagagatgggaactgatccgcatcatcatctacctcccaaggcaccccagattgtattaatgtacttagttagcctgttttatatatacttataagtaccaaat

SEQ ID NO: 92 - DNA, 4140 bp, Zea mays
  source 1..4140: mol_type = genomic DNA
atgtctagcagcgacccggaggagatcagggcgcgcgtcgtcgttctcggttcgccccatgccgacggcggcgacgagtgggcccggcccgagctcgaggccttccatctgccgtctcccgcccaccagcctcctggcttcctagccgggcaaccggaagcagcagagcaacccacgctccctgctcctgctggccgcagcagcagcagcagcaacacgcctactacatctgccggtggcggcgctgctcctcctcctccttcttcgcctccccctccgccggcttctctggagaccgagcagccgcccaatgccaggccagcctccgccggcgccaatgacagcaagaagcccaccccgcccgccgccctgcgcgacctcttccgcttcgccgacggcctcgactgcgcgctcatgctcatcggcaccctcggcgcgctcgtccacggctgctcgctccccgtcttcctccgcttcttcgccgacctcgtcgactccttcggctcccacgccgacgacccggacaccatggtccgcctcgtcgtcaagtacgccttctacttcctcgtcgtcggagcggcaatctgggcatcctcgtgggcagagatctcttgctggatgtggaccggcgagcggcagtcgacgcggatgcggatccggtacctggacgcggcgctgcggcaggacgtgtccttcttcgacaccgacgtgcgggcctcggacgtgatctacgccatcaacgcggacgcagtggtggtgcaggacgccatcagcgagaagctgggcaacctcatccactacatggccaccttcgtggccggcttcgtcgtggggttcacggccgcgtggcagctggcgctggtcacgctggccgtggtgccgctcatcgccgtcatcggcgggctgagcgccgccgcgctcgccaagctctcgtcccgcagccaggacgcgctctccggcgccagcggcatcgcggagcaggcgctcgcgcagatacggatcgtgcaggcgttcgtcggcgaggagcgcgagatgcgggcctactcggcggcgctggccgtggcgcagaggatcggctaccgcagcggcttcgccaagggtctcggcctcggcggcacctacttcaccgtcttctgctgctacgggctcctgctctggtacggcggccacctcgtgcgcgcccagcacaccaacggcgggctcgccatcgccaccatgttctccgtcatgatcggcggactggccctcgggcagtcggcgccgagcatggccgcgttcgccaaggcgcgtgtggcggctgccaagatcttccgcatcatcgaccacaggccgggcatctcctcgcgcgacggcgcggagccagagtcggtgacggggcgggtggagatgcggggcgtggacttcgcgtacccgtcgcggccggacgtccccatcctgcgcggcttctcgctgagcgtgcccgccgggaagaccatcgcgctggtgggcagctccggctccgggaagagcacggtggtttcgctcatcgagaggttctacgaccccagcgcagggcaaatcctgctggacgggcacgacctcaggtcgctgaagctgcgatggctgcggcggcagatcgggctggtgagccaggagccggcgctgttcgcgacgagcatcagggagaacctgctgctggggcgggacagccagagcgcgacgctggcggagatggaggaggcggccagggtggccaacgcccactccttcatcatcaaactccccgacggctacgacacgcaggttggggagcgcggcctgcagctctccggtgggcagaagcagcgcatcgccatcgcccgcgccatgctcaagaaccccgccatcctgctgctggacgaggccaccagcgcgctggactccgagtctgagaagctcgtgcaggaggcgctggaccgcttcatgatcgggcgcaccaccctggtgatcgcgcacaggctgtccaccatccgcaaggccgacgtggtggccgtgctgcagggcggcgccgtctccgagatgggcgcgcacgacgagctgatggccaagggcgagaacggcacctacgccaagctcatccgcatgcaggagcaggcgcacgaggcggcgctcgtcaacgcccgccgcagcagcgccaggccctccagcgcccgcaactccgtcagctcgcccatcatgacgcgcaactcctcctacggccgctccccctactcccgccgcctctccgacttctccacctccgacttcaccctctccatccacgacccgcaccaccaccaccggaccatggcggacaagcagctggcgttccgcgccggcgccagctccttcctgcgcctcgccaggatgaactcgcccgagtgggcctacgcgctcgccggctccatcggctccatggtctgcggctccttcagcgccatcttcgcctacatcctcagcgccgtgctcagcgtctactacgcgccggacccgcggtacatgaagcgcgagatcgcaaaatactgctacctgctcatcggcatgtcctccgcggcgctgctgttcaacacggtgcagcacgtgttctgggacacggtgggcgagaacctgaccaagcgggtgcgcgagaagatgttcgccgccgtgctccgcaacgagatcgcctggttcgacgcggacgagaacgccagcgcgcgcgtggccgccaggctagcgctggacgcccagaacgtgcgctccgccatcggggaccgcatctccgtcatcgtccagaactcggcgctgatgctggtggcctgcaccgcggggttcgtcctccagtggcgcctcgcgctcgtgctcctcgccgtgttcccgctcgtcgtgggcgccaccgtgctgcagaagatgttcatgaagggcttctcgggggacctggaggccgcgcacgccagggcgacgcagatcgcgggcgaggccgtggccaacctgcgcaccgtggccgcgttcaacgcggagcgcaagatcacggggctgttcgaggccaacctgcgcggcccgctccggcgctgcttctggaaggggcagatcgccggcagcggctacggcgtggcgcagttcctgctgtacgcgtcctacgcgctggggctgtggtacgcggcgtggctggtgaagcacggcgtgtccgacttctcgcgcaccatccgcgtgttcatggtgctgatggtgtccgcgaacggcgccgccgagacgctgacgctggcgccggacttcatcaagggcgggcgcgcgatgcggtcggtgttcgagacgatcgaccgcaagacggaggtggagcccgacgacgtggacgcggcgccggtgccggagcggccgaggggcgaggtggagctgaagcacgtggacttctcgtacccgtcgcggccggacatccaggtgttccgcgacctgagcctccgtgcgcgggccgggaagacgctggcgctggtggggccgagcgggtgcggcaagagctcggtgctggcgctggtgcagcggttctacgagcccacgtccgggcgcgtgctcctggacggcaaggacgtgcgcaagtacaacctgcgggcgctgcggcgcgtggtggcggtggtgccgcaggagccgttcctgttcgcggcgagcatccacgagaacatcgcgtacgggcgcgagggcgcgacggaggcggaggtggtggaggcggcggcgcaggcgaacgcgcaccggttcatcgcggcgctgccggaggggtaccggacgcaggtgggcgagcgcggggtgcagctgtcgggagggcagcggcagcggatcgcgatcgcgcgcgcgctggtgaagcaggcggccatcgtgctgctggacgaggcgaccagcgcgctggacgccgagtcggagcggtgcgtgcaggaggcgctggagcgcgcggggtccgggcgcaccaccatcgtggtggcgcaccggctggccacggtgcgcggcgcgcacaccatcgcggtcatcgacgacggcaaggtggcggagcaggggtcgcactcgcacctgctcaagcaccatcccgacgggtgctacgcgcggatgctgcagctgcagcggctgacgggcgcggcggccgggcccgggccgtcgacctcgtgcaacggggccgcgtag

SEQ ID NO: 93 - protein, 1379 aa, Zea mays
MSSSDPEEIRARVVVLGSPHADGGDEWARPELEAFHLPSPAHQPPGFLAGQPEAAEQPTLPAPAGRSSSSSNTPTTSAGGGAAPPPPSSPPPPPASLETEQPPNARPASAGANDSKKPTPPAALRDLFRFADGLDCALMLIGTLGALVHGCSLPVFLRFFADLVDSFGSHADDPDTMVRLVVKYAFYFLVVGAAIWASSWAEISCWMWTGERQSTRMRIRYLDAALRQDVSFFDTDVRASDVIYAINADAVVVQDAISEKLGNLIHYMATFVAGFVVGFTAAWQLALVTLAVVPLIAVIGGLSAAALAKLSSRSQDALSGASGIAEQALAQIRIVQAFVGEEREMRAYSAALAVAQRIGYRSGFAKGLGLGGTYFTVFCCYGLLLWYGGHLVRAQHTNGGLAIATMFSVMIGGLALGQSAPSMAAFAKARVAAAKIFRIIDHRPGISSRDGAEPESVTGRVEMRGVDFAYPSRPDVPILRGFSLSVPAGKTIALVGSSGSGKSTVVSLIERFYDPSAGQILLDGHDLRSLKLRWLRRQIGLVSQEPALFATSIRENLLLGRDSQSATLAEMEEAARVANAHSFIIKLPDGYDTQVGERGLQLSGGQKQRIAIARAMLKNPAILLLDEATSALDSESEKLVQEALDRFMIGRTTLVIAHRLSTIRKADVVAVLQGGAVSEMGAHDELMAKGENGTYAKLIRMQEQAHEAALVNARRSSARPSSARNSVSSPIMTRNSSYGRSPYSRRLSDFSTSDFTLSIHDPHHHHRTMADKQLAFRAGASSFLRLARMNSPEWAYALAGSIGSMVCGSFSAIFAYILSAVLSVYYAPDPRYMKREIAKYCYLLIGMSSAALLFNTVQHVFWDTVGENLTKRVREKMFAAVLRNEIAWFDADENASARVAARLALDAQNVRSAIGDRISVIVQNSALMLVACTAGFVLQWRLALVLLAVFPLVVGATVLQKMFMKGFSGDLEAAHARATQIAGEAVANLRTVAAFNAERKITGLFEANLRGPLRRCFWKGQIAGSGYGVAQFLLYASYALGLWYAAWLVKHGVSDFSRTIRVFMVLMVSANGAAETLTLAPDFIKGGRAMRSVFETIDRKTEVEPDDVDAAPVPERPRGEVELKHVDFSYPSRPDIQVFRDLSLRARAGKTLALVGPSGCGKSSVLALVQRFYEPTSGRVLLDGKDVRKYNLRALRRVVAVVPQEPFLFAASIHENIAYGREGATEAEVVEAAAQANAHRFIAALPEGYRTQVGERGVQLSGGQRQRIAIARALVKQAAIVLLDEATSALDAESERCVQEALERAGSGRTTIVVAHRLATVRGAHTIAVIDDGKVAEQGSHSHLLKHHPDGCYARMLQLQRLTGAAAGPGPSTSCNGAA

SEQ ID NO: 94 - DNA, 3220 bp, Zea mays
  source 1..3220: mol_type = genomic DNA
tattttaaaacatggtttatatttccaaaaaaatgtattggcggtttttaactcaggcgccaacaaaaattgatttccacggcagctgccaccgtcgttatcgctgaccaacccggctggtcgcctgtctgctccatccatgcatgttacaactatgcagatgcagccgaaacaaacactggctagaaaggcagccaacgggcctactgtcattcgctccggcatcctactggtgggcccagttgcaccggccgatgaccagttcatcatttttctcgacgaatttgtgcacagatcgaatttactaaaaattcttcgcacgtggcaaaaccaggggaaaaatcgagaactagtcgggtttttttatattccctgatagaatagtccctgctattcatccatgaaaaccaaacacgtacactacgtcaccgtcatggatgggagcgagtgaactgatgattttttccccaccccgcacgcaacagcatgggtgacaacaaccactcccgctgcggttgggcgagcacatctctacgcacttgacactcacgcaaacctaacgcatactagagtaatcatcgccaccaactatcggcgacagaaacgatgggccccgcttctcttaatcacggtgcttgaattagtgcgcgcatagtagtgaaaataatagtgaaaaataagcagtgcgtgttttggtgtggtggttggtgagccgtccggcccaataaaaaccctcgcaccacctcgtccctcttcgtcgcatcgcacgccatcagctagcagctagcgcgctcctcgagcccagcagagaaaggccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccggcgaccccgccgccgcggcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaagatttttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcctttttctgtatccgcaaaacggcagccgtcgatcgccggacggatcgacggctcacatgagtgtcgagtccaattccaaccacgagggcggcaaggaaaaccatccgtgctggtctggactttttgccaaactccattcagccattcgccgactgaaggtgaatcttcagacagccagattgtttggtgtctagtgtgtgcgaagatggcgtagaaaagactgagagacagttggctcacacagacaagtgacaactgactatagtatctgcctgcctggctgatgctgatagagatggggactcttgtcctgtctgtttcttgtatgcgctgatctgattctgatcactgccactctgccaggtactatctcaaggacgcggtgctggacggcggcatcccgttcaacaaggcgtacgggatgacggcgttcgagtaccacgggacggacccgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaacccaggcggcaaggagcggtacgagcgcgagttccgcgagctcgccaagggcgccggcttctccgggttcaaggccacctacatctacgccaacgcctgggccatcgagttcatcaagtgaatacggctaccaccgtcgccgcgatgagatgcatggctgccacatgcatgcttgcttgcttggtcctcgtatcgtacgtcgccgtcgtcgtcttcttctggttgcgctgctaccttgctgctctcgccctcgcgtatgcatgtacttttgcttaattttctttcttcatatcatgcactctggctggcctagactgcccccgatctatgctggccggtacgtcttgtcgagctcttgcatgtcgtggattctaaattcttcttctgcgtcgaattgtctctgccatgtgcgagtaataacaatcaaggttatacttaccatacaattacatggcggtttaattgctctcttttaatttggtgaagaatcgaatcgattctactgaattcttctctgtcgtttaatgaaatttcctttgtcttttggattatataatataacacagacactcacaaagacactcacaacgtgcgcacactcattcctataaacgcacacgcgcaaatcct

SEQ ID NO: 95 - DNA, 2551 bp, Zea mays
  source 1..2551: mol_type = genomic DNA
tactagattaatcatcgccaccaactatcggcgacagaaacgatgggccccgcttctcttaatcacggtgcttgaattagtgcgcgcatagtagtgaaaaataatagtgaaaaataagcagtgcgtgttttggtgtggtggttggtgagccgtccggcccaataaaaacccctcgcaccacctcgtccctcttcgtcgcatcgcacgccatcagcagctagcgcgctcctcgagcccagcagagaaaggccggcctacccactctctctctctctctctccagtctccaccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccggcgaccccgccgccgcggcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaaagattttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcctttttctgtatccgcaaaacggcagccgtcgatcgccggacggatcgacggctcacatgagtgtcgagtccaattccaaccacgagggcggcaaggaaaaccatccgtgctggtctggactttttgccaaactccattcagccattcgccgactgaaggtgaatcttcagacagccagattgtttggtgtctagtgtgtgcgaagatggcgtagaaaagactgagagacagttggctcacacagacaagtgacaactgactatagtatctgcctgcctggctgatgctgatagagatggggactcttgtcctgtctgtttcttgtatgcgctgatctgattctgatcactgccactctgccaggtactatctcaaggacgcggtgctggacggcggcatcccgttcaacaaggcgtacgggatgacggcgttcgagtaccacggcacggactcgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtcttccacgtcgacatgatcatgctcgcgcacaaccccggcggcaaggagcggtacgagcgcgagttccgcgagctcgcaaagggcgccggcttctccgggttcaaggccacctacatctacgccaacgcctgggccatcgagttcatcaagtgaaccaccgtcgccgcgatgagatggcatggctgccacatgctttgcttgcttggtcctcgtatcgtacgtcgccgtcgtcgtcttcttctggttattgcgctgctacctcgctgctctcgcgtatgcatgtacttttgcttaattttctttcttcatatcatgcactctggctggcctagactgcccccgatccatggtggccggtacgtcttgtcgagctcttgcatgtcgtggattctaaattcttcttctgcgtcgaattgtctctgccatgtgcgagtaataacaatcaaggttatacttacgatacaattacatggcggtttaattgctctctattaatttggtgaatcgaatcgattctac

SEQ ID NO: 96 - DNA, 2371 bp, Zea mays
  source 1..2371: mol_type = genomic DNA
atgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccggcgaccccgccgccgcggcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaagatttttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcctttttctgtatccgcaaaacggcagccgtcgatcgccggacggatcgacggctcacatgagtgtcgagtccaattccaaccacgagggcggcaaggaaaaccatccgtgctggtctggactttttgccaaactccattcagccattcgccgactgaaggtgaatcttcagacagccagattgtttggtgtctagtgtgtgcgaagatggcgtagaaaagactgagagacagttggctcacacagacaagtgacaactgactatagtatctgcctgcctggctgatgctgatagagatggggactcttgtcctgtctgtttcttgtatgcgctgatctgattctgatcactgccactctgccaggtactatctcaaggacgcggtgctggacggcggcatcccgttcaacaaggcgtacgggatgacggcgttcgagtaccacgggacggacccgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaacccaggcggcaaggagcggtacgagcgcgagttccgcgagctcgccaagggcgccggcttctccgggttcaaggccacctacatctacgccaacgcctgggccatcgagttcatcaagtgaatacggctaccaccgtcgccgcgatgagatgcatggctgccacatgcatgcttgcttgcttggtcctcgtatcgtacgtcgccgtcgtcgtcttcttctggttgcgctgctaccttgctgctctcgccctcgcgtatgcatgtacttttgcttaattttctttcttcatatcatgcactctggctggcctagactgcccccgatctatgctggccggtacgtcttgtcgagctcttgcatgtcgtggattctaaattcttcttctgcgtcgaattgtctctgccatgtgcgagtaataacaatcaaggttatacttaccatacaattacatggcggtttaattgctctcttttaatttggtgaagaatcgaatcgattctactgaattcttctctgtcgtttaatgaaatttcctttgtcttttggattatataatataacacagacactcacaaagacactcacaacgtgcgcacactcattcctataaacgcacacg